(19) United States
(12) Patent Application Publication
(10) Pub. No.: US 2025/0098524 A1
(43) Pub. Date: Mar. 20, 2025

(54) LIGHT EMITTING ELEMENT, AMINE COMPOUND FOR THE SAME, AND DISPLAY DEVICE INCLUDING THE SAME

(71) Applicant: Samsung Display Co., Ltd., Yongin-si (KR)

(72) Inventors: Naoya SAKAMOTO, Yokohama (JP); Ichinori TAKADA, Yokohama (JP); Masatsugu UENO, Yokohama (JP); Takao SAKUMA, Yokohama (JP); XiuLan JIN, Yokohama (JP)

(21) Appl. No.: 18/814,365

(22) Filed: Aug. 23, 2024

(30) Foreign Application Priority Data
Aug. 28, 2023 (KR) 10-2023-0112580

(51) Int. Cl. H10K 85/60; C07C 211/61; C07D 209/88; C07D 307/92; C07D 333/74; C09K 11/02; H10K 50/12; H10K 50/15

(52) U.S. Cl. CPC H10K 85/633; C07C 211/61; C07D 209/88; C07D 307/92; C07D 333/74; C09K 11/02; H10K 85/636; H10K 50/12; H10K 50/15; H10K 85/626; H10K 85/6574; H10K 85/6576

(57) ABSTRACT

A light emitting element including a first electrode, a second electrode arranged on the first electrode, a light emitting layer arranged between the first electrode and the second electrode is provided. A hole transport region is arranged between the first electrode and the light emitting layer, and includes an amine compound represented by Formula 1. The light emitting element may exhibit high-efficiency and long-lifespan properties.

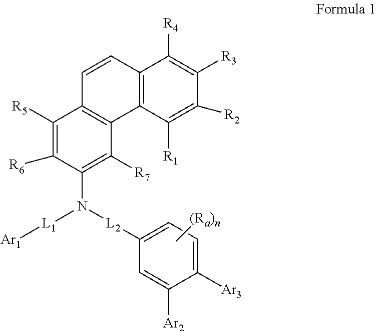

### CROSS-REFERENCE TO RELATED APPLICATION

This application claims priority to and the benefit of Korean Patent Application No. 10-2023-0112580, filed on Aug. 28, 2023, in the Korean Intellectual Property Office, the entire content of which is incorporated herein by reference.

### BACKGROUND

### 1. Field

The present disclosure herein relates to a light emitting element, an amine compound utilized for the light emitting element, and a display device including the light emitting element.

### 2. Description of the Related Art

Recently, as an image display device, an organic electroluminescence display device and/or the like have been actively developed. An organic electroluminescence display device and/or the like is a display device including a so-called “self-luminescence” light emitting element which achieves display when, in a light emitting layer, holes and electrons injected, respectively, from a first electrode and a second electrode combine in an emission layer of the display device. Subsequently, a light emitting material of the light emitting layer emits light to implement display (e.g., of an image).

The application of a light emitting element to a display device requires, or there is a desire for, light efficiency improvement, lifespan improvement, and/or the like. Therefore, the need or desire exists for the research and development of a material for a light emitting element capable of stably attaining such characteristics or desires, and such development is continuously being pursued.

In one or more embodiments, in order to implement a light emitting element having relatively high efficiency and relatively long lifespan, the development of a material for a hole transport region which has improved charge transport and material stability has been underway.

### SUMMARY

One or more aspects of embodiments of the present disclosure are directed toward a light emitting element with improved light emission efficiency and lifespan.

One or more aspects of embodiments of the present disclosure are directed toward an amine compound, which is a material for a light emitting element having high-efficiency and long-lifespan properties.

One or more aspects of embodiments of the present disclosure are directed toward a display device having excellent or suitable display quality by including a light emitting element with improved light emission efficiency and lifespan.

Additional aspects will be set forth in part in the description which follows and, in part, will be apparent from the description, or may be learned by practice of the presented embodiments of the disclosure.

One or more embodiments of the present disclosure provides an amine compound represented by Formula 1.

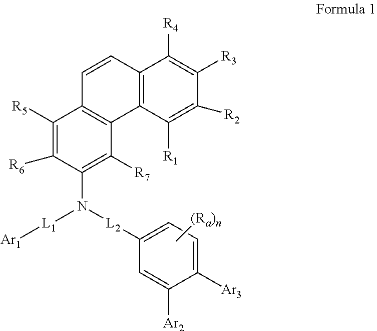

In Formula 1, R₁ to R₇ may each independently be a hydrogen atom, a deuterium atom, a halogen atom, a substituted or unsubstituted alkyl group having 1 to 15 carbons, a substituted or unsubstituted aryl group having 6 to 30 ring-forming carbon atoms, or a substituted or unsubstituted heteroaryl group having 2 to 30 ring-forming carbon atoms. n may be an integer of 1 to 3, and Rₐ may be a hydrogen atom, a deuterium atom, a halogen atom, a substituted or unsubstituted alkyl group having 1 to 15 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or forms a ring by being bonded to an adjacent group. L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. Ar₁ may be a substituted or unsubstituted aryl group having 6 to 30 ring-forming carbon atoms, or a substituted or unsubstituted heteroaryl group having 2 to 30 ring-forming carbon atoms, except that when Ar₁ is a fluorenyl group, then L₁ is a direct linkage. Ar₂ and Ar₃ may each independently be a substituted or unsubstituted phenyl group, a substituted or unsubstituted biphenyl group, a substituted or unsubstituted terphenyl group, a substituted or unsubstituted naphthyl group, or a substituted or unsubstituted phenanthrenyl group, except when Ar₂ and Ar₃ each include a carbazole group as a substituent. Ar₁, Ar₂, and Ar₃ do not each include benzophenanthrene.

In one or more embodiments, Formula 1 may be represented by Formula 1A.

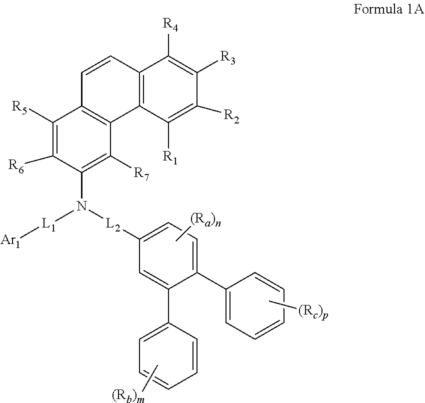

In Formula 1A, m and p may each independently be an integer of 1 to 5, R_{b} and R_{c} may each independently be a hydrogen atom, a deuterium atom, a halogen atom, a substituted or unsubstituted alkyl group having 1 to 15 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or form a ring by being bonded to an adjacent group, and Ar₁, L₁, L₂, R₁ to R₇, Rₐ, and n may each independently be as defined in Formula 1.

In one or more embodiments, Formula 1 may be represented by Formula 1-1 or Formula 1-2.

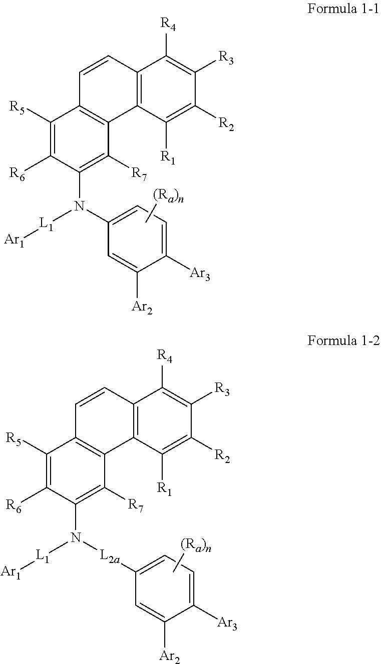

In Formula 1-2, L₂a may be a substituted or unsubstituted phenylene group, or a substituted or unsubstituted divalent biphenyl group, and in Formula 1-1 and Formula 1-2, Ar₁, Ar₂, Ar₃, L₁, R₁ to R₇, Rₐ, and n may each independently be as defined in Formula 1.

In one or more embodiments, the Ar₁ may be a substituted or unsubstituted phenyl group, a substituted or unsubstituted naphthyl group, a substituted or unsubstituted biphenyl group, a substituted or unsubstituted terphenyl group, a substituted or unsubstituted fluorenyl group, a substituted or unsubstituted dibenzoheterole group including O, S, N, or Si as a ring-forming hetero atom, or a substituted or unsubstituted benzonaphthoheterole group including O or S as a ring-forming hetero atom.

In one or more embodiments, in Formula 1, at least one hydrogen atom may be substituted with a deuterium atom.

In one or more embodiments, in Formula 1, R₁ to R₇, Rₐ, Ar₁, Ar₂, Ar₃, L₁, and L₂ may not include (e.g., may exclude) a substituted or unsubstituted amine group.

In one or more embodiments of the present disclosure, a light emitting element includes a first electrode, a second electrode arranged on the first electrode, a light emitting layer arranged between the first electrode and the second electrode, and a hole transport region arranged between the first electrode and the light emitting layer, and including the amine compound according to one or more embodiments described herein.

In one or more embodiments, the hole transport region may include at least one of a hole injection layer, a hole transport layer, an electron blocking layer, or a light emitting auxiliary layer, wherein at least one of the hole injection layer, the hole transport layer, the electron blocking layer, or the light emitting auxiliary layer may include the amine compound.

In one or more embodiments, the hole transport region may include a hole injection layer arranged on the first electrode, and a hole transport layer arranged on the hole injection layer, wherein the hole transport layer may include the amine compound.

In one or more embodiments, the light emitting layer may include a compound represented by Formula E-1.

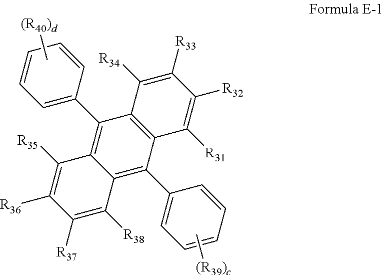

In Formula E-1, c and d may each independently be an integer of 0 to 5, and R₃₁ to R₄₀ may each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted an alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or form a ring by being bonded to an adjacent group.

In one or more embodiments, the light emitting layer may be to emit blue light.

In one or more embodiments of the present disclosure, a display device includes a base layer, a circuit layer arranged on the base layer, and a display element layer arranged on the circuit layer, and including a light emitting element, wherein the light emitting element includes a first electrode, a second electrode arranged on the first electrode, a light emitting layer arranged between the first electrode and the second electrode, and a hole transport region arranged between the first electrode and the light emitting layer, and including the amine compound according to one or more embodiments described herein.

In one or more embodiments, the light emitting element may be to emit blue light.

In one or more embodiments, the display device may further include a light control layer including a quantum dot.

### BRIEF DESCRIPTION OF THE DRAWINGS

The accompanying drawings are included to provide a further understanding of the present disclosure, and are incorporated in and constitute a part of this specification. The drawings illustrate embodiments of the present disclosure and, together with the description, serve to explain principles of the present disclosure. In the drawings:

FIG. 1 is a plan view showing a display device according to one or more embodiments of the present disclosure;

FIG. 2 is a cross-sectional view showing a portion corresponding to line I-I′ of FIG. 1;

FIG. 3 is a cross-sectional view schematically showing a light emitting element of one or more embodiments of the present disclosure;

FIG. 4 is a cross-sectional view schematically showing a light emitting element of one or more embodiments of the present disclosure;

FIG. 5 is a cross-sectional view schematically showing a light emitting element of one or more embodiments of the present disclosure;

FIG. 6 is a cross-sectional view schematically showing a light emitting element of one or more embodiments of the present disclosure;

FIG. 7 is a cross-sectional view schematically showing a light emitting element of one or more embodiments of the present disclosure;

FIG. 8 is a cross-sectional view showing a display device according to one or more embodiments of the present disclosure;

FIG. 9 is a cross-sectional view showing a display device according to one or more embodiments of the present disclosure;

FIG. 10 is a cross-sectional view showing a display device according to one or more embodiments of the present disclosure;

FIG. 11 is a cross-sectional view showing a display device according to one or more embodiments of the present disclosure; and

FIG. 12 is a view showing the interior of a vehicle in which a display device of one or more embodiments of the present disclosure is arranged.

### DETAILED DESCRIPTION

The present disclosure may be modified in one or more suitable manners and have many forms, and thus specific embodiments will be exemplified in the drawings and described in more detail in the detailed description of the present disclosure. It should be understood, however, that it is not intended to limit the present disclosure to the particular forms disclosed, but rather, is intended to cover all modifications, equivalents, and alternatives falling within the spirit and scope of the present disclosure.

When explaining each of drawings, like reference numbers are utilized for referring to like elements, and duplicative descriptions thereof may not be provided. In the accompanying drawings, the dimensions of each structure are exaggeratingly illustrated for clarity of the present disclosure.

It will be understood that, although the terms “first,” “second,” and/or the like, may be utilized herein to describe one or more suitable components, these components should not be limited by these terms. These terms are only utilized to distinguish one component from another. For example, a first component could be termed a second component, and, similarly, a second component could be termed a first component, without departing from the scope of example embodiments of the present disclosure. As utilized herein, the singular forms, “a,” “an,” and “the” are intended to include the plural forms as well, unless the context clearly indicates otherwise.

In the present application, it will be understood that the terms “include,” “includes,” “including,” “comprise,” “comprises”, “comprising,” “has,” “having,” and/or “have” and/or the like specify the presence of features, numbers, steps, operations, component, parts, or combinations thereof disclosed in the specification, but do not exclude the possibility of presence or addition of one or more other features, numbers, steps, operations, component, parts, or combinations thereof.

In the present application, when a layer, a film, a region, or a plate is referred to as being “on” or “in an upper portion of” another layer, film, region, or plate, it may be not only “directly on” the layer, film, region, or plate, but intervening layers, films, regions, or plates may also be present. On the contrary to this, when a layer, a film, a region, or a plate is referred to as being “below”, “in a lower portion of” another layer, film, region, or plate, it can be not only directly under the layer, film, region, or plate, but intervening layers, films, regions, or plates may also be present. It will be understood that when a part is referred to as being “on” another part, it can be arranged above the other part, or arranged under the other part as well.

For example, the terms, such as “lower”, “above”, “upper” and/or the like, are utilized herein for ease of description to describe one element's relation to another element(s) as illustrated in the drawings. The terms are relative concepts and are described based on the directions indicated in the drawings. It will be understood that the terms have a relative concept and are described on the basis of the orientation depicted in the drawings. It will be understood that the spatially relative terms are intended to encompass different orientations of the device in use or operation in addition to the orientation depicted in the drawings. For example, if the device in the drawings is turned over, elements described as “beneath” other elements or features would then be oriented “above” or “over” the other elements or features. Thus, the term “beneath” may encompass both an orientation of above and below. The device may be otherwise oriented (rotated 90 degrees or at other orientations), and the spatially relative descriptors used herein should be interpreted accordingly.

As utilized herein, the terms “use,” “using,” and “used” may be considered synonymous with the terms “utilize,” “utilizing,” and “utilized,” respectively.

As utilized herein, expressions such as “at least one of,” “one of,” “selected from,” and “selected from among,” when preceding a list of elements, modify the entire list of elements and do not modify the individual elements of the list. For example, the expressions “at least one of a to c,” “at least one of a, b or c,” and “at least one of a, b and/or c” may indicate only a, only b, only c, both (e.g., simultaneously) a and b, both (e.g., simultaneously) a and c, both (e.g., simultaneously) b and c, all of a, b, and c, or variations thereof.

The term “and/or” includes all combinations of one or more of the associated listed elements.

As utilized herein, the term “may” will be understood to refer to “one or more embodiments of the present disclosure,” some of which include the described element and some of which exclude that element and/or include an alternate element. Similarly, alternative language such as “or” refers to “one or more embodiments of the present disclosure,” each including a corresponding listed item.

Unless otherwise defined, all terms (including chemical, technical and scientific terms) utilized herein have the same meaning as commonly understood by one of ordinary skill in the art to which this present disclosure belongs. It will be further understood that terms, such as those defined in commonly utilized dictionaries, should be interpreted as having a meaning that is consistent with their meaning in the context of the relevant art and will not be interpreted in an idealized or overly formal sense unless expressly so defined herein.

As utilized herein, the phrase “consisting essentially of” means that any additional components will not materially affect the chemical, physical, optical, or electrical properties of the semiconductor film.

As utilized herein, the phrase “on a plane,” or “plan view,” refers to viewing a target portion from the top, and the phrase “on a cross-section” means viewing a cross-section formed by vertically cutting a target portion from the side.

In present disclosure, “not include a or any ‘component’” “exclude a or any ‘component’”, “‘component’-free”, and/or the like refers to that the “component” not being added, selected or utilized as a component in the composition, but the “component” of less than a suitable amount may still be included due to other impurities and/or external factor.

### Definitions

In the specification, the term “substituted or unsubstituted” may refer to substituted or unsubstituted with at least one substituent of (e.g., selected from among) the group including (e.g., consisting of) a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In one or more embodiments, each of the substituents exemplified herein may be substituted or unsubstituted. For example, a biphenyl group may be interpreted as an aryl group or a phenyl group substituted with a phenyl group.

In the specification, the phrase “bonded to an adjacent group to form a ring” may refer to that a group is bonded to an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocyclic or polycyclic. In one or more embodiments, the rings formed by being bonded to each other may be connected to another ring to form a spiro structure.

In the specification, the term “adjacent group” may refer to a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other and two ethyl groups in 1,1-diethylcyclopentane may be interpreted as “adjacent groups” to each other. In one or more embodiments, two methyl groups in 4,5-dimethylphenanthrene may be interpreted as “adjacent groups” to each other.

In the specification, examples of the halogen atom may include a fluorine atom, a chlorine atom, a bromine atom, or an iodine atom.

In the specification, the alkyl group may be linear or branched. The number of carbons in the alkyl group is 1 to 50, 1 to 30, 1 to 20, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, and/or the like, but the embodiment of the present disclosure is not limited thereto.

In the specification, a cycloalkyl group may refer to a cyclic alkyl group. The number of carbons in the cycloalkyl group is 3 to 50, 3 to 30, 3 to 20, or 3 to 10. Examples of the cycloalkyl group may include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, and/or the like, but the embodiment of the present disclosure is not limited thereto.

In the specification, an alkenyl group refers to a hydrocarbon group including at least one carbon double bond in the middle or terminal of an alkyl group having 2 or more carbon atoms. The alkenyl group may be linear or branched. The number of carbon atoms in the alkenyl group is not specifically limited, but is 2 to 30, 2 to 20, or 2 to 10. Examples of the alkenyl group include a vinyl group, a 1-butenyl group, a 1-pentenyl group, a 1,3-butadienyl aryl group, a styrenyl group, a styryl vinyl group, and/or the like, but the embodiment of the present disclosure is not limited thereto.

In the specification, an alkynyl group refers to a hydrocarbon group including at least one carbon triple bond in the middle or terminal of an alkyl group having 2 or more carbon atoms. The alkynyl group may be linear or branched. Although the number of carbon atoms is not specifically limited, it is 2 to 30, 2 to 20, or 2 to 10. Specific examples of the alkynyl group may include an ethynyl group, a propynyl group, and/or the like, but are not limited thereto.

In the specification, the hydrocarbon ring group refers to any functional group or substituent derived from an aliphatic hydrocarbon ring. The hydrocarbon ring group may be a saturated hydrocarbon ring group having 5 to 20 ring-forming carbon atoms.

In the specification, an aryl group refers to any functional group or substituent derived from an aromatic hydrocarbon ring. The aryl group may be a monocyclic aryl group or a polycyclic aryl group. The number of ring-forming carbon atoms in the aryl group may be 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, and/or the like, but the embodiment of the present disclosure is not limited thereto.

In the specification, the fluorenyl group may be substituted, and two substituents may be bonded to each other to form a spiro structure. Examples of the substituted fluorenyl group are as follows. However, the embodiment of the present disclosure is not limited thereto.

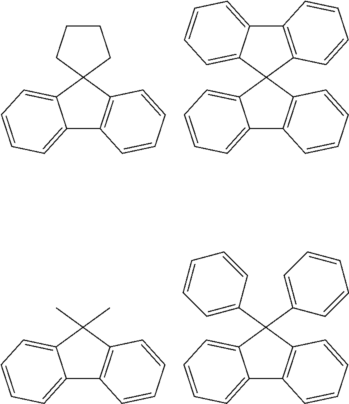

The heterocyclic group herein refers to any functional group or substituent derived from a ring containing at least one of B, O, N, P, Si, or Se as a heteroatom. The heterocyclic group includes an aliphatic heterocyclic group and an aromatic heterocyclic group. The aromatic heterocyclic group may be a heteroaryl group. The aliphatic heterocycle and the aromatic heterocycle may be monocyclic or polycyclic.

In the specification, the heterocyclic group may contain at least one of B, O, N, P, Si or S as a heteroatom. If the heterocyclic group contains two or more heteroatoms, the two or more heteroatoms may be the same as or different from each other. The heterocyclic group may be a monocyclic heterocyclic group or a polycyclic heterocyclic group, and includes a heteroaryl group. The number of ring-forming carbon atoms in the heterocyclic group may be 2 to 30, 2 to 20, or 2 to 10.

In the specification, the aliphatic heterocyclic group may include at least one of B, O, N, P, Si, or S as a heteroatom. The number of ring-forming carbon atoms in the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, and/or the like, but the embodiment of the present disclosure is not limited thereto.

In the specification, the heteroaryl group may contain at least one of B, O, N, P, Si, or S as a heteroatom. If the heteroaryl group contains two or more heteroatoms, the two or more heteroatoms may be the same as or different from each other. The heteroaryl group may be a monocyclic heterocyclic group or a polycyclic heterocyclic group. The number of ring-forming carbon atoms in the heteroaryl group may be 2 to 30, 2 to 20, or 2 to 10. Examples of the heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, and/or the like, but the embodiment of the present disclosure is not limited thereto.

In the specification, the preceding description of the aryl group may be applied to an arylene group except that the arylene group is a divalent group. The preceding description of the heteroaryl group may be applied to a heteroarylene group except that the heteroarylene group is a divalent group.

In the specification, the silyl group includes an alkylsilyl group and an arylsilyl group. Examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and/or the like, but the embodiment of the present disclosure is not limited thereto.

In the specification, the number of ring-forming carbon atoms in the carbonyl group is not specifically limited, but may be 1 to 40, 1 to 30, or 1 to 20. For example, the carbonyl group may have the following structures, but the embodiment of the present disclosure is not limited thereto.

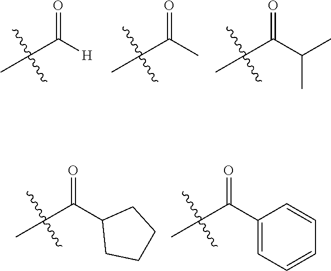

In the specification, the number of carbon atoms in the sulfinyl group and the sulfonyl group is not particularly limited, but may be 1 to 30. The sulfinyl group may include an alkyl sulfinyl group and an aryl sulfinyl group. The sulfonyl group may include an alkyl sulfonyl group and an aryl sulfonyl group.

In the specification, the thio group may include an alkylthio group and an arylthio group. The thio group may refer to that a sulfur atom is bonded to the alkyl group or the aryl group as defined herein. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but the embodiment of the present disclosure is not limited thereto.

In the specification, an oxy group may refer to that an oxygen atom is bonded to the alkyl group or the aryl group as defined herein. The oxy group may include an alkoxy group and an aryl oxy group. The alkoxy group may be a linear chain, a branched chain or a ring chain. The number of carbon atoms in the alkoxy group is not specifically limited, but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, and/or the like, but the embodiment of the present disclosure is not limited thereto.

The boron group herein may refer to that a boron atom is bonded to the alkyl group or the aryl group as defined herein. The boron group includes an alkyl boron group and an aryl boron group. Examples of the boron group may include a dimethylboron group, a trimethylboron group, a t-butyldimethylboron group, a diphenylboron group, a phenylboron group, and/or the like, but the embodiment of the present disclosure is not limited thereto.

In the specification, the number of carbon atoms in an amine group is not specifically limited, but may be 1 to 30. The amine group may include an alkyl amine group and an aryl amine group. Examples of the amine group may include a methylamine group, a dimethylamine group, a phenylamine group, a diphenylamine group, a naphthylamine group, a 9-methyl-anthracenylamine group, and/or the like, but the embodiment of the present disclosure is not limited thereto.

In the specification, the alkyl group among an alkylthio group, an alkylsulfoxy group, an alkylaryl group, an alkylamino group, an alkyl boron group, an alkyl silyl group, and an alkyl amine group is the same as the examples of the alkyl group described herein.

In the specification, the aryl group among an aryloxy group, an arylthio group, an arylsulfoxy group, an arylamino group, an arylboron group, an arylsilyl group, an arylamine group is the same as the examples of the aryl group described herein.

In the specification, a direct linkage may refer to a single bond.

In one or more embodiments, in the specification,

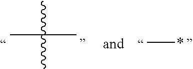

refer to a position to be connected.

Hereinafter, embodiments of the present disclosure will be described with reference to the accompanying drawings.

### Display Device

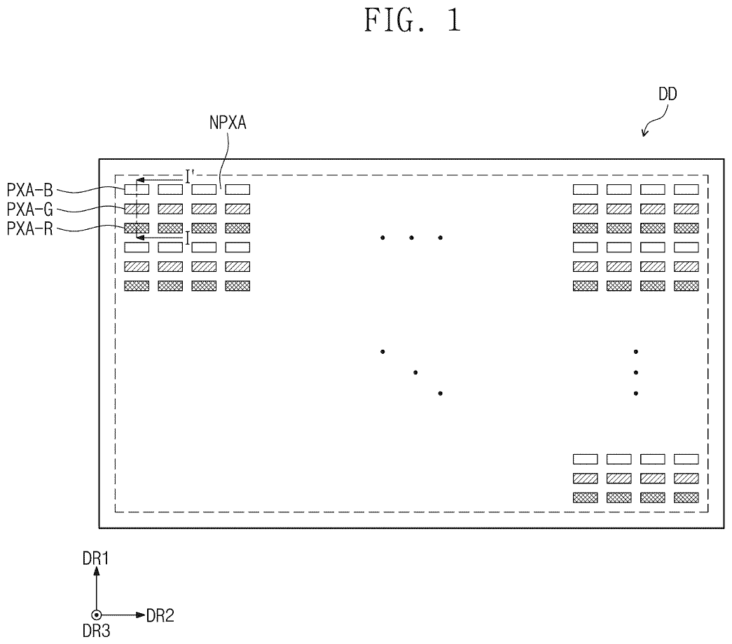

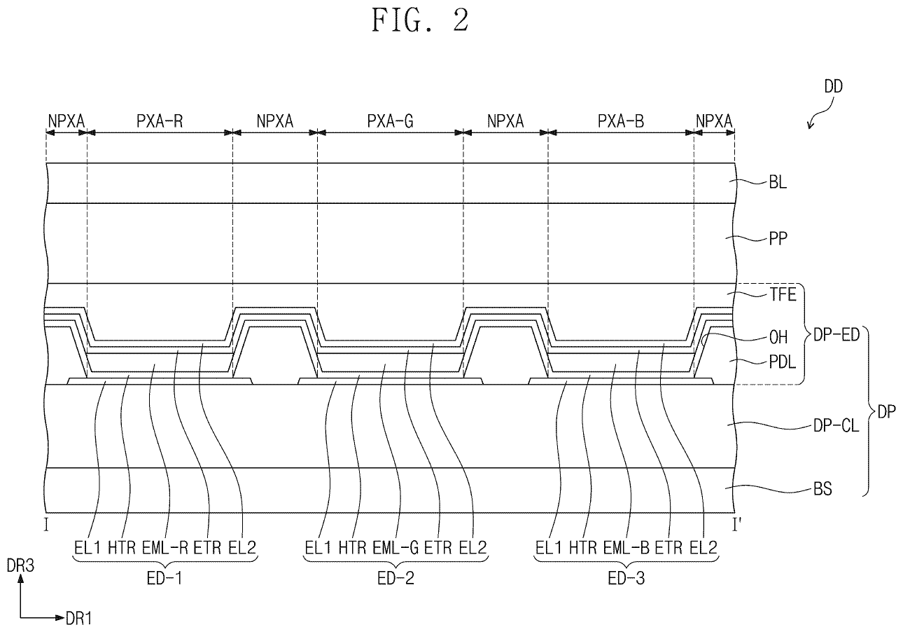

FIG. 1 is a plan view illustrating one or more embodiments of a display device DD. FIG. 2 is a cross-sectional view of the display device DD of the embodiment. FIG. 2 is a cross-sectional view illustrating a part taken along the line I-I′ of FIG. 1.

The display device DD may include a display panel DP and an optical layer PP arranged on the display panel DP. The display panel DP includes light emitting elements ED-1, ED-2, and ED-3. The display device DD may include a plurality of light emitting elements ED-1, ED-2, and ED-3. The optical layer PP may be arranged on the display panel DP to control reflected light in the display panel DP due to external light.

The optical layer PP may include, for example, a polarization layer or a color filter layer. In one or more embodiments, unlike the configuration illustrated in the drawing, the optical layer PP may not be provided from the display device DD of one or more embodiments.

A base substrate BL may be arranged on the optical layer PP. The base substrate BL may be a member which provides a base surface on which the optical layer PP arranged. The base substrate BL may be a glass substrate, a metal substrate, a plastic substrate, and/or the like. However, the embodiment of the present disclosure is not limited thereto, and the base substrate BL may be an inorganic layer, an organic layer, or a composite material layer. In one or more embodiments, unlike the configuration illustrated, in one or more embodiments, the base substrate BL may not be provided.

The display device DD according to one or more embodiments may further include a filling layer. The filling layer may be arranged between a display element layer DP-ED and the base substrate BL. The filling layer may be an organic material layer. The filling layer may include at least one of an acrylic-based resin, a silicone-based resin, or an epoxy-based resin.

The display panel DP may include a base layer BS, a circuit layer DP-CL provided on the base layer BS, and the display element layer DP-ED. The display element layer DP-ED may include a pixel defining layer PDL, the light emitting elements ED-1, ED-2, and ED-3 arranged between portions of the pixel defining layer PDL, and an encapsulation layer TFE arranged on the light emitting elements ED-1, ED-2, and ED-3.

The base layer BS may be a member which provides a base surface on which the display element layer DP-ED is arranged. The base layer BS may be a glass substrate, a metal substrate, a plastic substrate, and/or the like. However, the embodiment is not limited thereto, and the base layer BS may be an inorganic layer, an organic layer, or a composite material layer.

In one or more embodiments, the circuit layer DP-CL is arranged on the base layer BS, and the circuit layer DP-CL may include a plurality of transistors. Each of the transistors may include a control electrode, an input electrode, and an output electrode. For example, the circuit layer DP-CL may include a switching transistor and a driving transistor for driving the light emitting elements ED-1, ED-2, and ED-3 of the display element layer DP-ED.

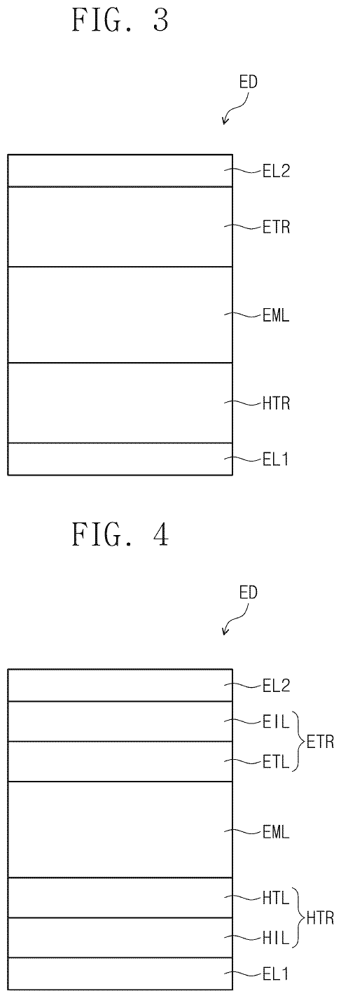

Each of the light emitting elements ED-1, ED-2, and ED-3 may have a structure of each light emitting element ED of embodiments according to FIGS. 3 to 7, which will be described later. Each of the light emitting elements ED-1, ED-2, and ED-3 may include a first electrode EL1, a hole transport region HTR, light emitting layers EML-R, EML-G, and EML-B, an electron transport region ETR, and a second electrode EL2.

FIG. 2 illustrates one or more embodiments in which the light emitting layers EML-R, EML-G, and EML-B of the light emitting elements ED-1, ED-2, and ED-3 are arranged in openings OH defined in the pixel defining layer PDL, and the hole transport region HTR, the electron transport region ETR, and the second electrode EL2 are provided as a common layer in the entire light emitting elements ED-1, ED-2, and ED-3. However, the embodiment of the present disclosure is not limited thereto, and unlike the configuration illustrated in FIG. 2, the hole transport region HTR and the electron transport region ETR in one or more embodiments may be provided by being patterned inside the openings OH defined in the pixel defining layer PDL. For example, the hole transport region HTR, the light emitting layers EML-R, EML-G, and EML-B, and the electron transport region ETR of the light emitting elements ED-1, ED-2, and ED-3 in one or more embodiments may be provided by being patterned in an inkjet printing method.

The encapsulation layer TFE may cover the light emitting elements ED-1, ED-2 and ED-3. The encapsulation layer TFE may seal the display element layer DP-ED. The encapsulation layer TFE may be a thin film encapsulation layer. The encapsulation layer TFE may be formed by laminating one layer or a plurality of layers. The encapsulation layer TFE includes at least one insulation layer. The encapsulation layer TFE according to one or more embodiments may include at least one inorganic film (hereinafter, an encapsulation-inorganic film). The encapsulation layer TFE according to one or more embodiments may also include at least one organic film (hereinafter, an encapsulation-organic film) and at least one encapsulation-inorganic film.

The encapsulation-inorganic film protects the display element layer DP-ED from moisture and/or oxygen, and the encapsulation-organic film protects the display element layer DP-ED from foreign substances such as dust particles. The encapsulation-inorganic film may include silicon nitride, silicon oxynitride, silicon oxide, titanium oxide, aluminum oxide, and/or the like, but the embodiment of the present disclosure is not particularly limited thereto. The encapsulation-organic film may include an acrylic-based compound, an epoxy-based compound, and/or the like. The encapsulation-organic film may include a photopolymerizable organic material, but the embodiment of the present disclosure is not particularly limited thereto.

The encapsulation layer TFE may be arranged on the second electrode EL2 and may be arranged filling the opening OH.
Referring to FIGS. 1 and 2, the display device DD may include one or more non-light emitting region(s) NPXA and also include light emitting regions PXA-R, PXA-G, and PXA-B. The light emitting regions PXA-R, PXA-G, and PXA-B may be regions in which light generated by the respective light emitting elements ED-1, ED-2, and ED-3 are emitted. The light emitting regions PXA-R, PXA-G, and PXA-B may be spaced and/or apart from each other on a plane.

Each of the light emitting regions PXA-R, PXA-G, and PXA-B may be a region divided (i.e., defined) by the pixel defining layer PDL. The non-light emitting regions NPXA may be areas between the adjacent light emitting regions PXA-R, PXA-G, and PXA-B, which correspond to the pixel defining layer PDL. In one or more embodiments, in the specification, the light emitting regions PXA-R, PXA-G, and PXA-B may respectively correspond to pixels. The pixel defining layer PDL may divide the light emitting elements ED-1, ED-2, and ED-3. The light emitting layers EML-R, EML-G, and EML-B of the light emitting elements ED-1, ED-2, and ED-3 may be arranged in openings OH defined in the pixel defining layer PDL and separated from each other.

The light emitting regions PXA-R, PXA-G, and PXA-B may be divided into a plurality of groups according to the color of light generated from the light emitting elements ED-1, ED-2, and ED-3. In the display device DD of one or more embodiments illustrated in FIGS. 1 and 2, three light emitting regions PXA-R, PXA-G, and PXA-B, which emit red light, green light, and blue light, respectively, are exemplarily illustrated. For example, the display device DD of one or more embodiments may include the red light emitting region PXA-R, the green light emitting region PXA-G, and the blue light emitting region PXA-B that are separated from each other.

In the display device DD according to one or more embodiments, the plurality of light emitting elements ED-1, ED-2 and ED-3 may be to emit light beams having wavelengths different from each other. For example, in one or more embodiments, the display device DD may include a first light emitting element ED-1 that emits red light, a second light emitting element ED-2 that emits green light, and a third light emitting element ED-3 that emits blue light. For example, the red light emitting region PXA-R, the green light emitting region PXA-G, and the blue light emitting region PXA-B of the display device DD may correspond to the first light emitting element ED-1, the second light emitting element ED-2, and the third light emitting element ED-3, respectively.

However, the embodiment of the present disclosure is not limited thereto, and the first to third light emitting elements ED-1, ED-2, and ED-3 may be to emit light beams in substantially the same wavelength range or at least one light emitting element may be to emit a light beam in a wavelength range different from the others. For example, the first to third light emitting elements ED-1, ED-2, and ED-3 may all emit blue light.

The light emitting regions PXA-R, PXA-G, and PXA-B in the display device DD according to one or more embodiments may be arranged in a stripe form. Referring to FIG. 1, the plurality of red light emitting regions PXA-R may be arranged with each other along a second directional axis DR2, the plurality of green light emitting regions PXA-G may be arranged with each other along the second directional axis DR2, and the plurality of blue light emitting regions PXA-B each may be arranged along the second directional axis DR2. In one or more embodiments, the red light emitting region PXA-R, the green light emitting region PXA-G, and the blue light emitting region PXA-B may be alternately arranged with each other in this order along a first directional axis DR1.

FIGS. 1 and 2 illustrate that all the light emitting regions PXA-R, PXA-G, and PXA-B have similar area, but the embodiment of the present disclosure is not limited thereto. Thus, the light emitting regions PXA-R, PXA-G, and PXA-B may have different areas from each other according to the wavelength range of the emitted light. In this case, the areas of the light emitting regions PXA-R, PXA-G, and PXA-B may refer to areas when viewed on a plane defined by the first directional axis DR1 and the second directional axis DR2.

In one or more embodiments, an arrangement form of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in one or more suitable combinations according to the characteristics of display quality desired or required in the display device DD. For example, the arrangement form of the light emitting regions PXA-R, PXA-G, and PXA-B may be a pentile (PENTILE™) arrangement form or a diamond (Diamond Pixel™) arrangement form, (PENTILE™ and Diamond Pixel™ are registered trademarks owned by Samsung Display Co., Ltd.).

In one or more embodiments, the areas of the light emitting regions PXA-R, PXA-G, and PXA-B may be different from each other. For example, in one or more embodiments, the area of the green light emitting region PXA-G may be smaller than that of the blue light emitting region PXA-B, but the embodiment of the present disclosure is not limited thereto.

Hereinafter, FIGS. 3 to 7 are cross-sectional views schematically illustrating light emitting elements according to one or more embodiments. Each of the light emitting elements ED according to one or more embodiments may include a first electrode EL1, a hole transport region HTR, an light emitting layer EML, an electron transport region ETR, and a second electrode EL2 that are sequentially stacked.

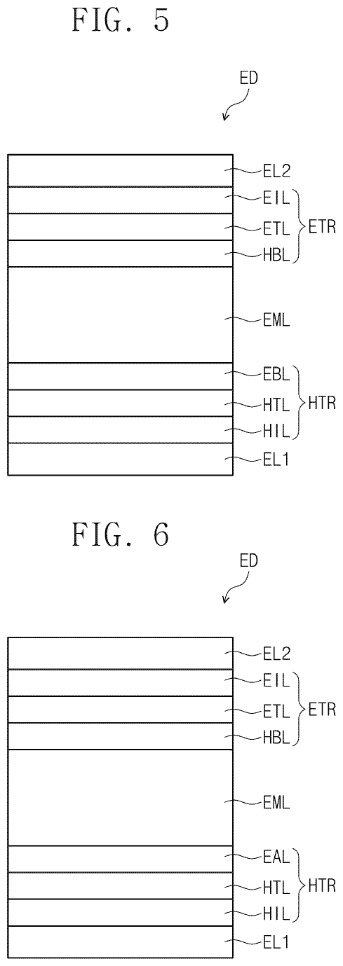

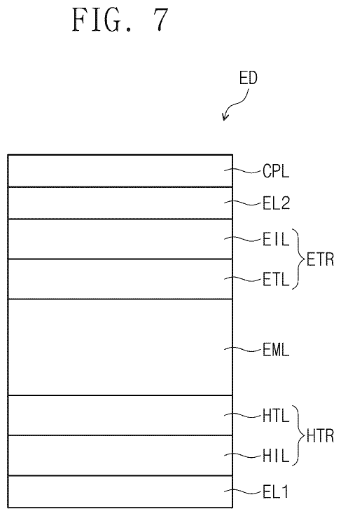

Compared with FIG. 3, FIG. 4 illustrates a cross-sectional view of a light emitting element ED of one or more embodiments, in which a hole transport region HTR includes a hole injection layer HIL and a hole transport layer HTL, and an electron transport region ETR includes an electron injection layer EIL and an electron transport layer ETL. In one or more embodiments, compared with FIG. 3, FIG. 5 illustrates a cross-sectional view of a light emitting element ED of one or more embodiments, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. As compared with FIG. 3, FIG. 6 illustrates a cross-sectional view of a light emitting element ED of one or more embodiments, in which a hole transport region HTR includes a hole injection layer HIL, a hole transport layer HTL, and a light emtting auxiliary layer EAL, and an electron transport region ETR includes an electron injection layer EIL, an electron transport layer ETL, and a hole blocking layer HBL. Compared with FIG. 4, FIG. 7 illustrates a cross-sectional view of a light emitting element ED of one or more embodiments including a capping layer CPL arranged on a second electrode EL2.

The first electrode EL1 has conductivity (e.g., is a conductor). The first electrode EL1 may be formed of a metal material, a metal alloy, or a conductive compound. The first electrode EL1 may be an anode or a cathode. However, the embodiment of the present disclosure is not limited thereto. In one or more embodiments, the first electrode EL1 may be a pixel electrode. The first electrode EL1 may be a transmissive electrode, a transflective electrode, or a reflective electrode. The first electrode EL1 may include at least one selected from among Ag, Mg, Cu, Al, Pt, Pd, Au, Ni, Nd, Ir, Cr, Li, Ca, LiF, Mo, Ti, W, In, Sn, and Zn, a compound of two or more selected from among these (thereof), a mixture of two or more selected from among these (thereof), and/or an oxide thereof.

If the first electrode EL1 is the transmissive electrode, the first electrode EL1 may include a transparent metal oxide such as indium tin oxide (ITO), indium zinc oxide (IZO), zinc oxide (ZnO), or indium tin zinc oxide (ITZO). If the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, a (e.g., any suitable) compound or mixture thereof (e.g., a mixture of Ag and Mg). In one or more embodiments, the first electrode EL1 may have a multilayer structure including a reflective film or a transflective film formed of the herein-described materials, and a transparent conductive film formed of ITO, IZO, ZnO, ITZO, and/or the like For example, the first electrode EL1 may have a three-layer structure of ITO/Ag/ITO, but the embodiment of the present disclosure is not limited thereto. In one or more embodiments, the embodiment of the present disclosure is not limited thereto, and the first electrode EL1 may include the herein-described metal materials, combinations of at least two metal materials of the herein-described metal materials, oxides of the herein-described metal materials, and/or the like. The thickness of the first electrode EL1 may be from about 700 angstrom (A) to about 10,000 Å. For example, the thickness of the first electrode EL1 may be from about 1,000 Å to about 3,000 Å.

The hole transport region HTR is provided on the first electrode EL1. The hole transport region HTR is arranged between the first electrode EL1 and the light emitting layer EML.

The hole transport region HTR may include at least one of a hole injection layer HIL, a hole transport layer HTL, a light emitting auxiliary layer EAL, or an electron blocking layer EBL. The light emitting auxiliary layer EAL may be referred to as a buffer layer. The thickness of the hole transport region HTR may be, for example, from about 50 Å to about 15,000 Å.

The hole transport region HTR may have a single layer formed of a single material, a single layer formed of a plurality of different materials, or a multilayer structure including a plurality of layers formed of a plurality of different materials.

For example, the hole transport region HTR may have a single layer structure of the hole injection layer HIL or the hole transport layer HTL, or may have a single layer structure formed of a hole injection material and a hole transport material. In one or more embodiments, the hole transport region HTR may have a single layer structure formed of a plurality of different materials, or a structure in which a hole injection layer HIL/hole transport layer HTL, a hole injection layer HIL/hole transport layer HTL/light emitting auxiliary layer EAL, a hole injection layer HIL/light emitting auxiliary layer EAL, a hole transport layer HTL/light emitting auxiliary layer EAL, or a hole injection layer HIL/hole transport layer HTL/electron blocking layer EBL are stacked in order from the first electrode EL1, but the embodiment of the present disclosure is not limited thereto. In one or more embodiments, the hole transport layer HTL may have a single layer or a multilayer structure having a plurality of layers.

The hole transport region HTR may be formed utilizing one or more suitable methods such as a vacuum deposition method, a spin coating method, a cast method, a Langmuir-Blodgett (LB) method, an inkjet printing method, a laser printing method, and a laser induced thermal imaging (LITI) method.

### Amine Compound

The light emitting element ED of one or more embodiments may include an amine compound of one or more embodiments represented by Formula 1 in the hole transport region HTR. In one or more embodiments, at least one of the hole injection layer HIL, the hole transport layer HTL, the electron blocking layer EBL, and the light emitting auxiliary layer EAL may include the amine compound of one or more embodiments represented by Formula 1. For example, the light emitting element ED of one or more embodiments may include the amine compound of one or more embodiments in the hole transport layer HTL.

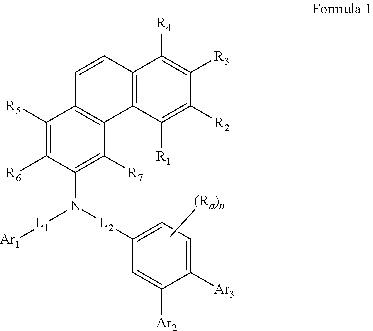

The amine compound of one or more embodiments may contain both (e.g., simultaneously) a phenanthrene group bonded to a nitrogen atom (N) and a substituted phenyl group. The amine compound of one or more embodiments may contain a substituted or unsubstituted phenanthrene group directly bonded to the nitrogen atom (N), and a substituted or unsubstituted ortho-terphenyl derivative directly bonded to the nitrogen atom or bonded thereto through a linker. In one or more embodiments, the amine compound of one or more embodiments may be a monoamine compound that excludes (e.g., does not contain) an additional amine substituent.

In the amine compound of one or more embodiments, the substituted or unsubstituted phenanthrene group may be directly bonded to the nitrogen atom at a c1 position as illustrated herein. In the present specification, the phenanthrene group, which is a component bonded to the nitrogen atom of the amine compound, may be referred to as a 3-phenanthrenyl group.

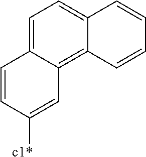

In one or more embodiments, in the amine compound of one or more embodiments, the substituted or unsubstituted ortho-terphenyl derivative may be directly bonded to the nitrogen atom at a c2 position or bonded to the nitrogen atom through a linker as illustrated herein. In the present specification, the ortho-terphenyl derivative, which is a component bonded to the nitrogen atom of the amine compound, may be referred to as a 3,4-substituted phenyl group. In the ortho-terphenyl derivative, Ar₂ and Ar₃ may be a substituted or unsubstituted aryl group.

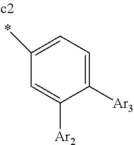

The amine compound of one or more embodiments may include the 3-phenanthrenyl group and the 3,4-substituted phenyl group, thereby having its charge balance adjusted by a three-dimensional aspect, and accordingly, may exhibit excellent or suitable charge transportability. For example, the amine compound of one or more embodiments may include a phenanthrene group and an ortho-terphenyl derivative, each of which may be bonded to a nitrogen atom at a specific position, thereby having excellent or suitable charge transportability and material stability, and thus, may contribute to high efficiency and long lifespan of a light emitting element.

In Formula 1, R₁ to R₇ may each independently be a hydrogen atom, a deuterium atom, a halogen atom, a substituted or unsubstituted alkyl group having 1 to 15 carbons, a substituted or unsubstituted aryl group having 6 to 30 ring-forming carbon atoms, or a substituted or unsubstituted heteroaryl group having 2 to 30 ring-forming carbon atoms. In one or more embodiments, R₁ to R₇ may not form a ring by being bonded to an adjacent group.

In one or more embodiments, R₁ to R₇ may each (e.g., all) be hydrogen atoms, or at least one thereof may be a deuterium atom, at least one thereof may be a halogen atom, at least one thereof may be a substituted or unsubstituted alkyl group, or at least one thereof may be a substituted or unsubstituted aryl group. For example, in one or more embodiments, R₁ to R₇ may each (e.g., all) be hydrogen atoms, or may each (e.g., all) be deuterium atoms, or at least one thereof may be a fluorine atom, at least one thereof may be a t-butyl group, or at least one thereof may be a substituted or unsubstituted phenyl group. However, the embodiment of the present disclosure is not limited thereto.

In Formula 1, n may be an integer of 1 to 3. In one or more embodiments, Rₐ may be a hydrogen atom, a deuterium atom, a halogen atom, a substituted or unsubstituted alkyl group having 1 to 15 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or may form a ring by being bonded to an adjacent group.

When n is 2 or greater, a plurality of Rₐ may each (e.g., all) be the same, or at least one thereof may be different from the rest thereof. The plurality of Rₐ may each (e.g., all) be hydrogen atoms. In one or more embodiments, in contrast, at least one of Rₐ may be a deuterium atom, at least one thereof may be a halogen atom, or at least one thereof may be a substituted or unsubstituted phenyl group. However, the embodiment of the present disclosure is not limited thereto.

In one or more embodiments, a plurality of adjacent Rₐ may form a ring by being bonded to each other. When Rₐ and an adjacent group are bonded to each other to form a ring, the Rₐ and the adjacent group may be bonded to each other to form a saturated hydrocarbon ring, or an unsaturated hydrocarbon ring. For example, Rₐ may be bonded to a benzene ring, which is to be substituted, to form a condensed ring.

In Formula 1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms.

For example, in Formula 1, L₁ may be a direct linkage, a substituted or unsubstituted arylene group, or a substituted or unsubstituted divalent dibenzoheterole group. In one or more embodiments, L₂ may be a direct linkage, or a substituted or unsubstituted arylene group.

In one or more embodiments, L₁ and L₂ may each independently be a direct linkage, a substituted or unsubstituted phenylene group, a substituted or unsubstituted divalent biphenyl group, or a substituted or unsubstituted naphthalene group. However, the embodiment of the present disclosure is not limited thereto.

In Formula 1, Ar₁ may be a substituted or unsubstituted aryl group having 6 to 30 ring-forming carbon atoms, or a substituted or unsubstituted heteroaryl group having 2 to 30 ring-forming carbon atoms. However, a case in which Ar₁ is a fluorenyl group when L₁ is a direct linkage is excluded. For example, in the amine compound of one or more embodiments, a case in which Ar₁ is represented by Formula Ar₁-a, as illustrated herein, when L₁ is a direct linkage is excluded.

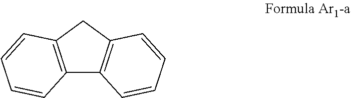 [SMILES: c1ccc2c(c1)Cc1ccccc1-2]

In one or more embodiments, Ar₁ may be a substituted or unsubstituted phenyl group, a substituted or unsubstituted naphthyl group, a substituted or unsubstituted biphenyl group, a substituted or unsubstituted terphenyl group, a substituted or unsubstituted fluorenyl group, a substituted or unsubstituted dibenzoheterole group including O, S, N, or Si as a ring-forming hetero atom, or a substituted or unsubstituted benzonaphthoheterol group including O or S as a ring-forming hetero atom.

When Ar₁ is a substituted or unsubstituted dibenzoheterole group, Ar₁ may be a substituted or unsubstituted dibenzofuran group, a substituted or unsubstituted dibenzothiophene group, a substituted or unsubstituted carbazol group, or a substituted or unsubstituted dibenzosilole group. However, the embodiment of the present disclosure is not limited thereto.

When Ar₁ is a substituted or unsubstituted benzonaphthoheterol group, Ar₁ may be a substituted or unsubstituted benzonaphthofuran group, or a substituted or unsubstituted benzonaphthothiophene group. However, the embodiment of the present disclosure is not limited thereto.

In Formula 1, Ar₂ and Ar₃ may each independently be a substituted or unsubstituted phenyl group, a substituted or unsubstituted biphenyl group, a substituted or unsubstituted terphenyl group, a substituted or unsubstituted naphthyl group, or a substituted or unsubstituted phenanthrenyl group. However, a case in which each (e.g., both) of Ar₂ and Ar₃ contain a carbazole group as a substituent is excluded.

In one or more embodiments, in the amine compound of one or more embodiments represented by Formula 1, each (e.g., all) of Ar₁, Ar₂, and Ar₃ do not contain benzophenanthrene. For example, the amine compound of one or more embodiments represented by Formula 1 does not contain benzophenanthrene represented by Formula Ar-x, as illustrated herein.

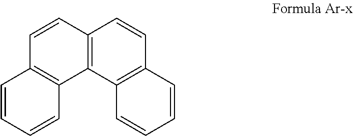 [SMILES: c1ccc2c(c1)ccc1ccc3ccccc3c12]

In the amine compound represented by Formula 1, at least one hydrogen atom may be substituted with a deuterium atom. In Formula 1, R₁ to R₇, and Rₐ may each independently be a deuterium atom, or may contain a deuterium atom as a substituent. In one or more embodiments, L₁ and L₂ may each independently contain a deuterium atom as a substituent. In one or more embodiments, Ar₁, Ar₂, and Ar₃ may each independently contain a deuterium atom as a substituent.

In Formula 1, R₁ to R₇, Rₐ, Ar₁, Ar₂, Ar₃, L₁, and L₂ may not contain (e.g., may exclude) a substituted or unsubstituted amine group. For example, the amine compound represented by Formula 1 may be a monoamine compound that excludes (e.g., does not contain) an additional amine group.

Formula 1 may be represented by Formula 1A.

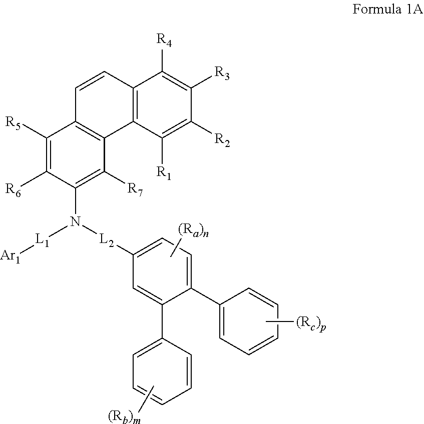

In Formula 1A, m and p may each independently be an integer of 1 to 5. In one or more embodiments, R_{b} and R_{c} may each independently be a hydrogen atom, a deuterium atom, a halogen atom, a substituted or unsubstituted alkyl group having 1 to 15 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or may form a ring by being bonded to an adjacent group.

When m is 2 or greater, a plurality of R_{b} may each (e.g., all) be the same, or at least one thereof may be different from the rest thereof. In one or more embodiments, when p is 2 or greater, a plurality of Rr may each (e.g., all) be the same, or at least one thereof may be different from the rest thereof. The plurality of R_{b} and Rr may each (e.g., all) be hydrogen atoms. In one or more embodiments, in contrast, at least one of the plurality of R_{b} and R_{c} may be a deuterium atom, at least one thereof may be a halogen atom, or at least one thereof may be a substituted or unsubstituted aryl group. However, the embodiment of the present disclosure is not limited thereto.

In one or more embodiments, a plurality of adjacent Rₐ or R_{c} may form a ring by being bonded to each other. When R_{b} and an adjacent group are bonded to each other to form a ring, the R_{b} and the adjacent group may be bonded to each other to form a saturated hydrocarbon ring, or an unsaturated hydrocarbon ring. When R_{c} and an adjacent group are bonded to each other to form a ring, the R_{c} and the adjacent group may be bonded to each other to form a saturated hydrocarbon ring, or an unsaturated hydrocarbon ring. In one or more embodiments, even when R_{b} and an adjacent group are bonded to each other to form a ring, or when R_{c} and an adjacent group are bonded to each other to form a ring, the amine compound of one or more embodiments does not contain benzophenanthrene.

In Formula 1A, the contents described herein with reference to Formula 1 may be equally applied to Ar₁, L₁, L₂, R₁ to R₇, Rₐ, and n.

In the amine compound of one or more embodiments represented by Formula 1, a 3,4-substituted phenyl group may be directly bonded to a nitrogen atom of amine or bonded to the nitrogen atom through a linker.

Formula 1 may be represented by Formula 1-1 or Formula 1-2, as described herein. Formula 1-1 shows the amine compound of one or more embodiments in which a 3,4-substituted phenyl group is directly bonded to a nitrogen atom, and Formula 1-2 shows the amine compound of one or more embodiments in which a 3,4-substituted phenyl group is bonded to a nitrogen atom through L₂ₐ, which is a linker.

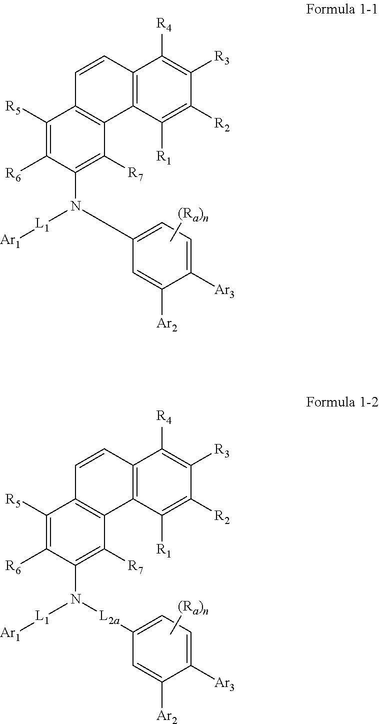

In Formula 1-2, L₂ₐ may be a substituted or unsubstituted phenylene group, or a substituted or unsubstituted divalent biphenyl group. In one or more embodiments, in Formula 1-1 and Formula 1-2, the contents described herein with reference to Formula 1 may be equally applied to Ar₁, Ar₂, Ar₃, L₁, R₁ to R₇, Rₐ, and n.

The amine compound of one or more embodiments represented by Formula 1 may be represented by an one (e.g., one) selected from among compounds of Compound Group 1. The hole transport region HTR of the light emitting element ED of one or more embodiments may include at least one selected from among amine compounds disclosed in Compound Group 1. In Compound Group 1, D is a deuterium atom.

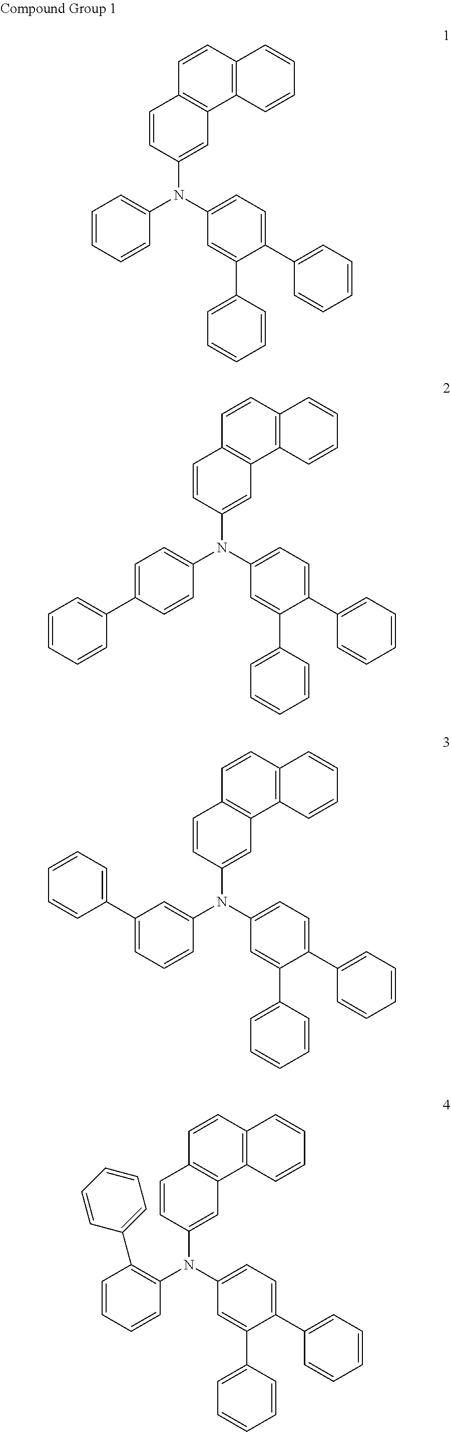

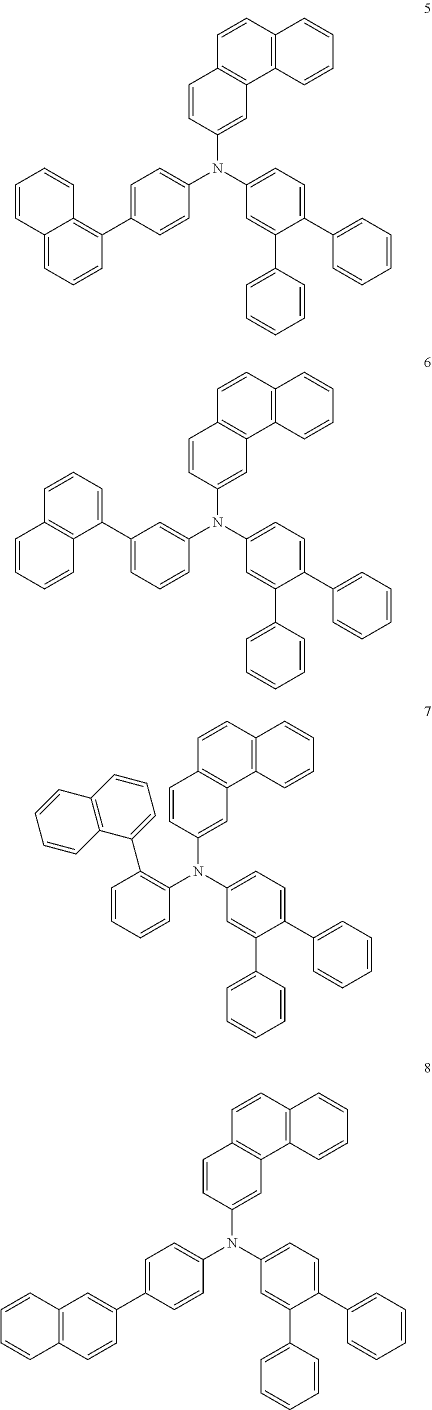

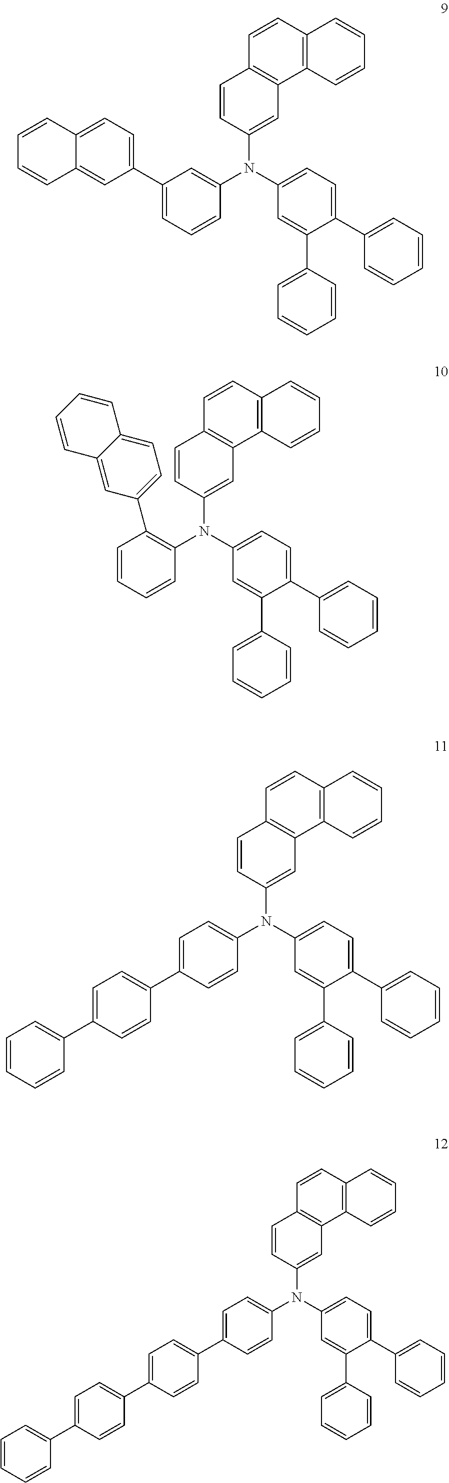 [SMILES: c1ccc(-c2ccc(N(c3cccc(-c4ccc5ccccc5c4)c3)c3ccc4ccc5ccccc5c4c3)cc2-c2ccccc2)cc1 ; c1ccc(-c2ccc(N(c3ccc4ccc5ccccc5c4c3)c3ccccc3-c3ccc4ccccc4c3)cc2-c2ccccc2)cc1 ; c1ccc(-c2ccc(-c3ccc(N(c4ccc(-c5ccccc5)c(-c5ccccc5)c4)c4ccc5ccc6ccccc6c5c4)cc3)cc2)cc1 ; c1ccc(-c2ccc(-c3ccc(-c4ccc(N(c5ccc(-c6ccccc6)c(-c6ccccc6)c5)c5ccc6ccc7ccccc7c6c5)cc4)cc3)cc2)cc1]

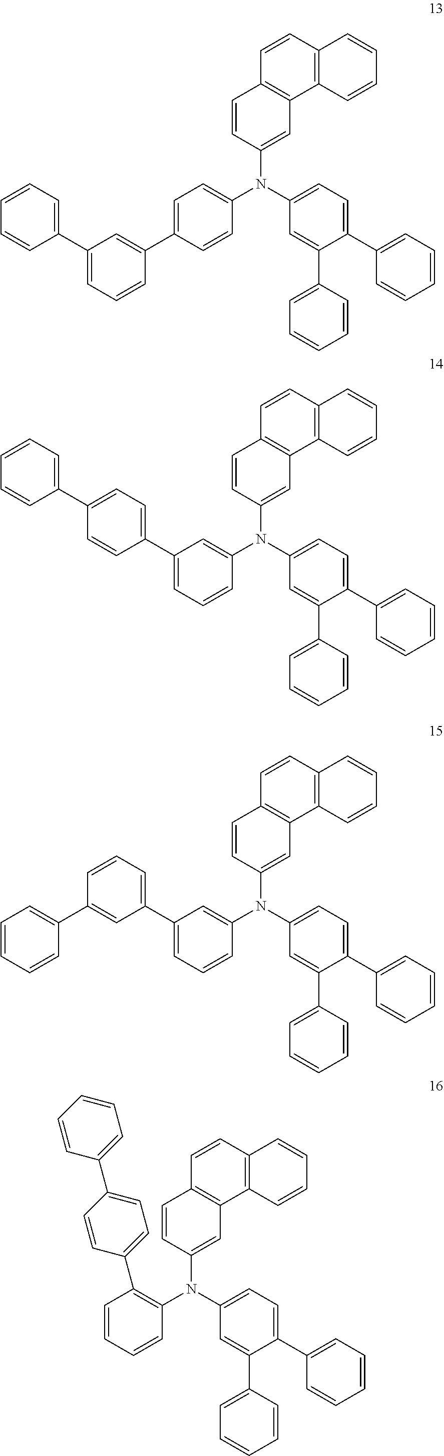 [SMILES: c1ccc(-c2cccc(-c3ccc(N(c4ccc(-c5ccccc5)c(-c5ccccc5)c4)c4ccc5ccc6ccccc6c5c4)cc3)c2)cc1 ; c1ccc(-c2ccc(-c3cccc(N(c4ccc(-c5ccccc5)c(-c5ccccc5)c4)c4ccc5ccc6ccccc6c5c4)c3)cc2)cc1 ; c1ccc(-c2cccc(-c3cccc(N(c4ccc(-c5ccccc5)c(-c5ccccc5)c4)c4ccc5ccc6ccccc6c5c4)c3)c2)cc1 ; c1ccc(-c2ccc(-c3ccccc3N(c3ccc(-c4ccccc4)c(-c4ccccc4)c3)c3ccc4ccc5ccccc5c4c3)cc2)cc1]

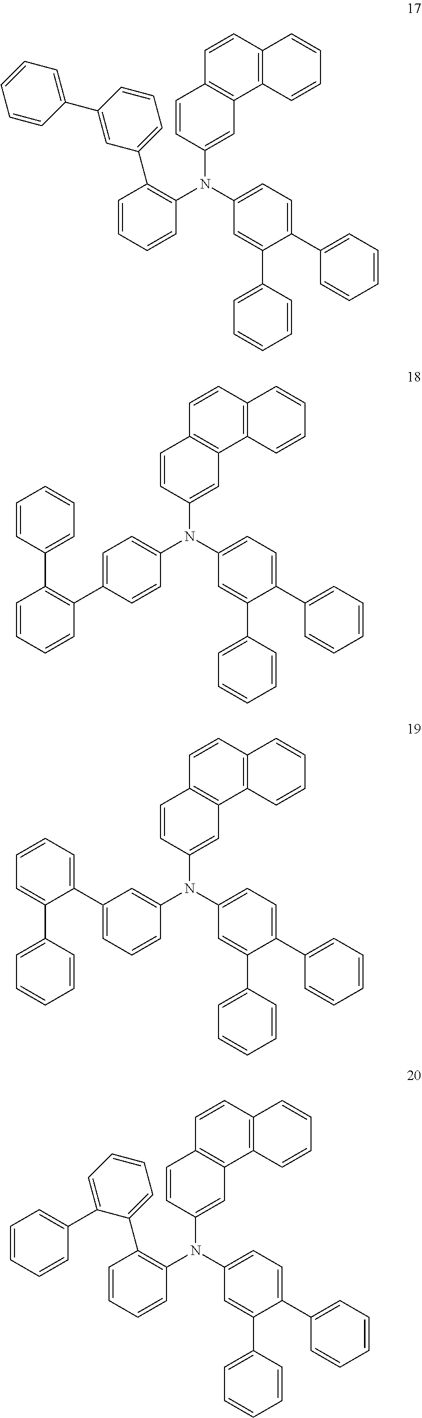

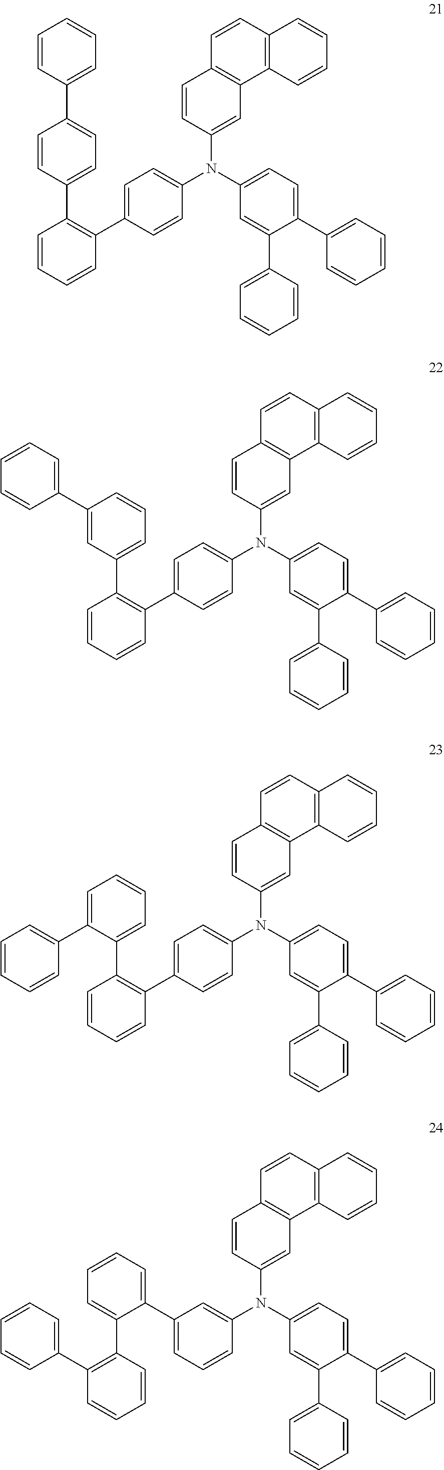 [SMILES: c1ccc(-c2ccc(-c3ccccc3-c3ccc(N(c4ccc(-c5ccccc5)c(-c5ccccc5)c4)c4ccc5ccc6ccccc6c5c4)cc3)cc2)cc1 ; c1ccc(-c2cccc(-c3ccccc3-c3ccc(N(c4ccc(-c5ccccc5)c(-c5ccccc5)c4)c4ccc5ccc6ccccc6c5c4)cc3)c2)cc1 ; c1ccc(-c2ccc(N(c3ccc(-c4ccccc4-c4ccccc4-c4ccccc4)cc3)c3ccc4ccc5ccccc5c4c3)cc2-c2ccccc2)cc1 ; c1ccc(-c2ccc(N(c3cccc(-c4ccccc4-c4ccccc4-c4ccccc4)c3)c3ccc4ccc5ccccc5c4c3)cc2-c2ccccc2)cc1]

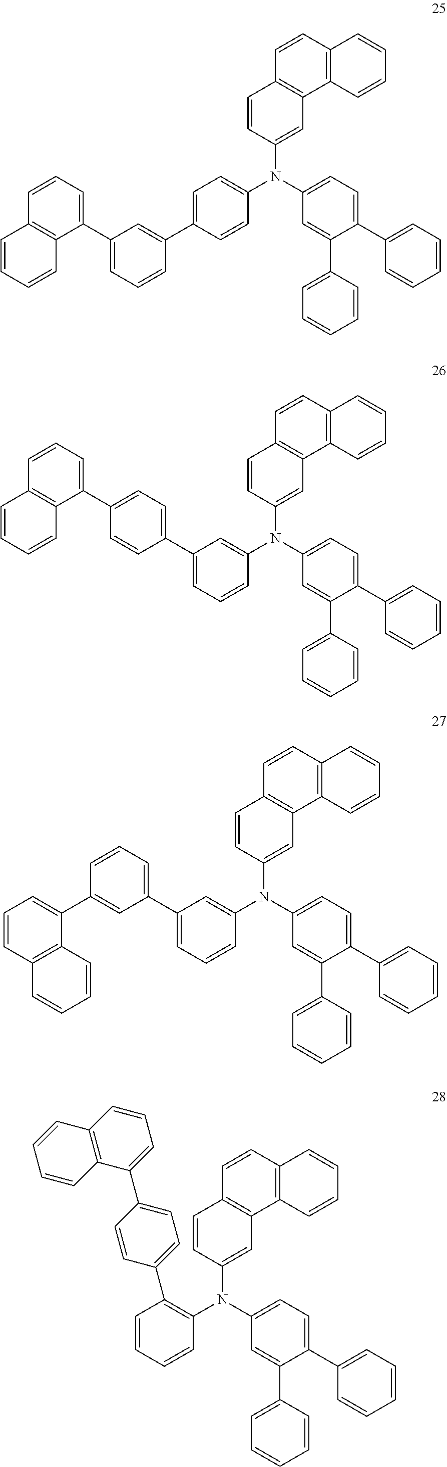 [SMILES: c1ccc(-c2ccc(N(c3ccc(-c4cccc(-c5cccc6ccccc56)c4)cc3)c3ccc4ccc5ccccc5c4c3)cc2-c2ccccc2)cc1 ; c1ccc(-c2ccc(N(c3cccc(-c4ccc(-c5cccc6ccccc56)cc4)c3)c3ccc4ccc5ccccc5c4c3)cc2-c2ccccc2)cc1 ; c1ccc(-c2ccc(N(c3cccc(-c4cccc(-c5cccc6ccccc56)c4)c3)c3ccc4ccc5ccccc5c4c3)cc2-c2ccccc2)cc1 ; c1ccc(-c2ccc(N(c3ccc4ccc5ccccc5c4c3)c3ccccc3-c3ccc(-c4cccc5ccccc45)cc3)cc2-c2ccccc2)cc1]

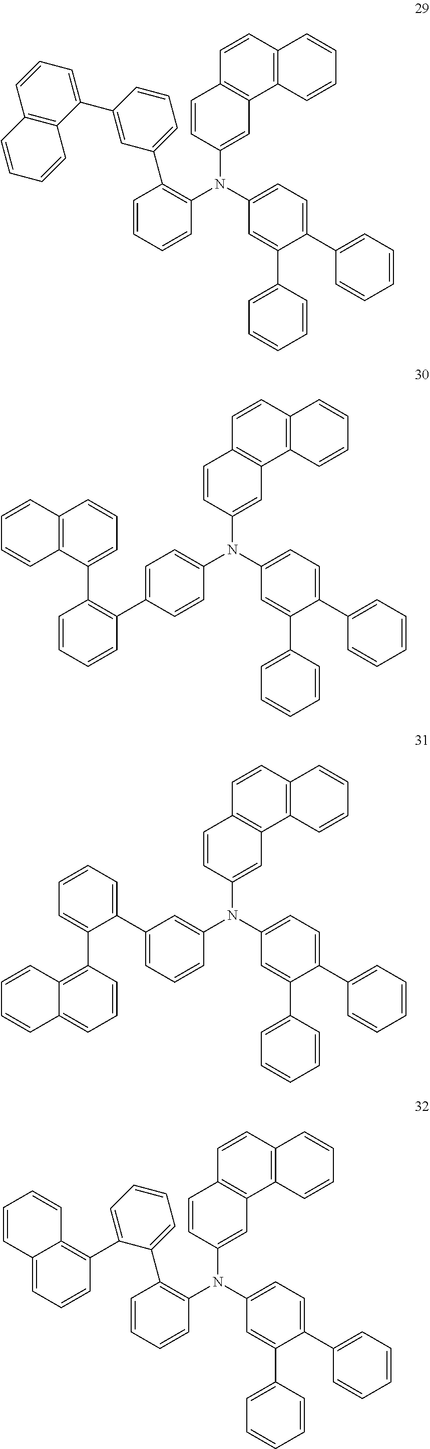 [SMILES: c1ccc(-c2ccc(N(c3ccc4ccc5ccccc5c4c3)c3ccccc3-c3cccc(-c4cccc5ccccc45)c3)cc2-c2ccccc2)cc1 ; c1ccc(-c2ccc(N(c3ccc(-c4ccccc4-c4cccc5ccccc45)cc3)c3ccc4ccc5ccccc5c4c3)cc2-c2ccccc2)cc1 ; c1ccc(-c2ccc(N(c3cccc(-c4ccccc4-c4cccc5ccccc45)c3)c3ccc4ccc5ccccc5c4c3)cc2-c2ccccc2)cc1 ; c1ccc(-c2ccc(N(c3ccc4ccc5ccccc5c4c3)c3ccccc3-c3ccccc3-c3cccc4ccccc34)cc2-c2ccccc2)cc1]

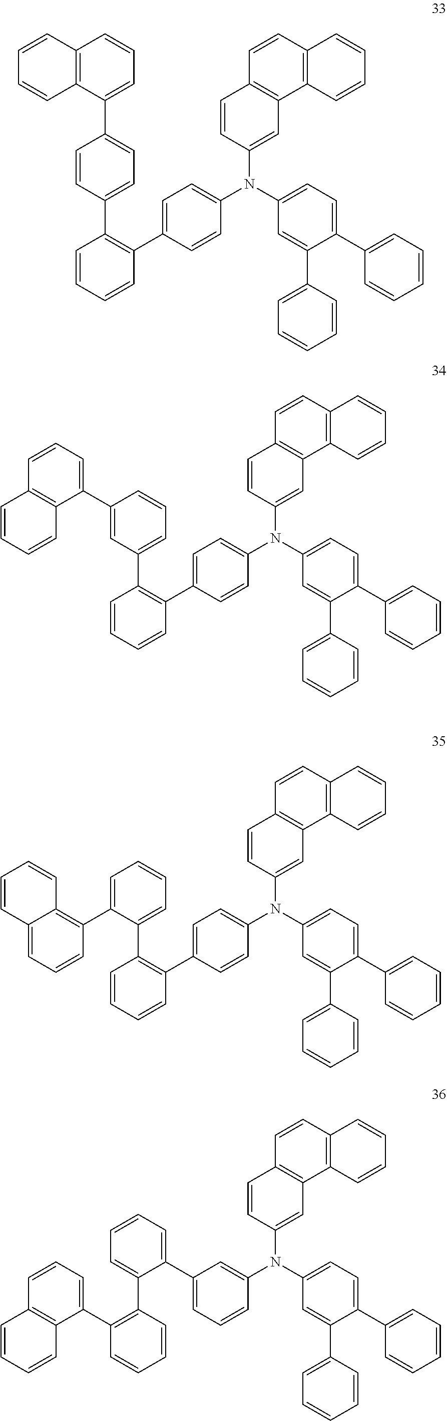 [SMILES: c1ccc(-c2ccc(N(c3ccc(-c4ccccc4-c4ccc(-c5cccc6ccccc56)cc4)cc3)c3ccc4ccc5ccccc5c4c3)cc2-c2ccccc2)cc1 ; c1ccc(-c2ccc(N(c3ccc(-c4ccccc4-c4cccc(-c5cccc6ccccc56)c4)cc3)c3ccc4ccc5ccccc5c4c3)cc2-c2ccccc2)cc1 ; c1ccc(-c2ccc(N(c3ccc(-c4ccccc4-c4ccccc4-c4cccc5ccccc45)cc3)c3ccc4ccc5ccccc5c4c3)cc2-c2ccccc2)cc1 ; c1ccc(-c2ccc(N(c3cccc(-c4ccccc4-c4ccccc4-c4cccc5ccccc45)c3)c3ccc4ccc5ccccc5c4c3)cc2-c2ccccc2)cc1]

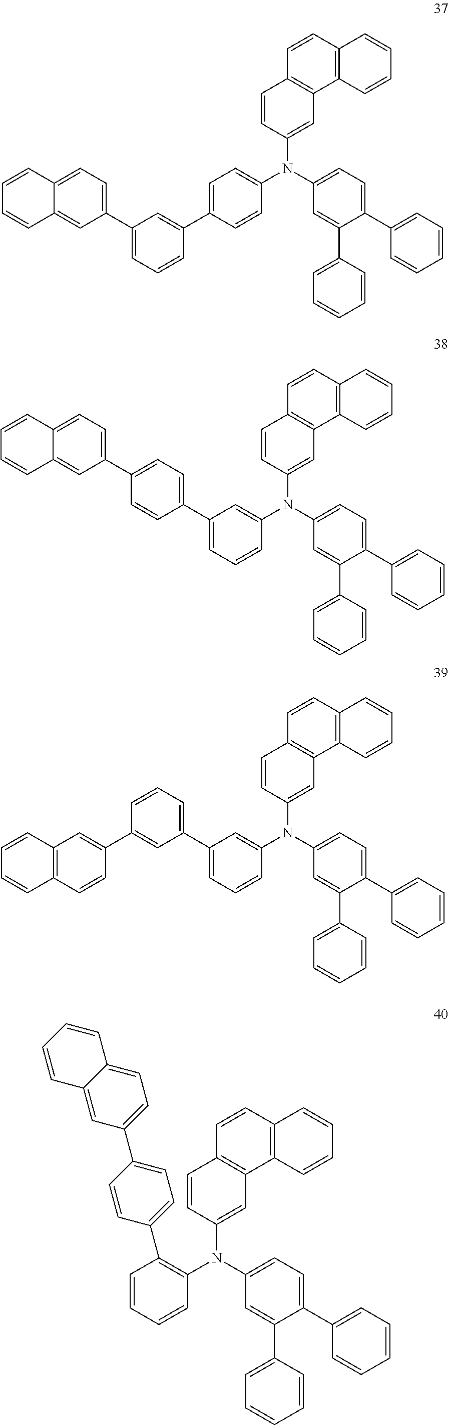 [SMILES: c1ccc(-c2ccc(N(c3ccc(-c4cccc(-c5ccc6ccccc6c5)c4)cc3)c3ccc4ccc5ccccc5c4c3)cc2-c2ccccc2)cc1 ; c1ccc(-c2ccc(N(c3cccc(-c4ccc(-c5ccc6ccccc6c5)cc4)c3)c3ccc4ccc5ccccc5c4c3)cc2-c2ccccc2)cc1 ; c1ccc(-c2ccc(N(c3cccc(-c4cccc(-c5ccc6ccccc6c5)c4)c3)c3ccc4ccc5ccccc5c4c3)cc2-c2ccccc2)cc1 ; c1ccc(-c2ccc(N(c3ccc4ccc5ccccc5c4c3)c3ccccc3-c3ccc(-c4ccc5ccccc5c4)cc3)cc2-c2ccccc2)cc1]

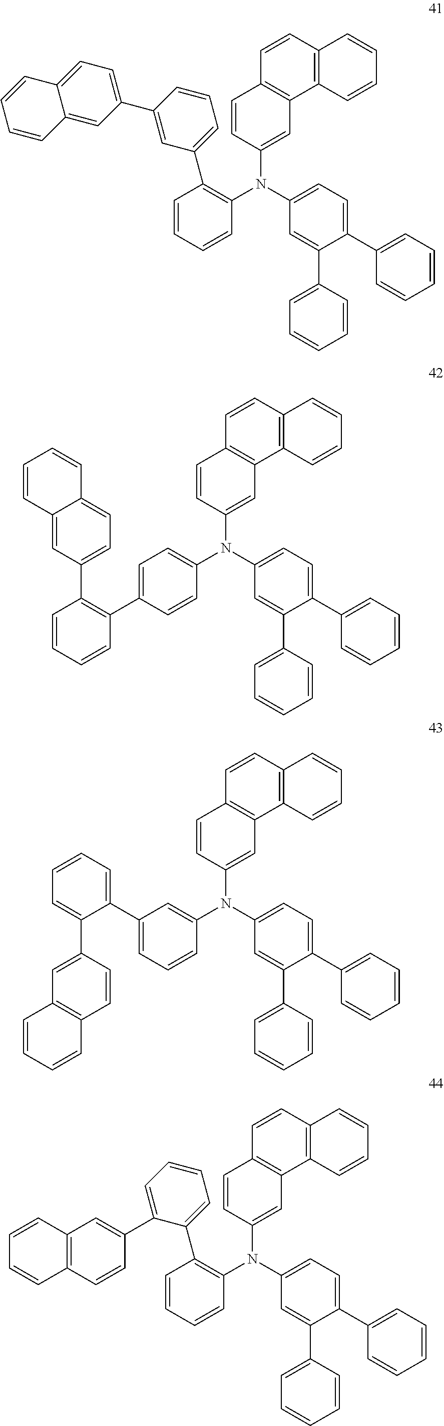

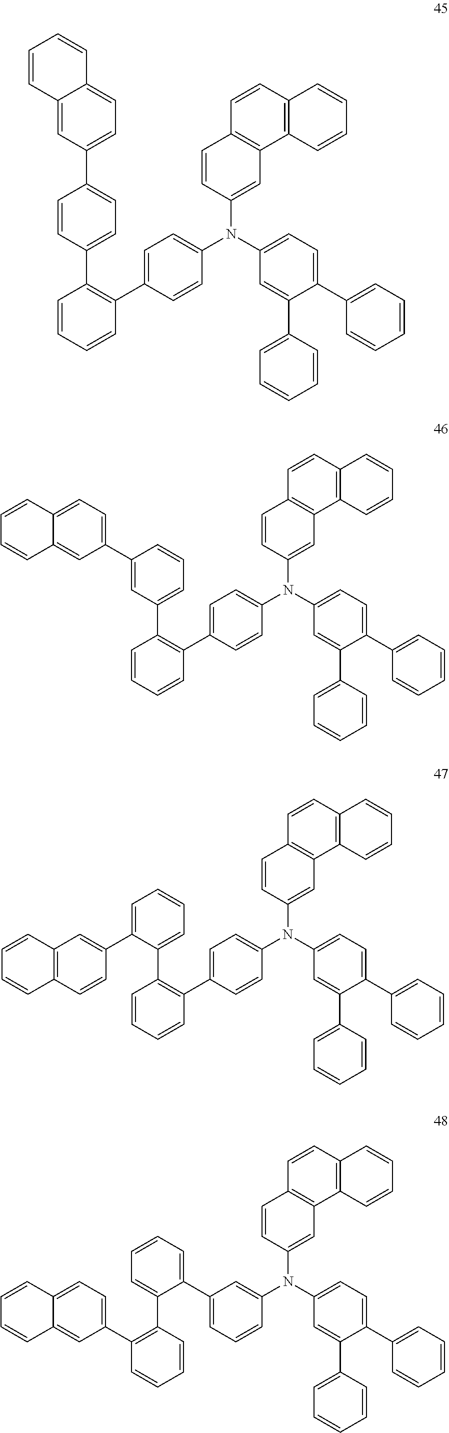 [SMILES: c1ccc(-c2ccc(N(c3ccc(-c4ccccc4-c4ccc(-c5ccc6ccccc6c5)cc4)cc3)c3ccc4ccc5ccccc5c4c3)cc2-c2ccccc2)cc1 ; c1ccc(-c2ccc(N(c3ccc(-c4ccccc4-c4cccc(-c5ccc6ccccc6c5)c4)cc3)c3ccc4ccc5ccccc5c4c3)cc2-c2ccccc2)cc1 ; c1ccc(-c2ccc(N(c3ccc(-c4ccccc4-c4ccccc4-c4ccc5ccccc5c4)cc3)c3ccc4ccc5ccccc5c4c3)cc2-c2ccccc2)cc1 ; c1ccc(-c2ccc(N(c3cccc(-c4ccccc4-c4ccccc4-c4ccc5ccccc5c4)c3)c3ccc4ccc5ccccc5c4c3)cc2-c2ccccc2)cc1]

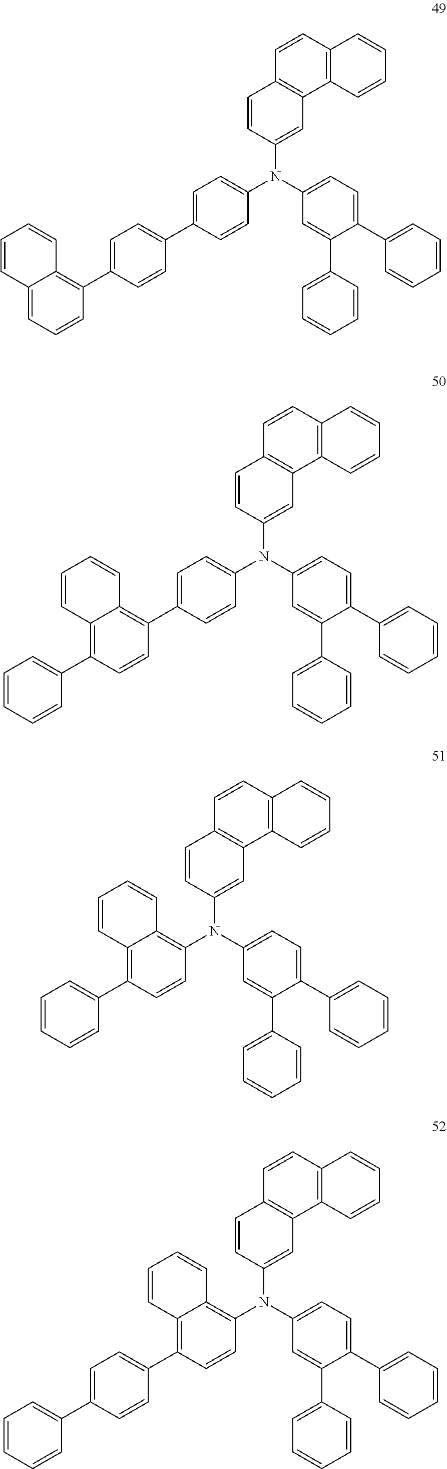 [SMILES: c1ccc(-c2ccc(N(c3ccc(-c4ccc(-c5cccc6ccccc56)cc4)cc3)c3ccc4ccc5ccccc5c4c3)cc2-c2ccccc2)cc1 ; c1ccc(-c2ccc(N(c3ccc(-c4ccc(-c5ccccc5)c5ccccc45)cc3)c3ccc4ccc5ccccc5c4c3)cc2-c2ccccc2)cc1 ; c1ccc(-c2ccc(N(c3ccc4ccc5ccccc5c4c3)c3ccc(-c4ccccc4)c4ccccc34)cc2-c2ccccc2)cc1 ; c1ccc(-c2ccc(-c3ccc(N(c4ccc(-c5ccccc5)c(-c5ccccc5)c4)c4ccc5ccc6ccccc6c5c4)c4ccccc34)cc2)cc1]

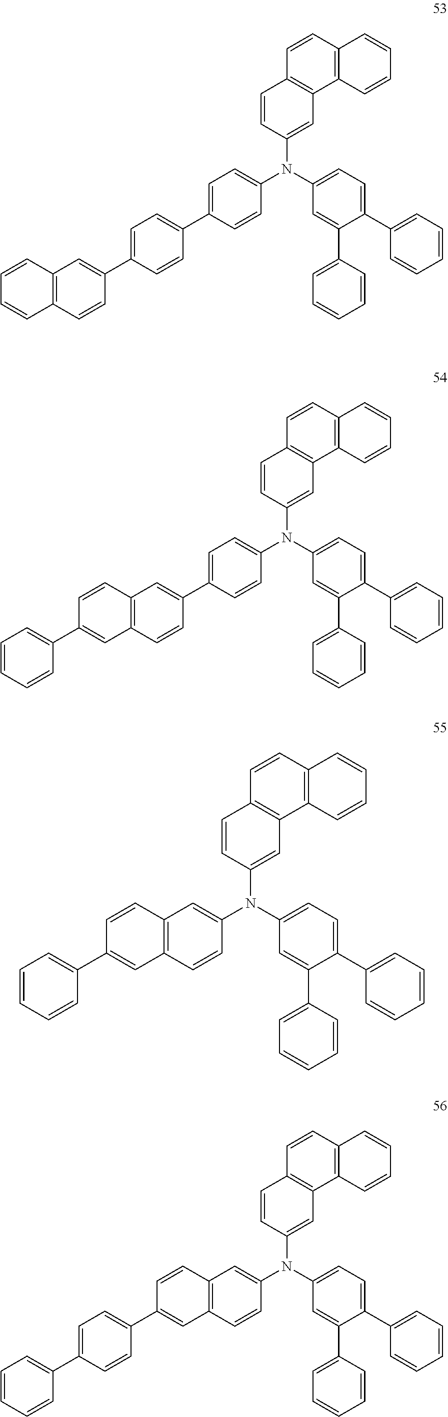 [SMILES: c1ccc(-c2ccc(N(c3ccc(-c4ccc(-c5ccc6ccccc6c5)cc4)cc3)c3ccc4ccc5ccccc5c4c3)cc2-c2ccccc2)cc1 ; c1ccc(-c2ccc3cc(-c4ccc(N(c5ccc(-c6ccccc6)c(-c6ccccc6)c5)c5ccc6ccc7ccccc7c6c5)cc4)ccc3c2)cc1 ; c1ccc(-c2ccc3cc(N(c4ccc(-c5ccccc5)c(-c5ccccc5)c4)c4ccc5ccc6ccccc6c5c4)ccc3c2)cc1 ; c1ccc(-c2ccc(-c3ccc4cc(N(c5ccc(-c6ccccc6)c(-c6ccccc6)c5)c5ccc6ccc7ccccc7c6c5)ccc4c3)cc2)cc1]

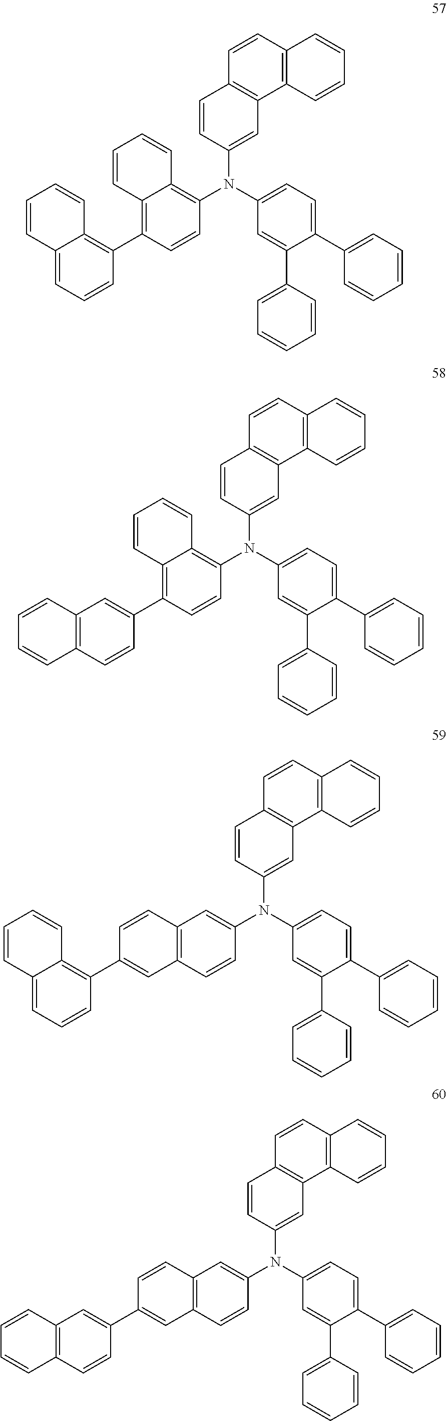

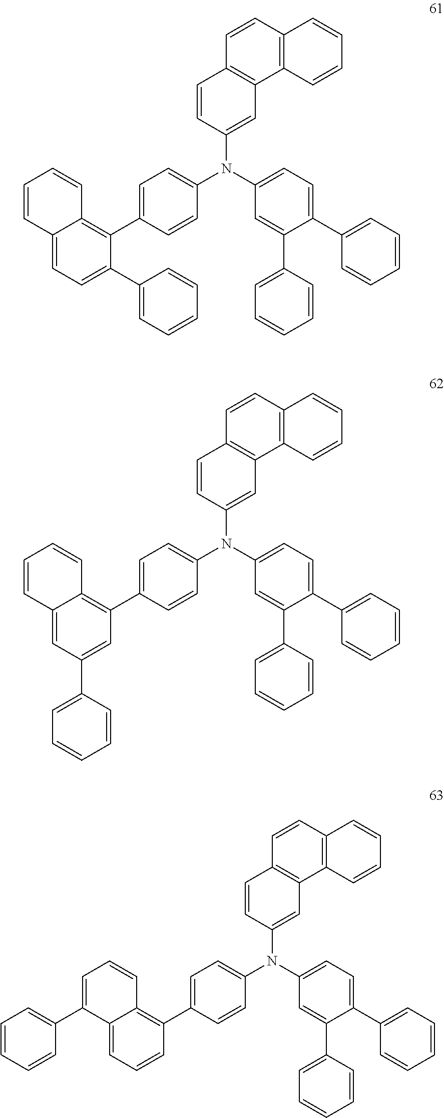 [SMILES: c1ccc(-c2ccc(N(c3ccc(-c4c(-c5ccccc5)ccc5ccccc45)cc3)c3ccc4ccc5ccccc5c4c3)cc2-c2ccccc2)cc1 ; c1ccc(-c2cc(-c3ccc(N(c4ccc(-c5ccccc5)c(-c5ccccc5)c4)c4ccc5ccc6ccccc6c5c4)cc3)c3ccccc3c2)cc1 ; c1ccc(-c2ccc(N(c3ccc(-c4cccc5c(-c6ccccc6)cccc45)cc3)c3ccc4ccc5ccccc5c4c3)cc2-c2ccccc2)cc1]

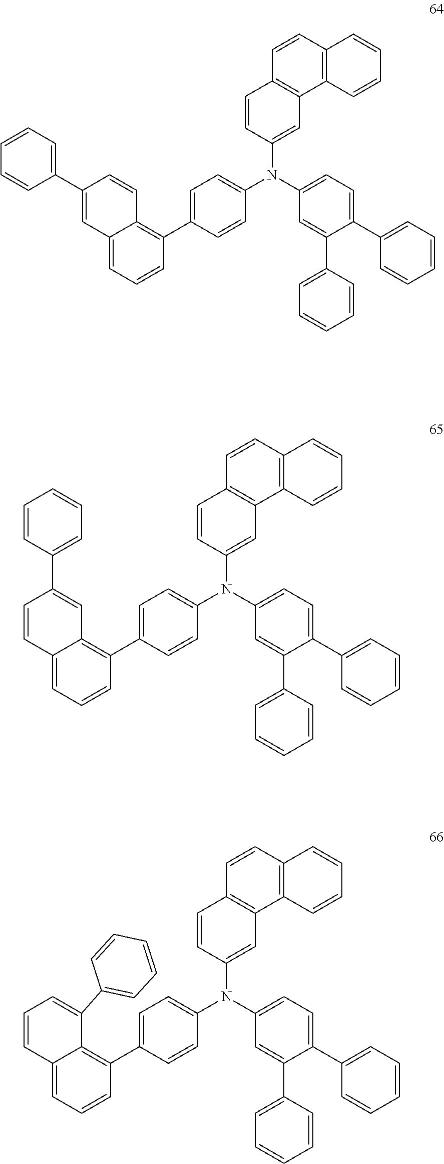 [SMILES: c1ccc(-c2ccc3c(-c4ccc(N(c5ccc(-c6ccccc6)c(-c6ccccc6)c5)c5ccc6ccc7ccccc7c6c5)cc4)cccc3c2)cc1 ; c1ccc(-c2ccc3cccc(-c4ccc(N(c5ccc(-c6ccccc6)c(-c6ccccc6)c5)c5ccc6ccc7ccccc7c6c5)cc4)c3c2)cc1 ; c1ccc(-c2ccc(N(c3ccc(-c4cccc5cccc(-c6ccccc6)c45)cc3)c3ccc4ccc5ccccc5c4c3)cc2-c2ccccc2)cc1]

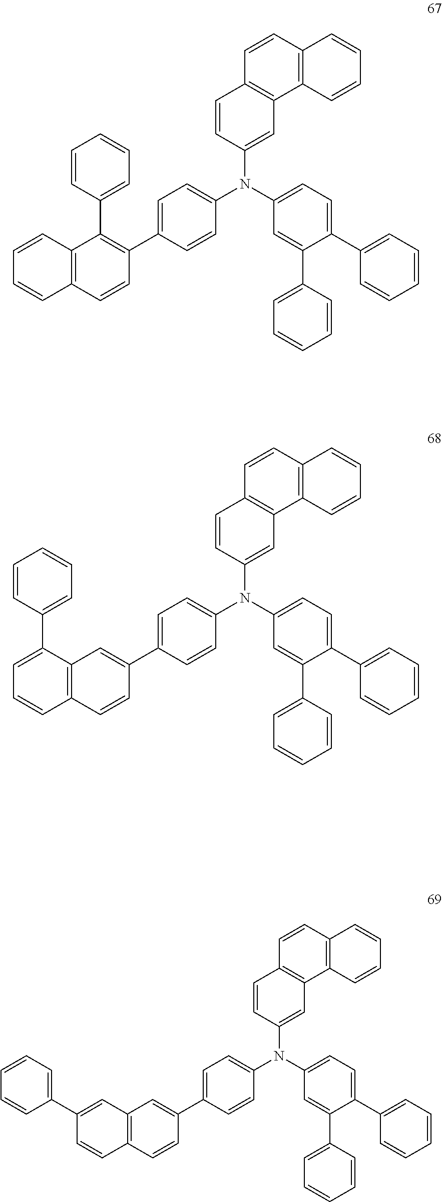 [SMILES: c1ccc(-c2ccc(N(c3ccc(-c4ccc5ccccc5c4-c4ccccc4)cc3)c3ccc4ccc5ccccc5c4c3)cc2-c2ccccc2)cc1 ; c1ccc(-c2ccc(N(c3ccc(-c4ccc5cccc(-c6ccccc6)c5c4)cc3)c3ccc4ccc5ccccc5c4c3)cc2-c2ccccc2)cc1 ; c1ccc(-c2ccc3ccc(-c4ccc(N(c5ccc(-c6ccccc6)c(-c6ccccc6)c5)c5ccc6ccc7ccccc7c6c5)cc4)cc3c2)cc1]

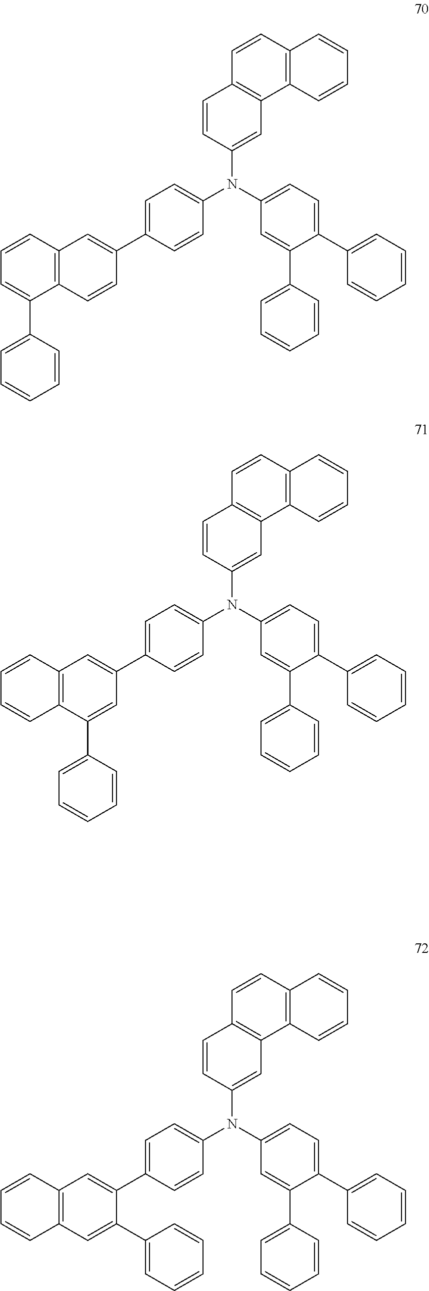 [SMILES: c1ccc(-c2ccc(N(c3ccc(-c4ccc5c(-c6ccccc6)cccc5c4)cc3)c3ccc4ccc5ccccc5c4c3)cc2-c2ccccc2)cc1 ; c1ccc(-c2ccc(N(c3ccc(-c4cc(-c5ccccc5)c5ccccc5c4)cc3)c3ccc4ccc5ccccc5c4c3)cc2-c2ccccc2)cc1 ; c1ccc(-c2ccc(N(c3ccc(-c4cc5ccccc5cc4-c4ccccc4)cc3)c3ccc4ccc5ccccc5c4c3)cc2-c2ccccc2)cc1]

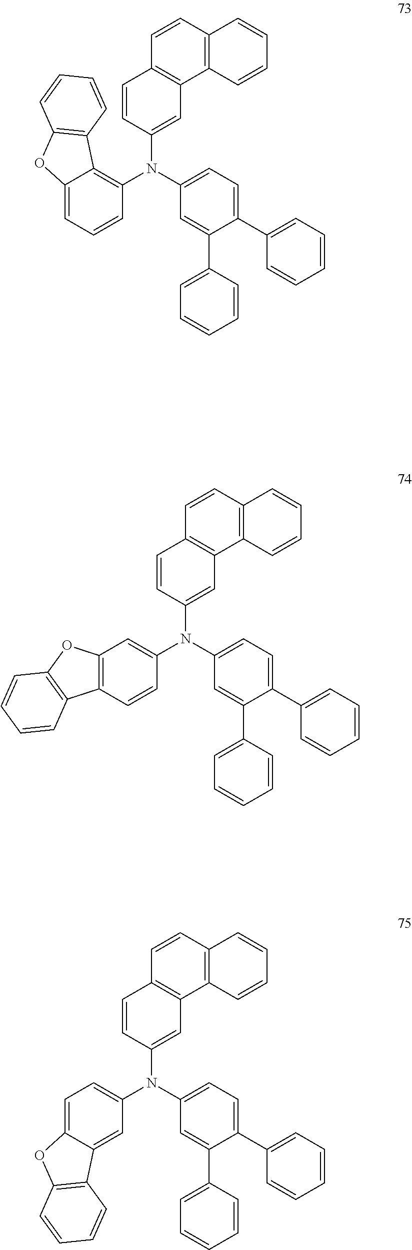 [SMILES: c1ccc(-c2ccc(N(c3ccc4ccc5ccccc5c4c3)c3cccc4oc5ccccc5c34)cc2-c2ccccc2)cc1 ; c1ccc(-c2ccc(N(c3ccc4c(c3)oc3ccccc34)c3ccc4ccc5ccccc5c4c3)cc2-c2ccccc2)cc1 ; c1ccc(-c2ccc(N(c3ccc4ccc5ccccc5c4c3)c3ccc4oc5ccccc5c4c3)cc2-c2ccccc2)cc1]

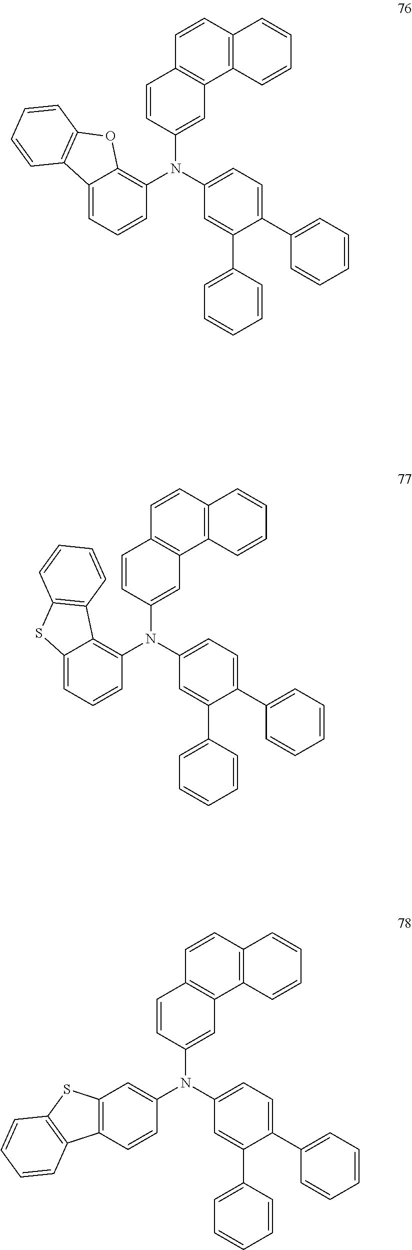 [SMILES: c1ccc(-c2ccc(N(c3ccc4ccc5ccccc5c4c3)c3cccc4c3oc3ccccc34)cc2-c2ccccc2)cc1 ; c1ccc(-c2ccc(N(c3ccc4ccc5ccccc5c4c3)c3cccc4sc5ccccc5c34)cc2-c2ccccc2)cc1 ; c1ccc(-c2ccc(N(c3ccc4c(c3)sc3ccccc34)c3ccc4ccc5ccccc5c4c3)cc2-c2ccccc2)cc1]

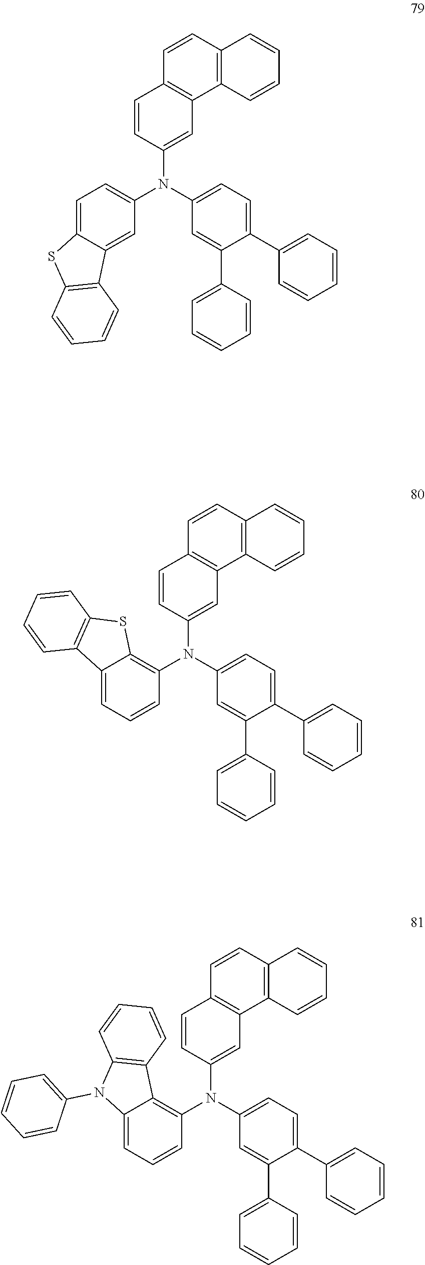 [SMILES: c1ccc(-c2ccc(N(c3ccc4ccc5ccccc5c4c3)c3ccc4sc5ccccc5c4c3)cc2-c2ccccc2)cc1 ; c1ccc(-c2ccc(N(c3ccc4ccc5ccccc5c4c3)c3cccc4c3sc3ccccc34)cc2-c2ccccc2)cc1 ; c1ccc(-c2ccc(N(c3ccc4ccc5ccccc5c4c3)c3cccc4c3c3ccccc3n4-c3ccccc3)cc2-c2ccccc2)cc1]

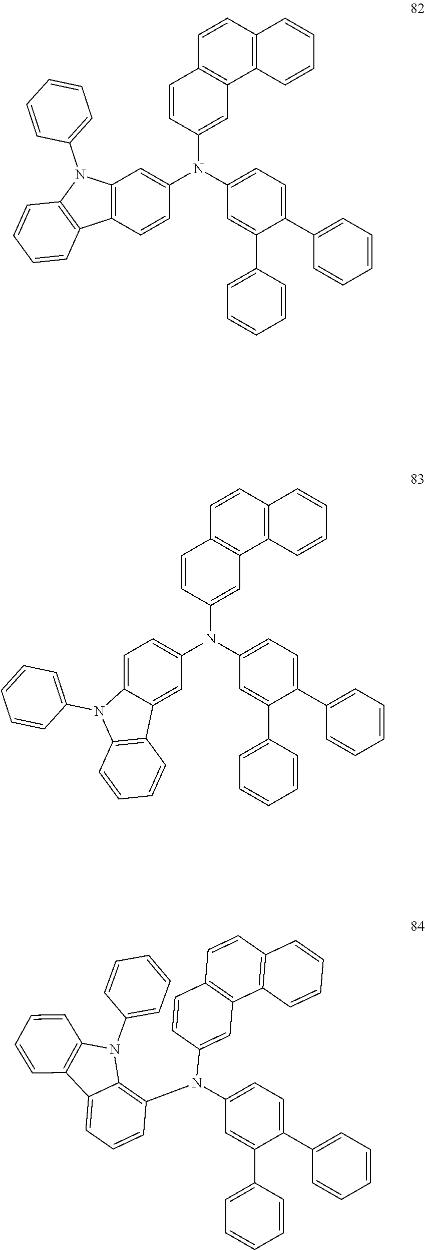 [SMILES: c1ccc(-c2ccc(N(c3ccc4ccc5ccccc5c4c3)c3ccc4c5ccccc5n(-c5ccccc5)c4c3)cc2-c2ccccc2)cc1 ; c1ccc(-c2ccc(N(c3ccc4ccc5ccccc5c4c3)c3ccc4c(c3)c3ccccc3n4-c3ccccc3)cc2-c2ccccc2)cc1 ; c1ccc(-c2ccc(N(c3ccc4ccc5ccccc5c4c3)c3cccc4c5ccccc5n(-c5ccccc5)c34)cc2-c2ccccc2)cc1]

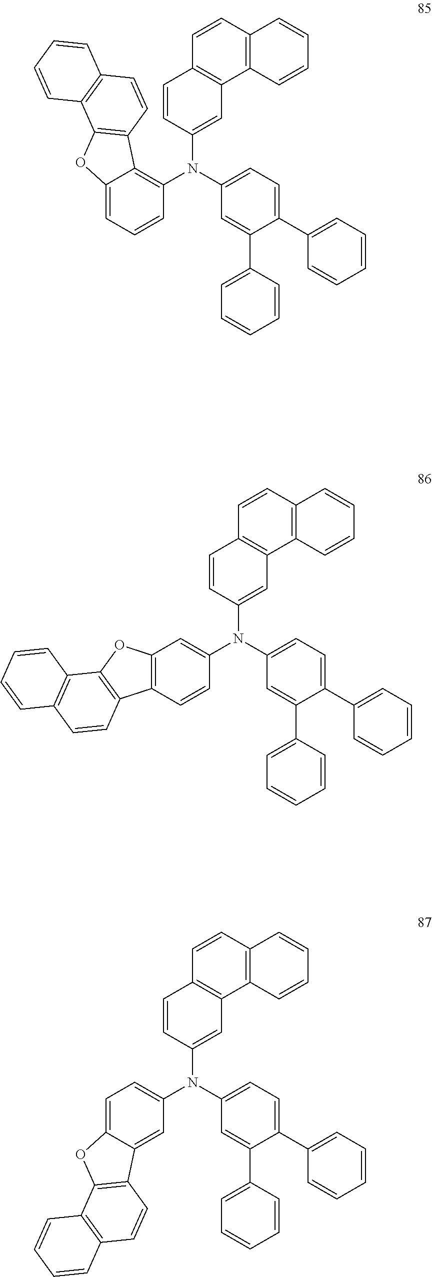 [SMILES: c1ccc(-c2ccc(N(c3ccc4ccc5ccccc5c4c3)c3cccc4oc5c6ccccc6ccc5c34)cc2-c2ccccc2)cc1 ; c1ccc(-c2ccc(N(c3ccc4c(c3)oc3c5ccccc5ccc43)c3ccc4ccc5ccccc5c4c3)cc2-c2ccccc2)cc1 ; c1ccc(-c2ccc(N(c3ccc4ccc5ccccc5c4c3)c3ccc4oc5c6ccccc6ccc5c4c3)cc2-c2ccccc2)cc1]

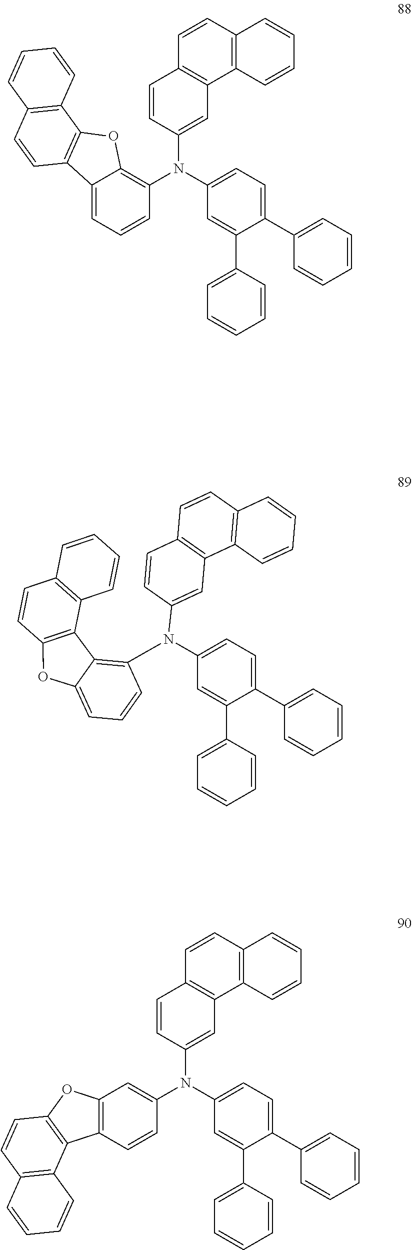 [SMILES: c1ccc(-c2ccc(N(c3ccc4ccc5ccccc5c4c3)c3cccc4c3oc3c5ccccc5ccc43)cc2-c2ccccc2)cc1 ; c1ccc(-c2ccc(N(c3ccc4ccc5ccccc5c4c3)c3cccc4oc5ccc6ccccc6c5c34)cc2-c2ccccc2)cc1 ; c1ccc(-c2ccc(N(c3ccc4c(c3)oc3ccc5ccccc5c34)c3ccc4ccc5ccccc5c4c3)cc2-c2ccccc2)cc1]

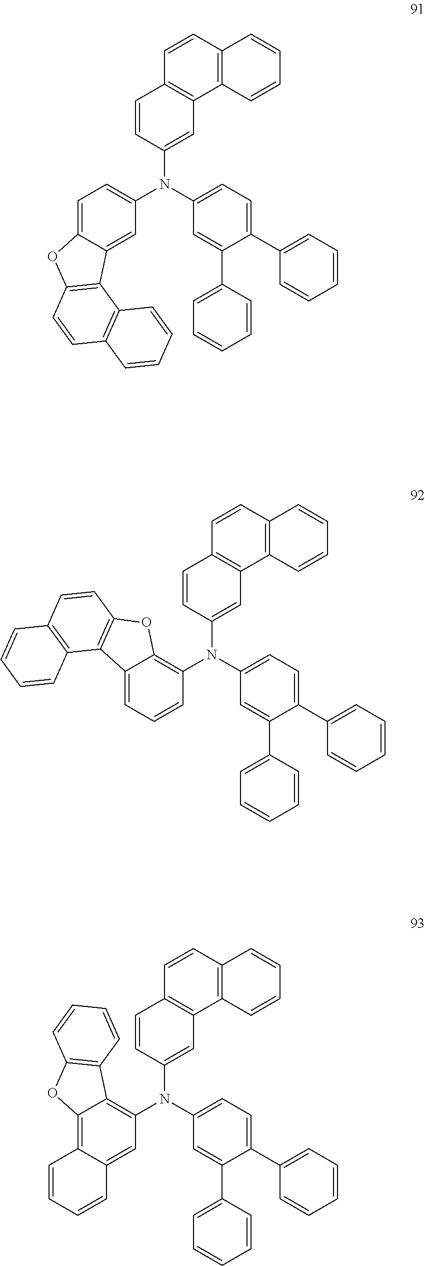 [SMILES: c1ccc(-c2ccc(N(c3ccc4ccc5ccccc5c4c3)c3ccc4oc5ccc6ccccc6c5c4c3)cc2-c2ccccc2)cc1 ; c1ccc(-c2ccc(N(c3ccc4ccc5ccccc5c4c3)c3cccc4c3oc3ccc5ccccc5c34)cc2-c2ccccc2)cc1 ; c1ccc(-c2ccc(N(c3ccc4ccc5ccccc5c4c3)c3cc4ccccc4c4oc5ccccc5c34)cc2-c2ccccc2)cc1]

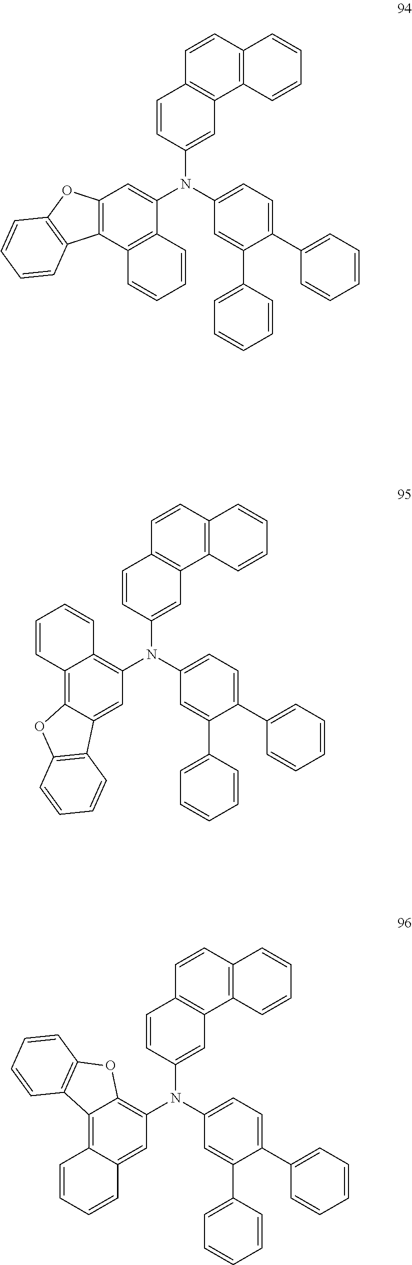 [SMILES: c1ccc(-c2ccc(N(c3ccc4ccc5ccccc5c4c3)c3cc4oc5ccccc5c4c4ccccc34)cc2-c2ccccc2)cc1 ; c1ccc(-c2ccc(N(c3ccc4ccc5ccccc5c4c3)c3cc4c5ccccc5oc4c4ccccc34)cc2-c2ccccc2)cc1 ; c1ccc(-c2ccc(N(c3ccc4ccc5ccccc5c4c3)c3cc4ccccc4c4c3oc3ccccc34)cc2-c2ccccc2)cc1]

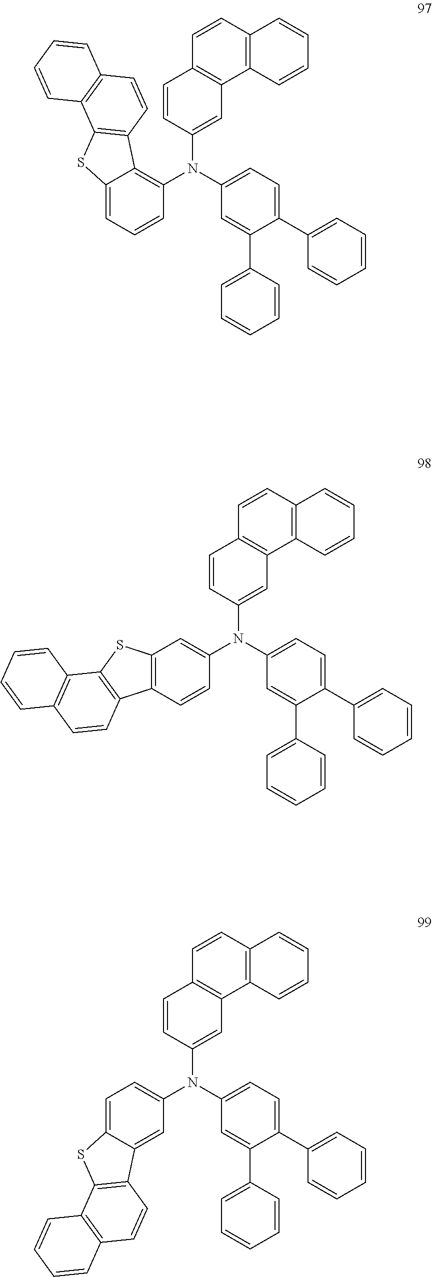 [SMILES: c1ccc(-c2ccc(N(c3ccc4ccc5ccccc5c4c3)c3cccc4sc5c6ccccc6ccc5c34)cc2-c2ccccc2)cc1 ; c1ccc(-c2ccc(N(c3ccc4c(c3)sc3c5ccccc5ccc43)c3ccc4ccc5ccccc5c4c3)cc2-c2ccccc2)cc1 ; c1ccc(-c2ccc(N(c3ccc4ccc5ccccc5c4c3)c3ccc4sc5c6ccccc6ccc5c4c3)cc2-c2ccccc2)cc1]

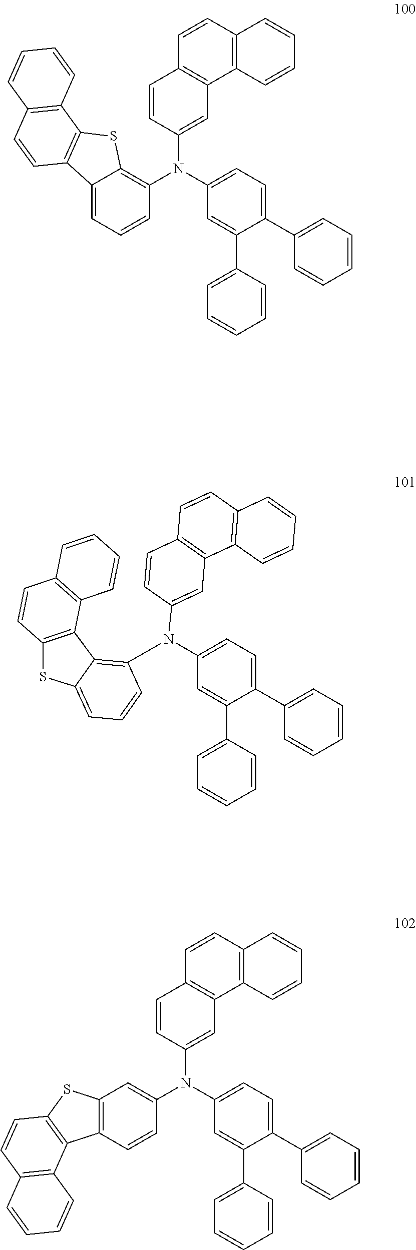 [SMILES: c1ccc(-c2ccc(N(c3ccc4ccc5ccccc5c4c3)c3cccc4c3sc3c5ccccc5ccc43)cc2-c2ccccc2)cc1 ; c1ccc(-c2ccc(N(c3ccc4ccc5ccccc5c4c3)c3cccc4sc5ccc6ccccc6c5c34)cc2-c2ccccc2)cc1 ; c1ccc(-c2ccc(N(c3ccc4c(c3)sc3ccc5ccccc5c34)c3ccc4ccc5ccccc5c4c3)cc2-c2ccccc2)cc1]

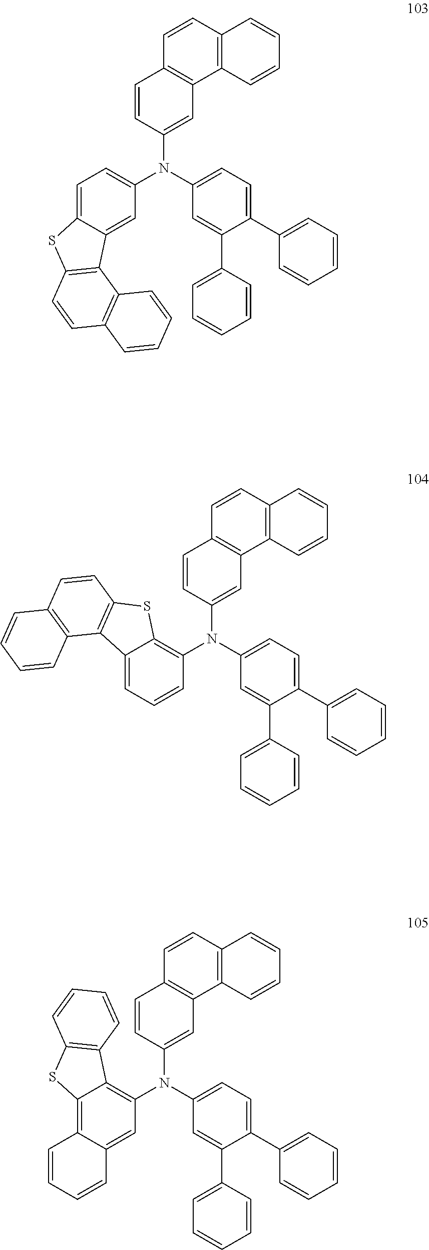 [SMILES: c1ccc(-c2ccc(N(c3ccc4ccc5ccccc5c4c3)c3ccc4sc5ccc6ccccc6c5c4c3)cc2-c2ccccc2)cc1 ; c1ccc(-c2ccc(N(c3ccc4ccc5ccccc5c4c3)c3cccc4c3sc3ccc5ccccc5c34)cc2-c2ccccc2)cc1 ; c1ccc(-c2ccc(N(c3ccc4ccc5ccccc5c4c3)c3cc4ccccc4c4sc5ccccc5c34)cc2-c2ccccc2)cc1]

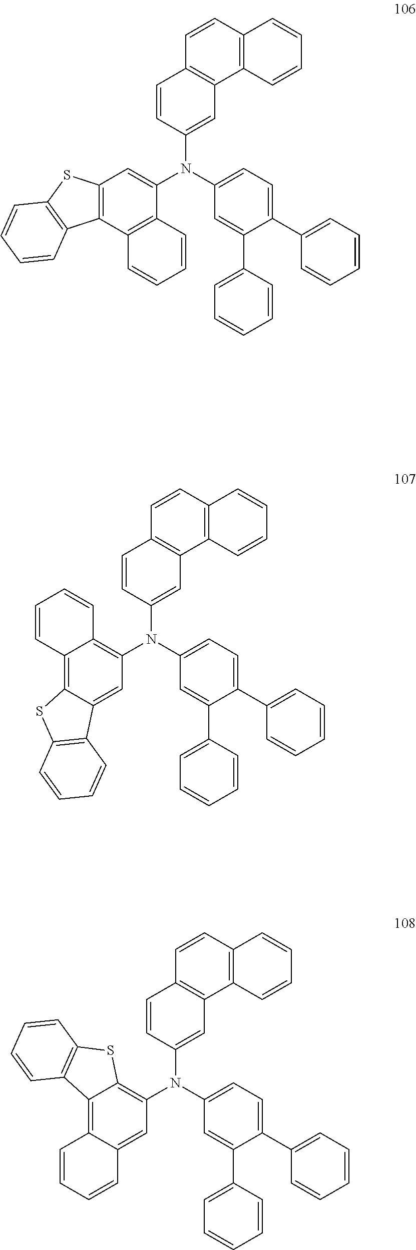 [SMILES: c1ccc(-c2ccc(N(c3ccc4ccc5ccccc5c4c3)c3cc4sc5ccccc5c4c4ccccc34)cc2-c2ccccc2)cc1 ; c1ccc(-c2ccc(N(c3ccc4ccc5ccccc5c4c3)c3cc4c5ccccc5sc4c4ccccc34)cc2-c2ccccc2)cc1 ; c1ccc(-c2ccc(N(c3ccc4ccc5ccccc5c4c3)c3cc4ccccc4c4c3sc3ccccc34)cc2-c2ccccc2)cc1]

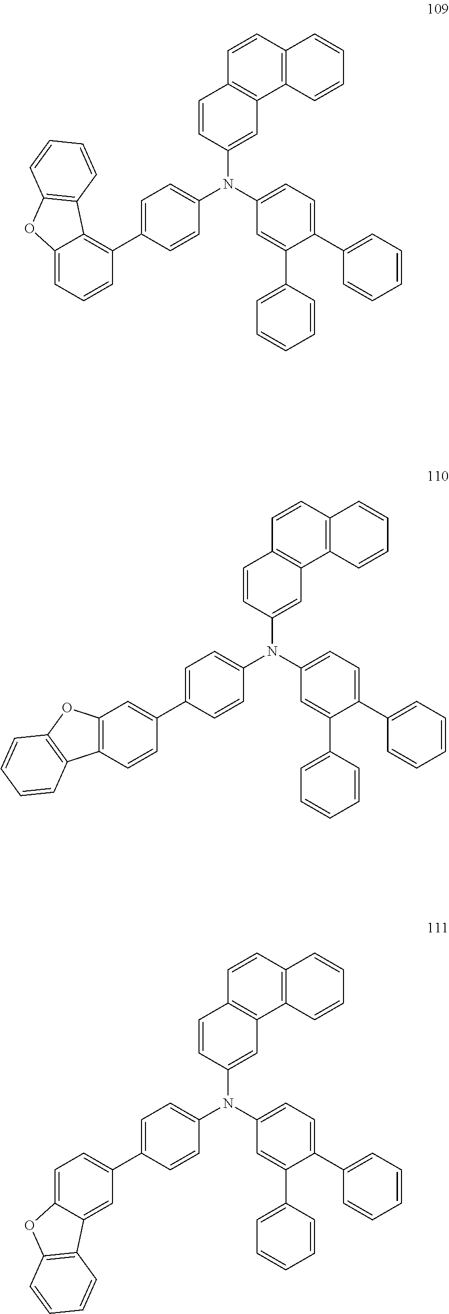 [SMILES: c1ccc(-c2ccc(N(c3ccc(-c4cccc5oc6ccccc6c45)cc3)c3ccc4ccc5ccccc5c4c3)cc2-c2ccccc2)cc1 ; c1ccc(-c2ccc(N(c3ccc(-c4ccc5c(c4)oc4ccccc45)cc3)c3ccc4ccc5ccccc5c4c3)cc2-c2ccccc2)cc1 ; c1ccc(-c2ccc(N(c3ccc(-c4ccc5oc6ccccc6c5c4)cc3)c3ccc4ccc5ccccc5c4c3)cc2-c2ccccc2)cc1]

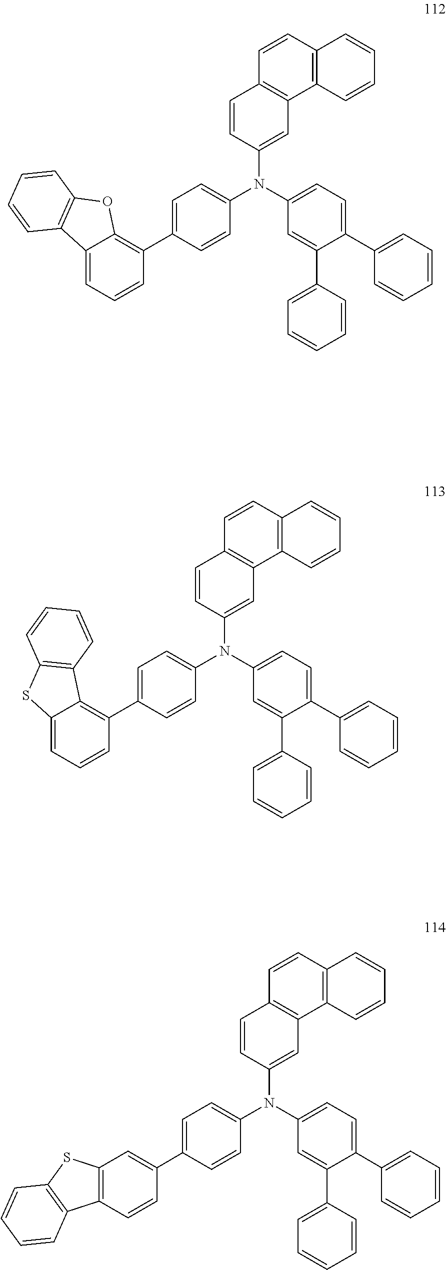 [SMILES: c1ccc(-c2ccc(N(c3ccc(-c4cccc5c4oc4ccccc45)cc3)c3ccc4ccc5ccccc5c4c3)cc2-c2ccccc2)cc1 ; c1ccc(-c2ccc(N(c3ccc(-c4cccc5sc6ccccc6c45)cc3)c3ccc4ccc5ccccc5c4c3)cc2-c2ccccc2)cc1 ; c1ccc(-c2ccc(N(c3ccc(-c4ccc5c(c4)sc4ccccc45)cc3)c3ccc4ccc5ccccc5c4c3)cc2-c2ccccc2)cc1]

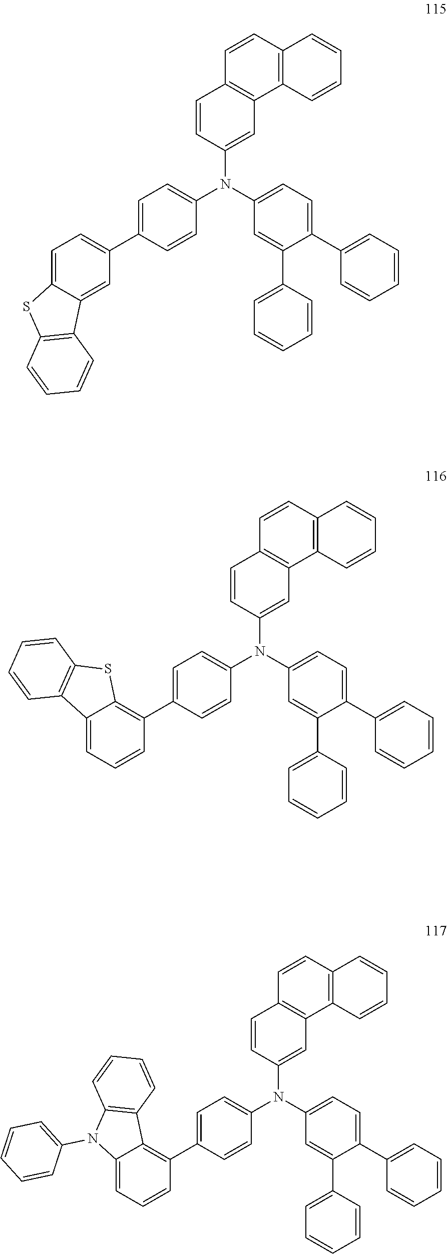 [SMILES: c1ccc(-c2ccc(N(c3ccc(-c4ccc5sc6ccccc6c5c4)cc3)c3ccc4ccc5ccccc5c4c3)cc2-c2ccccc2)cc1 ; c1ccc(-c2ccc(N(c3ccc(-c4cccc5c4sc4ccccc45)cc3)c3ccc4ccc5ccccc5c4c3)cc2-c2ccccc2)cc1 ; c1ccc(-c2ccc(N(c3ccc(-c4cccc5c4c4ccccc4n5-c4ccccc4)cc3)c3ccc4ccc5ccccc5c4c3)cc2-c2ccccc2)cc1]

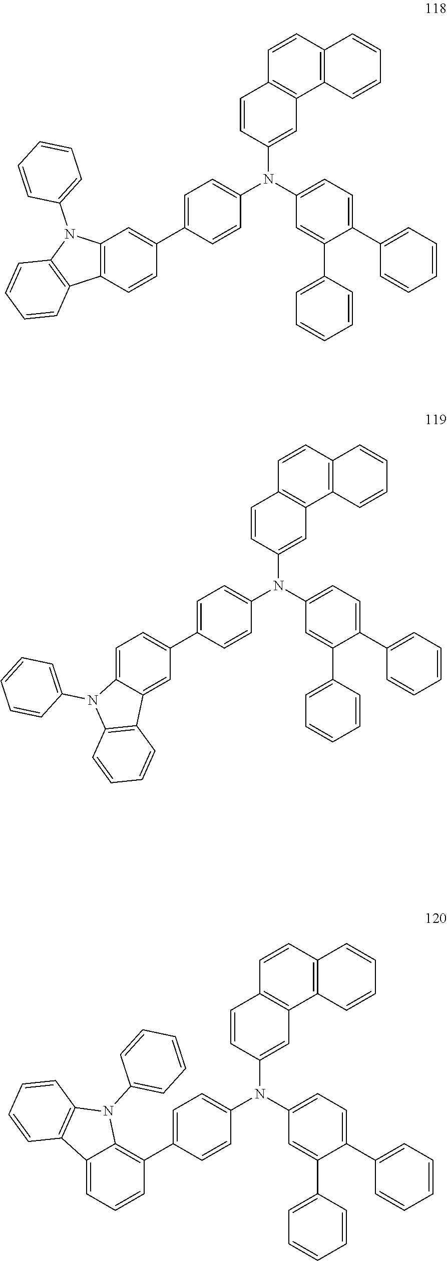 [SMILES: c1ccc(-c2ccc(N(c3ccc(-c4ccc5c6ccccc6n(-c6ccccc6)c5c4)cc3)c3ccc4ccc5ccccc5c4c3)cc2-c2ccccc2)cc1 ; c1ccc(-c2ccc(N(c3ccc(-c4ccc5c(c4)c4ccccc4n5-c4ccccc4)cc3)c3ccc4ccc5ccccc5c4c3)cc2-c2ccccc2)cc1 ; c1ccc(-c2ccc(N(c3ccc(-c4cccc5c6ccccc6n(-c6ccccc6)c45)cc3)c3ccc4ccc5ccccc5c4c3)cc2-c2ccccc2)cc1]

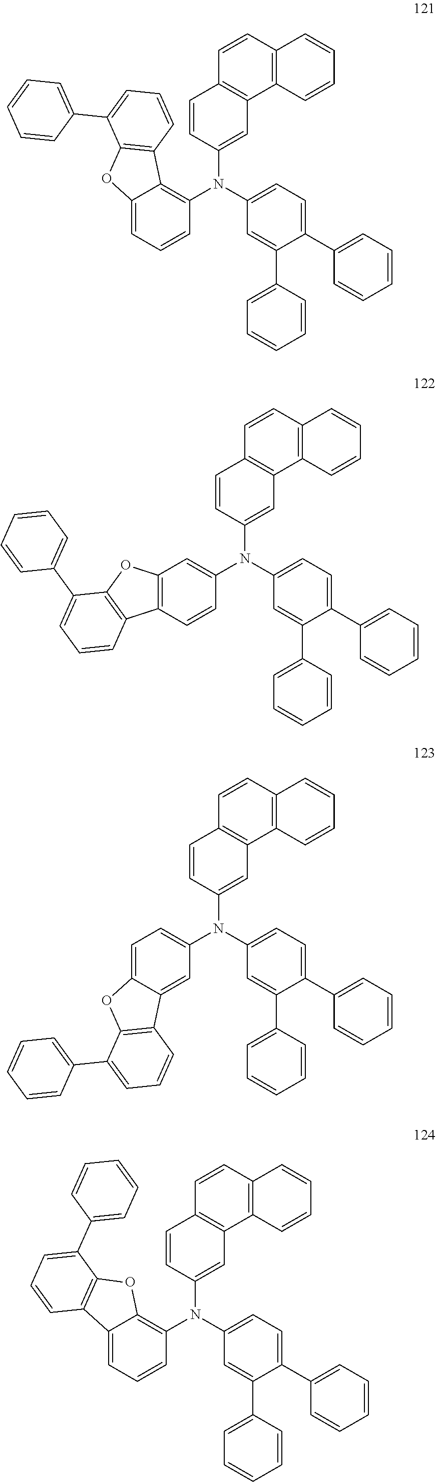 [SMILES: c1ccc(-c2ccc(N(c3ccc4ccc5ccccc5c4c3)c3cccc4oc5c(-c6ccccc6)cccc5c34)cc2-c2ccccc2)cc1 ; c1ccc(-c2ccc(N(c3ccc4c(c3)oc3c(-c5ccccc5)cccc34)c3ccc4ccc5ccccc5c4c3)cc2-c2ccccc2)cc1 ; c1ccc(-c2ccc(N(c3ccc4ccc5ccccc5c4c3)c3ccc4oc5c(-c6ccccc6)cccc5c4c3)cc2-c2ccccc2)cc1 ; c1ccc(-c2ccc(N(c3ccc4ccc5ccccc5c4c3)c3cccc4c3oc3c(-c5ccccc5)cccc34)cc2-c2ccccc2)cc1]

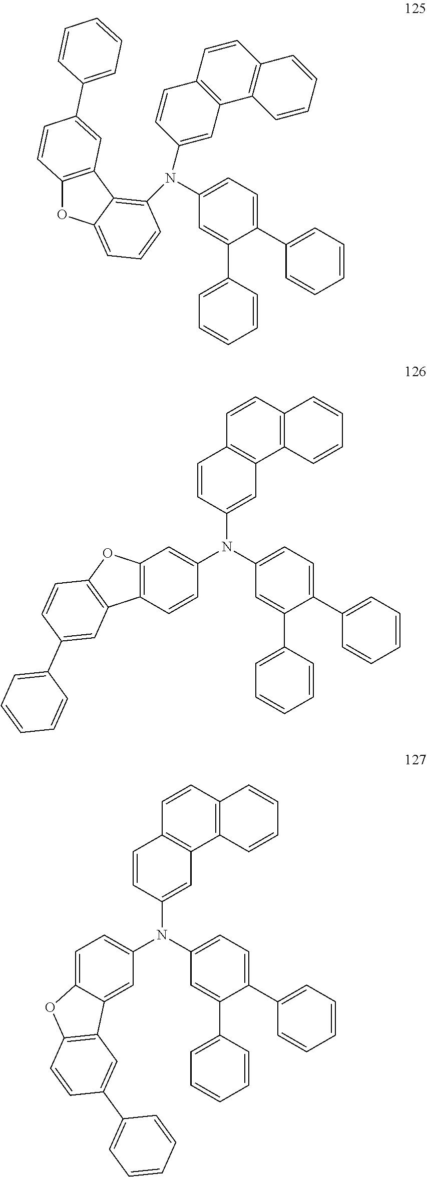 [SMILES: c1ccc(-c2ccc3oc4cccc(N(c5ccc(-c6ccccc6)c(-c6ccccc6)c5)c5ccc6ccc7ccccc7c6c5)c4c3c2)cc1 ; c1ccc(-c2ccc3oc4cc(N(c5ccc(-c6ccccc6)c(-c6ccccc6)c5)c5ccc6ccc7ccccc7c6c5)ccc4c3c2)cc1 ; c1ccc(-c2ccc3oc4ccc(N(c5ccc(-c6ccccc6)c(-c6ccccc6)c5)c5ccc6ccc7ccccc7c6c5)cc4c3c2)cc1]

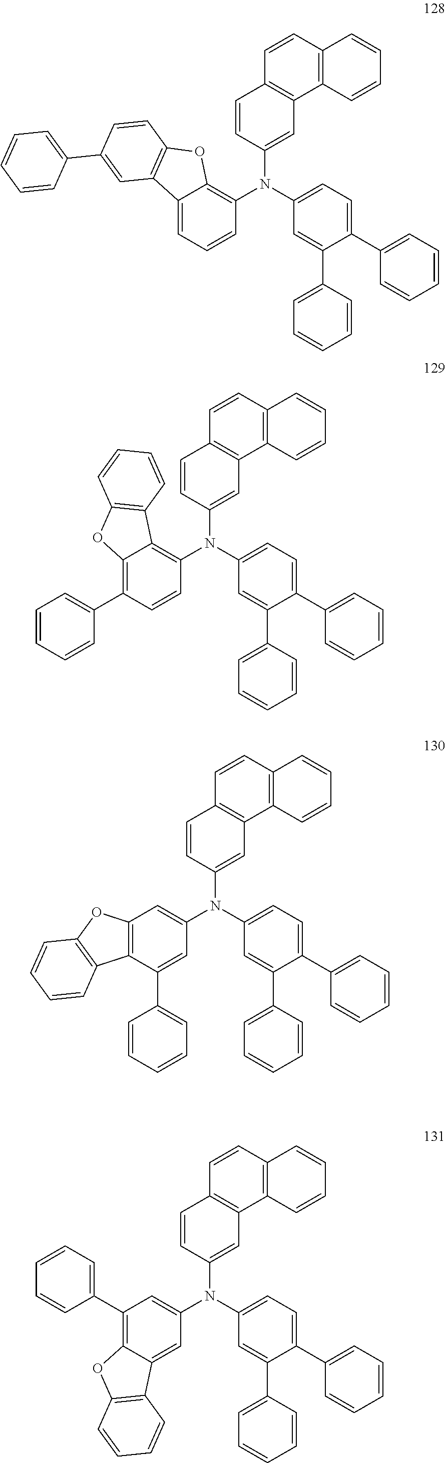 [SMILES: c1ccc(-c2ccc3oc4c(N(c5ccc(-c6ccccc6)c(-c6ccccc6)c5)c5ccc6ccc7ccccc7c6c5)cccc4c3c2)cc1 ; c1ccc(-c2ccc(N(c3ccc4ccc5ccccc5c4c3)c3ccc(-c4ccccc4)c4oc5ccccc5c34)cc2-c2ccccc2)cc1 ; c1ccc(-c2ccc(N(c3cc(-c4ccccc4)c4c(c3)oc3ccccc34)c3ccc4ccc5ccccc5c4c3)cc2-c2ccccc2)cc1 ; c1ccc(-c2ccc(N(c3ccc4ccc5ccccc5c4c3)c3cc(-c4ccccc4)c4oc5ccccc5c4c3)cc2-c2ccccc2)cc1]

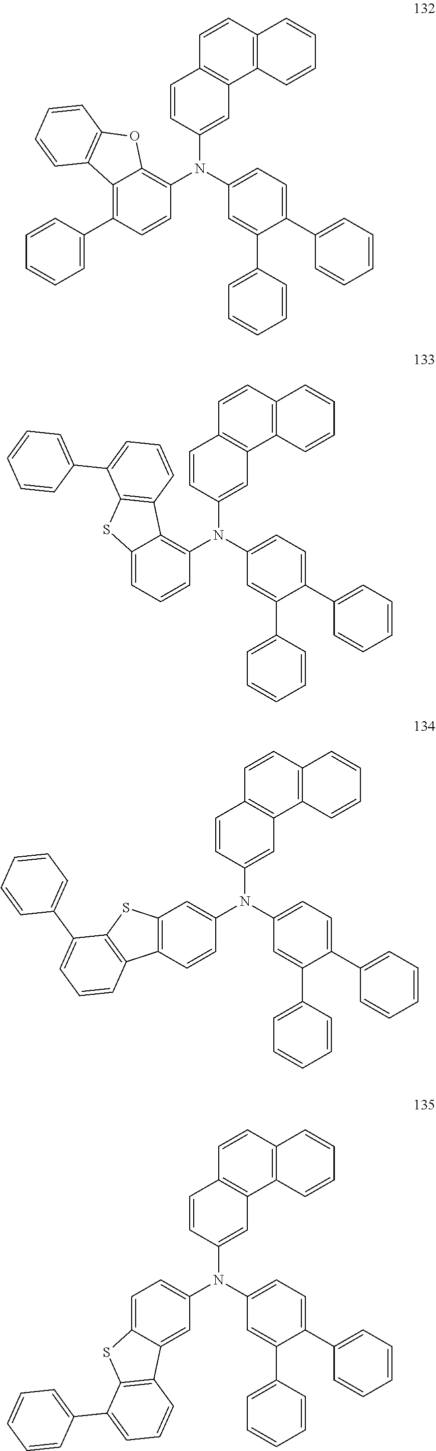 [SMILES: c1ccc(-c2ccc(N(c3ccc4ccc5ccccc5c4c3)c3ccc(-c4ccccc4)c4c3oc3ccccc34)cc2-c2ccccc2)cc1 ; c1ccc(-c2ccc(N(c3ccc4ccc5ccccc5c4c3)c3cccc4sc5c(-c6ccccc6)cccc5c34)cc2-c2ccccc2)cc1 ; c1ccc(-c2ccc(N(c3ccc4c(c3)sc3c(-c5ccccc5)cccc34)c3ccc4ccc5ccccc5c4c3)cc2-c2ccccc2)cc1 ; c1ccc(-c2ccc(N(c3ccc4ccc5ccccc5c4c3)c3ccc4sc5c(-c6ccccc6)cccc5c4c3)cc2-c2ccccc2)cc1]

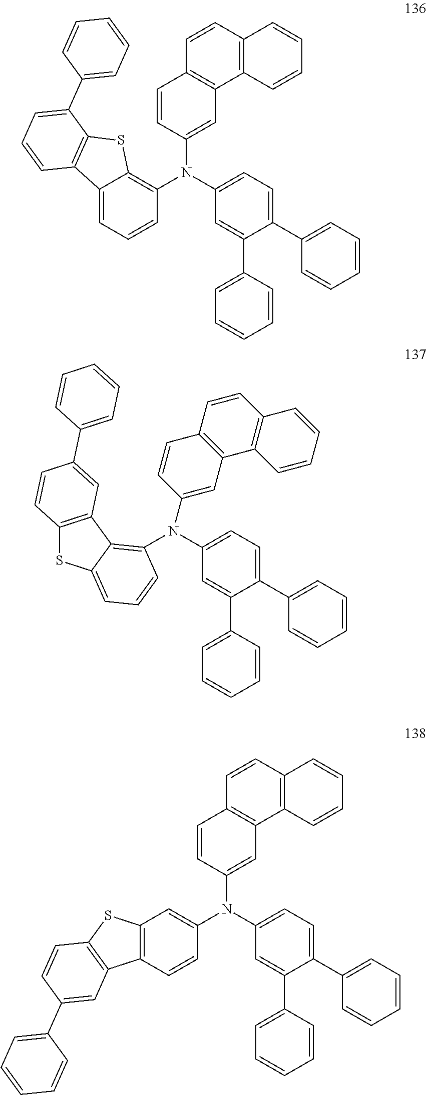 [SMILES: c1ccc(-c2ccc(N(c3ccc4ccc5ccccc5c4c3)c3cccc4c3sc3c(-c5ccccc5)cccc34)cc2-c2ccccc2)cc1 ; c1ccc(-c2ccc3sc4cccc(N(c5ccc(-c6ccccc6)c(-c6ccccc6)c5)c5ccc6ccc7ccccc7c6c5)c4c3c2)cc1 ; c1ccc(-c2ccc3sc4cc(N(c5ccc(-c6ccccc6)c(-c6ccccc6)c5)c5ccc6ccc7ccccc7c6c5)ccc4c3c2)cc1]

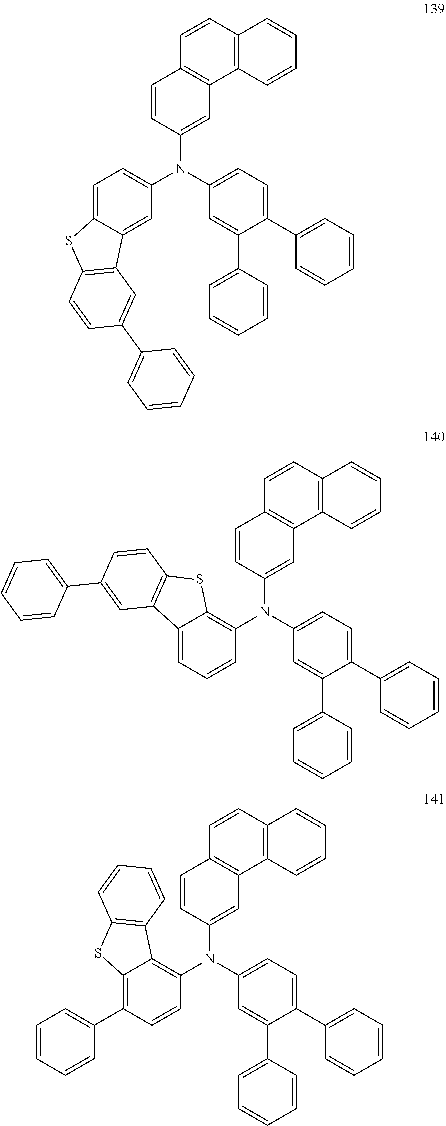

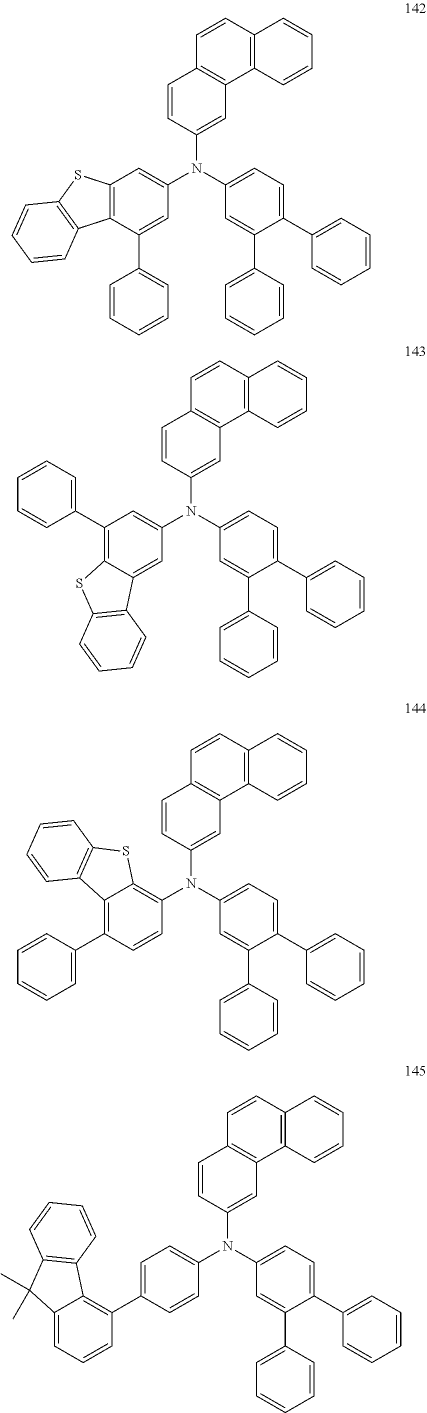

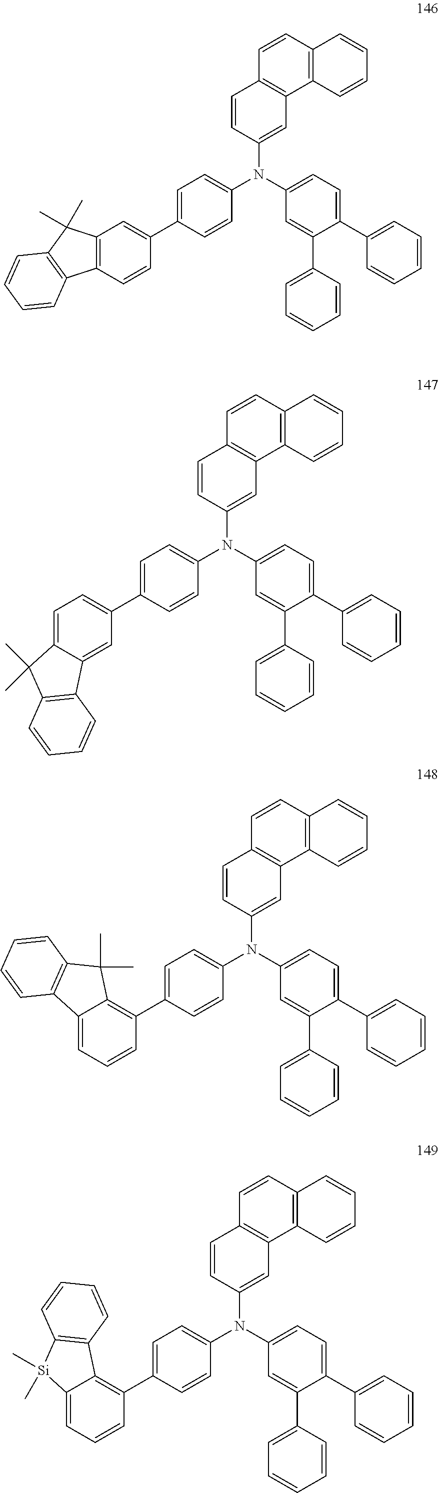 [SMILES: CC1(C)c2ccccc2-c2ccc(-c3ccc(N(c4ccc(-c5ccccc5)c(-c5ccccc5)c4)c4ccc5ccc6ccccc6c5c4)cc3)cc21 ; CC1(C)c2ccccc2-c2cc(-c3ccc(N(c4ccc(-c5ccccc5)c(-c5ccccc5)c4)c4ccc5ccc6ccccc6c5c4)cc3)ccc21 ; CC1(C)c2ccccc2-c2cccc(-c3ccc(N(c4ccc(-c5ccccc5)c(-c5ccccc5)c4)c4ccc5ccc6ccccc6c5c4)cc3)c21 ; C[Si]1(C)c2ccccc2-c2c(-c3ccc(N(c4ccc(-c5ccccc5)c(-c5ccccc5)c4)c4ccc5ccc6ccccc6c5c4)cc3)cccc21]

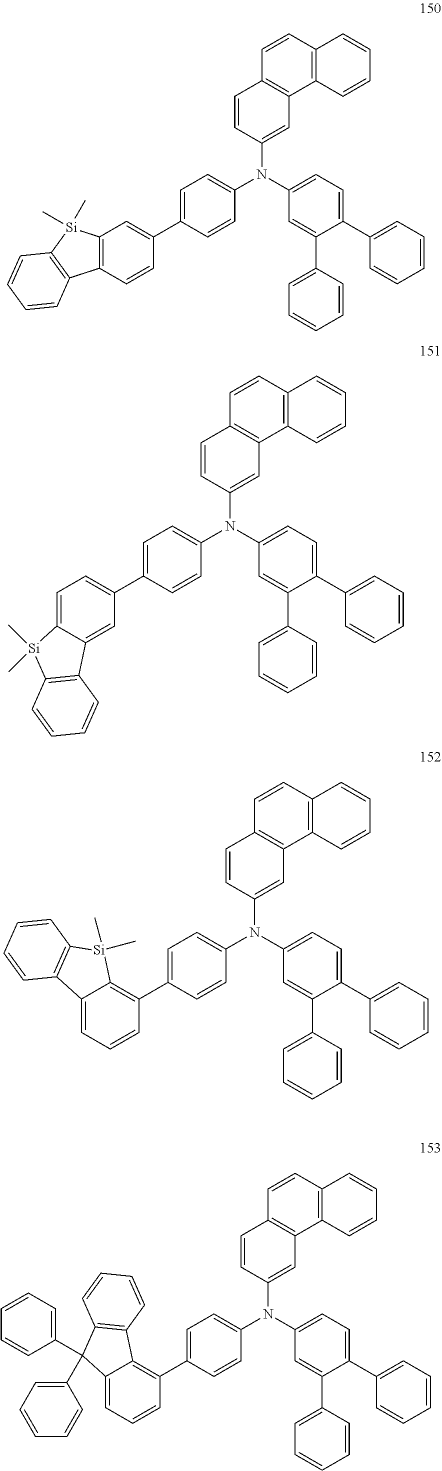 [SMILES: C[Si]1(C)c2ccccc2-c2ccc(-c3ccc(N(c4ccc(-c5ccccc5)c(-c5ccccc5)c4)c4ccc5ccc6ccccc6c5c4)cc3)cc21 ; C[Si]1(C)c2ccccc2-c2cc(-c3ccc(N(c4ccc(-c5ccccc5)c(-c5ccccc5)c4)c4ccc5ccc6ccccc6c5c4)cc3)ccc21 ; C[Si]1(C)c2ccccc2-c2cccc(-c3ccc(N(c4ccc(-c5ccccc5)c(-c5ccccc5)c4)c4ccc5ccc6ccccc6c5c4)cc3)c21 ; c1ccc(-c2ccc(N(c3ccc(-c4cccc5c4-c4ccccc4C5(c4ccccc4)c4ccccc4)cc3)c3ccc4ccc5ccccc5c4c3)cc2-c2ccccc2)cc1]

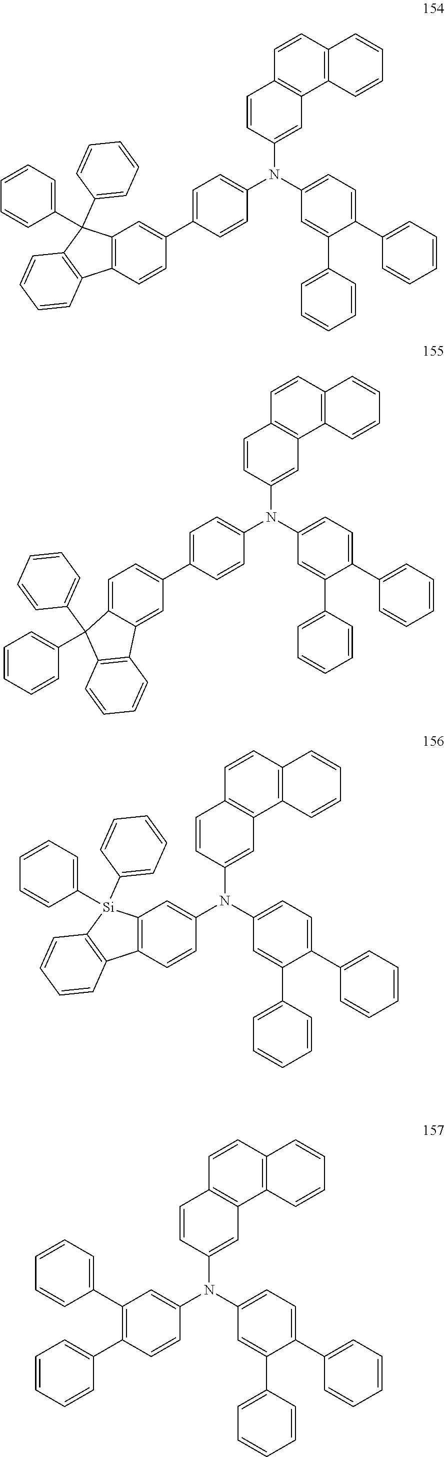 [SMILES: c1ccc(-c2ccc(N(c3ccc(-c4ccc5c(c4)C(c4ccccc4)(c4ccccc4)c4ccccc4-5)cc3)c3ccc4ccc5ccccc5c4c3)cc2-c2ccccc2)cc1 ; c1ccc(-c2ccc(N(c3ccc(-c4ccc5c(c4)-c4ccccc4C5(c4ccccc4)c4ccccc4)cc3)c3ccc4ccc5ccccc5c4c3)cc2-c2ccccc2)cc1 ; c1ccc(-c2ccc(N(c3ccc4c(c3)[Si](c3ccccc3)(c3ccccc3)c3ccccc3-4)c3ccc4ccc5ccccc5c4c3)cc2-c2ccccc2)cc1 ; c1ccc(-c2ccc(N(c3ccc(-c4ccccc4)c(-c4ccccc4)c3)c3ccc4ccc5ccccc5c4c3)cc2-c2ccccc2)cc1]

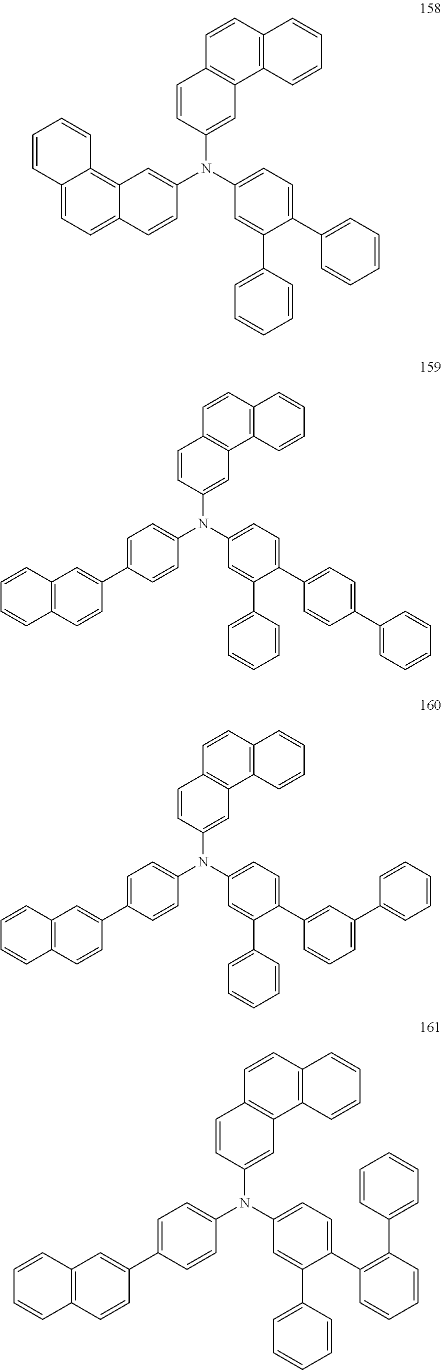 [SMILES: c1ccc(-c2ccc(N(c3ccc4ccc5ccccc5c4c3)c3ccc4ccc5ccccc5c4c3)cc2-c2ccccc2)cc1 ; c1ccc(-c2ccc(-c3ccc(N(c4ccc(-c5ccc6ccccc6c5)cc4)c4ccc5ccc6ccccc6c5c4)cc3-c3ccccc3)cc2)cc1 ; c1ccc(-c2cccc(-c3ccc(N(c4ccc(-c5ccc6ccccc6c5)cc4)c4ccc5ccc6ccccc6c5c4)cc3-c3ccccc3)c2)cc1 ; c1ccc(-c2ccccc2-c2ccc(N(c3ccc(-c4ccc5ccccc5c4)cc3)c3ccc4ccc5ccccc5c4c3)cc2-c2ccccc2)cc1]

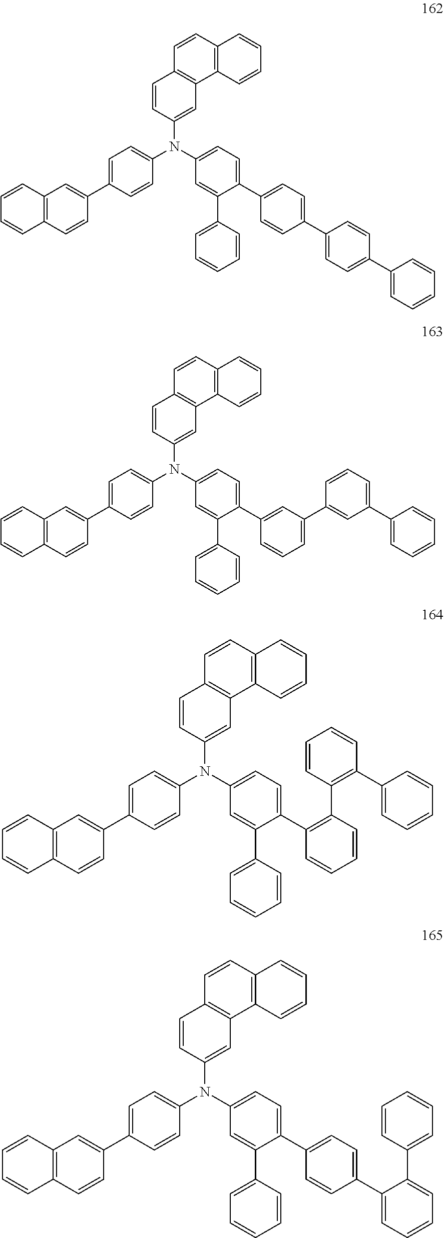 [SMILES: c1ccc(-c2ccc(-c3ccc(-c4ccc(N(c5ccc(-c6ccc7ccccc7c6)cc5)c5ccc6ccc7ccccc7c6c5)cc4-c4ccccc4)cc3)cc2)cc1 ; c1ccc(-c2cccc(-c3cccc(-c4ccc(N(c5ccc(-c6ccc7ccccc7c6)cc5)c5ccc6ccc7ccccc7c6c5)cc4-c4ccccc4)c3)c2)cc1 ; c1ccc(-c2ccccc2-c2ccccc2-c2ccc(N(c3ccc(-c4ccc5ccccc5c4)cc3)c3ccc4ccc5ccccc5c4c3)cc2-c2ccccc2)cc1 ; c1ccc(-c2ccccc2-c2ccc(-c3ccc(N(c4ccc(-c5ccc6ccccc6c5)cc4)c4ccc5ccc6ccccc6c5c4)cc3-c3ccccc3)cc2)cc1]

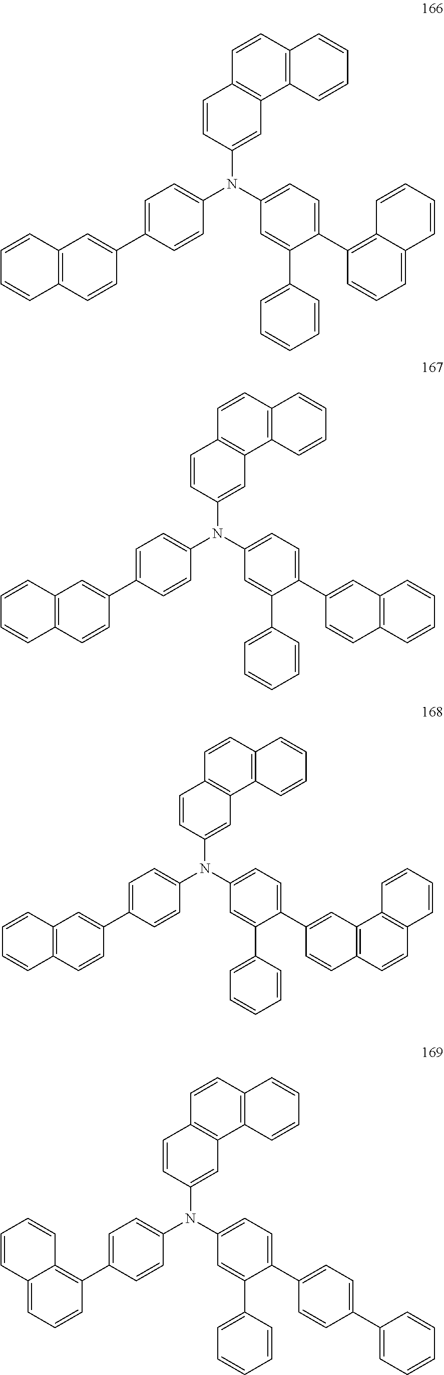

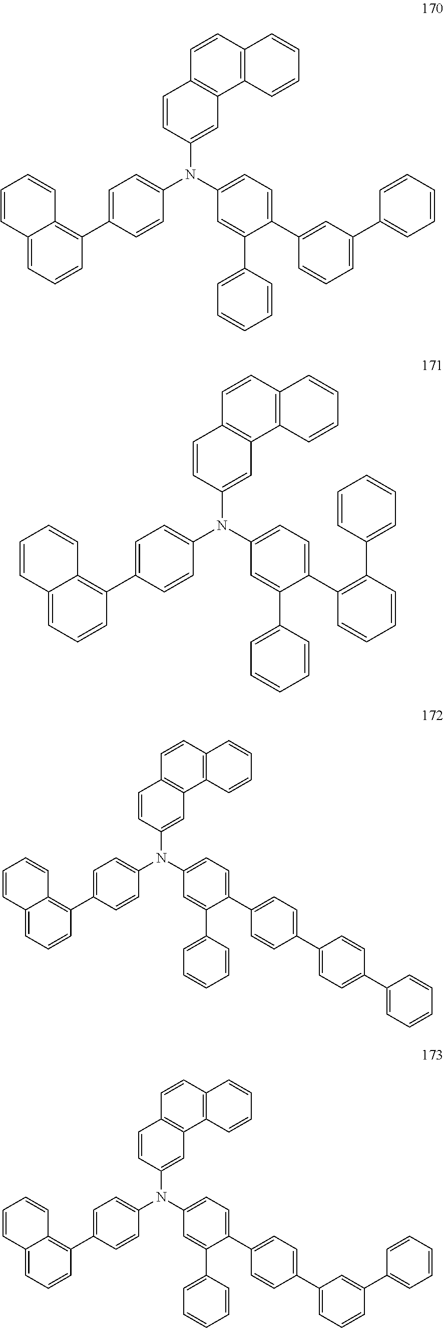 [SMILES: c1ccc(-c2cccc(-c3ccc(N(c4ccc(-c5cccc6ccccc56)cc4)c4ccc5ccc6ccccc6c5c4)cc3-c3ccccc3)c2)cc1 ; c1ccc(-c2ccccc2-c2ccc(N(c3ccc(-c4cccc5ccccc45)cc3)c3ccc4ccc5ccccc5c4c3)cc2-c2ccccc2)cc1 ; c1ccc(-c2ccc(-c3ccc(-c4ccc(N(c5ccc(-c6cccc7ccccc67)cc5)c5ccc6ccc7ccccc7c6c5)cc4-c4ccccc4)cc3)cc2)cc1 ; c1ccc(-c2cccc(-c3ccc(-c4ccc(N(c5ccc(-c6cccc7ccccc67)cc5)c5ccc6ccc7ccccc7c6c5)cc4-c4ccccc4)cc3)c2)cc1]

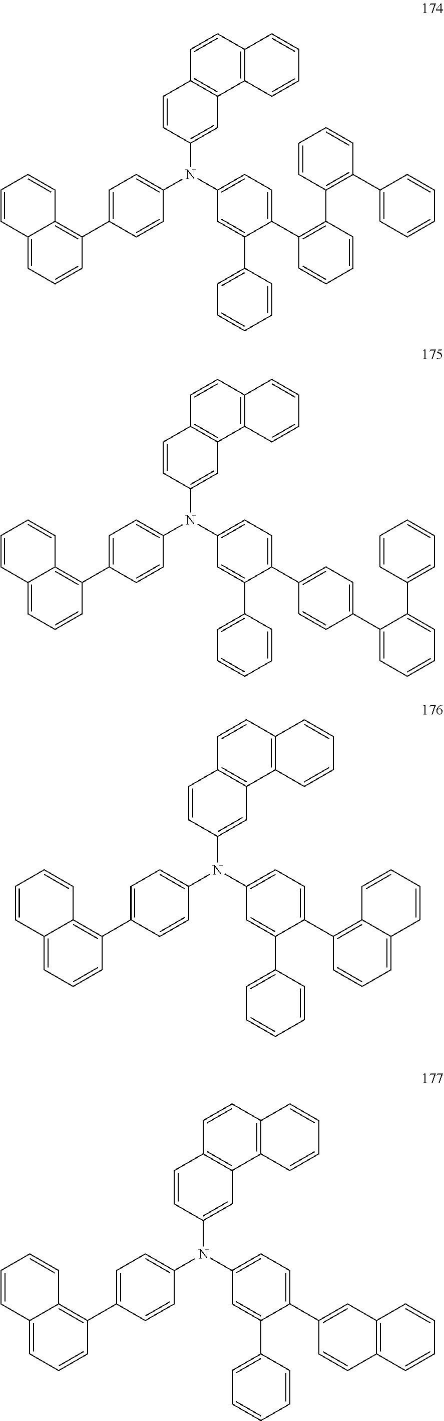

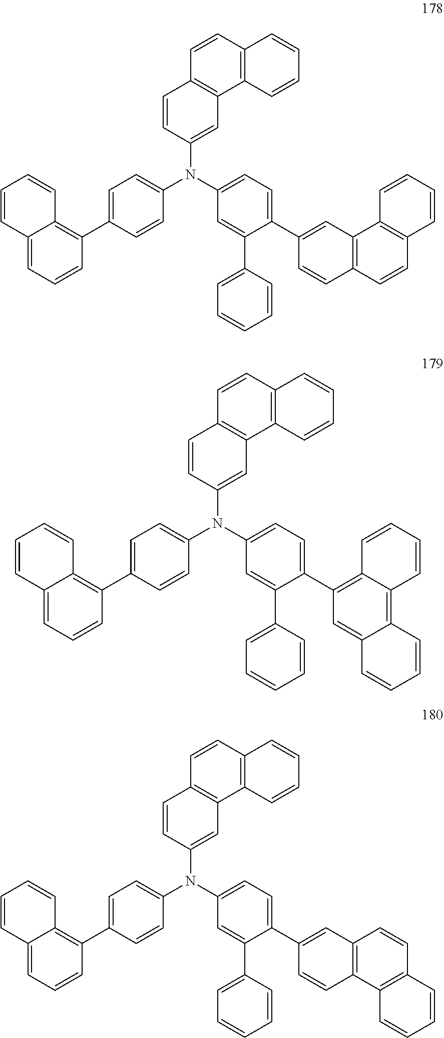

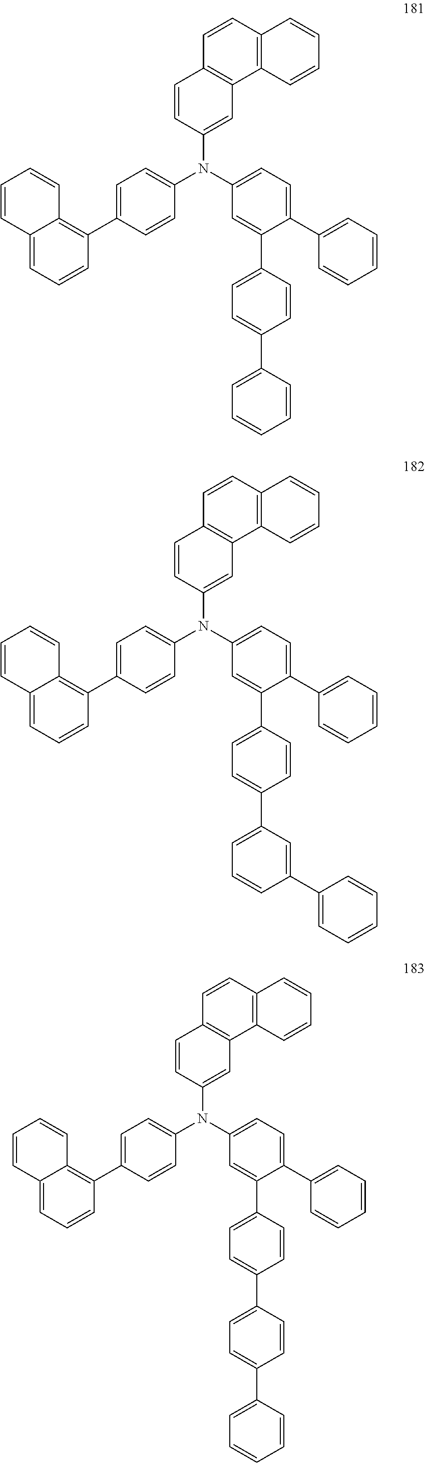 [SMILES: c1ccc(-c2ccc(-c3cc(N(c4ccc(-c5cccc6ccccc56)cc4)c4ccc5ccc6ccccc6c5c4)ccc3-c3ccccc3)cc2)cc1 ; c1ccc(-c2cccc(-c3ccc(-c4cc(N(c5ccc(-c6cccc7ccccc67)cc5)c5ccc6ccc7ccccc7c6c5)ccc4-c4ccccc4)cc3)c2)cc1 ; c1ccc(-c2ccc(-c3ccc(-c4cc(N(c5ccc(-c6cccc7ccccc67)cc5)c5ccc6ccc7ccccc7c6c5)ccc4-c4ccccc4)cc3)cc2)cc1]

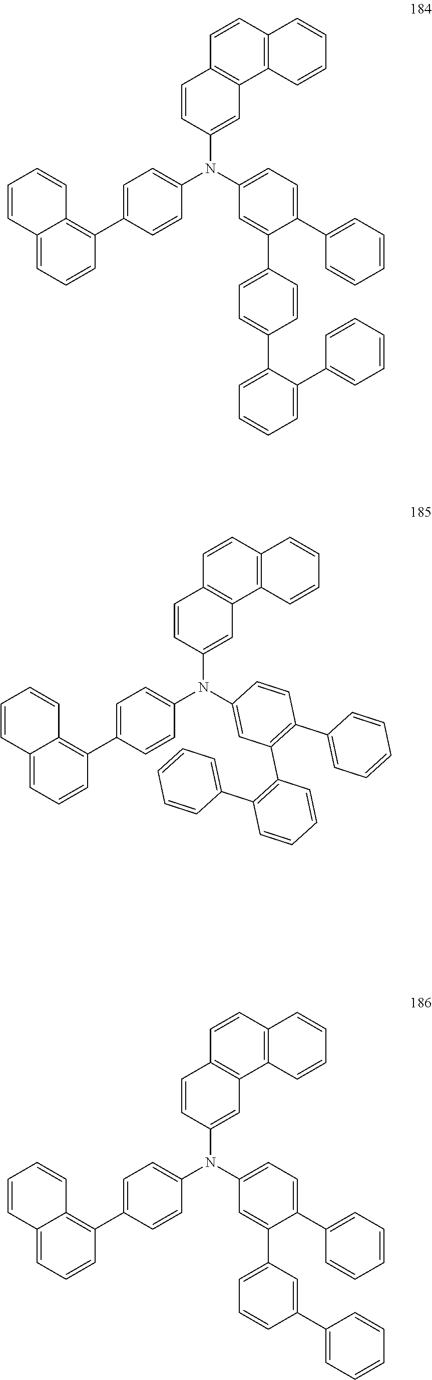 [SMILES: c1ccc(-c2ccccc2-c2ccc(-c3cc(N(c4ccc(-c5cccc6ccccc56)cc4)c4ccc5ccc6ccccc6c5c4)ccc3-c3ccccc3)cc2)cc1 ; c1ccc(-c2ccccc2-c2cc(N(c3ccc(-c4cccc5ccccc45)cc3)c3ccc4ccc5ccccc5c4c3)ccc2-c2ccccc2)cc1 ; c1ccc(-c2cccc(-c3cc(N(c4ccc(-c5cccc6ccccc56)cc4)c4ccc5ccc6ccccc6c5c4)ccc3-c3ccccc3)c2)cc1]

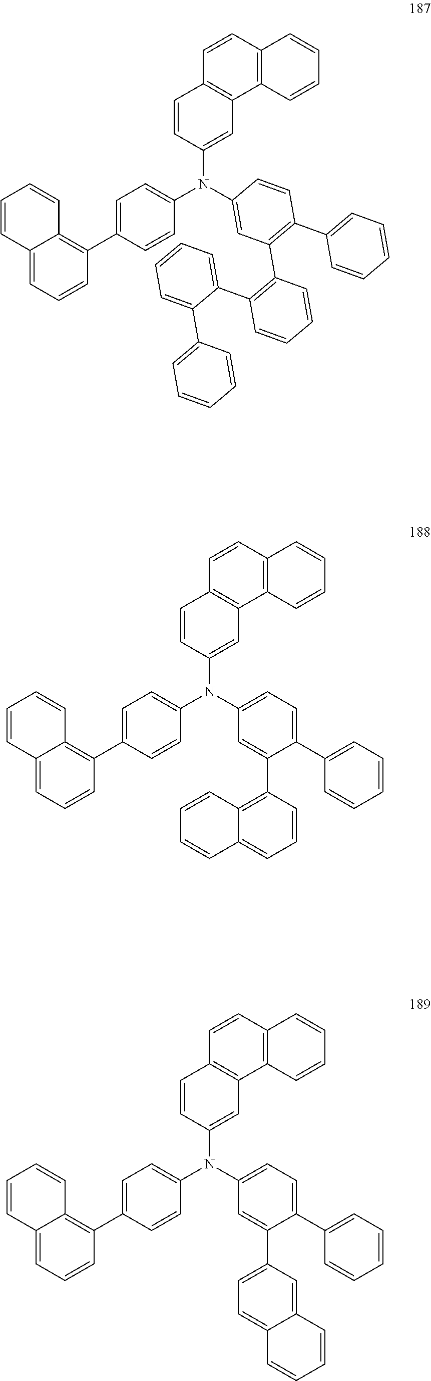 [SMILES: c1ccc(-c2ccccc2-c2ccccc2-c2cc(N(c3ccc(-c4cccc5ccccc45)cc3)c3ccc4ccc5ccccc5c4c3)ccc2-c2ccccc2)cc1 ; c1ccc(-c2ccc(N(c3ccc(-c4cccc5ccccc45)cc3)c3ccc4ccc5ccccc5c4c3)cc2-c2cccc3ccccc23)cc1 ; c1ccc(-c2ccc(N(c3ccc(-c4cccc5ccccc45)cc3)c3ccc4ccc5ccccc5c4c3)cc2-c2ccc3ccccc3c2)cc1]

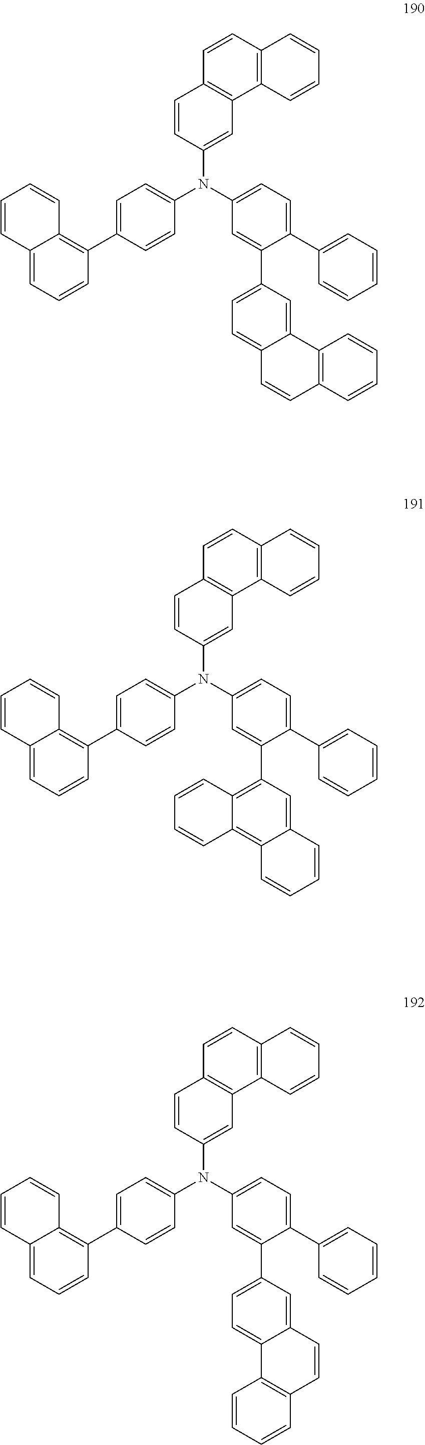 [SMILES: c1ccc(-c2ccc(N(c3ccc(-c4cccc5ccccc45)cc3)c3ccc4ccc5ccccc5c4c3)cc2-c2ccc3ccc4ccccc4c3c2)cc1 ; c1ccc(-c2ccc(N(c3ccc(-c4cccc5ccccc45)cc3)c3ccc4ccc5ccccc5c4c3)cc2-c2cc3ccccc3c3ccccc23)cc1 ; c1ccc(-c2ccc(N(c3ccc(-c4cccc5ccccc45)cc3)c3ccc4ccc5ccccc5c4c3)cc2-c2ccc3c(ccc4ccccc43)c2)cc1]

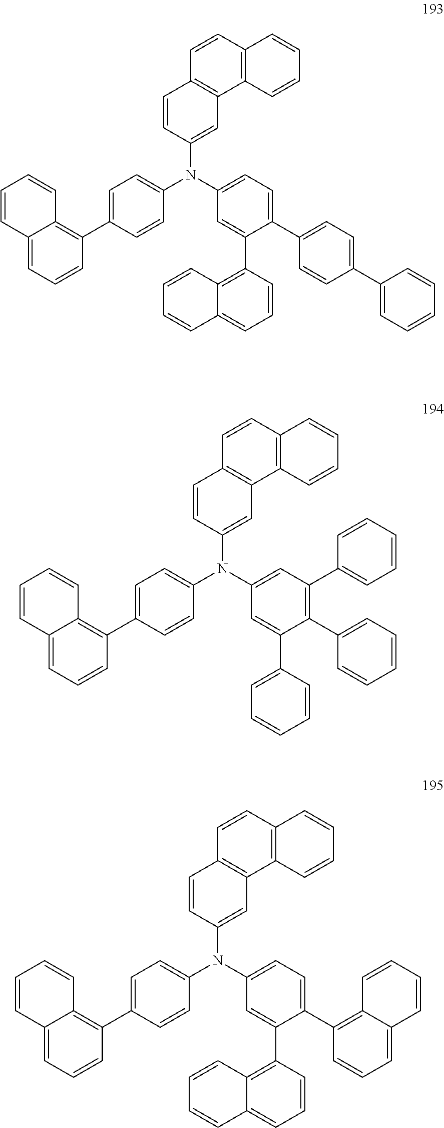 [SMILES: c1ccc(-c2ccc(-c3ccc(N(c4ccc(-c5cccc6ccccc56)cc4)c4ccc5ccc6ccccc6c5c4)cc3-c3cccc4ccccc34)cc2)cc1 ; c1ccc(-c2cc(N(c3ccc(-c4cccc5ccccc45)cc3)c3ccc4ccc5ccccc5c4c3)cc(-c3ccccc3)c2-c2ccccc2)cc1 ; c1ccc2c(-c3ccc(N(c4ccc(-c5cccc6ccccc56)c(-c5cccc6ccccc56)c4)c4ccc5ccc6ccccc6c5c4)cc3)cccc2c1]

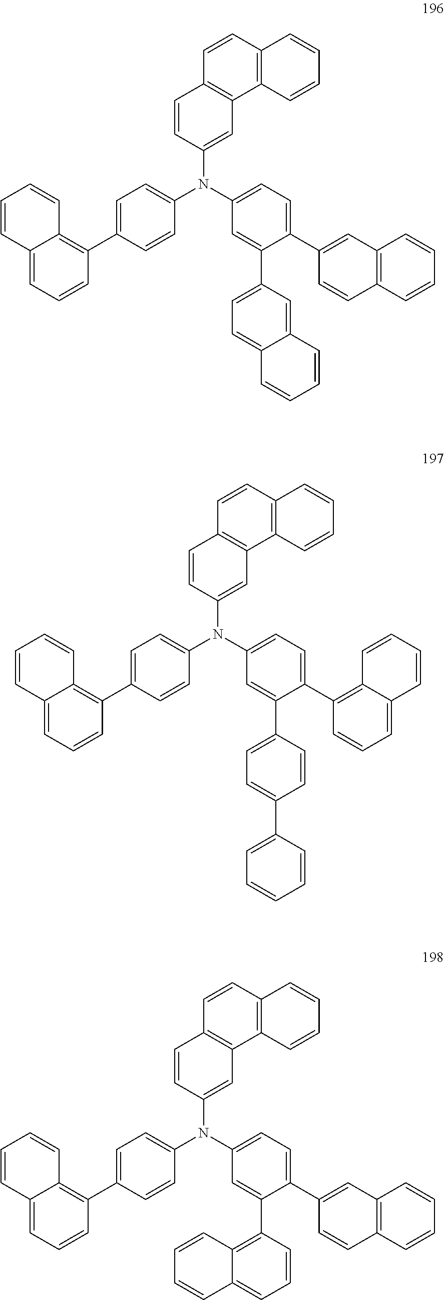 [SMILES: c1ccc2cc(-c3ccc(N(c4ccc(-c5cccc6ccccc56)cc4)c4ccc5ccc6ccccc6c5c4)cc3-c3ccc4ccccc4c3)ccc2c1 ; c1ccc(-c2ccc(-c3cc(N(c4ccc(-c5cccc6ccccc56)cc4)c4ccc5ccc6ccccc6c5c4)ccc3-c3cccc4ccccc34)cc2)cc1 ; c1ccc2cc(-c3ccc(N(c4ccc(-c5cccc6ccccc56)cc4)c4ccc5ccc6ccccc6c5c4)cc3-c3cccc4ccccc34)ccc2c1]

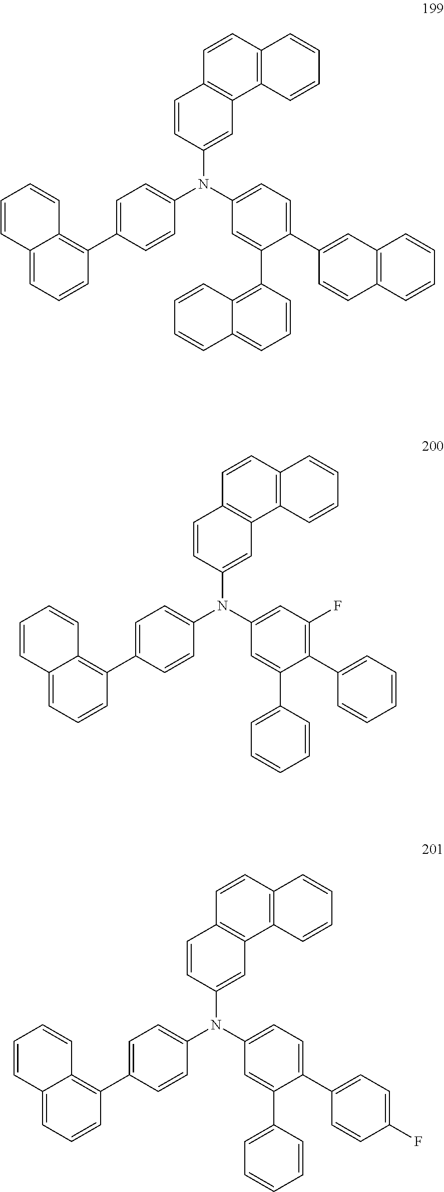 [SMILES: c1ccc2cc(-c3ccc(N(c4ccc(-c5cccc6ccccc56)cc4)c4ccc5ccc6ccccc6c5c4)cc3-c3cccc4ccccc34)ccc2c1 ; Fc1cc(N(c2ccc(-c3cccc4ccccc34)cc2)c2ccc3ccc4ccccc4c3c2)cc(-c2ccccc2)c1-c1ccccc1 ; Fc1ccc(-c2ccc(N(c3ccc(-c4cccc5ccccc45)cc3)c3ccc4ccc5ccccc5c4c3)cc2-c2ccccc2)cc1]

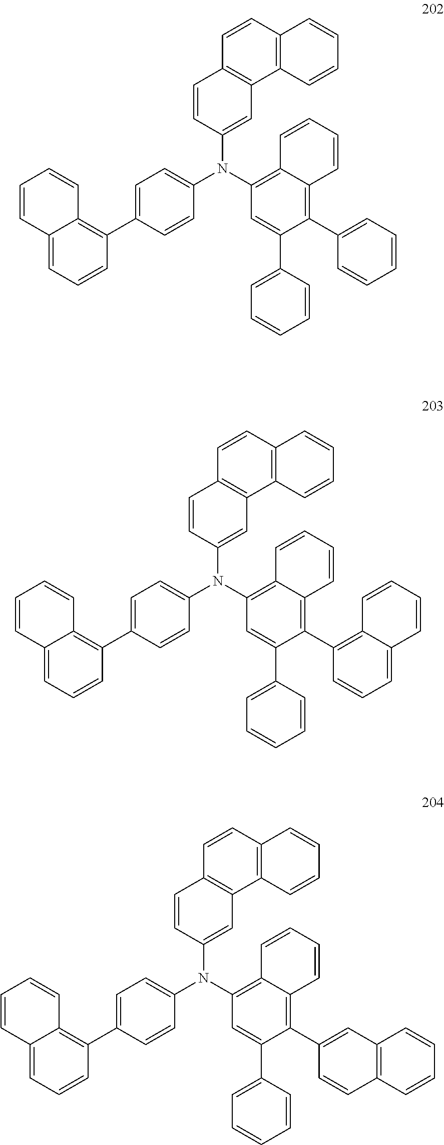 [SMILES: c1ccc(-c2cc(N(c3ccc(-c4cccc5ccccc45)cc3)c3ccc4ccc5ccccc5c4c3)c3ccccc3c2-c2ccccc2)cc1 ; c1ccc(-c2cc(N(c3ccc(-c4cccc5ccccc45)cc3)c3ccc4ccc5ccccc5c4c3)c3ccccc3c2-c2cccc3ccccc23)cc1 ; c1ccc(-c2cc(N(c3ccc(-c4cccc5ccccc45)cc3)c3ccc4ccc5ccccc5c4c3)c3ccccc3c2-c2ccc3ccccc3c2)cc1]

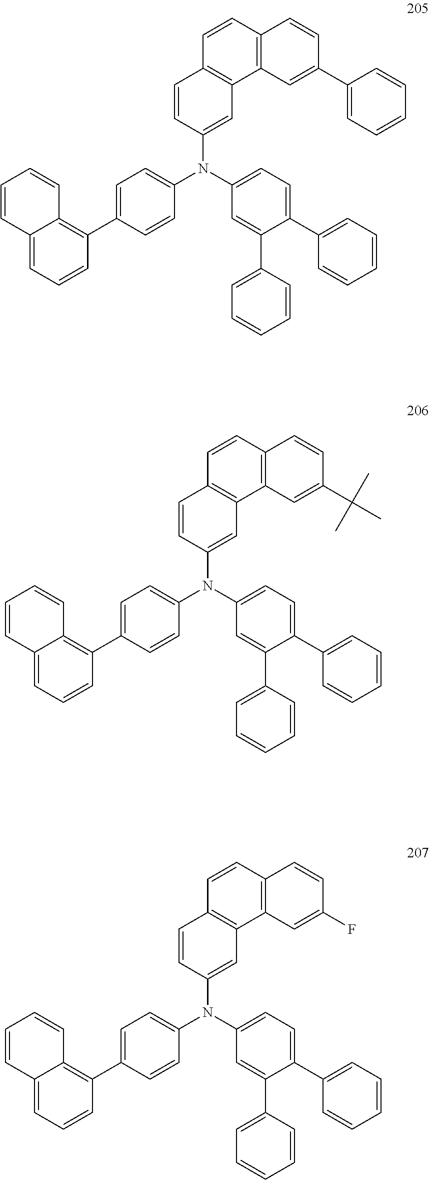 [SMILES: c1ccc(-c2ccc3ccc4ccc(N(c5ccc(-c6cccc7ccccc67)cc5)c5ccc(-c6ccccc6)c(-c6ccccc6)c5)cc4c3c2)cc1 ; CC(C)(C)c1ccc2ccc3ccc(N(c4ccc(-c5cccc6ccccc56)cc4)c4ccc(-c5ccccc5)c(-c5ccccc5)c4)cc3c2c1 ; Fc1ccc2ccc3ccc(N(c4ccc(-c5cccc6ccccc56)cc4)c4ccc(-c5ccccc5)c(-c5ccccc5)c4)cc3c2c1]

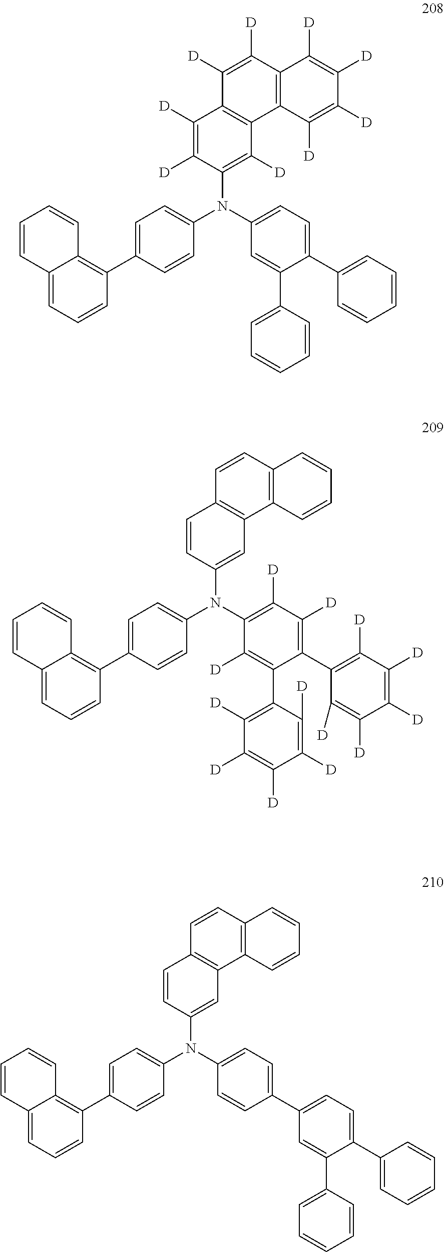 [SMILES: [2H]c1c([2H])c([2H])c2c(c1[2H])c([2H])c([2H])c1c([2H])c([2H])c(N(c3ccc(-c4cccc5ccccc45)cc3)c3ccc(-c4ccccc4)c(-c4ccccc4)c3)c([2H])c12 ; [2H]c1c([2H])c([2H])c(-c2c([2H])c([2H])c(N(c3ccc(-c4cccc5ccccc45)cc3)c3ccc4ccc5ccccc5c4c3)c([2H])c2-c2c([2H])c([2H])c([2H])c([2H])c2[2H])c([2H])c1[2H] ; c1ccc(-c2ccc(-c3ccc(N(c4ccc(-c5cccc6ccccc56)cc4)c4ccc5ccc6ccccc6c5c4)cc3)cc2-c2ccccc2)cc1]

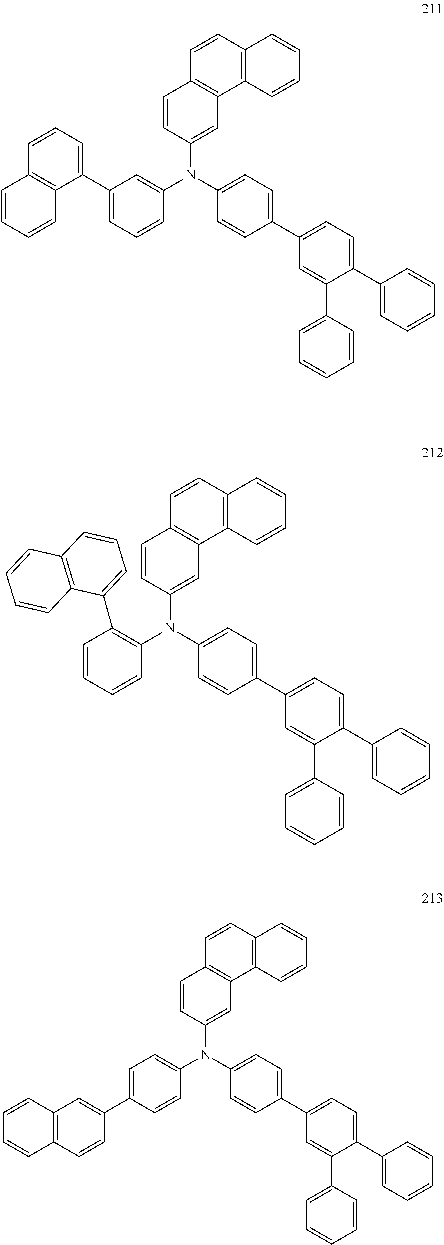 [SMILES: c1ccc(-c2ccc(-c3ccc(N(c4cccc(-c5cccc6ccccc56)c4)c4ccc5ccc6ccccc6c5c4)cc3)cc2-c2ccccc2)cc1 ; c1ccc(-c2ccc(-c3ccc(N(c4ccc5ccc6ccccc6c5c4)c4ccccc4-c4cccc5ccccc45)cc3)cc2-c2ccccc2)cc1 ; c1ccc(-c2ccc(-c3ccc(N(c4ccc(-c5ccc6ccccc6c5)cc4)c4ccc5ccc6ccccc6c5c4)cc3)cc2-c2ccccc2)cc1]

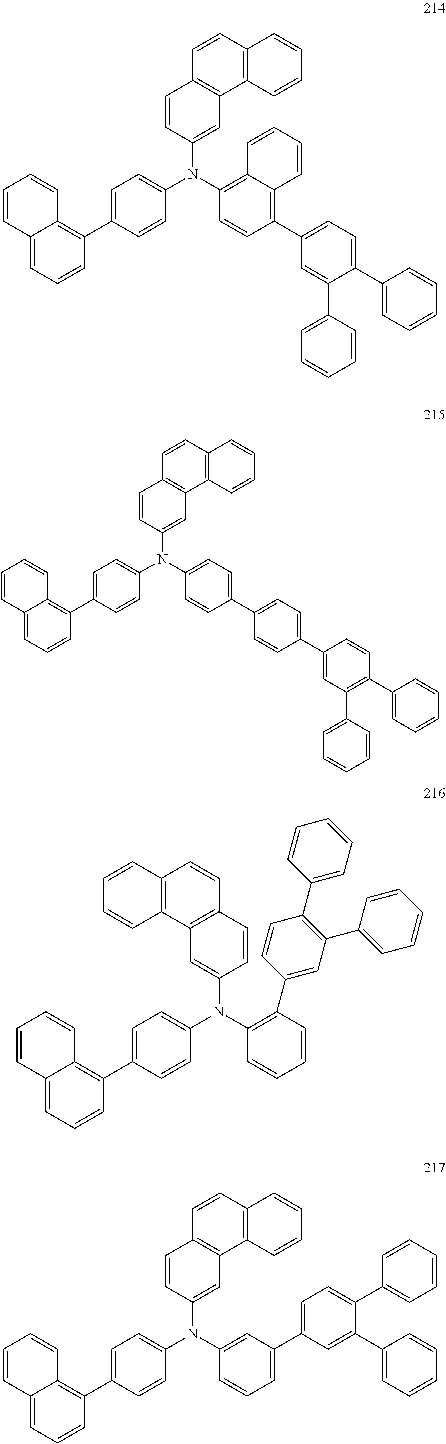 [SMILES: c1ccc(-c2ccc(-c3ccc(N(c4ccc(-c5cccc6ccccc56)cc4)c4ccc5ccc6ccccc6c5c4)c4ccccc34)cc2-c2ccccc2)cc1 ; c1ccc(-c2ccc(-c3ccc(-c4ccc(N(c5ccc(-c6cccc7ccccc67)cc5)c5ccc6ccc7ccccc7c6c5)cc4)cc3)cc2-c2ccccc2)cc1 ; c1ccc(-c2ccc(-c3ccccc3N(c3ccc(-c4cccc5ccccc45)cc3)c3ccc4ccc5ccccc5c4c3)cc2-c2ccccc2)cc1 ; c1ccc(-c2ccc(-c3cccc(N(c4ccc(-c5cccc6ccccc56)cc4)c4ccc5ccc6ccccc6c5c4)c3)cc2-c2ccccc2)cc1]

The amine compound of one or more embodiments represented by Formula 1 includes a phenanthrene moiety and a 3,4-substituted phenyl group(ortho-terphenyl moiety), and particularly, may be characterized in that the phenanthrene moiety and the 3-4-substituted phenyl group are each linked to a nitrogen atom of amine at a specific position. The amine compound of one or more embodiments may have excellent or suitable electrical stability and high charge transport capability by introducing such a substituent and specifying a substitution position. Accordingly, the lifespan of the amine compound of one or more embodiments may be improved. In one or more embodiments, the light emission efficiency and lifespan of a light emitting element containing the amine compound of one or more embodiments may also be improved.

In the light emitting element ED of one or more embodiments, the hole transport region HTR may further include a compound represented by Formula H-1. For example, the light emitting element ED of one or more embodiments may include the compound represented by Formula H-1 in another layer of the hole transport region HTR which does not contain the amine compound of one or more embodiments of Formula 1 described herein. However, the embodiment of the present disclosure is not limited thereto.

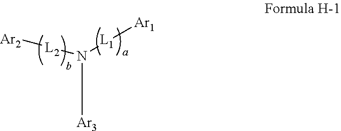

In Formula H-1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. a and b may each independently be an integer of 0 to 10. In one or more embodiments, when a or b is an integer of 2 or greater, a plurality of L₁ and L₂ may each independently be a substituted or unsubstituted arylene group having 6 to 30 ring-forming carbon atoms, or a substituted or unsubstituted heteroarylene group having 2 to 30 ring-forming carbon atoms.

In Formula H-1, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. In one or more embodiments, in Formula H-1, Ar₃ may be a substituted or unsubstituted aryl group having 6 to 30 ring-forming carbon atoms, or a substituted or unsubstituted heteroaryl group having 2 to 30 ring-forming carbon atoms.

The compound represented by Formula H-1 may be a monoamine compound. In one or more embodiments, the compound represented by Formula H-1 may a diamine compound in which at least one among Ar₁ to Ar₃ contains an amine group as a substituent. In one or more embodiments, the compound represented by Formula H-1 may be a carbazole-based compound containing a substituted or unsubstituted carbazole group in at least one of Ar₁ and Ar₂, or a fluorene-based compound containing a substituted or unsubstituted fluorene group in at least one of Ar₁ and Ar₂.

The compound represented by Formula H-1 may be represented by any one (e.g., one) selected from among compounds of Compound Group H. However, the compounds listed in Compound Group H are only examples. The compound represented by Formula H-1 is not limited to what is listed in Compound Group H.

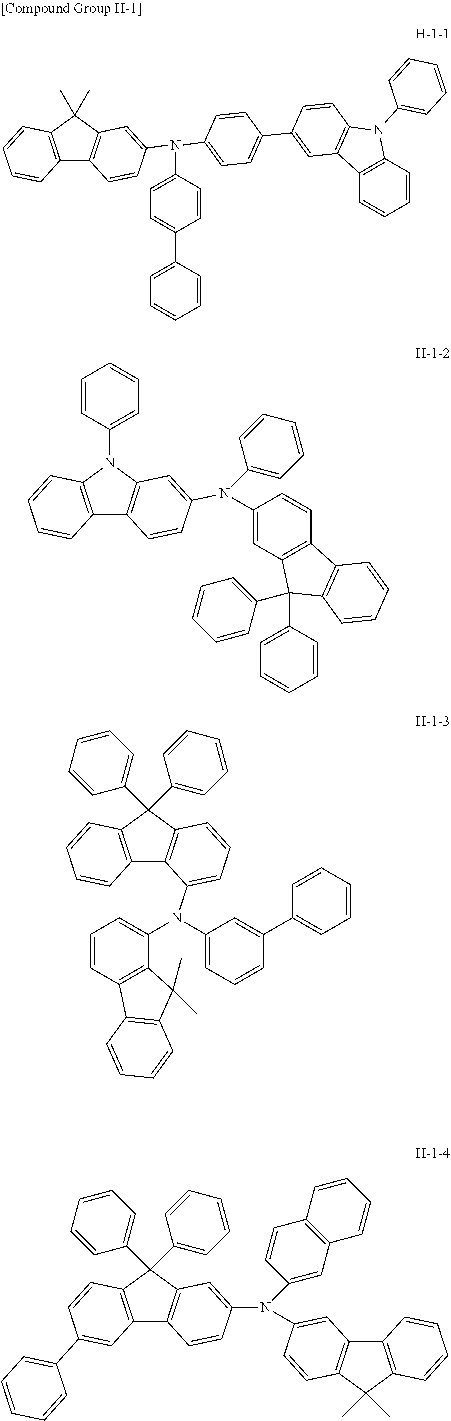 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

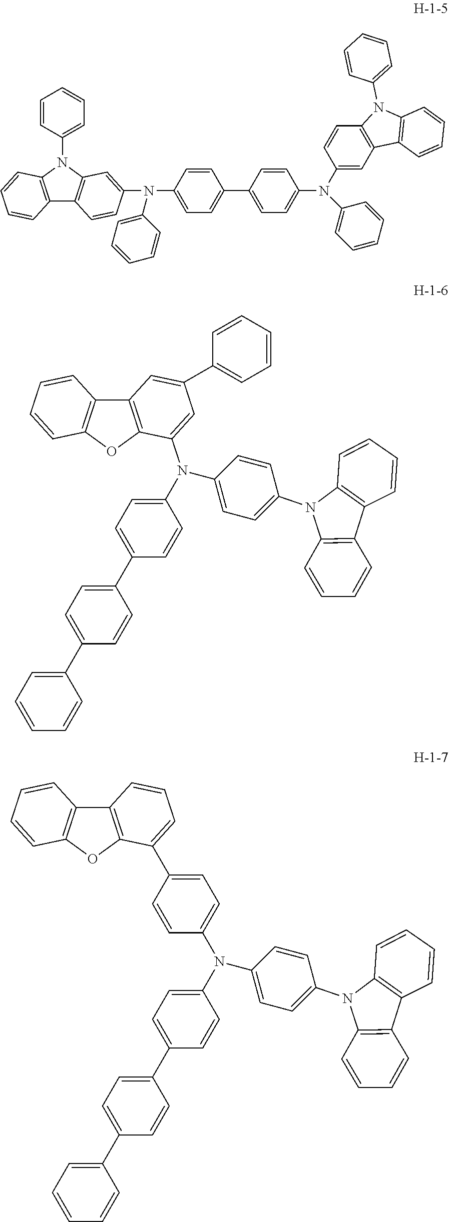 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

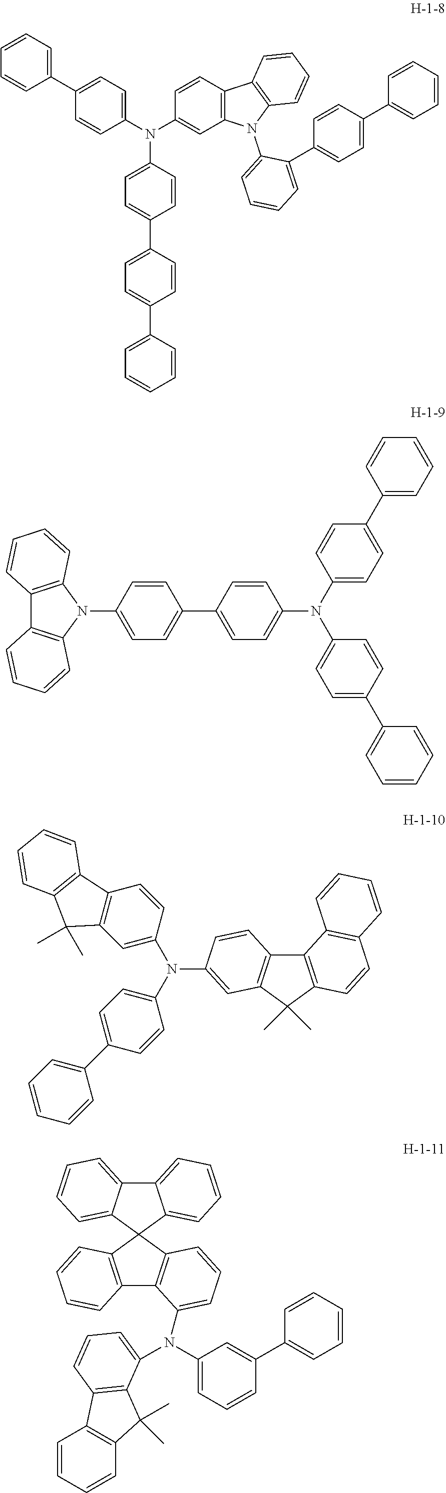

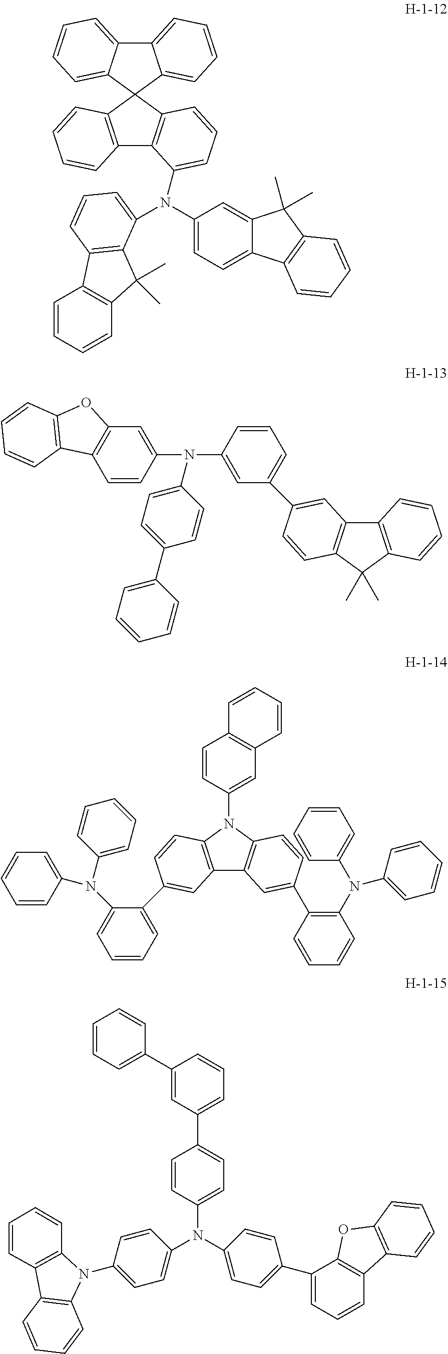 [SMILES: CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21 ; c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1]

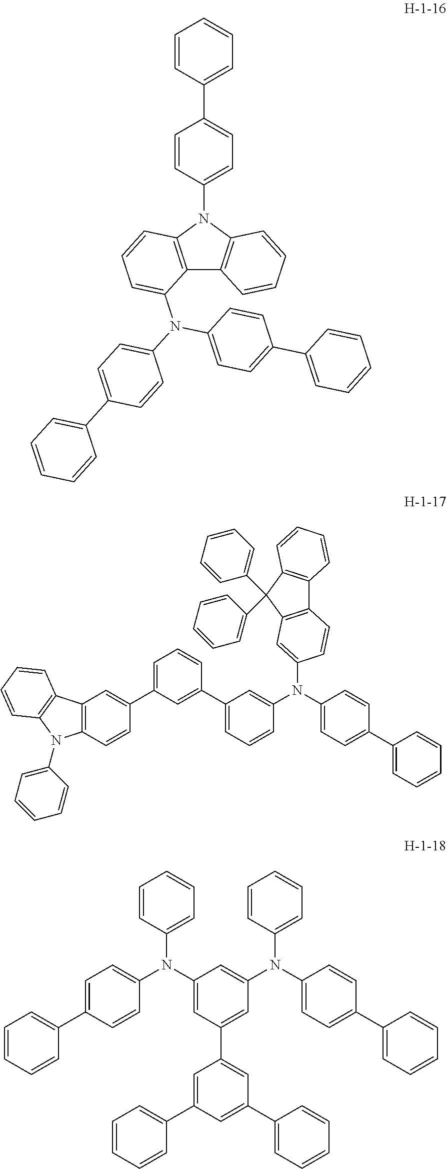 [SMILES: c1ccc(-c2ccc(N(c3ccc(-c4ccccc4)cc3)c3cccc4c3c3ccccc3n4-c3ccc(-c4ccccc4)cc3)cc2)cc1 ; c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1]

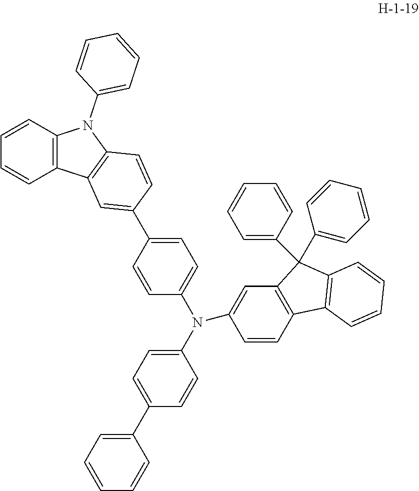 [SMILES: c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may further include at least one selected from among a phthalocyanine compound such as copper phthalocyanine, N¹,N¹′-([1,1′-biphenyl]-4,4′-diyl)bis(N1-phenyl-N4,N4-di-m-tolylbenzene-1,4-diamine) (DNTPD), 4,4′,4″-[tris(3-methyl phenyl)phenylamino]triphenylamine (m-MTDATA), 4,4′4″-Tris(N,N-diphenylamino)triphenylamine (TDATA), 4,4′,4″-tris[N(2-naphthyl)-N-phenylamino]-triphenylamine (2-TNATA), Poly(3,4-ethylenedioxythiophene)/Poly(4-styrenesulfonate) (PEDOT/PSS), Polyaniline/Dodecylbenzenesulfonic acid (PANI/DBSA), Polyaniline/Camphor sulfonicacid (PANI/CSA), Polyaniline/Poly(4-styrenesulfonate) (PANI/PSS), N,N′-di(naphthalene-1-yl)-N,N′-diphenyl-benzidine (NPB), triphenylamine-containing polyether ketone (TPAPEK), 4-Isopropyl-4′-methyldiphenyliodonium [Tetrakis(pentafluorophenyl)borate], dipyrazino[2,3-f: 2′,3′-h]quinoxaline-2,3,6,7,10,11-hexacarbonitrile (HATCN), and/or the like.

The hole transport region HTR may further include at least one selected from among a carbazole-based derivative such as N-phenylcarbazole and polyvinylcarbazole, a fluorene-based derivative, a triphenylamine-based derivative such as N,N′-bis(3-methylphenyl)-N,N′-diphenyl-[1,1-biphenyl]-4,4′-diamine (TPD) and 4,4′,4″-tris(N-carbazolyl)triphenylamine (TCTA), N,N′-di(naphthalene-1-yl)-N,N′-diplienyl-benzidine (NPB), 4,4′-Cyclohexylidene bis[N,N-bis(4-methylphenyl)benzenamine](TAPC), 4,4′-Bis[N,N′-(3-tolyl)amino]-3,3′-dimethylbiphenyl (HMTPD), (1,3-Bis(N-carbazolyl)benzene (mCP), and/or the like.

In one or more embodiments, the hole transport region HTR may further include 9-(4-tert-Butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), and/or the like.

The hole transport region HTR may include the herein-described compounds of the hole transport region in at least one of the hole injection layer HIL, the hole transport layer HTL, the light emitting auxiliary layer EAL, or the electron blocking layer EBL.

The thickness of the hole transport region HTR may be about 100 Å to about 10000 Å, for example, about 100 Å to about 5000 Å. When the hole transport region HTR includes the hole injection layer HIL, the thickness of the hole injection layer HIL may be, for example, about 30 Å to about 1000 Å. When the hole transport region HTR includes the hole transport layer HTL, the thickness of the hole transport layer HTL may be about 30 Å to about 1000 Å. When the hole transport region HTR includes the electron blocking layer EBL, the thickness of the electron blocking layer EBL may be about 10 Å to about 1000 Å. When the thicknesses of the hole transport region HTR, the hole injection layer HIL, the hole transport layer HTL, and the electron blocking layer EBL satisfy the herein-described ranges, satisfactory hole transport properties may be obtained without a substantial increase in driving voltage.

The hole transport region HTR may further include a charge generating material to improve conductivity in addition to the herein-mentioned materials. The charge generating material may be uniformly (substantially uniformly) or non-uniformly (substantially non-uniformly) dispersed in the hole transport region HTR. The charge generating material may be, for example, a p-dopant. The p-dopant may include at least one of a halogenated metal compound, a quinone derivative, a metal oxide, or a cyano group-containing compound, but the embodiment of the present disclosure is not limited thereto. For example, the p-dopant may be a halogenated metal compound such as CuI and RbI, a quinone derivative such as tetracyanoquinodimethane (TCNQ) and 2,3,5,6-tetrafluoro-7,7′,8,8′-tetracyanoquinodimethane (F4-TCNQ), a metal oxide such as a tungsten oxide and a molybdenum oxide, a cyano group-containing compound such as dipyrazino[2,3-f: 2′,3′-h]quinoxaline-2,3,6,7,10,11-hexacarbonitrile (HATCN) and 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), and/or the like, but the embodiment of the present disclosure is not limited thereto.

As described herein, the hole transport region HTR may further include at least one of the light emitting auxiliary layer EAL and the electron blocking layer EBL, in addition to the hole injection layer HIL and the hole transport layer HTL. The light emitting auxiliary layer EAL may increase light emission efficiency by compensating for a resonance distance according to the wavelength of light emitted from the light emitting layer EML, and controlling hole charge balance. In one or more embodiments, the light emitting auxiliary layer EAL may also serve to prevent or reduce electron injection into the hole transport region HTR. The light emitting auxiliary layer EAL may include a material which may be included in the hole transport region HTR. The electron blocking layer EBL is a layer serving to prevent or reduce electron injection from the electron transport region ETR to the hole transport region HTR.

A light-emitting element according to one or more embodiments, the light-emitting layer EML is provided on the hole transport region HTR. The light-emitting layer EML may have, for example, a thickness of about 100 Å to about 1000 Å, or about 100 Å to about 300 Å. The light-emitting layer EML may have a single layer formed of a single material, a single layer formed of a plurality of different materials, or a multilayer structure having a plurality of layers formed of a plurality of different materials.

In the light emitting element ED of one or more embodiments, the light emitting layer EML may be to emit blue light. The light emitting element ED of one or more embodiments may include the amine compound of one or more embodiments in a hole transport region HTR and may show high efficiency and long-life characteristics in a blue emission region. The light emitting element ED of one or more embodiments includes the amine compound of one or more embodiments in the hole transport region HTR, and the light emitting layer EML may be to emit blue fluorescence. However, one or more embodiments of the present disclosure is not limited thereto.

In the light emitting element ED of one or more embodiments, the light emitting layer EML may include anthracene derivatives, pyrene derivatives, fluoranthene derivatives, chrysene derivatives, dihydrobenzanthracene derivatives, or triphenylene derivatives. For example, the light emitting layer EML may include anthracene derivatives or pyrene derivatives.

In the light emitting elements ED of embodiments, shown in FIG. 3 to FIG. 7, the light emitting layer EML may include a host and a dopant, and the light emitting layer EML may include a compound represented by Formula E-1. The compound represented by Formula E-1 may be utilized as a fluorescence host material.

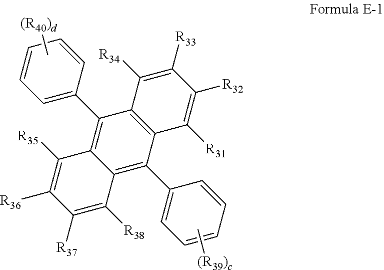

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. In one or more embodiments, R₃₁ to R₄₀ may be combined with an adjacent group to form a saturated hydrocarbon ring, an unsaturated hydrocarbon ring, a saturated heterocycle, or an unsaturated heterocycle.

In Formula E-1, “c” and “d” may each independently be an integer of 0 to 5.

Formula E-1 may be represented by any one selected from among Compound E1 to Compound E19.

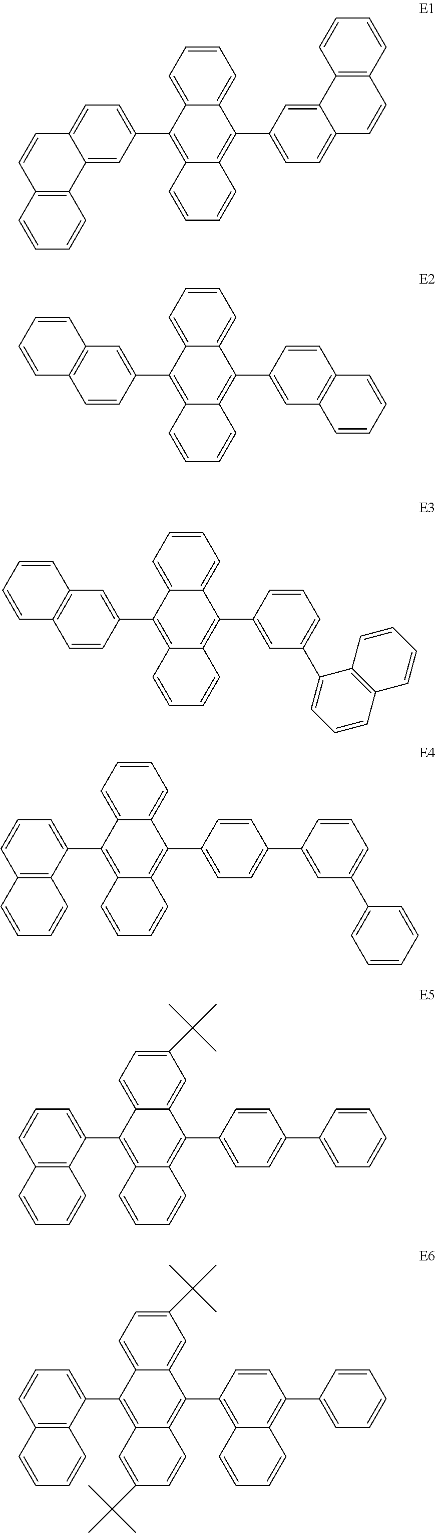

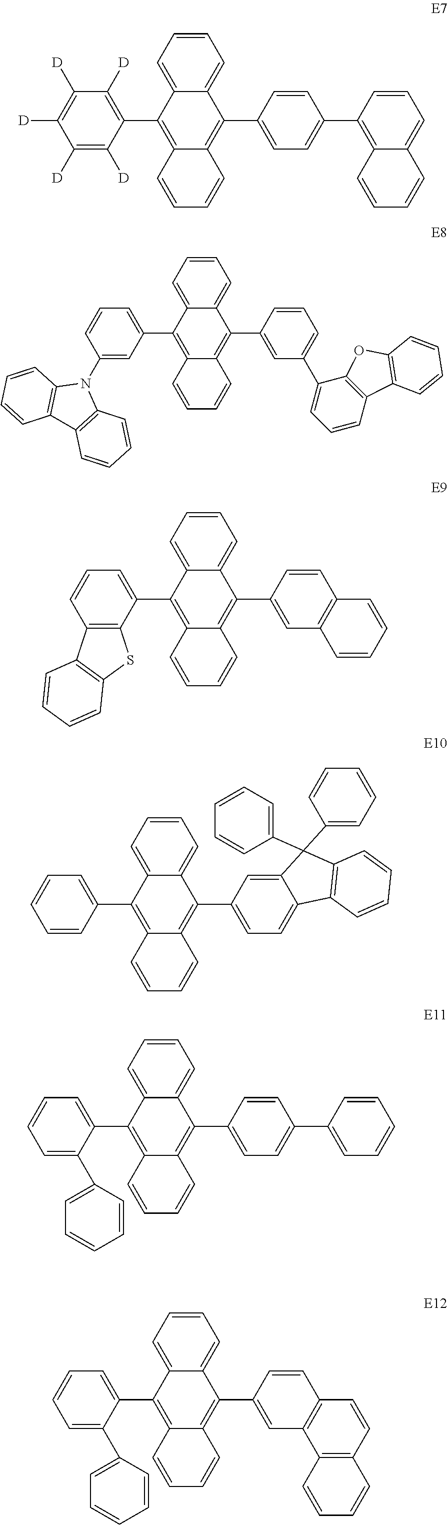

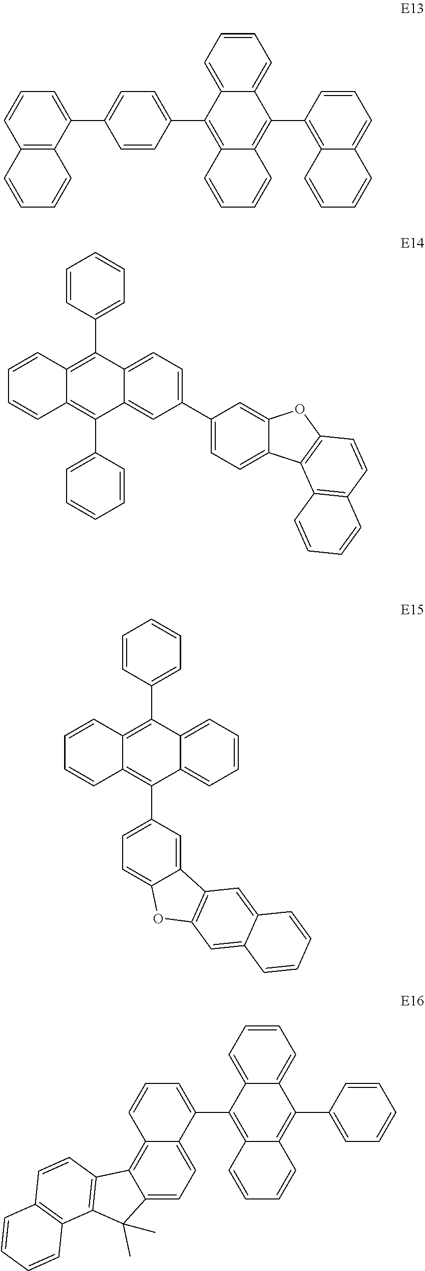

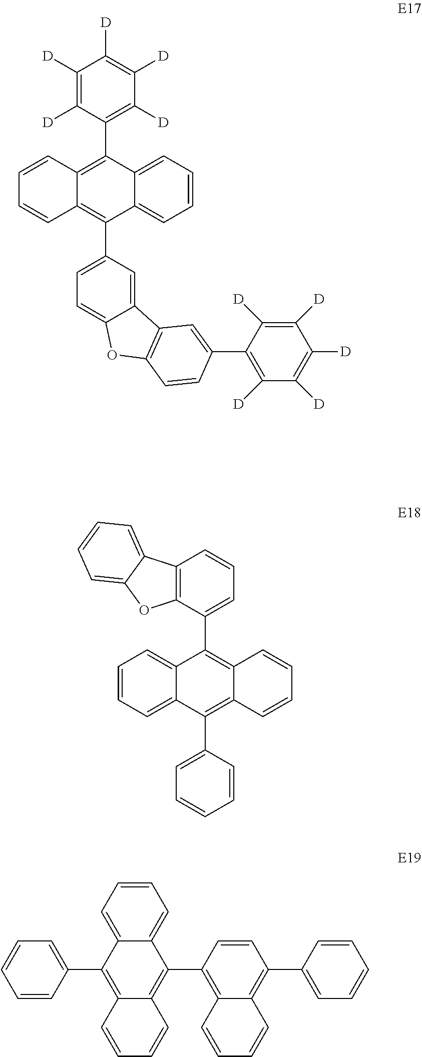

In one or more embodiments, the light emitting layer EML may include a compound represented by Formula E-2a or Formula E-2b. The compound represented by Formula E-2a or Formula E-2b may be utilized as a phosphorescence host material.

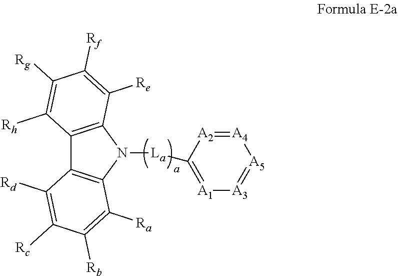

In Formula E-2a, “a” may be an integer of 0 to 10, La may be a direct linkage, a substituted or unsubstituted arylene group of 6 to 30 ring-forming carbon atoms, or a substituted or unsubstituted heteroarylene group of 2 to 30 ring-forming carbon atoms. In one or more embodiments, if “a” is an integer of 2 or more, multiple La may each independently be a substituted or unsubstituted arylene group of 6 to 30 ring-forming carbon atoms, or a substituted or unsubstituted heteroarylene group of 2 to 30 ring-forming carbon atoms.

In one or more embodiments, in Formula E-2a, A₁ to A5 may each independently be N or CRi. Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. Rₐ to Rᵢ may be combined with an adjacent group to form a hydrocarbon ring or a heterocycle including N, O, S, and/or the like as a ring-forming atom.

In one or more embodiments, in Formula E-2a, two or three of (e.g., selected from among) A₁ to A5 may be N, and the remainder may be CRᵢ.

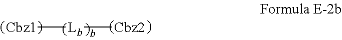

In Formula E-2b, Cbz1 and Cbz2 may each independently be an unsubstituted carbazole group, or a carbazole group substituted with an aryl group of 6 to 30 ring-forming carbon atoms. L_{b} may be a direct linkage, a substituted or unsubstituted arylene group of 6 to 30 ring-forming carbon atoms, or a substituted or unsubstituted heteroarylene group of 2 to 30 ring-forming carbon atoms. “b” is an integer of 0 to 10, and if “b” is an integer of 2 or more, multiple L_{b} may each independently be a substituted or unsubstituted arylene group of 6 to 30 ring-forming carbon atoms, or a substituted or unsubstituted heteroarylene group of 2 to 30 ring-forming carbon atoms.

The compound represented by Formula E-2a or Formula E-2b may be represented by any one selected from among the compounds in Compound Group E-2. However, the compounds listed in Compound Group E-2 are only illustrations, and the compound represented by Formula E-2a or Formula E-2b is not limited to the compounds represented in Compound Group E-2.

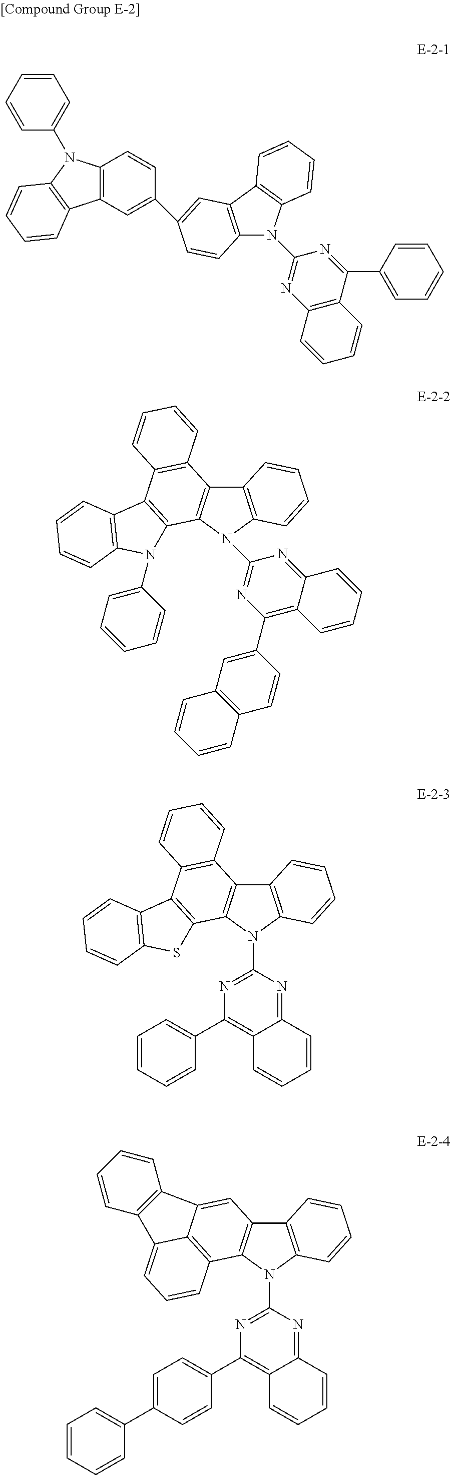 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1 ; c1ccc(-c2ccc(-c3nc(-n4c5ccccc5c5cc6c7c(cccc7c54)-c4ccccc4-6)nc4ccccc34)cc2)cc1]

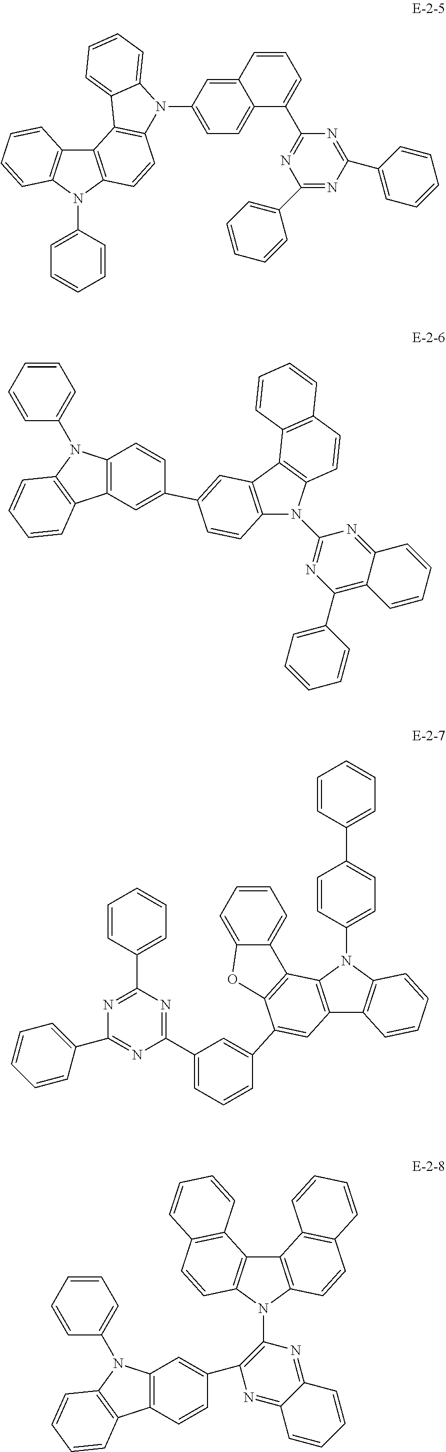 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1]

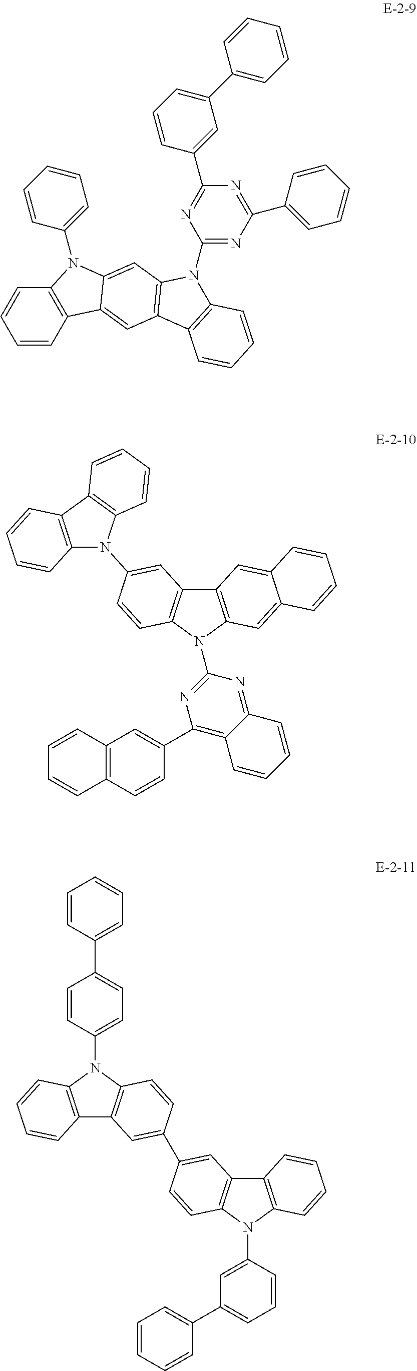 [SMILES: c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1]

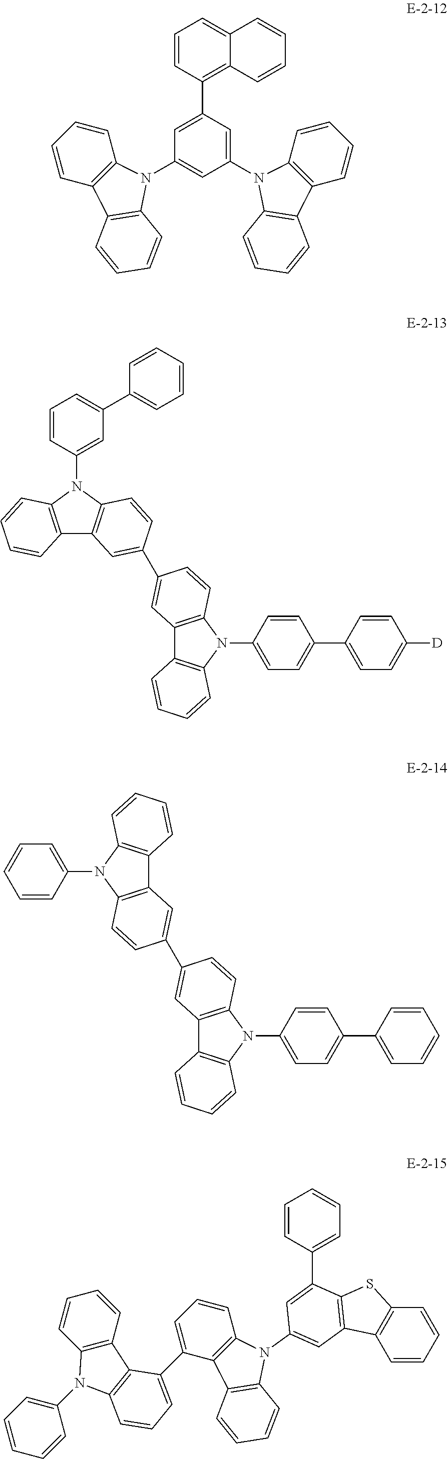 [SMILES: c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

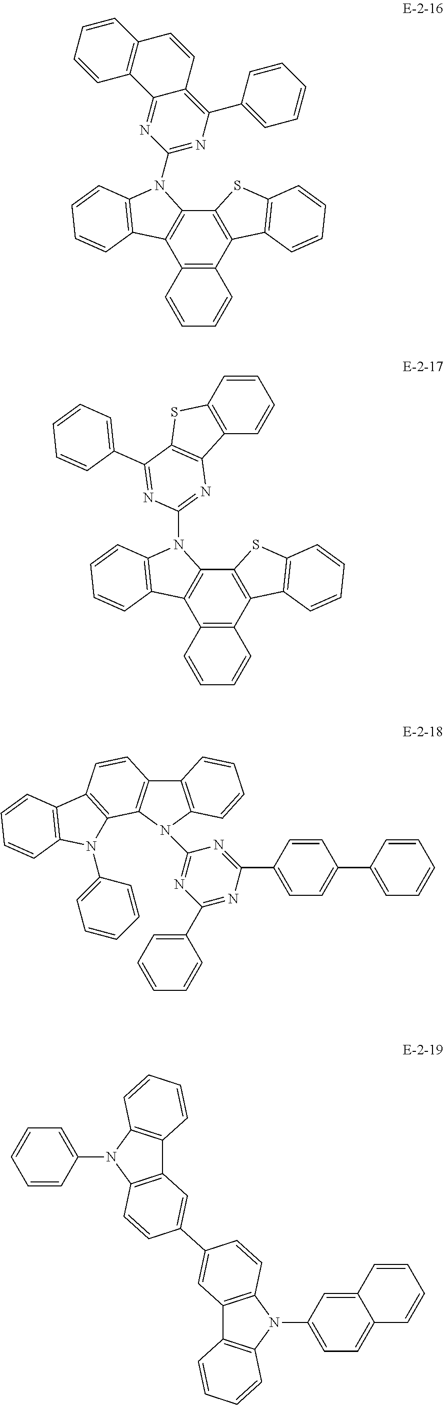 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1]

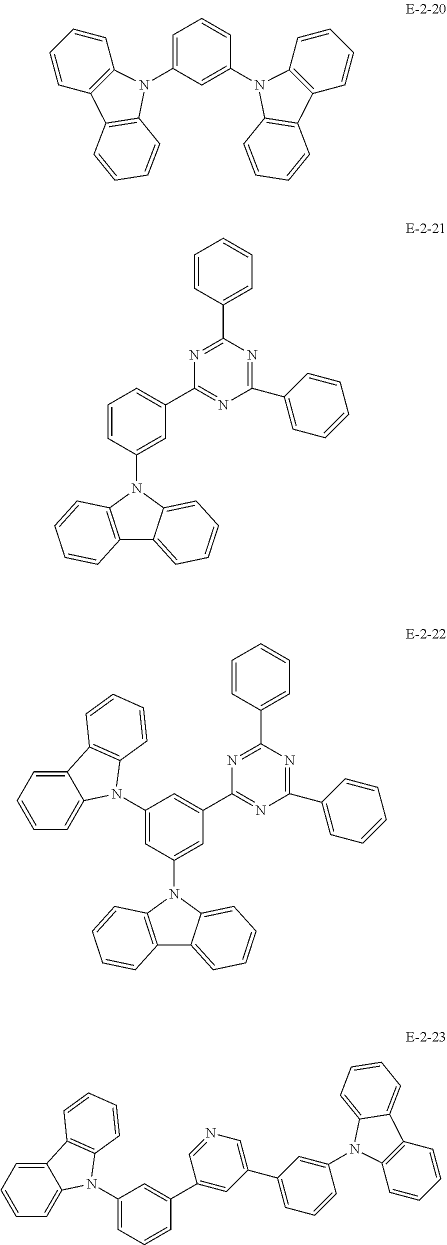 [SMILES: c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1]

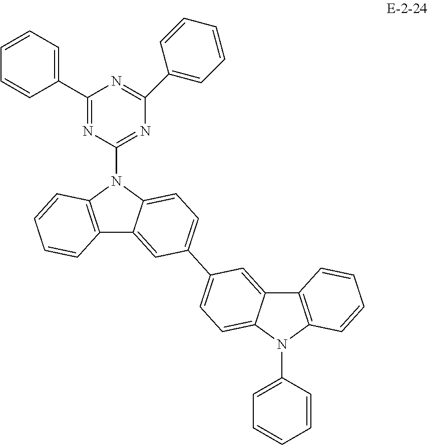 [SMILES: c1ccc(-c2nc(-c3ccccc3)nc(-n3c4ccccc4c4cc(-c5ccc6c(c5)c5ccccc5n6-c5ccccc5)ccc43)n2)cc1]

The light emitting layer EML may further include a common material well-suitable in the art as a host material. For example, the light emitting layer EML may include as a host material, at least one of bis(4-(9H-carbazol-9-yl) phenyl) diphenylsilane (BCPDS), (4-(1-(4-(diphenylamino) phenyl) cyclohexyl) phenyl) diphenyl-phosphine oxide (POPCPA), bis[2-(diphenylphosphino)phenyl]ether oxide (DPEPO), 4,4′-bis(carbazol-9-yl)biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, one or more embodiments of the present disclosure is not limited thereto. For example, tris(8-hydroxyquinolino)aluminum (Alq3), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO3), octaphenylcyclotetra siloxane (DPSiO4), and/or the like may be utilized as the host material.

The light emitting layer EML may include a compound represented by Formula M-a or Formula M-b. The compound represented by Formula M-a or Formula M-b may be utilized as a phosphorescence dopant material.

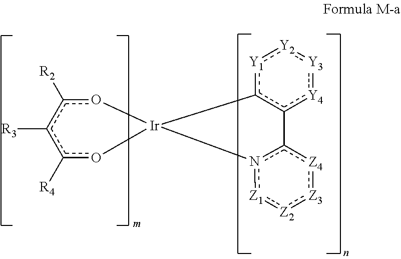

In Formula M-a, Y1 to Y4, and Z1 to Z4 may each independently be CR₁ or N, and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. In Formula M-a, “m” is 0 or 1, and “n” is 2 or 3. In Formula M-a, if “m” is 0, “n” is 3, and if “m” is 1, “n” is 2.

The compound represented by Formula M-a may be represented by any one selected from among Compounds M-a1 to M-a25. However, Compounds M-a1 to M-a25 are illustrations, and the compound represented by Formula M-a is not limited to the compounds represented by Compounds M-a1 to M-a25.

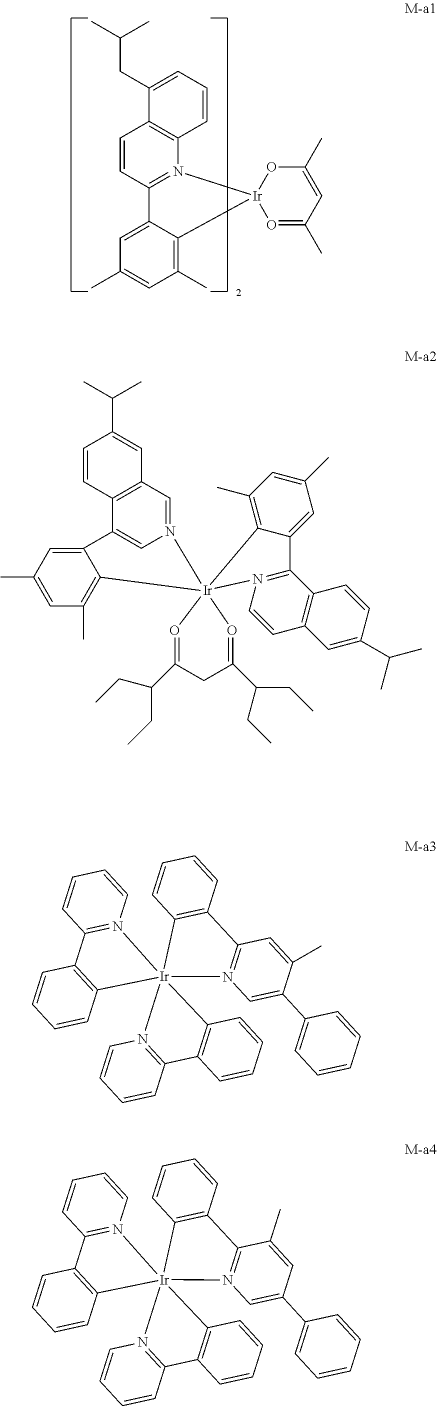

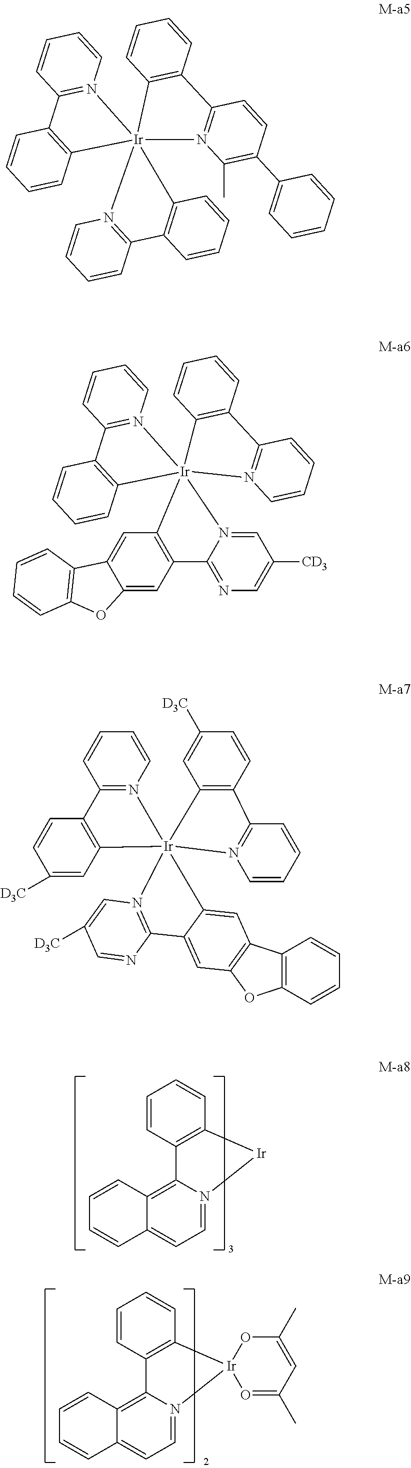

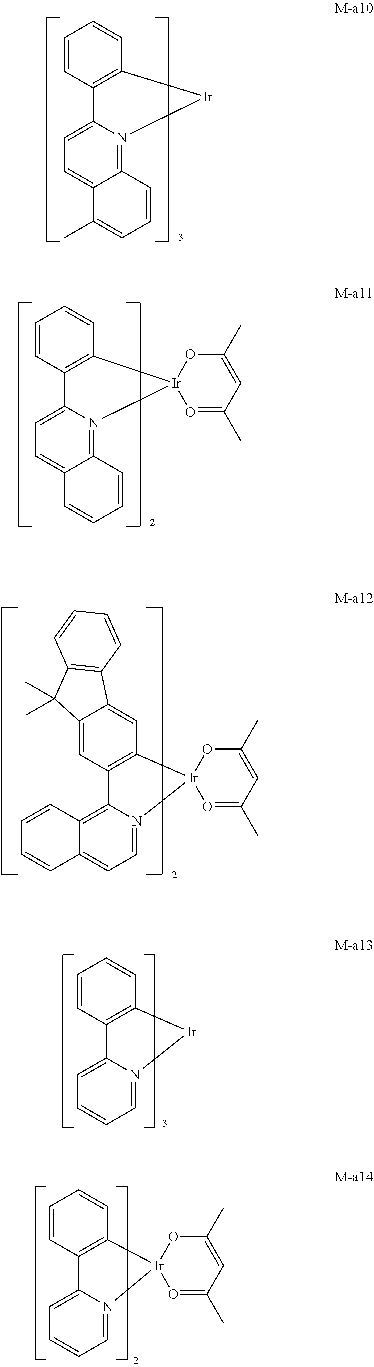

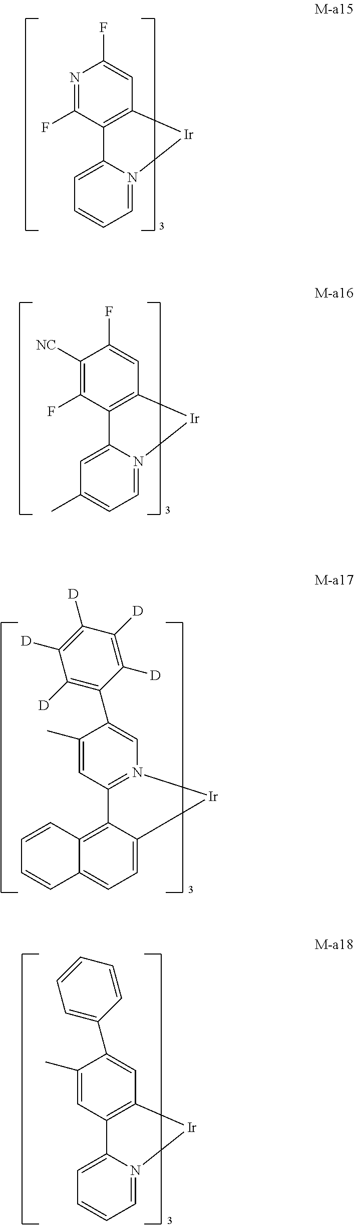

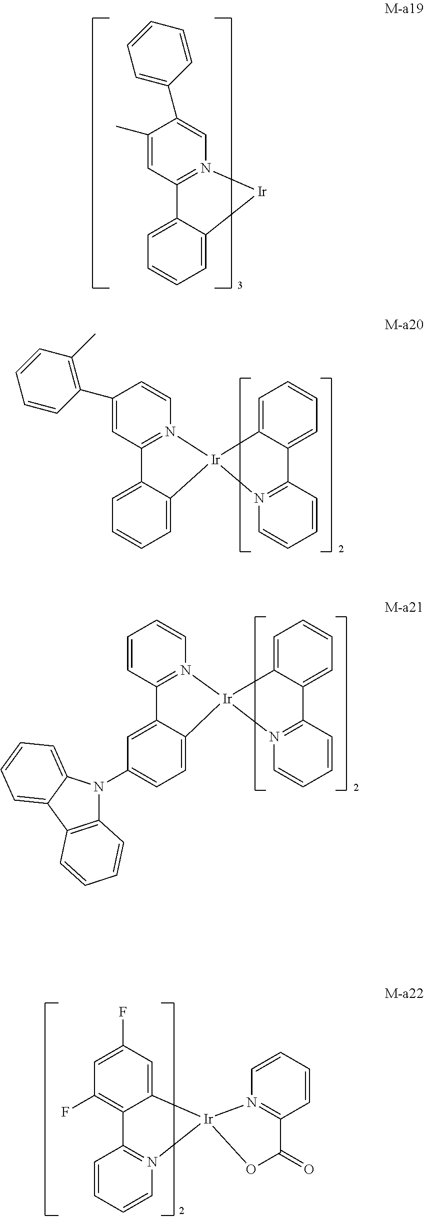

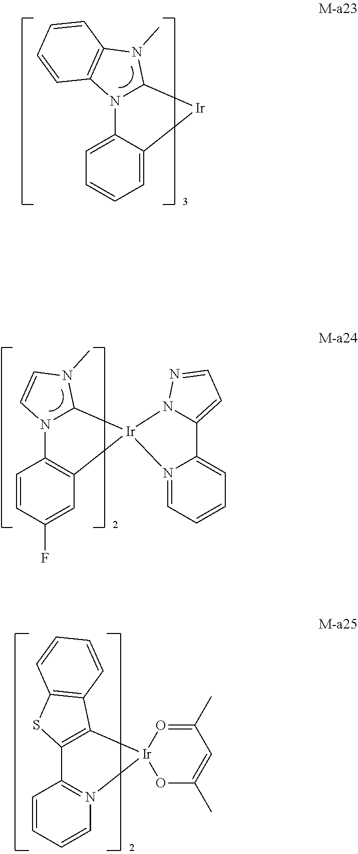

Compound M-a1 and Compound M-a2 may be utilized as red dopant materials, and Compound M-a3 to Compound M-a5 may be utilized as green dopant materials.

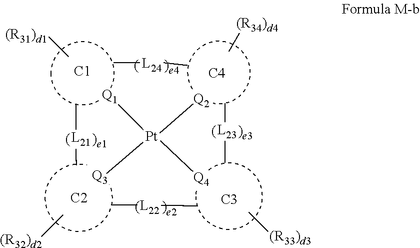

In Formula M-b, Q₁ to Q₄ may each independently be C or N, C₁ to C₄ may each independently be a substituted or unsubstituted hydrocarbon ring of 5 to 30 ring-forming carbon atoms, or a substituted or unsubstituted heterocycle of 2 to 30 ring-forming carbon atoms. L₂₁ to L₂₄ may each independently be a direct linkage,

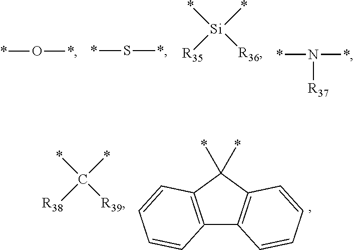

a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms, and e1 to e4 may each independently be 0 or 1. R₃₁ to R₃₉ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring, and d1 to d4 may each independently be an integer of 0 to 4.

The compound represented by Formula M-b may be utilized as a blue phosphorescence dopant or a green phosphorescence dopant.

The compound represented by Formula M-b may be represented by any one selected from among the following compounds. However, the following compounds are merely illustrations, and the compound represented by Formula M-b is not limited to the following compounds.

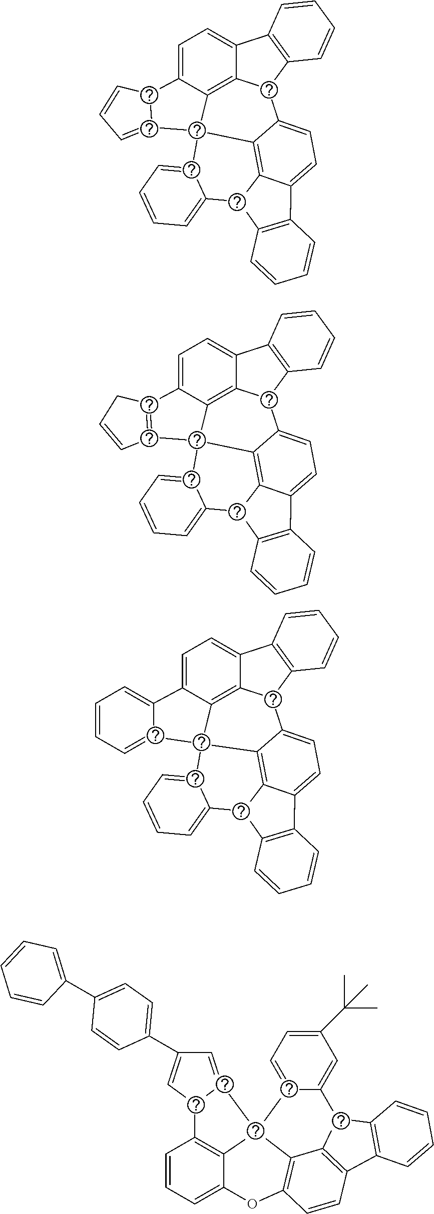

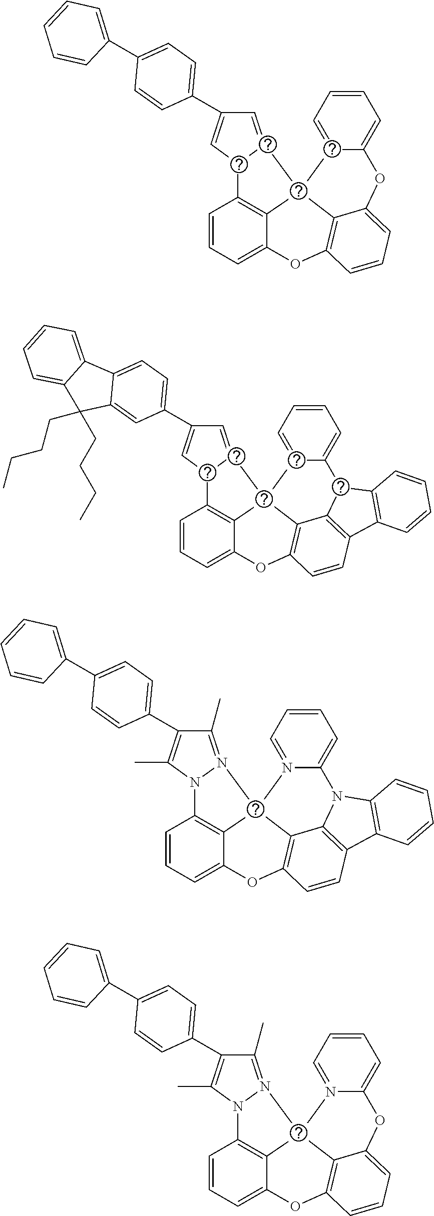

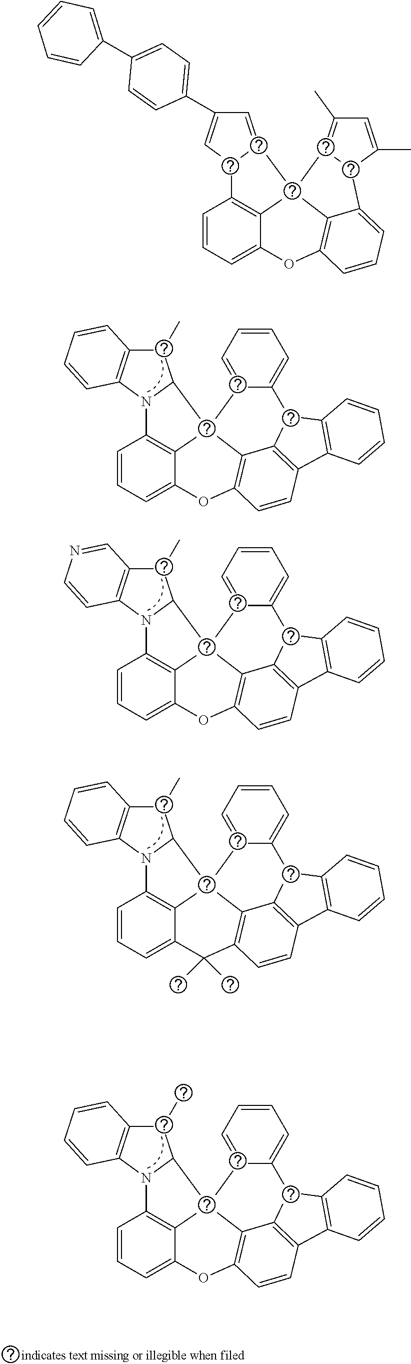

In the compounds herein, R, R₃₈,and R₃₉ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

The light emitting layer EML may include any one selected from among Formula F-a to Formula F-c. The compounds represented by Formula F-a to Formula F-c may be utilized as fluorescence dopant materials.

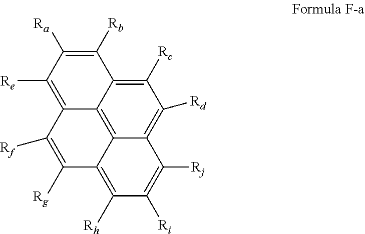

In Formula F-a, two of (e.g., selected from among) Rₐ to Rⱼ may each independently be substituted with

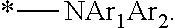

The remainder not substituted with

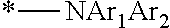

selected from among Rₐ to Rⱼ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In

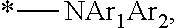

Ar₁ and Ar₂ may each independently be a substituted or unsubstituted aryl group of 6 to 30 ring-forming carbon atoms, or a substituted or unsubstituted heteroaryl group of 2 to 30 ring-forming carbon atoms. For example, at least one selected from among Ar₁ and Ar₂ may be a heteroaryl group including 0 or S as a ring-forming atom.

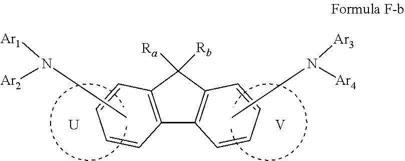

In Formula F-b, Rₐ and R_{b} may each independently be a hydrogen atom, a deuterium atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. Ar₁ to Ar₄ may each independently be a substituted or unsubstituted aryl group of 6 to 30 ring-forming carbon atoms, or a substituted or unsubstituted heteroaryl group of 2 to 30 ring-forming carbon atoms.

In Formula F-b, U and V may each independently be a substituted or unsubstituted hydrocarbon ring of 5 to 30 ring-forming carbon atoms, or a substituted or unsubstituted heterocycle of 2 to 30 ring-forming carbon atoms. at least one among Ar₁ to Ar₄ may be a heteroaryl group including 0 or S as a ring-forming atom.

In Formula F-b, the number of rings represented by U and V may each independently be 0 or 1. For example, in Formula F-b, if the number of U or V is 1, one ring forms a fused ring at the designated part by U or V, and if the number of U or V is 0, a ring is not present at the designated part by U or V. For example, if the number of U is 0, and the number of V is 1, or if the number of U is 1, and the number of V is 0, a fused ring having the fluorene core of Formula F-b may be a ring compound with four rings. In one or more embodiments, if the number of both (e.g., simultaneously) U and V is 0, the fused ring of Formula F-b may be a ring compound with three rings. In one or more embodiments, if the number of both (e.g., simultaneously) U and V is 1, a fused ring having the fluorene core of Formula F-b may be a ring compound with five rings.

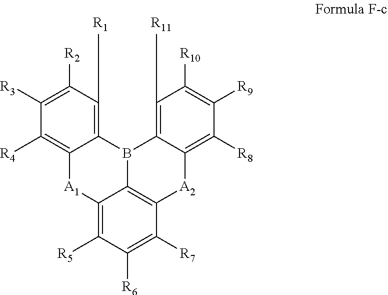

In Formula F-c, A₁ and A2 may each independently be O, S, Se, or NRₘ, and μm may be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring.

In Formula F-c, A₁ and A2 may each independently be combined with the substituents of an adjacent ring to form a fused ring. For example, if A₁ and A₂ may each independently be NRₘ, A₁ may be combined with R₄ or R₅ to form a ring. In one or more embodiments, A₂ may be combined with R₇ or R₈ to form a ring.

In one or more embodiments, the light emitting layer EML may include as a suitable dopant material, styryl derivatives (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and 4,4′-bis[2-(4-(N,N-diphenylamino)phenyl)vinyl]biphenyl (DPAVBi)), perylene and the derivatives thereof (for example, 2,5,8,11-tetra-t-butylperylene (TBP)), pyrene and the derivatives thereof (for example, 1,1-dipyrene, 1,4-dipyrenylbenzene, and 1,4-bis(N,N-diphenylamino)pyrene), and/or the like.

In one or more embodiments, if multiple light emitting layers EML are included, at least one light emitting layer EML may include a suitable phosphorescence dopant material. For example, the phosphorescence dopant may utilize a metal complex including iridium (Ir), platinum (Pt), osmium (Os), gold (Au), titanium (Ti), zirconium (Zr), hafnium (Hf), europium (Eu), terbium (Tb) and/or thulium (Tm). For example, iridium(III) bis(4,6-difluorophenylpyridinato-N,C2′)picolinate (Flrpic), bis(2,4-difluorophenylpyridinato)-tetrakis(1-pyrazolyl)borate iridium(III) (Fir6), or platinum octaethyl porphyrin (PtOEP) may be utilized as the phosphorescence dopant. However, one or more embodiments of the present disclosure is not limited thereto.

In one or more embodiments, in the light emitting element ED according to one or more embodiments, the light emitting layer EML may be a delayed fluorescence light emitting layer including a host and a dopant. More specifically, the light emitting layer EML may be to emit thermally activated delayed fluorescence (TADF). In the light emitting element ED according to one or more embodiments, the light emitting layer EML may include a suitable thermally active delayed fluorescence dopant.

In one or more embodiments, the light emitting layer EML of the light emitting element ED may include a plurality of host materials, a thermally activated delayed fluorescent dopant, and a phosphorescent sensitizer.

The light emitting layer EML may include a quantum dot material. The core of the quantum dots may be selected from among a Group II-VI compound, a Group I-II-VI compound, a Group II-IV-VI compound, a Group I-II-IV-VI compound, a Group II-IV-V compound, a Group III-VI compound, a Group I-Ill-VI compound, a Group III-V compound, a Group III-II-V compound, a Group IV-VI compound, a Group IV element, a Group IV compound, and/or any suitable combination thereof.

The Group II-VI compound may be selected from the group consisting of a binary compound selected from the group consisting of CdSe, CdTe, CdS, ZnS, ZnSe, ZnTe, ZnO, HgS, HgSe, HgTe, MgSe, MgS, and a mixture thereof, a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a mixture thereof, and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, and a mixture thereof.

The Group III-VI compound may include a binary compound such as In₂S₃ or In₂Se₃, a ternary compound such as InGaS₃ or InGaSe₃, or any combination thereof.

The Group I-III-VI compound may be of (e.g., selected from among) a ternary compound selected from the group consisting of AgInS, AgInS₂, CuInS, CuInS₂, AgGaS₂, CuGaS₂ CuGaO₂, AgGaO₂, AgAlO₂, and a mixture thereof, or a quaternary compound such as AgInGaS₂ or CuInGaS₂.

The Group III-V compound may be selected from the group consisting of a binary compound selected from the group consisting of GaN, GaP, GaAs, GaSb, AlN, AlP, AlAs, AlSb, InN, InP, InAs, InSb, and a mixture thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and a mixture thereof, and a quaternary compound selected from the group consisting of GaAlNP, GaAlNAs, GaAlNSb, GaAlPAs, GaAlPSb, GaInNP, GaInNAs, GalnNSb, GaInPAs, GalnPSb, InAlNP, InAlNAs, InAlNSb, InAlPAs, InAlPSb, and a mixture thereof. In one or more embodiments, the Group III-V compound may further include a Group II metal. For example, InZnP, and/or the like, may be selected as the Group III-II-V compound.

The Group IV-VI compound may be selected from the group consisting of a binary compound selected from the group consisting of SnS, SnSe, SnTe, PbS, PbSe, PbTe, and a mixture thereof, a ternary compound selected from the group consisting of SnSeS, SnSeTe, SnSTe, PbSeS, PbSeTe, PbSTe, SnPbS, SnPbSe, SnPbTe, and a mixture thereof, and a quaternary compound selected from the group consisting of SnPbSSe, SnPbSeTe, SnPbSTe, and a mixture thereof. The Group IV element may be selected from the group consisting of Si, Ge, and a mixture thereof. The Group IV compound may be a binary compound selected from the group consisting of SiC, SiGe, and a mixture thereof.

Each element included in a polynary compound such as the binary compound, the ternary compound, or the quaternary compound may be present in a particle with a substantially uniform or non-substantially uniform concentration distribution. For example, the formulae may refer to the types (kinds) of elements included in the compounds, and the elemental ratio in the compound may be different. For example, AgInGaS₂ may refer to AgInₓGa₁₋ₓS₂ (where x is a real number of 0 to 1).

In one or more embodiments, the quantum dot may have a single structure or a double structure of core-shell in which the concentration of each element included in the quantum dot is substantially uniform. For example, the material included in the core may be different from the material included in the shell.

The shell of the quantum dot may serve as a protection layer to prevent or reduce the chemical deformation of the core to maintain semiconductor properties, and/or a charging layer to impart electrophoresis properties to the quantum dot. The shell may be a single layer or a multilayer. An interface between the core and the shell may have a concentration gradient in which the concentration of an element present in the shell becomes lower towards the center.

An example of the shell of the quantum dots may include a metal or non-metal oxide, a semiconductor compound, and/or any suitable combination thereof. For example, the metal or non-metal oxide may be a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, or NiO, or a ternary compound such as MgAl₂O₄, CoFe₂O₄, NiFe₂O₄, or CoMn₂O₄, but the embodiment of the present disclosure is not limited thereto.

Also, examples of the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AIP, AlSb, and/or the like, but the embodiment of the present disclosure is not limited thereto.

Each element included in a polynary compound such as the binary compound, or the ternary compound may be present in a particle with a substantially uniform or non-substantially uniform concentration distribution. For example, the formulae may refer to the types (kinds) of elements included in the compounds, and the elemental ratio in the compound may be different.

The quantum dot may have a full width of half maximum (FWHM) of an emission wavelength spectrum of about 45 nm or less, about 40 nm or less, or about 30 nm or less, and color purity or color reproducibility may be improved in the described ranges. In one or more embodiments, light emitted through such a quantum dot is emitted in each (e.g., any or all) direction(s), and thus a wide viewing angle may be improved.

In one or more embodiments, although the form of the quantum dot is not particularly limited as long as it is a form commonly utilized in the art, more specifically, the quantum dot in the form of spherical nanoparticles, pyramidal nanoparticles, multi-arm nanoparticles, cubic nanoparticles, nanotubes, nanowires, nanofibers, nanoplate particles, and/or the like may be utilized.

As the size of the quantum dot is adjusted or the elemental ratio in the quantum dot compound is adjusted, it is possible to control the energy band gap, and thus light in one or more suitable wavelength ranges may be obtained in the quantum dot light emitting layer. Therefore, the quantum dot as described (utilizing different sizes of quantum dots or different elemental ratios in the quantum dot compound) is utilized, and thus the light emitting element, which emits light in one or more suitable wavelengths, may be implemented. For example, the adjustment of the size of the quantum dot or the elemental ratio in the quantum dot compound may be selected to emit red, green, and/or blue light. In one or more embodiments, the quantum dots may be configured to emit white light by combining one or more suitable colors of light.

In each of the light emitting elements ED of embodiments illustrated in FIGS. 3 to 7, the electron transport region ETR is provided on the light emitting layer EML. The electron transport region ETR may include at least one of the hole blocking layer HBL, the electron transport layer ETL, or the electron injection layer EIL, but the embodiment of the present disclosure is not limited thereto.

The electron transport region ETR may have a single layer formed of a single material, a single layer formed of a plurality of different materials, or a multilayer structure including a plurality of layers formed of a plurality of different materials.

For example, the electron transport region ETR may have a single layer structure of the electron injection layer EIL or the electron transport layer ETL, and may have a single layer structure formed of an electron injection material and an electron transport material. In one or more embodiments, the electron transport region ETR may have a single layer structure formed of a plurality of different materials, or may have a structure in which an electron transport layer ETL/electron injection layer EIL, a hole blocking layer HBL/electron transport layer ETL/electron injection layer EIL are stacked in order from the light emitting layer EML, but the embodiment of the present disclosure is not limited thereto. The electron transport region ETR may have a thickness, for example, from about 1,000 Å to about 1,500 Å.

The electron transport region ETR may be formed utilizing one or more suitable methods such as a vacuum deposition method, a spin coating method, a cast method, a Langmuir-Blodgett (LB) method, an inkjet printing method, a laser printing method, and a laser induced thermal imaging (LITI) method.

The electron transport region ETR may include a compound represented by Formula ET-2.

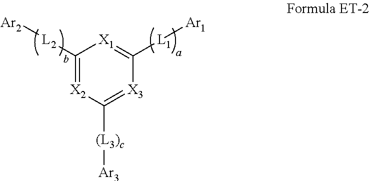

In Formula ET-2, at least one selected from among X₁ to X₃ is N, and the rest (e.g., any remaining selected from among X₁ to X₃) may be CRₐ. Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-2, a to c may each independently be an integer of 0 to 10. In Formula ET-2, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. In one or more embodiments, if (e.g., when) a to c may each independently be an integer of 2 or more, L₁ to L₃ may each independently be a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound. However, the embodiment of the present disclosure is not limited thereto, and the electron transport region ETR may include, for example, tris(8-hydroxyquinolinato)aluminum (Alq3), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenyl-4-yl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenyl-4-yl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), beryllium bis(benzoquinolin-10-olate) (Bebq₂), 9,10-di(naphthalen-2-yl)anthracene (ADN), 1,3-bis[3,5-di(pyridin-3-yl)phenyl]benzene (BmPyPhB), and/or mixtures thereof.

The electron transport region ETR may include at least one selected from among Compound ET1 to Compound ET36.

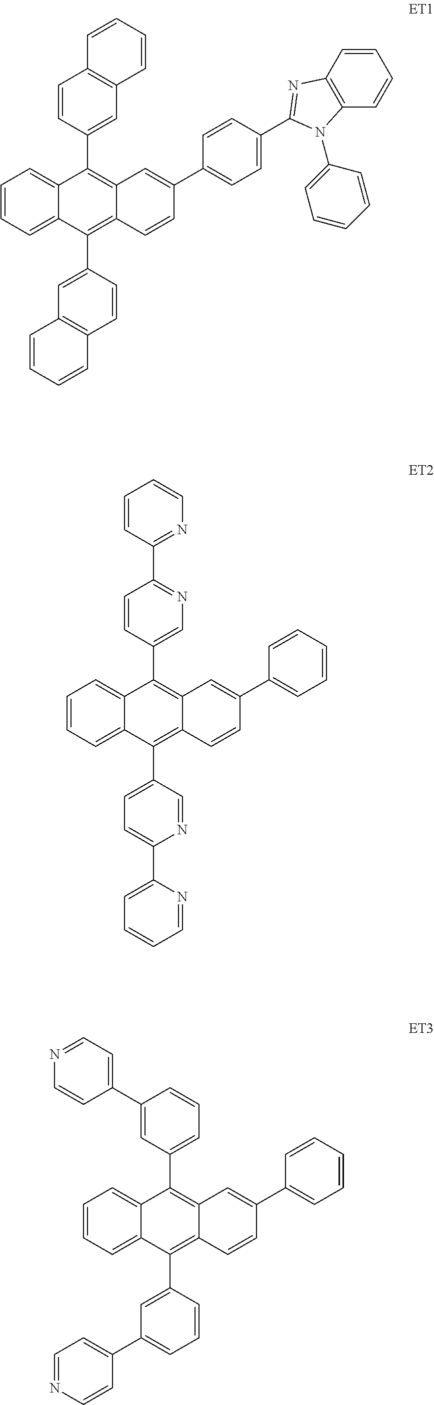 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

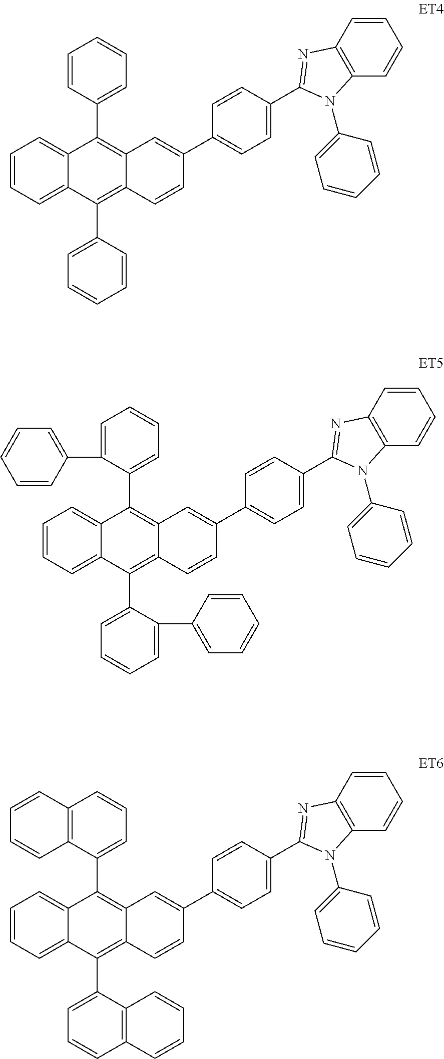 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

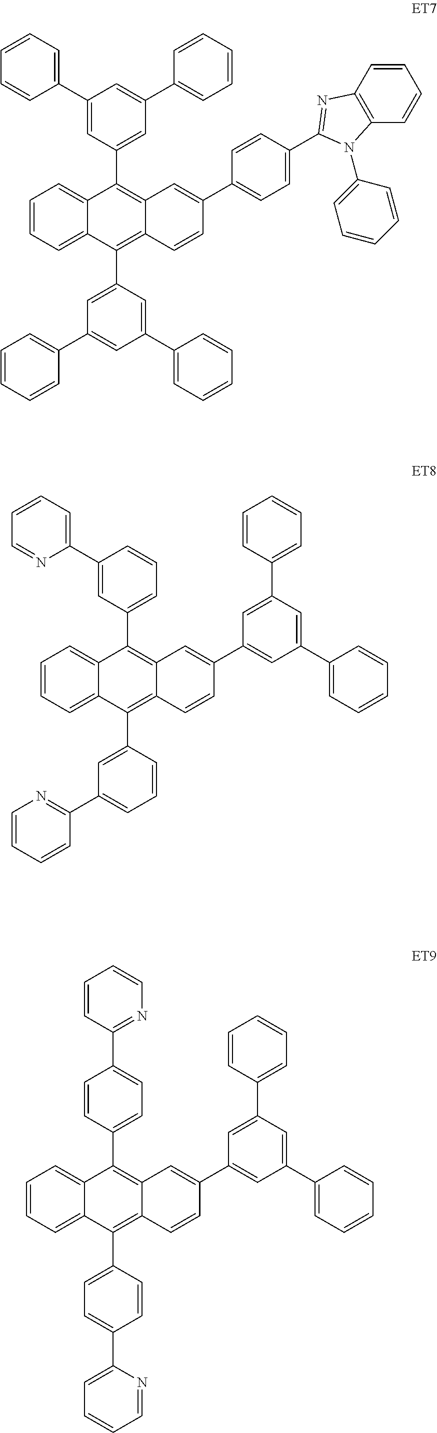 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

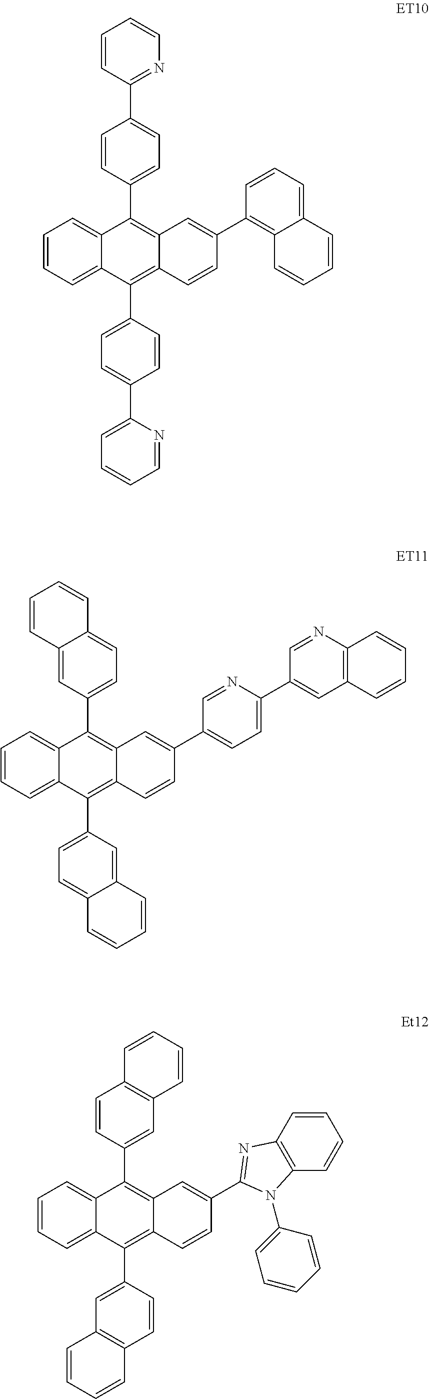 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

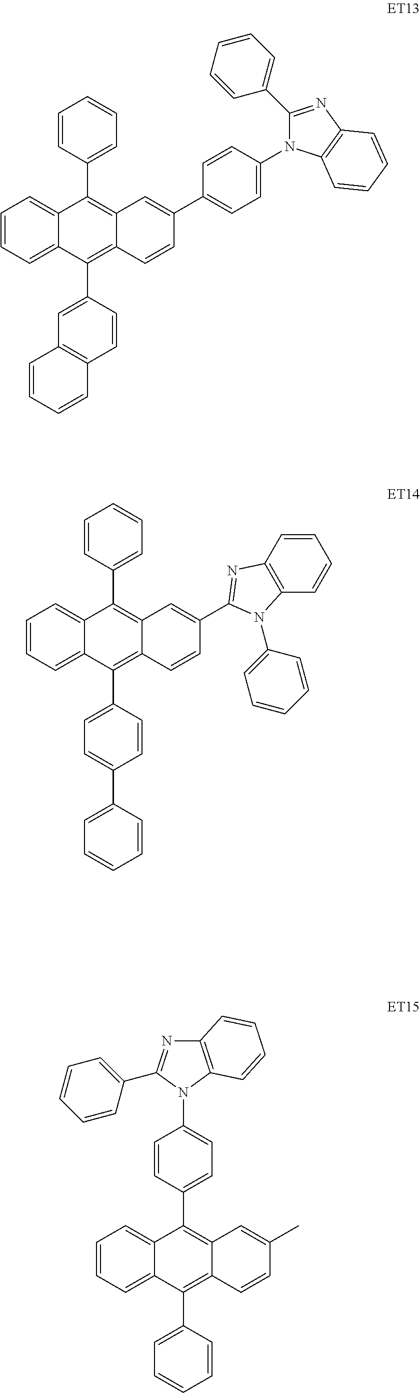 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

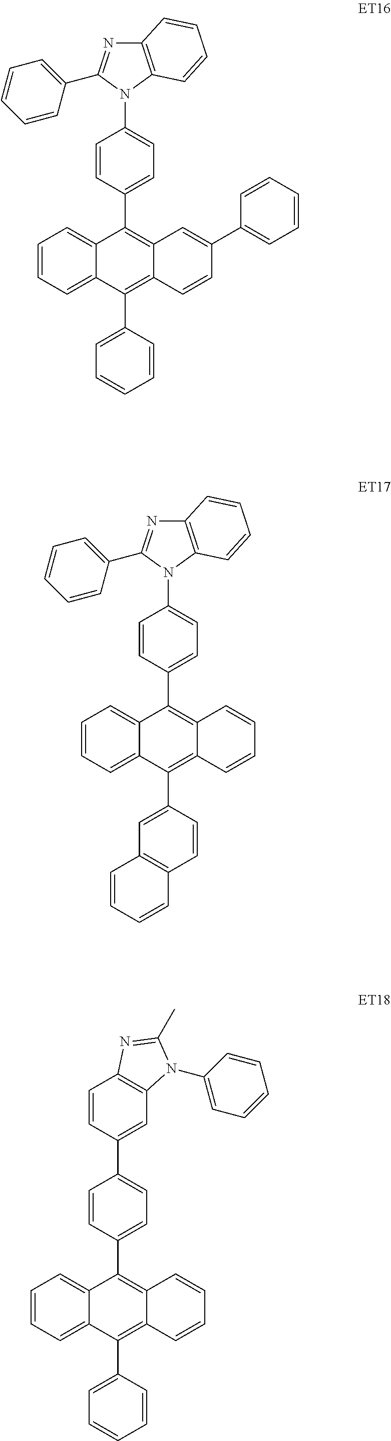 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

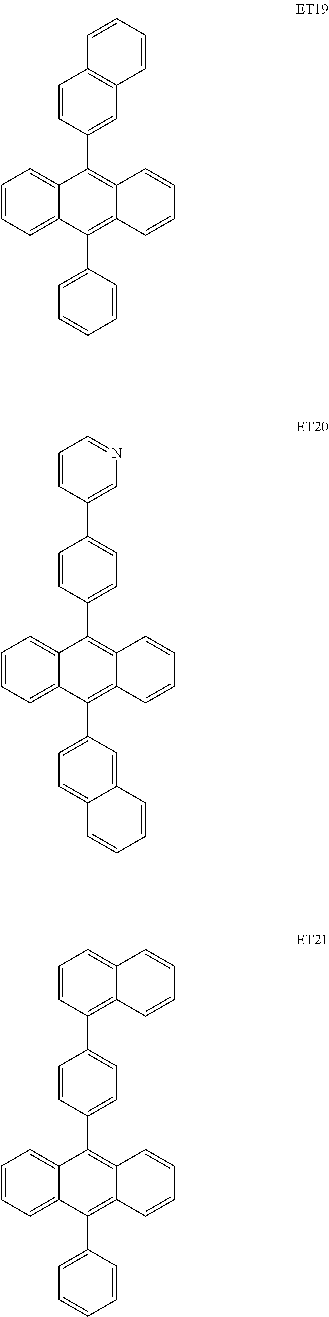

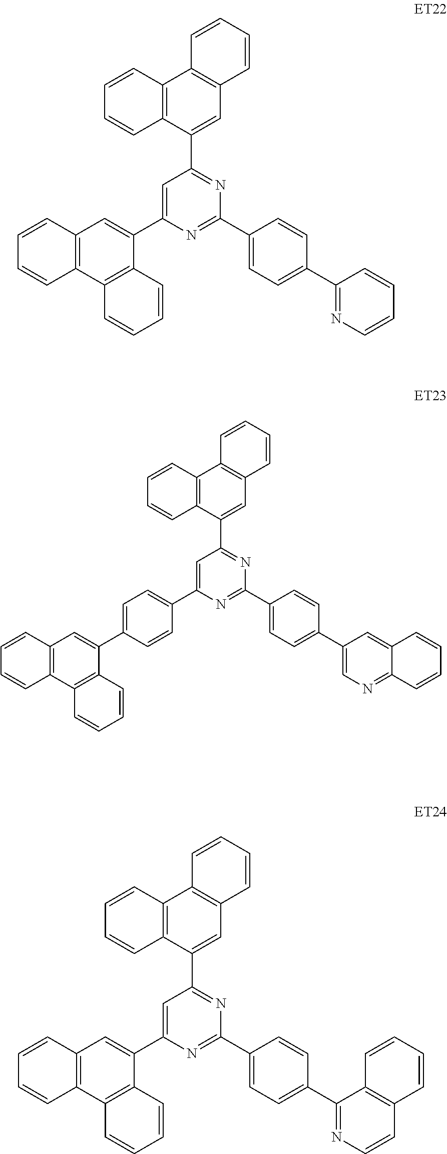 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

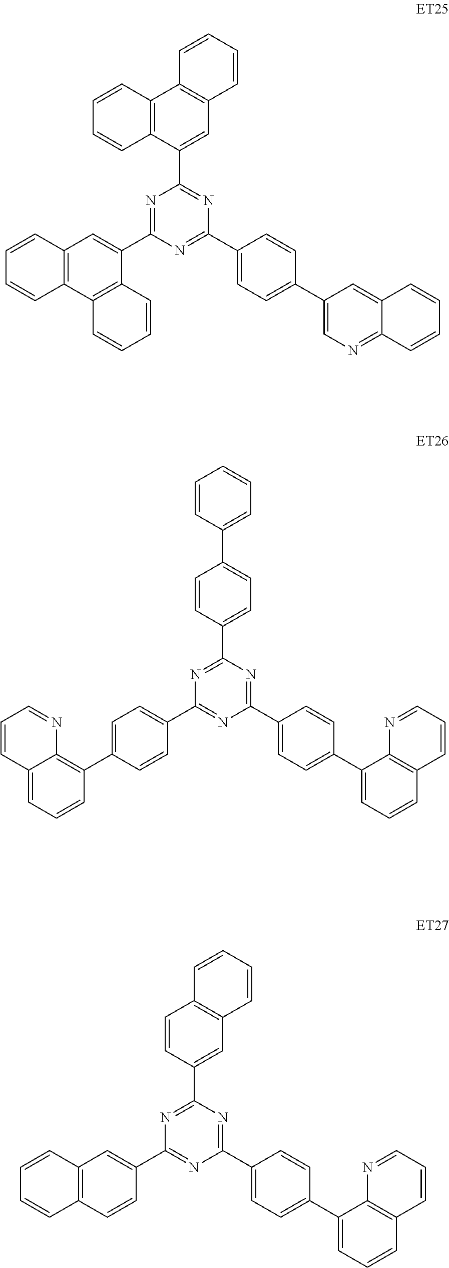 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

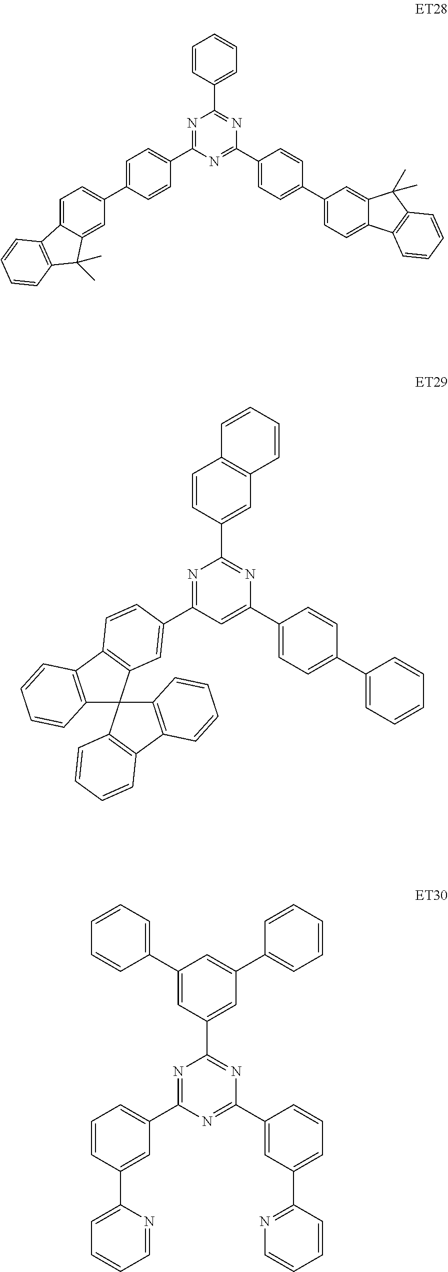 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

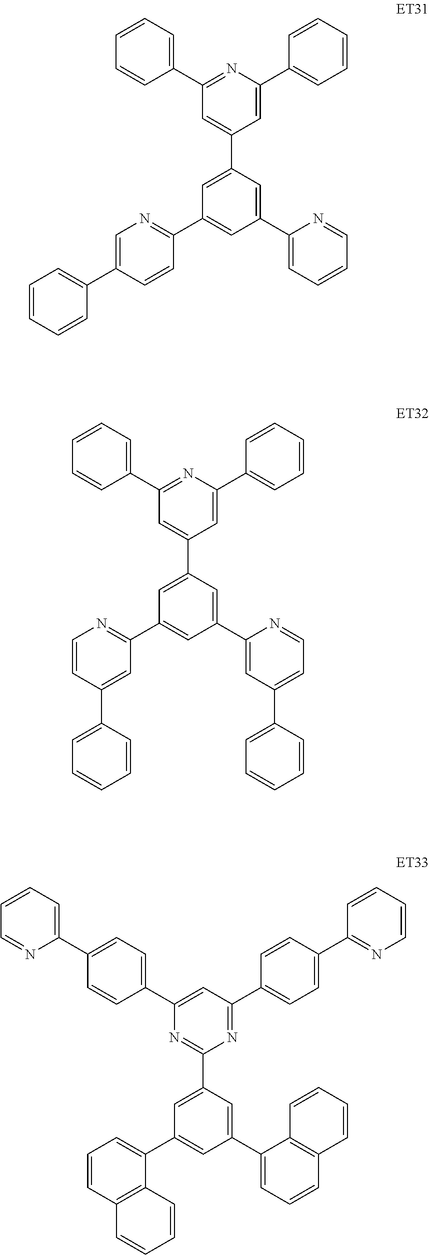 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

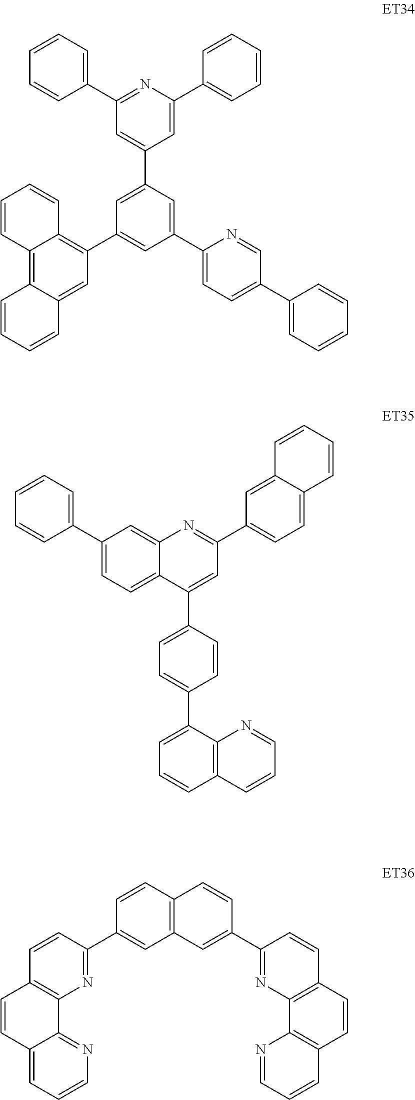 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In one or more embodiments, the electron transport region ETR may include a metal halide such as LiF, NaCl, CsF, RbCl, RbI, CuI, and KI, a lanthanide metal such as Yb, and a co-deposited material of the metal halide and the lanthanide metal. For example, the electron transport region ETR may include KI:Yb, RbI:Yb, LiF:Yb, and/or the like, as a co-deposited material. In one or more embodiments, the electron transport region ETR may be formed utilizing a metal oxide such as Li₂O or BaO, or 8-hydroxyl-lithium quinolate (Liq), and/or the like, but the embodiment of the present disclosure is not limited thereto. The electron transport region ETR may also be formed of a mixture material of an electron transport material and an insulating organometallic salt. The organometallic salt may be a material having an energy band gap of about 4 electron volt (eV) or more. For example, the organometallic salt may include, for example, a metal acetate, a metal benzoate, a metal acetoacetate, a metal acetylacetonate, or a metal stearate.

The electron transport region ETR may further include at least one of 2,9-dimethyl-4,7-diphenyl-1,10-phenanthroline (BCP), diphenyl(4-(triphenylsilyl)phenyl)phosphine oxide (TSPO1), or 4,7-diphenyl-1,10-phenanthroline (Bphen) (e.g., at least one selected from among 2,9-dimethyl-4,7-diphenyl-1,10-phenanthroline (BCP), diphenyl(4-(triphenylsilyl)phenyl)phosphine oxide (TSPO1), or 4,7-diphenyl-1,10-phenanthroline (Bphen)) in addition to the herein-described materials, but the embodiment of the present disclosure is not limited thereto.

The electron transport region ETR may include the herein-described compounds of the hole transport region in at least one of the electron injection layer EIL, the electron transport layer ETL, or the hole blocking layer HBL.

When the electron transport region ETR includes the electron transport layer ETL, the electron transport layer ETL may have a thickness of about 100 Å to about 1,000 Å, for example, about 150 Å to about 500 Å. If the thickness of the electron transport layer ETL satisfies the aforementioned range, satisfactory electron transport characteristics may be obtained without a substantial increase in driving voltage. When the electron transport region ETR includes the electron injection layer EIL, the electron injection layer EIL may have a thickness of about 1 Å to about 100 Å, for example, about 3 Å to about 90 Å. If the thickness of the electron injection layer EIL satisfies the herein-described ranges, satisfactory electron injection characteristics may be obtained without a substantial increase in driving voltage.

The second electrode EL2 is provided on the electron transport region ETR. The second electrode EL2 may be a common electrode. The second electrode EL2 may be a cathode or an anode, but the embodiment of the present disclosure is not limited thereto. For example, if (e.g., when) the first electrode EL1 is an anode, the second electrode EL2 may be a cathode, and if (e.g., when) the first electrode EL1 is a cathode, the second electrode EL2 may be an anode.

The second electrode EL2 may be a transmissive electrode, a transflective electrode, or a reflective electrode. When the second electrode EL2 is the transmissive electrode, the second electrode EL2 may be formed of a transparent metal oxide, for example, indium tin oxide (ITO), indium zinc oxide (IZO), zinc oxide (ZnO), indium tin zinc oxide (ITZO), and/or the like.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, or a compound or mixture thereof (e.g., AgMg, AgYb, or MgAg). In one or more embodiments, the second electrode EL2 may have a multilayer structure including a reflective film or a transflective film formed of the herein-described materials, and a transparent conductive film formed of ITO, IZO, ZnO, ITZO, and/or the like. For example, the second electrode EL2 may include the herein-described metal materials, combinations of at least two metal materials of the herein-described metal materials, oxides of the herein-described metal materials, and/or the like.

In one or more embodiments, the second electrode EL2 may be connected with an auxiliary electrode. If the second electrode EL2 is connected with the auxiliary electrode, the resistance of the second electrode EL2 may be decreased.

In one or more embodiments, a capping layer CPL may further be arranged on the second electrode EL2 of the light emitting element ED of one or more embodiments. The capping layer CPL may include a multilayer or a single layer.

In one or more embodiments, the capping layer CPL may be an organic layer or an inorganic layer. For example, if (e.g., when) the capping layer CPL contains an inorganic material, the inorganic material may include an alkaline metal compound (e.g., LiF), an alkaline earth metal compound (e.g., MgF₂), SiON, SiNₓ, SiOy, and/or the like.

For example, if (e.g., when) the capping layer CPL includes an organic material, the organic material may include a-NPD, NPB, TPD, m-MTDATA, Alq3, CuPc, N4,N4,N4′,N4′-tetra(biphenyl-4-yl)biphenyl-4,4′-diamine (TPD15), 4,4′,4″-tris(carbazol-9-yl)triphenylamine (TCTA), and/or the like, or an epoxy resin, or acrylate such as methacrylate. However, the embodiment of the present disclosure is not limited thereto, and the capping layer CPL may include at least one among Compounds P1 to P5.

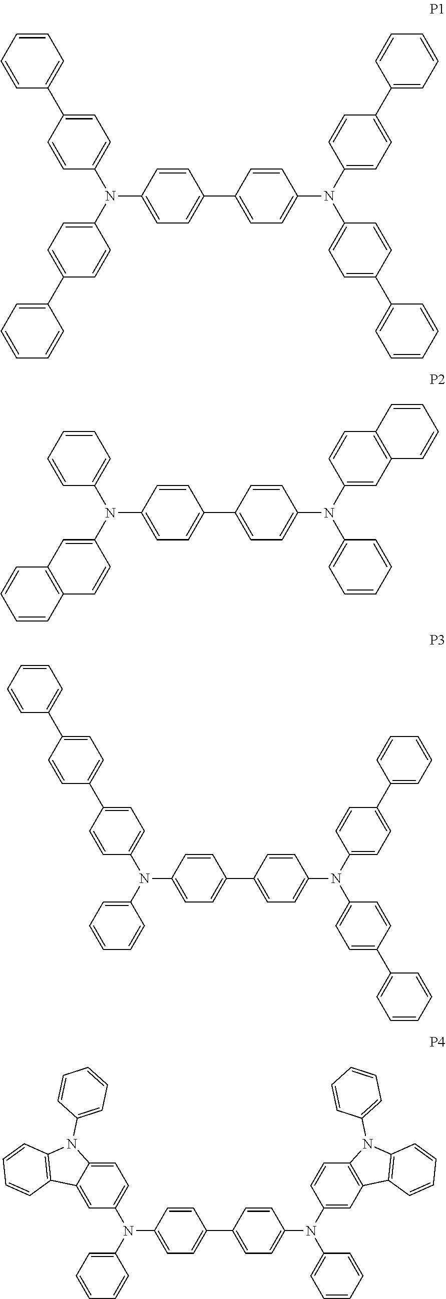 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

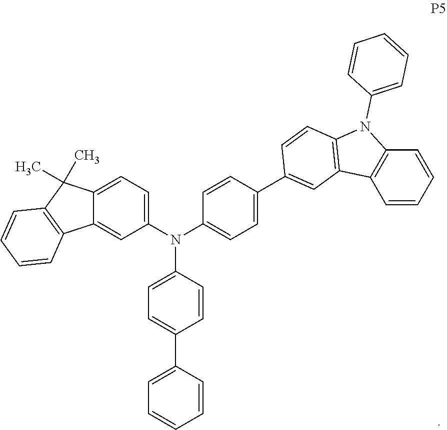 [SMILES: CC1(C)c2ccccc2-c2cc(N(c3ccc(-c4ccccc4)cc3)c3ccc(-c4ccc5c(c4)c4ccccc4n5-c4ccccc4)cc3)ccc21]

In one or more embodiments, the refractive index of the capping layer CPL may be about 1.6 or more. For example, the refractive index of the capping layer CPL may be about 1.6 or more with respect to light in a wavelength range of about 550 nanometer (nm) to about 660 nm.

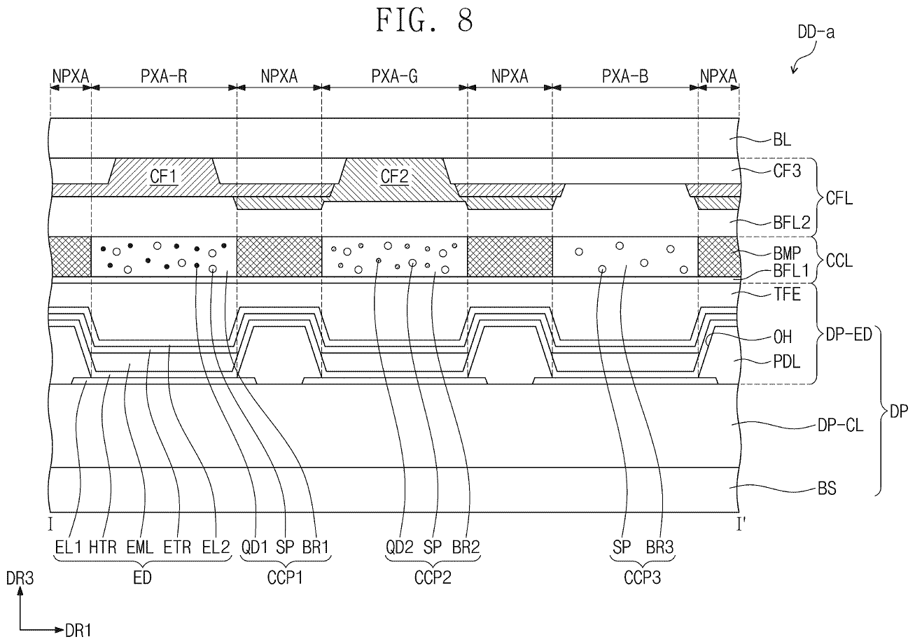

Each of FIGS. 8 to 11 is a cross-sectional view of a display device according to one or more embodiments of the present disclosure. Hereinafter, in describing the display devices of embodiments with reference to FIGS. 8 to 11, the duplicated features which have been described in FIGS. 1 to 7 are not described again, but their differences will be mainly described, in more detail.

Referring to FIG. 8, the display device DD-a according to one or more embodiments may include a display panel DP including a display element layer DP-ED, a light control layer CCL arranged on the display panel DP, and a color filter layer CFL. In one or more embodiments illustrated in FIG. 8, the display panel DP may include a base layer BS, a circuit layer DP-CL provided on the base layer BS, and the display element layer DP-ED, and the display element layer DP-ED may include a light emitting element ED.

The light emitting element ED may include a first electrode EL1, a hole transport region HTR arranged on the first electrode EL1, a light emitting layer EML arranged on the hole transport region HTR, an electron transport region ETR arranged on the light emitting layer EML, and a second electrode EL2 arranged on the electron transport region ETR. In one or more embodiments, the structure of the light emitting element of FIG. 3 to FIG. 7 described herein may be equally applied to the structure of the light emitting element ED illustrated in FIG. 8.

The light emitting element ED illustrated in FIG. 8 may include the amine compound of one or more embodiments in the hole transport region HTR. Accordingly, the light emitting element ED may exhibit high-efficiency and long-lifespan properties. In one or more embodiments, the light emitting element ED of one or more embodiments may exhibit high light efficiency and improved lifespan properties in a blue light emission region. The light emitting element ED according to one or more embodiments includes the amine compound of one or more embodiments in the hole transport region HTR, and thus, exhibits high-efficiency and long-lifespan properties, and accordingly, the display device DD-a of one or more embodiments may exhibit excellent or suitable display quality.

Referring to FIG. 8, the light emitting layer EML may be arranged in an opening OH defined in a pixel defining layer PDL. For example, the light emitting layer EML which is divided by the pixel defining layer PDL and provided corresponding to each light emitting regions PXA-R, PXA-G, and PXA-B may be to emit light in substantially the same wavelength range. In the display device DD-a of one or more embodiments, the light emitting layer EML may be to emit blue light. In one or more embodiments, unlike the configuration illustrated, in one or more embodiments, the light emitting layer EML may be provided as a common layer in the entire light emitting regions PXA-R, PXA-G, and PXA-B.

The light control layer CCL may be arranged on the display panel DP. Although the light control layer CCL is shown as being arranged above the display element layer DP-ED, the embodiment is not limited to this, and the light control layer CCL may be arranged below the display element layer DP-ED. The light control layer CCL may include a light conversion body. The light conversion body may be a quantum dot, a phosphor, and/or the like. The light conversion body may be to emit provided light by converting the wavelength thereof. For example, the light control layer CCL may a layer containing the quantum dot or a layer containing the phosphor.

The light control layer CCL may include a plurality of light control parts CCP1, CCP2 and CCP3. The light control parts CCP1, CCP2, and CCP3 may be spaced and/or apart from each other.

Referring to FIG. 8, divided patterns BMP may be arranged between the light control parts CCP1, CCP2 and CCP3 which are spaced and/or apart from each other, but the embodiment of the present disclosure is not limited thereto. FIG. 8 illustrates that the divided patterns BMP do not overlap the light control parts CCP1, CCP2 and CCP3, but at least a portion of the edges of the light control parts CCP1, CCP2 and CCP3 may overlap the divided patterns BMP.

The light control layer CCL may include a first light control part CCP1 containing a first quantum dot QD1 which converts first color light provided from the light emitting element ED into second color light, a second light control part CCP2 containing a second quantum dot QD2 which converts the first color light into third color light, and a third light control part CCP3 which transmits the first color light.

In one or more embodiments, the first light control part CCP1 may provide red light that is the second color light, and the second light control part CCP2 may provide green light that is the third color light. The third light control part CCP3 may provide blue light by transmitting the blue light that is the first color light provided from the light emitting element ED. For example, the first quantum dot QD1 may be a red quantum dot, and the second quantum dot QD2 may be a green quantum dot. The same as described herein may be applied with respect to the quantum dots QD1 and QD2.

In one or more embodiments, the light control layer CCL may further include a scatterer SP. The first light control part CCP1 may include the first quantum dot QD1 and the scatterer SP, the second light control part CCP2 may include the second quantum dot QD2 and the scatterer SP, and the third light control part CCP3 may not include (e.g., may exclude) any quantum dot but include the scatterer SP.

The scatterer SP may be inorganic particles. For example, the scatterer SP may include at least one of TiO₂, ZnO, Al₂O₃, SiO₂, or hollow sphere silica. The scatterer SP may include any one among TiO₂, ZnO, Al₂O₃, SiO₂, and hollow sphere silica, or may be a mixture of at least two materials selected from among TiO₂, ZnO, Al₂O₃, SiO₂, and hollow sphere silica.

The first light control part CCP1, the second light control part CCP2, and the third light control part CCP3 each may include base resins BR1, BR2, and BR3 in which the quantum dots QD1 and QD2 and the scatterer SP are dispersed. In one or more embodiments, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3.

The base resins BR1, BR2, and BR3 are media in which the quantum dots QD1 and QD2 and the scatterer SP are dispersed, and may be formed of one or more suitable resin compositions, which may be generally referred to as a binder. For example, the base resins BR1, BR2, and BR3 may be acrylic-based resins, urethane-based resins, silicone-based resins, epoxy-based resins, and/or the like. The base resins BR1, BR2, and BR3 may be transparent resins. In one or more embodiments, the first base resin BR1, the second base resin BR2, and the third base resin BR3 may be the same as or different from each other.

The light control layer CCL may include a barrier layer BFL1. The barrier layer BFL1 may serve to prevent or reduce the penetration of moisture and/or oxygen (hereinafter, referred to as ‘moisture/oxygen’). The barrier layer BFL1 may block or reduce the light control parts CCP1, CCP2 and CCP3 from being exposed to moisture/oxygen. In one or more embodiments, the barrier layer BFL1 may cover the light control parts CCP1, CCP2, and CCP3. In one or more embodiments, the barrier layer BFL2 may be provided between the light control parts CCP1, CCP2, and CCP3 and the color filter layer CFL.

The barrier layers BFL1 and BFL2 may include at least one inorganic layer. For example, the barrier layers BFL1 and BFL2 may include an inorganic material. For example, the barrier layers BFL1 and BFL2 may include a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a metal thin film which secures a transmittance, and/or the like. In one or more embodiments, the barrier layers BFL1 and BFL2 may further include an organic film. The barrier layers BFL1 and BFL2 may be formed of a single layer or a plurality of layers.

In the display device DD-a of one or more embodiments, the color filter layer CFL may be arranged on the light control layer CCL. For example, the color filter layer CFL may be directly arranged on the light control layer CCL. In this case, the barrier layer BFL2 may not be provided.

The color filter layer CFL may include color filters CF1, CF2, and CF3. The color filter layer CFL may include a first filter CF1 configured to transmit the second color light, a second filter CF2 configured to transmit the third color light, and a third filter CF3 configured to transmit the first color light. For example, the first filter CF1 may be a red filter, the second filter CF2 may be a green filter, and the third filter CF3 may be a blue filter. The filters CF1, CF2, and CF3 each may include a polymeric photosensitive resin and a pigment or dye. The first filter CF1 may include a red pigment or dye, the second filter CF2 may include a green pigment or dye, and the third filter CF3 may include a blue pigment or dye.

In one or more embodiments, the embodiment of the present disclosure is not limited thereto, and the third filter CF3 may not include (e.g., may exclude) a pigment or dye. The third filter CF3 may include a polymeric photosensitive resin and may not include (e.g., may exclude) a pigment or dye. The third filter CF3 may be transparent. The third filter CF3 may be formed of a transparent photosensitive resin.

Furthermore, in one or more embodiments, the first filter CF1 and the second filter CF2 may be a yellow filter. The first filter CF1 and the second filter CF2 may not be separated but be provided as one filter.

In one or more embodiments, the color filter layer CFL may further include a light shielding part. The light shielding part may be a black matrix. The light shielding part may include an organic light shielding material or an inorganic light shielding material containing a black pigment or dye. The light shielding part may prevent or reduce light leakage, and may separate boundaries between the adjacent filters CF1, CF2, and CF3. Also, in one or more embodiments, the light shielding part may be formed of a blue filter.

The first to third filters CF1, CF2, and CF3 may be arranged corresponding to the red light emitting region PXA-R, the green light emitting region PXA-G, and the blue light emitting region PXA-B, respectively.

A base substrate BL may be arranged on the color filter layer CFL. The base substrate BL may be a member which provides a base surface in which the color filter layer CFL, the light control layer CCL, and/or the like are arranged. The base substrate BL may be a glass substrate, a metal substrate, a plastic substrate, and/or the like.

However, the embodiment of the present disclosure is not limited thereto, and the base substrate BL may be an inorganic layer, an organic layer, or a composite material layer. In one or more embodiments, unlike the configuration illustrated, in one or more embodiments, the base substrate BL may not be provided.

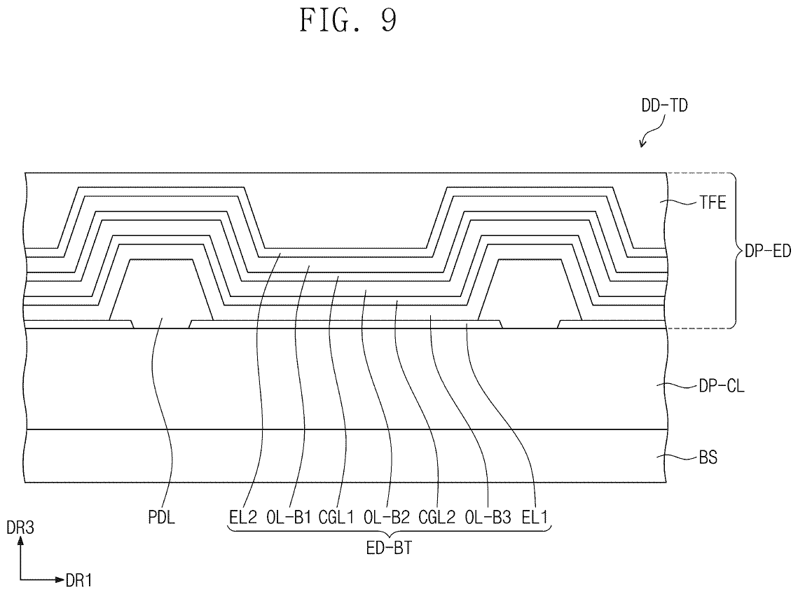

FIG. 9 is a cross-sectional view illustrating a portion of the display device according to one or more embodiments. In a display device DD-TD according to one or more embodiments, a light-emitting element ED-BT may include a plurality of light-emitting structures OL-B1, OL-B2, and OL-B3.

At least one of the plurality of light emitting structures OL-B1, OL-B2, and OL-B3 may include the amine compound of one or more embodiments. Accordingly, the light emitting element ED-BT may exhibit high-efficiency and long-lifespan properties.

The light emitting element ED-BT may include a first electrode EL1 and a second electrode EL2 which face each other, and the plurality of light emitting structures OL-B1, OL-B2, and OL-B3 sequentially stacked in the thickness direction between the first electrode EL1 and the second electrode EL2. The light emitting structures OL-B1, OL-B2, and OL-B3 each may include a light emitting layer EML (FIG. 8) and a hole transport region HTR and an electron transport region ETR arranged with the light emitting layer EML (FIG. 8) located therebetween. For example, the light emitting element ED-BT included in the display device DD-TD of one or more embodiments may be a light emitting element having a tandem structure and including a plurality of light emitting layers.

In one or more embodiments illustrated in FIG. 9, all light beams respectively emitted from the light emitting structures OL-B1, OL-B2, and OL-B3 may be blue light. However, the embodiment of the present disclosure is not limited thereto, and the light beams respectively emitted from the light emitting structures OL-B1, OL-B2, and OL-B3 may have wavelength ranges different from each other. For example, the light emitting element ED-BT including the plurality of light emitting structures OL-B1, OL-B2, and OL-B3 which emit light beams having wavelength ranges different from each other may be to emit white light.

A charge generating layer CGL may be arranged between the neighboring light-emitting structures OL-B1, OL-B2 and OL-B3. The charge generating layer CGL may include a p-type or kind charge generating layer (e.g., p-charge generating layer) and/or an n-type or kind charge generating layer (e.g., n-charge generating layer).

In the embodiment illustrated in FIG. 9, at least one of the light emitting structures OL-B1, OL-B2, and OL-B3 may include the amine compound of one or more embodiments in the hole transport region HTR (see FIG. 8). Accordingly, at least one of the light emitting structures OL-B1, OL-B2, and OL-B3 may exhibit high-efficiency and long-lifespan properties.

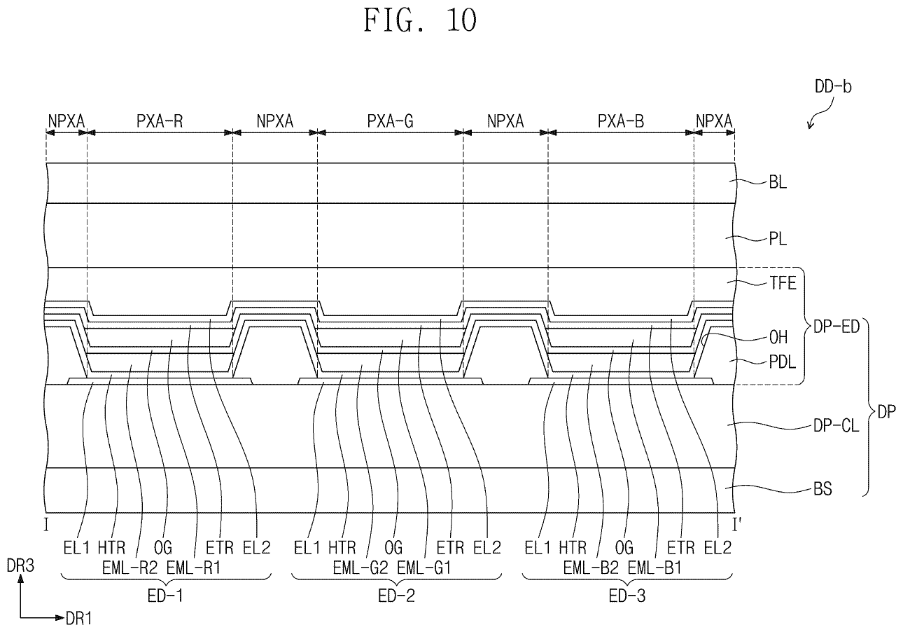

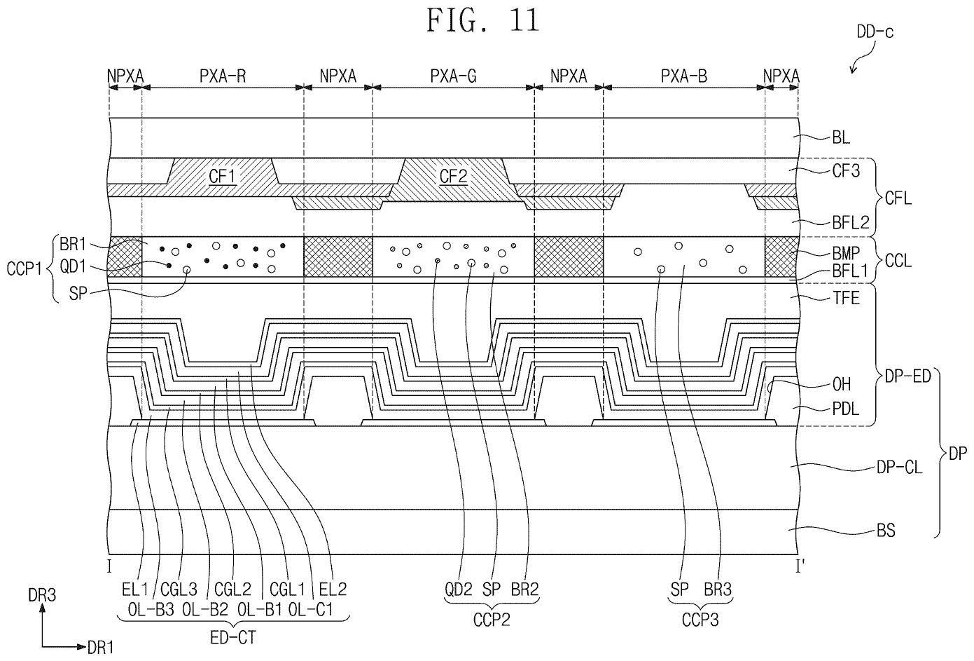

Referring to FIG. 10, a display device DD-b according to one or more embodiments may include light emitting elements ED-1, ED-2, and ED-3 in which two light emitting layers are stacked. At least one of the light emitting elements ED-1, ED-2, and ED-3 may include the amine compound of one or more embodiments. Accordingly, the light emitting elements ED-1, ED-2, and ED-3 may exhibit high-efficiency and long-lifespan properties. In one or more embodiments, the light emitting element ED-3 of one or more embodiments may exhibit high efficiency and improved lifespan properties in a blue light emission region.

Compared with the display device DD of one or more embodiments illustrated in FIG. 2, one or more embodiments illustrated in FIG. 10 has a difference in that the first to third light emitting elements ED-1, ED-2, and ED-3 each include two light emitting layers stacked in the thickness direction. In each of the first to third light emitting elements ED-1, ED-2, and ED-3, the two light emitting layers may be to emit light in substantially the same wavelength region.

The first light emitting element ED-1 may include a first red light emitting layer EML-R1 and a second red light emitting layer EML-R2. The second light emitting element ED-2 may include a first green light emitting layer EML-G1 and a second green light emitting layer EML-G2. In one or more embodiments, the third light emitting element ED-3 may include a first blue light emitting layer EML-B1 and a second blue light emitting layer EML-B2. An emission auxiliary part OG may be arranged between the first red light emitting layer EML-R1 and the second red light emitting layer EML-R2, between the first green light emitting layer EML-G1 and the second green light emitting layer EML-G2, and between the first blue light emitting layer EML-B1 and the second blue light emitting layer EML-B2.

The emission auxiliary part OG may include a single layer or a multilayer. The emission auxiliary part OG may include a charge generation layer. More specifically, the emission auxiliary part OG may include an electron transport region, a charge generation layer, and a hole transport region that are sequentially stacked. The emission auxiliary part OG may be provided as a common layer in the whole of the first to third light emitting elements ED-1, ED-2, and ED-3. However, the embodiment of the present disclosure is not limited thereto, and the emission auxiliary part OG may be provided by being patterned within the openings OH defined in the pixel defining layer PDL.

The first red light emitting layer EML-R1, the first green light emitting layer EML-G1, and the first blue light emitting layer EML-B1 may be arranged between the electron transport region ETR and the emission auxiliary part OG. The second red light emitting layer EML-R2, the second green light emitting layer EML-G2, and the second blue light emitting layer EML-B2 may be arranged between the emission auxiliary part OG and the hole transport region HTR.

For example, the first light emitting element ED-1 may include the first electrode EL1, the hole transport region HTR, the second red light emitting layer EML-R2, the emission auxiliary part OG, the first red light emitting layer EML-R1, the electron transport region ETR, and the second electrode EL2 that are sequentially stacked. The second light emitting element ED-2 may include the first electrode EL1, the hole transport region HTR, the second green light emitting layer EML-G2, the emission auxiliary part OG, the first green light emitting layer EML-G1, the electron transport region ETR, and the second electrode EL2 that are sequentially stacked. The third light emitting element ED-3 may include the first electrode EL1, the hole transport region HTR, the second blue light emitting layer EML-B2, the emission auxiliary part OG, the first blue light emitting layer EML-B1, the electron transport region ETR, and the second electrode EL2 that are sequentially stacked.

The light emitting elements ED-1, ED-2, and ED-3 according to one or more embodiments includes the amine compound of one or more embodiments in the hole transport region HTR and exhibits high efficiency and long lifespan characteristics, and accordingly, the display device of one or more embodiments DD-b can exhibit excellent or suitable display quality.

In one or more embodiments, an optical auxiliary layer PL may be arranged on the display element layer DP-ED. The optical auxiliary layer PL may include a polarizing layer. The optical auxiliary layer PL may be arranged on the display panel DP and control reflected light in the display panel DP due to external light. Unlike the configuration illustrated, the optical auxiliary layer PL in the display device according to one or more embodiments may not be provided.

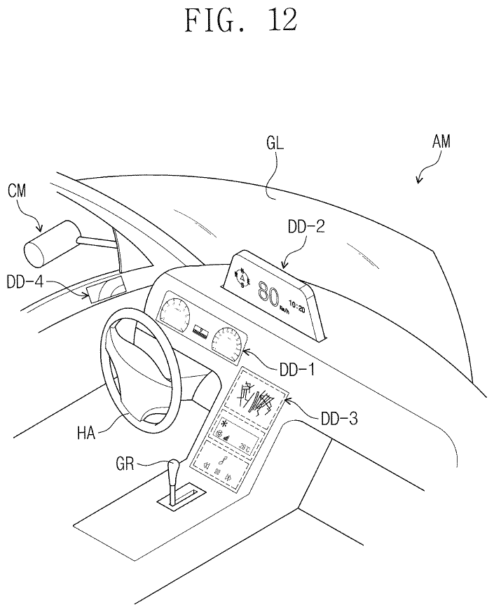

Unlike FIGS. 9 and 10, the display device DD-c of FIG. 11 is illustrated to include four light emitting structures OL-B1, OL-B2, OL-B3, and OL-C1. The light emitting element ED-CT may include the first electrode EL1 and the second electrode EL2 facing each other, and the first to fourth light emitting structures OL-B1, OL-B2, OL-B3, and OL-C1 sequentially stacked in a thickness direction between the first electrode EL1 and the second electrode EL2.

At least one of the first to fourth light emitting structures OL-B1, OL-B2, OL-B3, and OL-C1 may include the amine compound of one or more embodiments. Accordingly, the light emitting element ED-CT may exhibit high-efficiency and long-lifespan properties. In one or more embodiments, the display device DD-c of one or more embodiments may exhibit excellent or suitable display quality.

Charge generation layers CGL1, CGL2, and CGL3 may be arranged between the first to fourth light emitting structures OL-B1, OL-B2, OL-B3, and OL-C1. Among the four light emitting structures, the first to third light emitting structures OL-B1, OL-B2, and OL-B3 may be to emit blue light, and the fourth light emitting structure OL-C1 may be to emit green light. However, the embodiment of the present disclosure is not limited thereto, and the first to fourth light emitting structures OL-B1, OL-B2, OL-B3, and OL-C1 may be to emit light beams in different wavelength regions. The charge generation layers CGL1, CGL2, and CGL3 arranged between adjacent light emitting structures OL-B1, OL-B2, OL-B3, and OL-C1 may include a p-type or kind charge generation layer (e.g., p-charge generating layer) and/or an n-type or kind charge generation layer (e.g., n-charge generating layer).

In one or more embodiments, the electronic apparatus may include a display device including a plurality of light emitting elements, and a control part which controls the display device. The electronic apparatus of one or more embodiments may be a device that is activated according to an electrical signal. The electronic apparatus may include display devices of one or more suitable embodiments. For example, the electronic apparatus may include not only large-sized electronic apparatuses such as a television set, a monitor, or an outdoor billboard but also include small- and medium-sized electronic apparatuses such as a personal computer, a laptop computer, a personal digital terminal, a display device for a vehicle, a game console, a portable electronic device, or a camera.

FIG. 12 is a view illustrating a vehicle AM in which first to fourth display devices DD-1, DD-2, DD-3, and DD-4 are arranged. At least one among the first to fourth display devices DD-1, DD-2, DD-3, and DD-4 may include the same constitution of display devices DD, DD-TD, DD-a, DD-b, and DD-c, according to one or more embodiments, described with reference to FIGS. 1, 2, and 8 to 11.

FIG. 12 illustrates a vehicle AM, but this is merely an example, and the first to fourth display devices DD-1, DD-2, DD-3, and DD-4 may be arranged in other transportation vehicles, such as bicycles, motorcycles, trains, ships, and airplanes. In one or more embodiments, at least one among the first to fourth display devices DD-1, DD-2, DD-3, and DD-4 including the same configuration as the display devices DD, DD-TD, DD-a, DD-b, and DD-c of one or more embodiments may be employed in a personal computer, a laptop computer, a personal digital terminal, a game console, a portable electronic device, a television, a monitor, an outdoor billboard, and/or the like. In one or more embodiments, these are merely provided as embodiments, and thus may be employed in other electronic apparatuses unless departing from the present disclosure.

At least one selected from among the first to fourth display devices DD-1, DD-2, DD-3, and DD-4 may include the light emitting element ED described with reference to FIG. 3 to FIG. 7. At least one among the first to fourth display devices DD-1, DD-2, DD-3, and DD-4 may include the amine compound of one or more embodiments. Accordingly, the first to fourth display devices DD-1, DD-2, DD-3, and DD-4 including the amine-based compound of one or more embodiments may have improved display efficiency and display lifespan. In one or more embodiments, the first to fourth display devices DD-1, DD-2, DD-3, and DD-4 including the amine-based compound of one or more embodiments may exhibit excellent or suitable display quality.

Referring to FIG. 12, the vehicle AM may include a steering wheel HA and a gear GR for driving the vehicle AM. In one or more embodiments, the vehicle AM may include a front window GL arranged so as to face the driver.

The first display device DD-1 may be arranged in a first region overlapping the steering wheel HA. For example, the first display device DD-1 may be a digital cluster which displays first information of the vehicle AM. The first information may include a first scale which indicates a driving speed of the vehicle AM, a second scale which indicates an engine speed (that is, revolutions per minute (RPM)), an image which indicates a fuel state, and/or the like. A first scale and a second scale may be indicated as a digital image.

The second display device DD-2 may be arranged in a second region facing the driver's seat and overlapping the front window GL. The driver's seat may be a seat in which the steering wheel HA is arranged. For example, the second display device DD-2 may be a head up display (HUD) which displays second information of the vehicle AM. The second display device DD-2 may be optically transparent. The second information may include digital numbers which indicate a driving speed, and may further include information such as the current time. Unlike the configuration illustrated, the second information of the second display device DD-2 may be projected to the front window GL to be displayed.

The third display device DD-3 may be arranged in a third region adjacent to the gear GR. For example, the third display device DD-3 may be arranged between the driver's seat and the passenger seat and may be a center information display (CID) for a vehicle for displaying third information. The passenger seat may be a seat spaced and/or apart from the driver's seat with the gear GR arranged therebetween. The third information may include information about traffic (e.g., navigation information), playing music or radio or a video (or an image), temperatures inside the vehicle AM, and/or the like.

The fourth display device DD-4 may be spaced and/or apart from the steering wheel HA and the gear GR, and may be arranged in a fourth region adjacent to the side of the vehicle AM. For example, the fourth display device DD-4 may be a digital side-view mirror which displays fourth information. The fourth display device DD-4 may display an image outside the vehicle AM taken by a camera module CM arranged outside the vehicle AM. The fourth information may include an image outside the vehicle AM.

The herein-described first to fourth information may be examples, and the first to fourth display devices DD-1, DD-2, DD-3, and DD-4 may further display information about the inside and outside of the vehicle AM. The first to fourth information may include different information. However, the embodiment of the present disclosure is not limited thereto, and a part of the first to fourth information may include the same information as one another.

Terms such as “substantially,” “about,” and “approximately” are used as relative terms and not as terms of degree, and are intended to account for the inherent deviations in measured or calculated values that would be recognized by those of ordinary skill in the art. They may be inclusive of the stated value and an acceptable range of deviation as determined by one of ordinary skill in the art, considering the limitations and error associated with measurement of that quantity. For example, “about” may refer to one or more standard deviations, or ±30%, 20%, 10%, 5% of the stated value.

Numerical ranges disclosed herein include and are intended to disclose all subsumed sub-ranges of the same numerical precision. For example, a range of “1.0 to 10.0” includes all subranges having a minimum value equal to or greater than 1.0 and a maximum value equal to or less than 10.0, such as, for example, 2.4 to 7.6. Applicant therefore reserves the right to amend this specification, including the claims, to expressly recite any sub-range subsumed within the ranges expressly recited herein.

The light emitting device, light emitting element, and/or any other relevant devices or components according to embodiments of the present disclosure described herein may be implemented utilizing any suitable hardware, firmware (e.g., an application-specific integrated circuit), software, or a combination of software, firmware, and hardware. For example, the various components of the light emitting device and/or light emitting element may be formed on one integrated circuit (IC) chip or on separate IC chips. Further, the various components of the light emitting device and/or light emitting element may be implemented on a flexible printed circuit film, a tape carrier package (TCP), a printed circuit board (PCB), or formed on one substrate. Further, the various components of the device and/or element may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

In the present disclosure, when particles are spherical, “diameter” indicates an average particle diameter, and when the particles are non-spherical, the “diameter” indicates a major axis length. The diameter (or size) of the particles may be measured by particle size analysis, dynamic light scattering, scanning electron microscopy, and/or transmission electron microscope photography. When the size of the particles is measured utilizing a particle size analyzer, the average particle diameter (or size) may be referred to as D50. The term “D50” as utilized herein refers to the average diameter (or size) of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size. Particle size analysis may be performed with a HORIBA LA-950 laser particle size analyzer.

Hereinafter, referring to Examples and Comparative Examples, the amine compound according to one or more embodiments of the present disclosure and the light emitting element of one or more embodiments will be described in more detail. In one or more embodiments, Examples shown are for illustrative purposes only to facilitate the understanding of the present disclosure, and thus, the scope of the present disclosure is not limited thereto.

### Examples

### 1. Synthesis of Amine Compound of Embodiment

A method for synthesizing an amine compound according to the present embodiment will be described in more detail with reference to methods for synthesizing Compounds 5, 8, 11, 49, 68, 82, 88, 90, 100, 112, 130, 154, 166, 169, 177, and 178. In one or more embodiments, the methods for synthesizing the amine compound described are only examples, and the methods for synthesizing the amine compound according to one or more embodiments of the present disclosure is not limited to the following examples.

### (1) Synthesis of Compound 5

Amine Compound 5 according to one or more embodiments may be synthesized, for example, by steps (e.g., tasks or acts) of Reaction Formula 1.

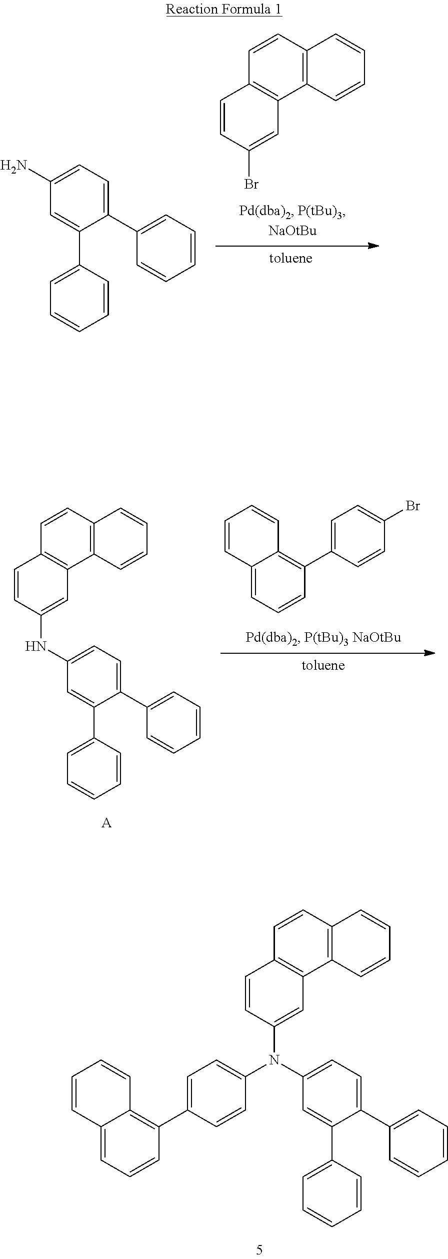

Under an argon (Ar) atmosphere, [1,1′:2′,1″-Terphenyl]-4′-amine (30.0 g), 3-Bromophenanthrene (31.4 g), Bis(dibenzylideneacetone)palladium(0) (Pd(dba)₂, 0.7 g), and Sodium tert-butoxide (NaOtBu, 14.1 g) were added to a 1 L three-neck flask and dissolved in toluene (400 mL), followed by adding Tri-tert-butylphosphine (P(tBu)₂, 2.0 M in toluene, 2.0 mL) thereto, and the mixture was stirred at room temperature for 4 hours. Thereafter, water was added thereto, and extraction was performed with CH₂Cl₂ to collect an organic layer, followed by drying the same with MgSO₄, and then the solvent was removed by distillation under reduced pressure. The obtained crude product was purified by silica gel column chromatography to obtain 34.1 g (yield 66%) of Intermediate Compound A. The molecular weight of Intermediate Compound A measured by FAB-MS measurement was 421.

Under an argon (Ar) atmosphere, Intermediate Compound A (5.0 g), 1-(4-Bromophenyl)naphthalene (3.4 g), Pd(dba)₂ (0.35 g), and NaOtBu (1.7 g) were added to a 300 mL three-neck flask and dissolved in toluene (100 mL), followed by adding P(tBu)₃ (2.0 M in toluene, 0.6 mL) thereto, and the mixture was heated and stirred at 100° C. for 4 hours. Thereafter, water was added thereto, and extraction was performed with CH₂Cl₂ to collect an organic layer, followed by drying the same with MgSO₄, and then the solvent was removed by distillation under reduced pressure. The obtained crude product was purified by silica gel column chromatography to obtain 5.9 g (yield 80%) of Compound 5. The molecular weight of Compound 5 measured by FAB-MS measurement was 623.

### (2) Synthesis of Compound 8

Amine Compound 8 according to one or more embodiments may be synthesized, for example, by steps of Reaction Formula 2.

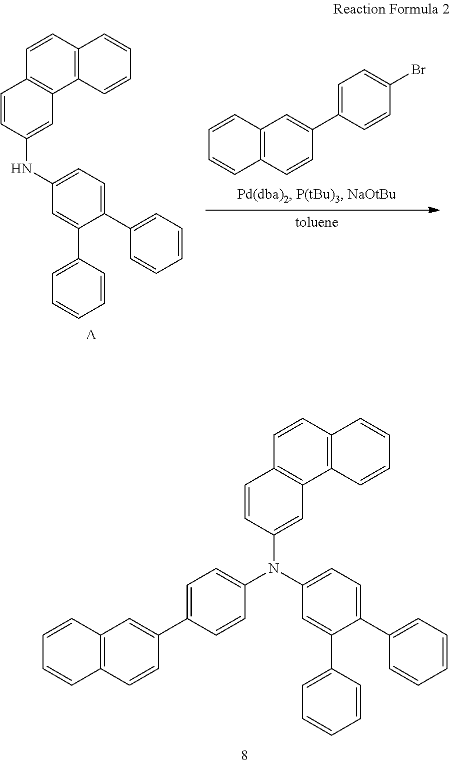

In the manner as the synthesis of Compound 5 described herein, 6.2 g (yield 85%) of Compound 8 was obtained from Intermediate Compound A (5.0 g) and 2-(4-Bromophenyl)naphthalene (3.4 g). The molecular weight of Compound 8 measured by FAB-MS measurement was 623.

### (3) Synthesis of Compound 11

Amine Compound 11 according to one or more embodiments may be synthesized, for example, by steps of Reaction Formula 3.

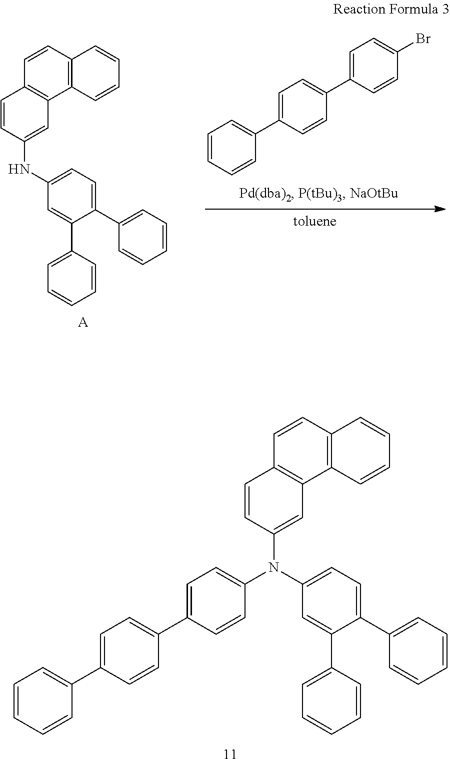

In the manner as the synthesis of Compound 5 described herein, 6.0 g (yield 79%) of Compound 11 was obtained from Intermediate Compound A (5.0 g) and 4-Bromo-1,1′:4′,1″-terphenyl (3.7 g). The molecular weight of Compound 11 measured by FAB-MS measurement was 649.

### (4) Synthesis of Compound 49

Amine Compound 49 according to one or more embodiments may be synthesized, for example, by steps of Reaction Formula 4.

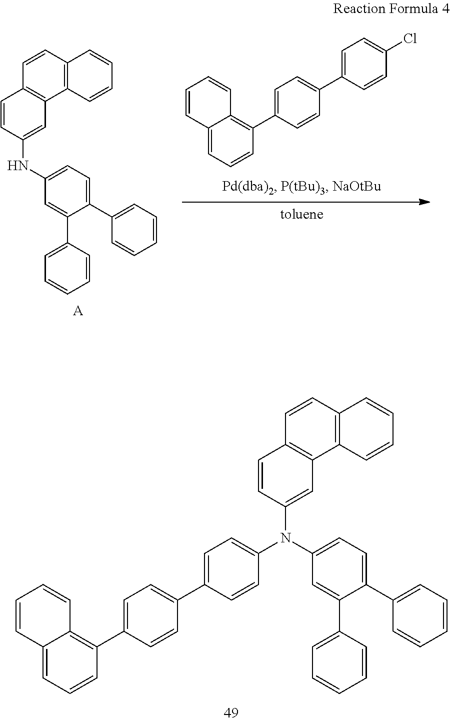

In the manner as the synthesis of Compound 5 described herein, 6.2 g (yield 75%) of Compound 49 was obtained from Intermediate Compound A (5.0 g) and 1-(4′-Chloro-[1,1′-biphenyl]-4-yl)naphthalene (3.7 g). The molecular weight of Compound 49 measured by FAB-MS measurement was 699.

### (5) Synthesis of Compound 68

Amine Compound 68 according to one or more embodiments may be synthesized, for example, by steps of Reaction Formula 5.

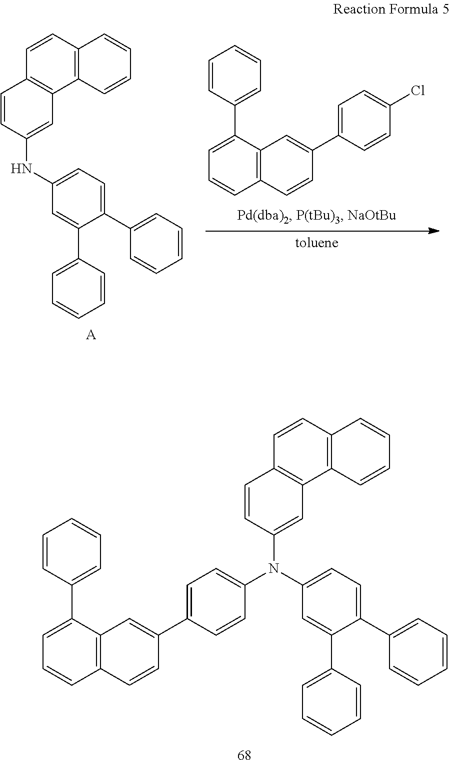

In the manner as the synthesis of Compound 5 described herein, 6.8 g (yield 82%) of Compound 68 was obtained from Intermediate Compound A (5.0 g) and 7-(4-Chlorophenyl)-1-phenylnaphthalene (3.7 g). The molecular weight of Compound 68 measured by FAB-MS measurement was 699.

### (6) Synthesis of Compound 82

Amine Compound 82 according to one or more embodiments may be synthesized, for example, by steps of Reaction Formula 6.

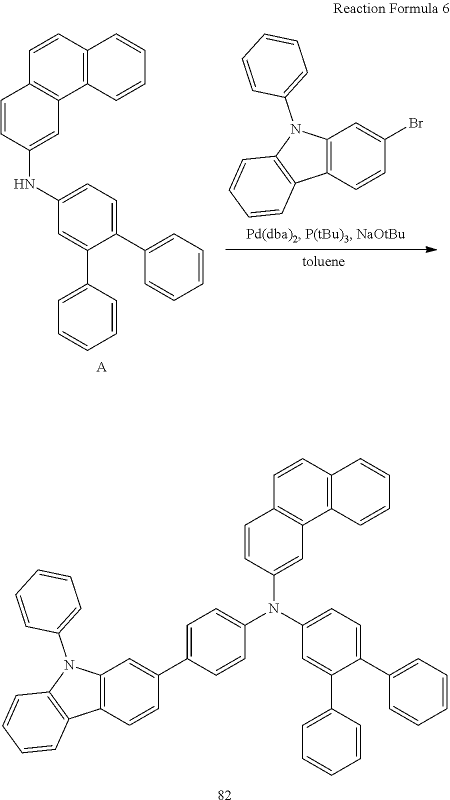

In the manner as the synthesis of Compound 5 described herein, 5.7 g (yield 73%) of Compound 82 was obtained from Intermediate Compound A (5.0 g) and 2-Bromo-9-phenyl-9H-carbazole (3.8 g). The molecular weight of Compound 82 measured by FAB-MS measurement was 662.

### (7) Synthesis of Compound 88

Amine Compound 88 according to one or more embodiments may be synthesized, for example, by steps of Reaction Formula 7.

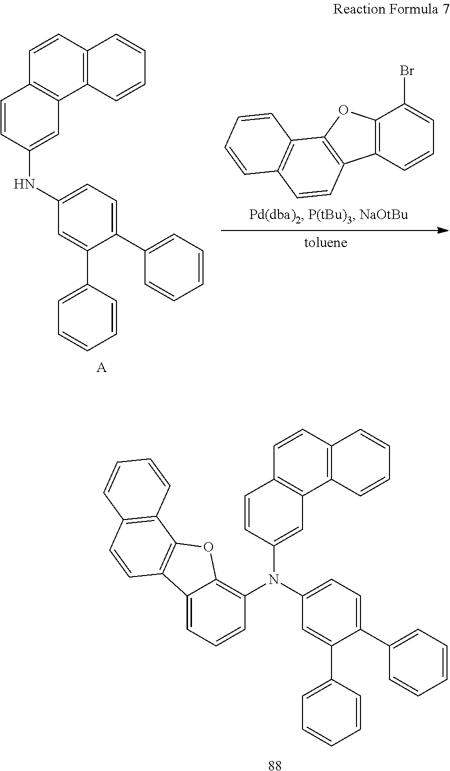

In the manner as the synthesis of Compound 5 described herein, 5.5 g (yield 73%) of Compound 88 was obtained from Intermediate Compound A (5.0 g) and 10-Bromonaphtho[1,2-b]benzofuran (3.5 g). The molecular weight of Compound 88 measured by FAB-MS measurement was 637.

### (8) Synthesis of Compound 90

Amine Compound 90 according to one or more embodiments may be synthesized, for example, by steps of Reaction Formula 8.

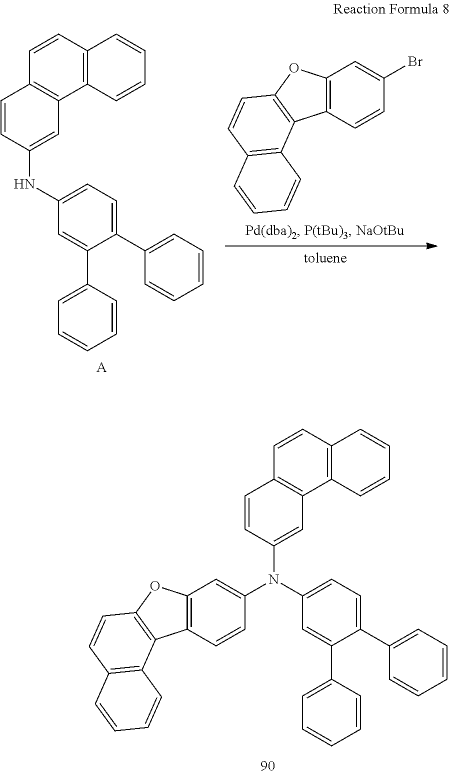

In the manner as the synthesis of Compound 5 described herein, 5.7 g (yield 76%) of Compound 90 was obtained from Intermediate Compound A (5.0 g) and 9-Bromonaphtho[2,1-b]benzofuran (3.5 g). The molecular weight of Compound 90 measured by FAB-MS measurement was 637.

### (9) Synthesis of Compound 100

Amine Compound 100 according to one or more embodiments may be synthesized, for example, by steps of Reaction Formula 9.

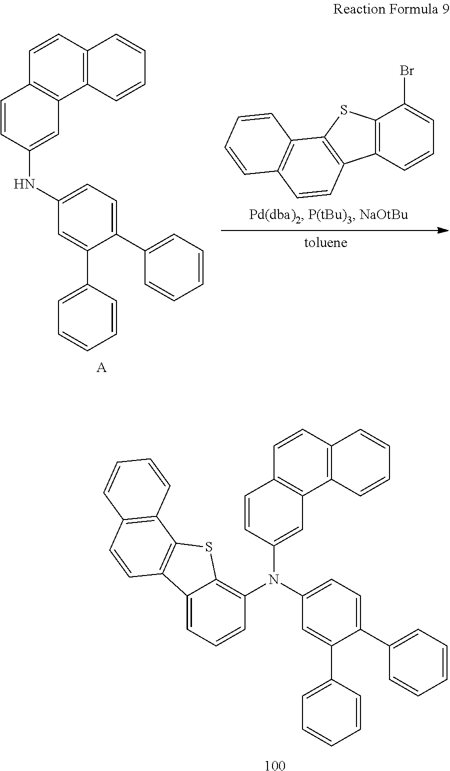

In the manner as the synthesis of Compound 5 described herein, 6.2 g (yield 80%) of Compound 100 was obtained from Intermediate Compound A (5.0 g) and 10-Bromobenzo[b]naphtho[2,1-d]thiophene (3.7 g). The molecular weight of Compound 100 measured by FAB-MS measurement was 653.

### (10) Synthesis of Compound 112

Amine Compound 112 according to one or more embodiments may be synthesized, for example, by steps of Reaction Formula 10.

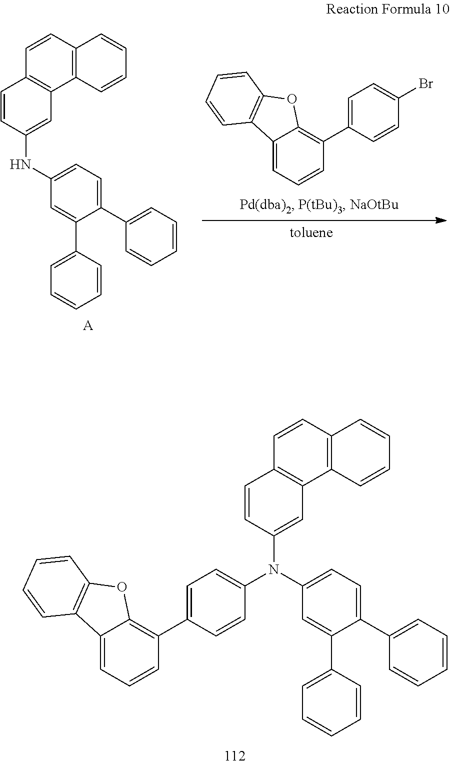

In the manner as the synthesis of Compound 5 described herein, 6.6 g (yield 84%) of Compound 112 was obtained from Intermediate Compound A (5.0 g) and 4-(4-Bromophenyl)dibenzo[b,d]furan (3.8 g). The molecular weight of Compound 112 measured by FAB-MS measurement was 663.

### (11) Synthesis of Compound 130

Amine Compound 130 according to one or more embodiments may be synthesized, for example, by steps of Reaction Formula 11.

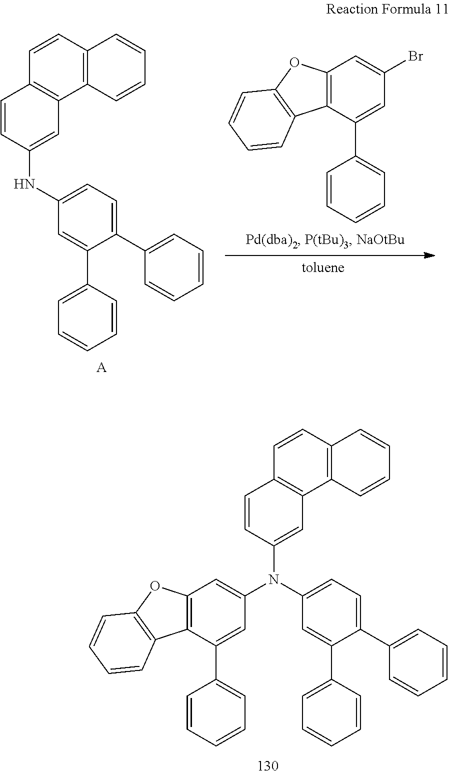

In the manner as the synthesis of Compound 5 described herein, 5.4 g (yield 69%) of Compound 130 was obtained from Intermediate Compound A (5.0 g) and 3-Bromo-1-phenyldibenzo[b,d]furan (3.8 g). The molecular weight of Compound 130 measured by FAB-MS measurement was 663.

### (12) Synthesis of Compound 154

Amine Compound 154 according to one or more embodiments may be synthesized, for example, by steps of Reaction Formula 12.

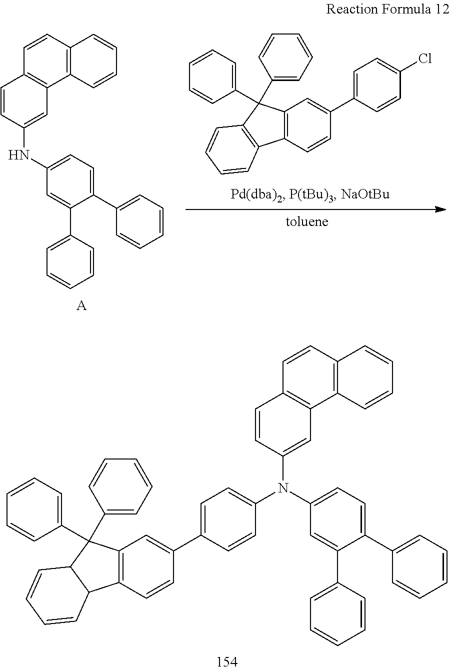

In the manner as the synthesis of Compound 5 described herein, 6.2 g (yield 65%) of Compound 154 was obtained from Intermediate Compound A (5.0 g) and 2-(4-Chlorophenyl)-9,9-diphenyl-9H-fluorene (5.1 g). The molecular weight of Compound 154 measured by FAB-MS measurement was 814.

### (13) Synthesis of Compound 166

Amine Compound 166 according to one or more embodiments may be synthesized, for example, by steps of Reaction Formula 13.

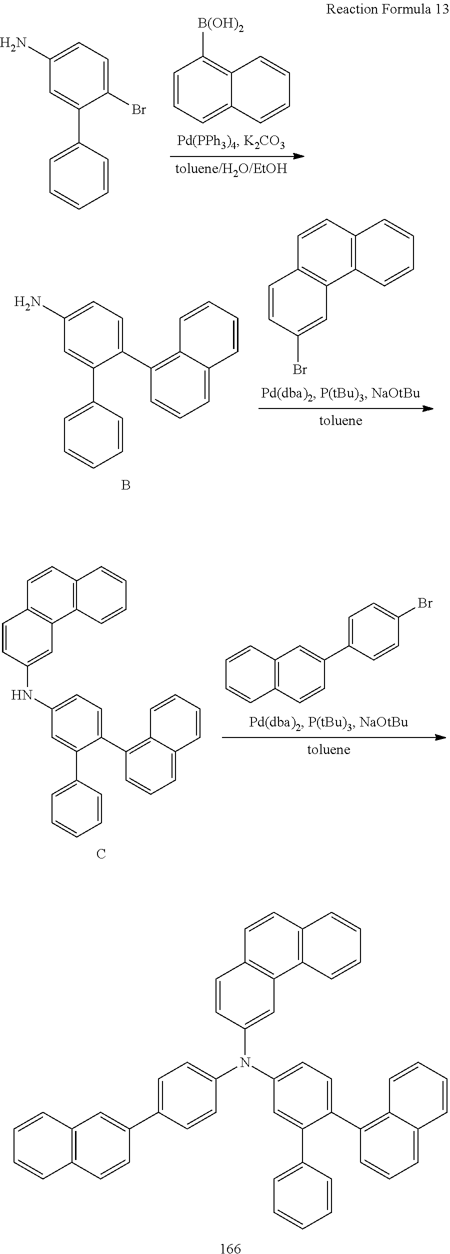

Under an argon (Ar) atmosphere, 6-Bromo-[1,1′-biphenyl]-3-amine (10.0 g), Naphthalen-1-ylboronic acid (6.9 g), Tetrakis(triphenylphosphine)palladium(0) (Pd(PPh₃)₄, 2.3 g), and Potassium carbonate (K₂CO₃, 11.1 g) were added to a 500 mL three-neck flask and dissolved in a mixed solvent of toluene, water, and ethanol (toluene:water:ethanol=10:2:1, 200 mL), and then heated and stirred at 80° C. for 8 hours. Thereafter, water was added thereto, and extraction was performed with CH₂Cl₂ to collect an organic layer, followed by drying the same with MgSO₄, and then the solvent was removed by distillation under reduced pressure. The obtained crude product was purified by silica gel column chromatography to obtain 7.0 g (yield 59%) of Intermediate Compound B. The molecular weight of Intermediate Compound B measured by FAB-MS measurement was 295.

In the same manner as the synthesis of Intermediate Compound A described herein, 5.2 g (yield 65%) of Intermediate Compound C was obtained from Intermediate Compound B (5.0 g) and 3-Bromophenanthrene (4.3 g). The molecular weight of Intermediate Compound C measured by FAB-MS measurement was 471.

In the manner as the synthesis of Compound 5 described herein, 5.0 g (yield 70%) of Compound 166 was obtained from Intermediate Compound C (5.0 g) and 2-(4-Bromophenyl)naphthalene (3.0 g). The molecular weight of Compound 166 measured by FAB-MS measurement was 673.

### (14) Synthesis of Compound 169

Amine Compound 169 according to one or more embodiments may be synthesized, for example, by steps of Reaction Formula 14.

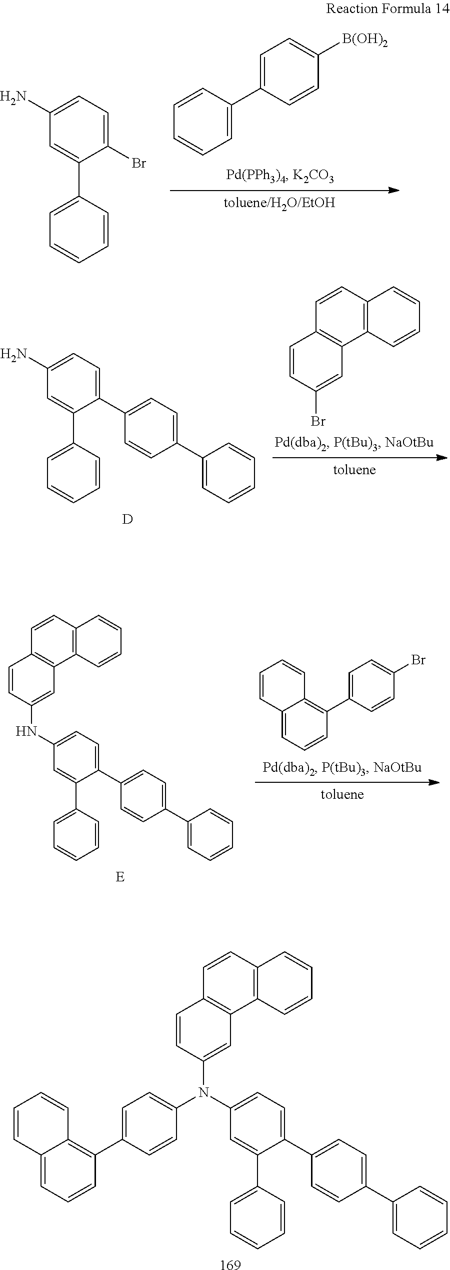

In the manner as the synthesis of Intermediate Compound B described herein, 7.9 g (yield 61%) of Intermediate Compound D was obtained from 6-Bromo-[1,1′-biphenyl]-3-amine (10.0 g) and [1,1′-Biphenyl]-4-ylboronic acid (8.0 g). The molecular weight of Intermediate Compound D measured by FAB-MS measurement was 321.

In the manner as the synthesis of Intermediate Compound A described herein, 5.4 g (yield 70%) of Intermediate Compound E was obtained from Intermediate Compound D (5.0 g) and 3-Bromophenanthrene (4.0 g). The molecular weight of Intermediate Compound E measured by FAB-MS measurement was 497.

In the manner as the synthesis of Compound 5 described herein, 4.9 g (yield 70%) of Compound 169 was obtained from Intermediate Compound E (5.0 g) and 1-(4-Bromophenyl)naphthalene (2.8 g). The molecular weight of Compound 169 measured by FAB-MS measurement was 699.

### (15) Synthesis of Compound 177

Amine Compound 177 according to one or more embodiments may be synthesized, for example, by steps of Reaction Formula 15.

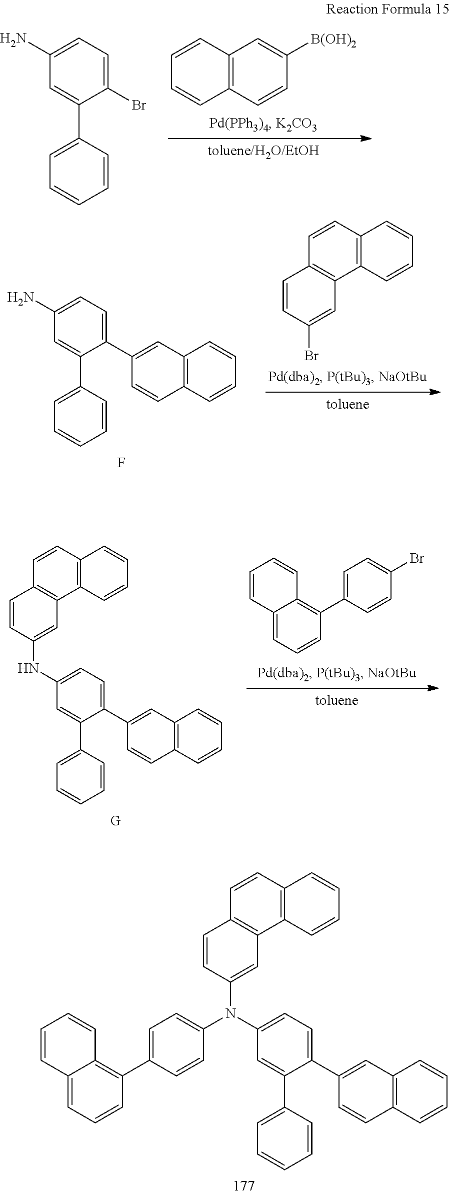

In the manner as the synthesis of Intermediate Compound B described herein, 8.8 g (yield 74%) of Intermediate Compound F was obtained from 6-Bromo-[1,1′-biphenyl]-3-amine (10.0 g) and Naphthalen-2-ylboronic acid (6.9 g). The molecular weight of Intermediate Compound F measured by FAB-MS measurement was 295.

In the manner as the synthesis of Intermediate Compound A described herein, 5.4 g (yield 68%) of Intermediate Compound G was obtained from Intermediate Compound F (5.0 g) and 3-Bromophenanthrene (4.3 g). The molecular weight of Intermediate Compound G measured by FAB-MS measurement was 471.

In the manner as the synthesis of Compound 5 described herein, 4.7 g (yield 66%) of Compound 177 was obtained from Intermediate Compound G (5.0 g) and 1-(4-Bromophenyl)naphthalene (3.0 g). The molecular weight of Compound 177 measured by FAB-MS measurement was 673.

### (16) Synthesis of Compound 178

Amine Compound 178 according to one or more embodiments may be synthesized, for example, by steps of Reaction Formula 16.

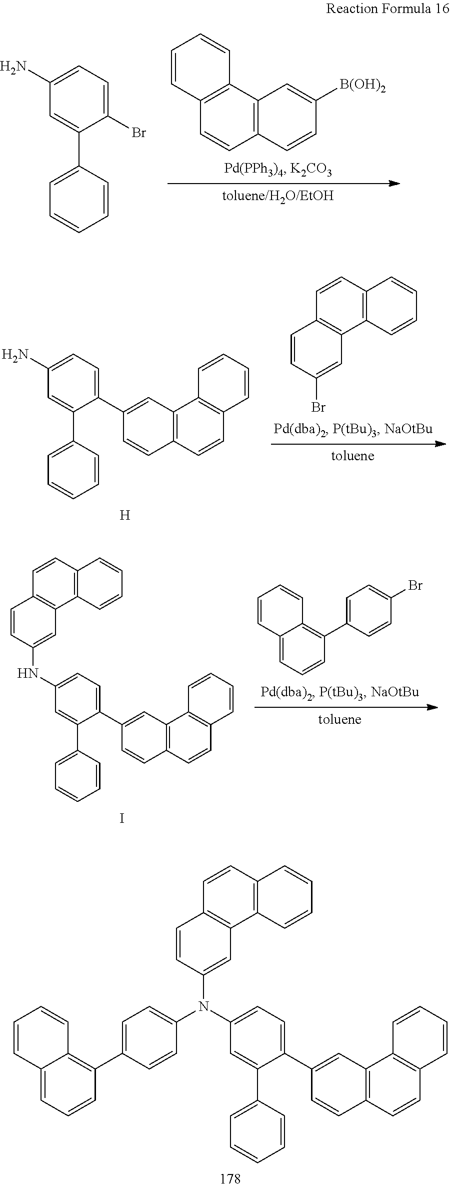

In the manner as the synthesis of Intermediate Compound B described herein, 7.3 g (yield 53%) of Intermediate Compound H was obtained from 6-Bromo-[1,1′-biphenyl]-3-amine (10.0 g) and Phenanthren-3-ylboronic acid (8.9 g). The molecular weight of Intermediate Compound H measured by FAB-MS measurement was 345.

In the manner as the synthesis of Intermediate Compound A described herein, 5.0 g (yield 67%) of Intermediate Compound I was obtained from Intermediate Compound H (5.0 g) and 3-Bromophenanthrene (3.7 g). The molecular weight of Intermediate Compound I measured by FAB-MS measurement was 521.

In the manner as the synthesis of Compound 5 described herein, 4.9 g (yield 71%) of Compound 178 was obtained from Intermediate Compound I (5.0 g) and 1-(4-Bromophenyl)naphthalene (2.7 g). The molecular weight of Compound 178 measured by FAB-MS measurement was 723.

### 2. Manufacturing and Evaluation of Light Emitting Element

### (1) Manufacturing of Light Emitting Element

A light emitting element including the amine compound of one or more embodiments, or including a Comparative Example Compound in a hole transport layer was manufactured in the following manner. Light emitting elements of Examples 1 to 16 were manufactured utilizing the amine compounds of one or more embodiments as hole transport layer materials, respectively. Light emitting elements of Comparative Example 1 to Comparative Example 20 were manufactured utilizing Comparative Example Compounds X-1 to X-20 as hole transport layer materials, respectively. Example Compounds utilized in Examples 1 to 16 and Comparative Example Compounds utilized in Comparative Examples 1 to 20 are as follows.

### Example Compounds

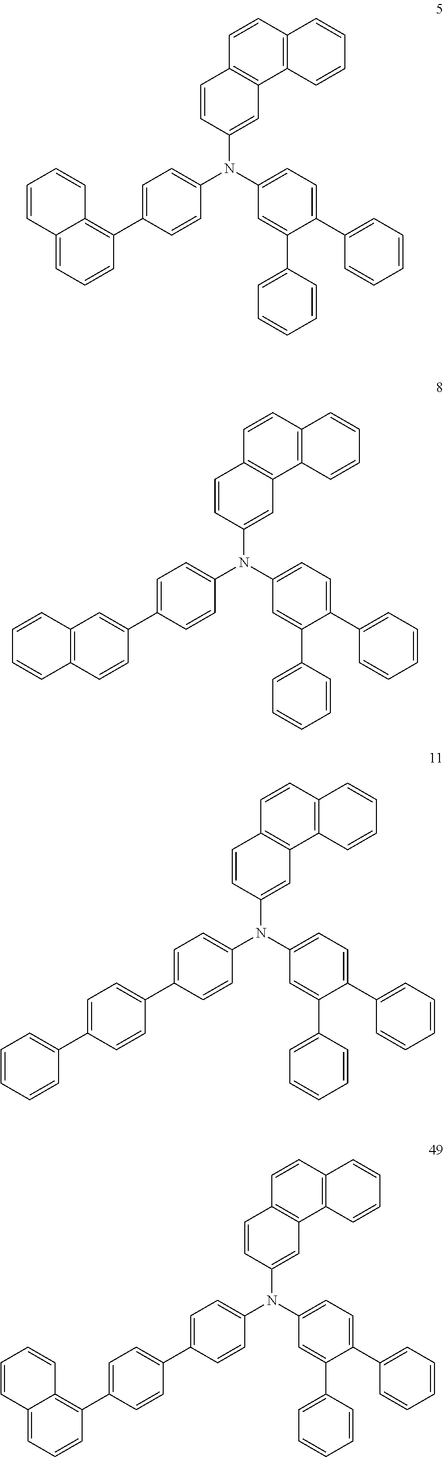 [SMILES: c1ccc(-c2ccc(N(c3ccc(-c4cccc5ccccc45)cc3)c3ccc4ccc5ccccc5c4c3)cc2-c2ccccc2)cc1 ; c1ccc(-c2ccc(N(c3ccc(-c4ccc5ccccc5c4)cc3)c3ccc4ccc5ccccc5c4c3)cc2-c2ccccc2)cc1 ; c1ccc(-c2ccc(-c3ccc(N(c4ccc(-c5ccccc5)c(-c5ccccc5)c4)c4ccc5ccc6ccccc6c5c4)cc3)cc2)cc1 ; c1ccc(-c2ccc(N(c3ccc(-c4ccc(-c5cccc6ccccc56)cc4)cc3)c3ccc4ccc5ccccc5c4c3)cc2-c2ccccc2)cc1]

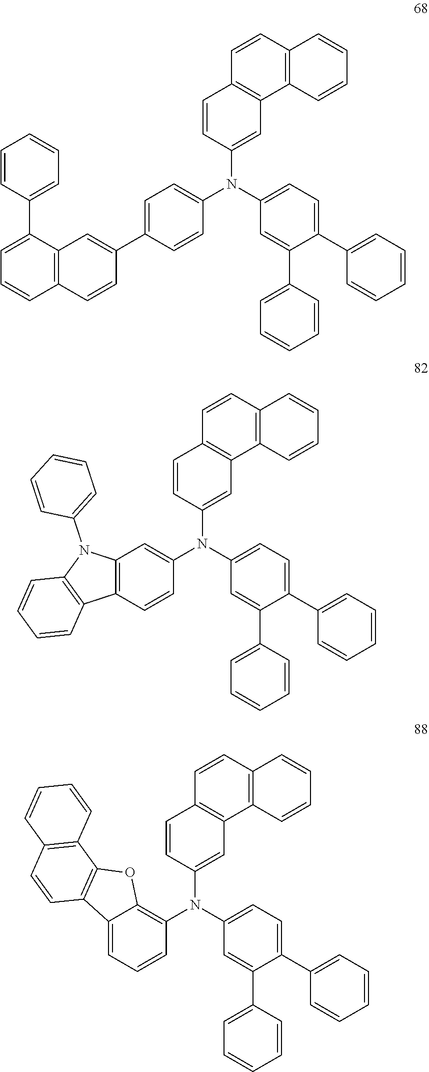

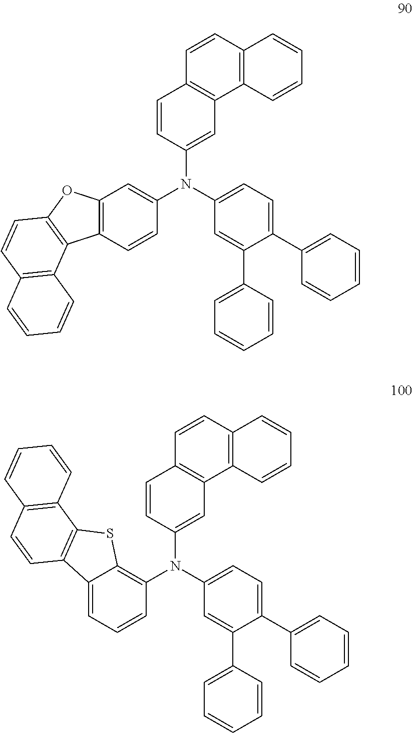 [SMILES: c1ccc(-c2ccc(N(c3ccc4c(c3)oc3ccc5ccccc5c34)c3ccc4ccc5ccccc5c4c3)cc2-c2ccccc2)cc1 ; c1ccc(-c2ccc(N(c3ccc4ccc5ccccc5c4c3)c3cccc4c3sc3c5ccccc5ccc43)cc2-c2ccccc2)cc1]

### Comparative Example Compounds

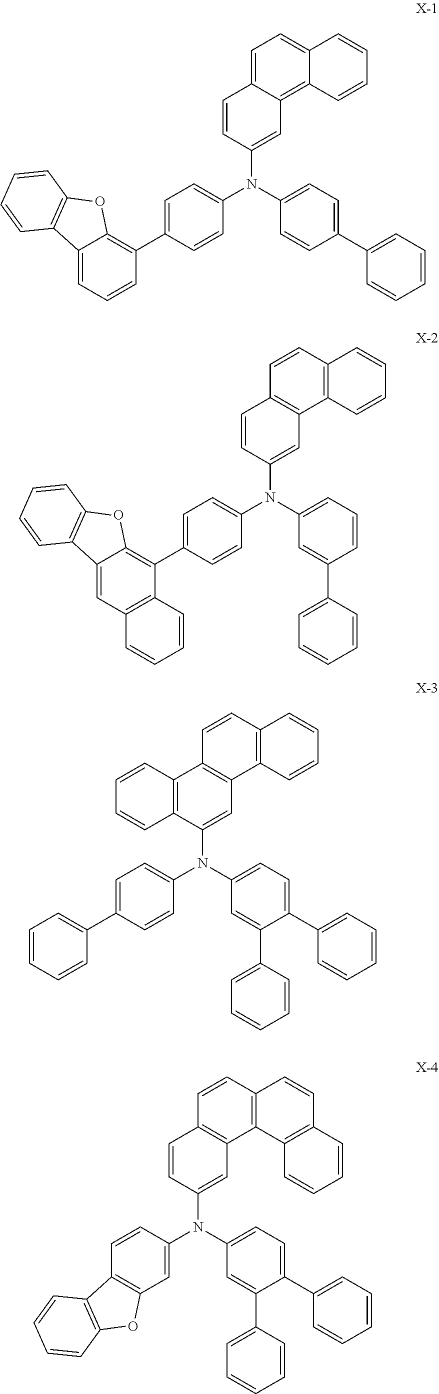 [SMILES: c1ccc(-c2ccc(N(c3ccc(-c4cccc5c4oc4ccccc45)cc3)c3ccc4ccc5ccccc5c4c3)cc2)cc1 ; c1ccc(-c2cccc(N(c3ccc(-c4c5ccccc5cc5c4oc4ccccc45)cc3)c3ccc4ccc5ccccc5c4c3)c2)cc1 ; c1ccc(-c2ccc(N(c3ccc(-c4ccccc4)c(-c4ccccc4)c3)c3cc4c5ccccc5ccc4c4ccccc34)cc2)cc1 ; c1ccc(-c2ccc(N(c3ccc4c(c3)oc3ccccc34)c3ccc4ccc5ccc6ccccc6c5c4c3)cc2-c2ccccc2)cc1]

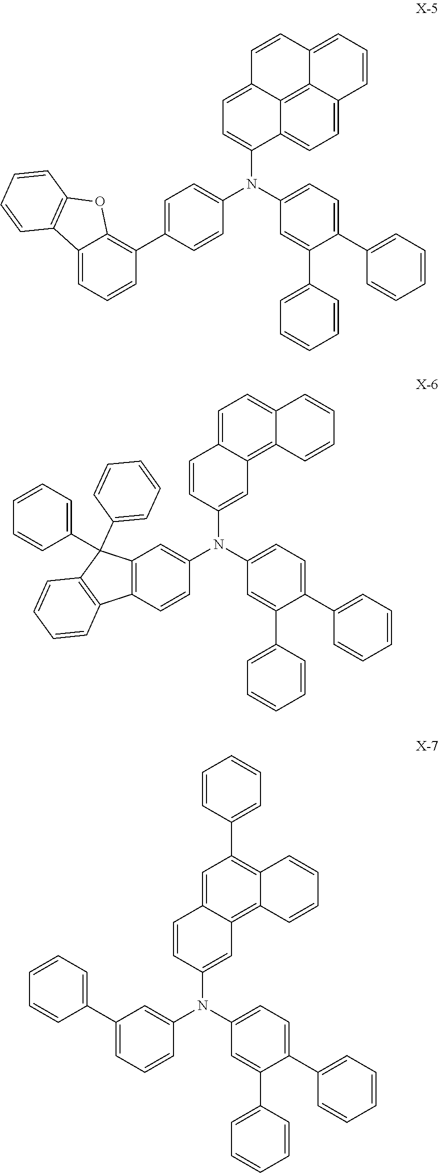

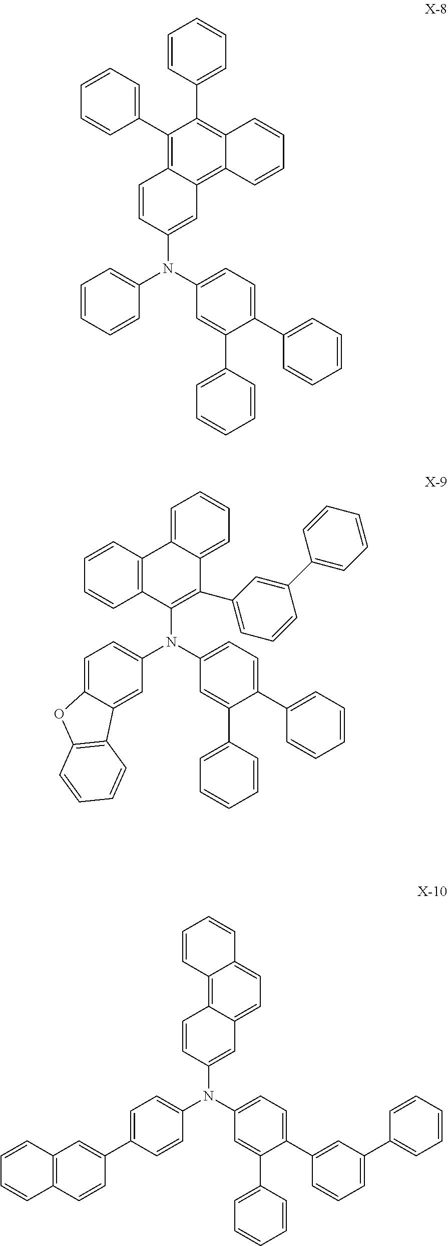 [SMILES: c1ccc(-c2ccc(N(c3ccccc3)c3ccc4c(-c5ccccc5)c(-c5ccccc5)c5ccccc5c4c3)cc2-c2ccccc2)cc1 ; c1ccc(-c2cccc(-c3c(N(c4ccc(-c5ccccc5)c(-c5ccccc5)c4)c4ccc5oc6ccccc6c5c4)c4ccccc4c4ccccc34)c2)cc1 ; c1ccc(-c2cccc(-c3ccc(N(c4ccc(-c5ccc6ccccc6c5)cc4)c4ccc5c(ccc6ccccc65)c4)cc3-c3ccccc3)c2)cc1]

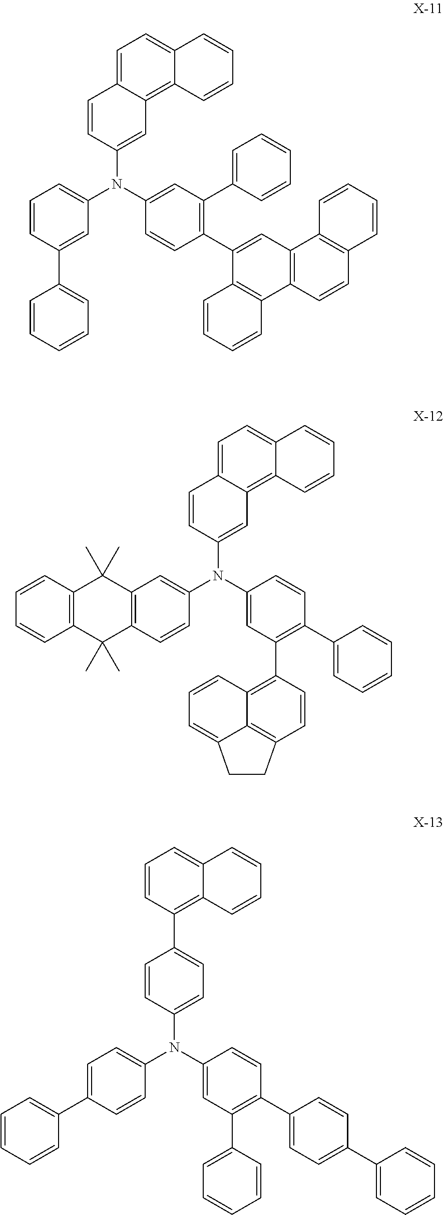 [SMILES: c1ccc(-c2cccc(N(c3ccc(-c4cc5c6ccccc6ccc5c5ccccc45)c(-c4ccccc4)c3)c3ccc4ccc5ccccc5c4c3)c2)cc1 ; CC1(C)c2ccccc2C(C)(C)c2cc(N(c3ccc(-c4ccccc4)c(-c4ccc5c6c(cccc46)CC5)c3)c3ccc4ccc5ccccc5c4c3)ccc21 ; c1ccc(-c2ccc(-c3ccc(N(c4ccc(-c5ccccc5)cc4)c4ccc(-c5cccc6ccccc56)cc4)cc3-c3ccccc3)cc2)cc1]

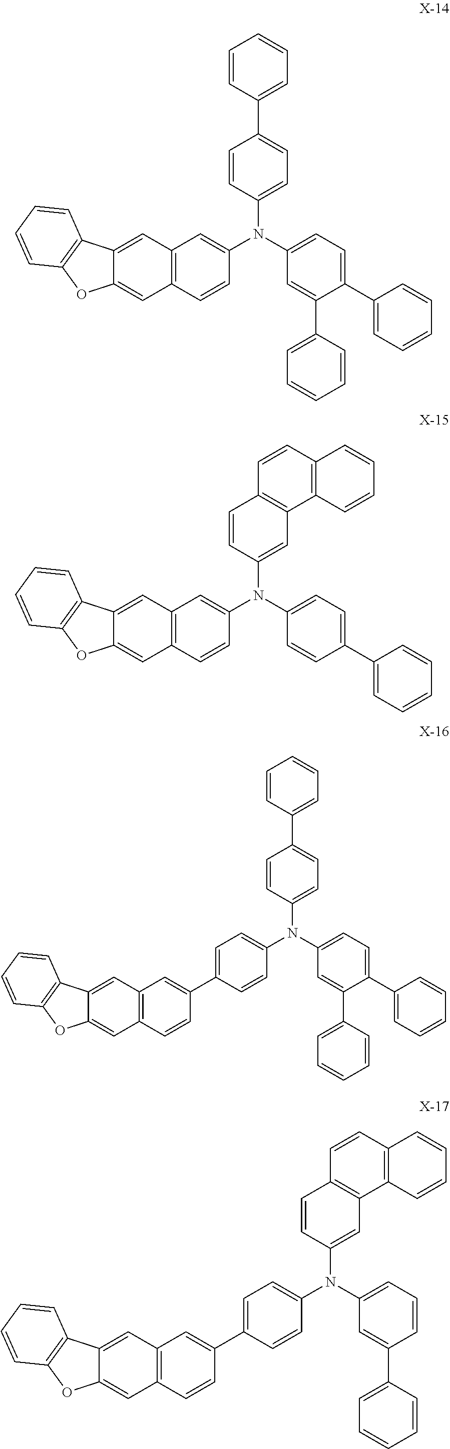 [SMILES: c1ccc(-c2ccc(N(c3ccc(-c4ccccc4)c(-c4ccccc4)c3)c3ccc4cc5oc6ccccc6c5cc4c3)cc2)cc1 ; c1ccc(-c2ccc(N(c3ccc4cc5oc6ccccc6c5cc4c3)c3ccc4ccc5ccccc5c4c3)cc2)cc1 ; c1ccc(-c2ccc(N(c3ccc(-c4ccc5cc6oc7ccccc7c6cc5c4)cc3)c3ccc(-c4ccccc4)c(-c4ccccc4)c3)cc2)cc1 ; c1ccc(-c2cccc(N(c3ccc(-c4ccc5cc6oc7ccccc7c6cc5c4)cc3)c3ccc4ccc5ccccc5c4c3)c2)cc1]

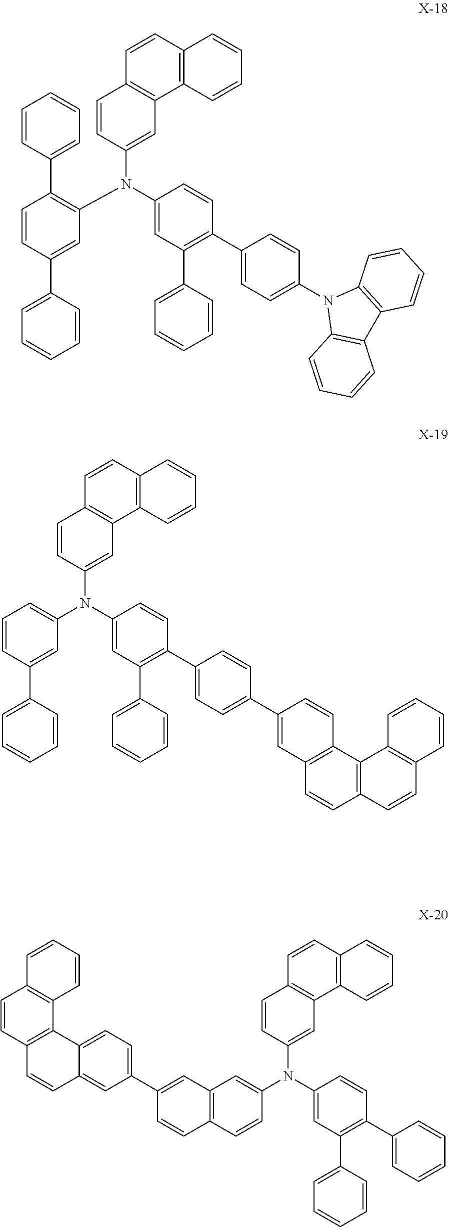 [SMILES: c1ccc(-c2ccc(-c3ccccc3)c(N(c3ccc(-c4ccc(-n5c6ccccc6c6ccccc65)cc4)c(-c4ccccc4)c3)c3ccc4ccc5ccccc5c4c3)c2)cc1 ; c1ccc(-c2cccc(N(c3ccc(-c4ccc(-c5ccc6c(ccc7ccc8ccccc8c76)c5)cc4)c(-c4ccccc4)c3)c3ccc4ccc5ccccc5c4c3)c2)cc1 ; c1ccc(-c2ccc(N(c3ccc4ccc(-c5ccc6c(ccc7ccc8ccccc8c76)c5)cc4c3)c3ccc4ccc5ccccc5c4c3)cc2-c2ccccc2)cc1]

As a first electrode, a glass substrate patterned with indium tin oxide (ITO) 150 nanometer (nm) was subjected to ultrasonic cleaning utilizing isopropyl alcohol and pure water for 5 minutes each. After the ultrasonic cleaning, UV irradiation was performed for 30 minutes, and ozone treatment was performed. Thereafter, 2-TNATA was deposited at a thickness of 60 nm to form (or provide) a hole injection layer. An Example Compound or a Comparative Example Compound was deposited on the hole injection layer at a thickness of 30 nm to form (or provide) a hole transport layer.

TBP and ADN were co-deposited on the hole transport layer to form (or provide) a light emitting layer at a thickness of 25 nm. The TBP and the ADN were co-deposited at a weight ratio of 3:97. Thereafter, Alq3 and LiF were sequentially deposited at a thickness of 25 nm and at thickness of 1 nm, respectively, to form (or provide) an electron transport region.

Next, Al was deposited at a thickness of 100 nm to form (or provide) a second electrode.

In Examples, the hole transport region, the light emitting layer, the electron transport region, and the second electrode were formed utilizing a vacuum deposition device.

Compounds utilized in manufacturing the light emitting elements are as follows.

### Materials Utilized in Manufacturing Light Emitting Elements

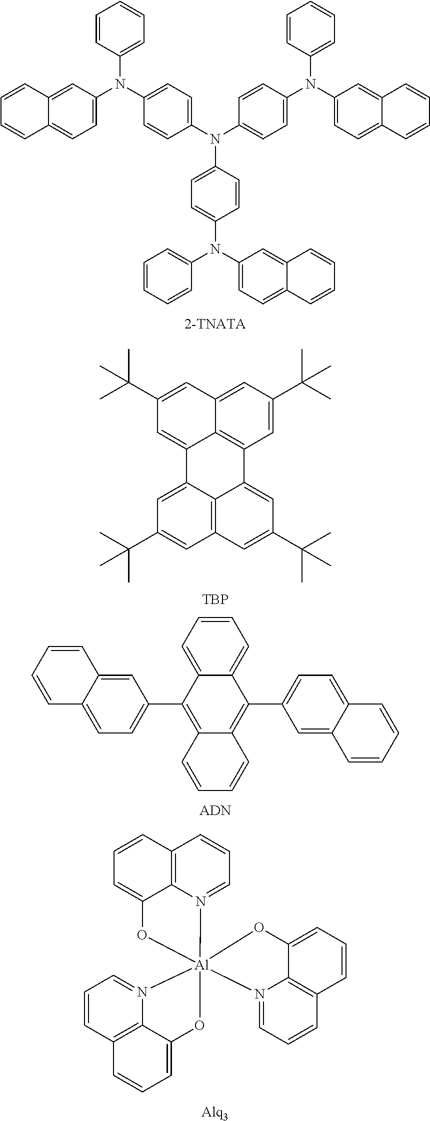

### (2) Evaluation of Light Emitting Elements

Table 1 shows the evaluation of light emitting elements of Examples and Comparative Examples. Table 1 shows the light emission efficiency and element lifespan for the light emitting elements of Examples and Comparative Examples. The light emission efficiency and element lifespan were evaluated utilizing C9920-11 luminance orientation characteristic measurement equipment of Hamamatsu Photonics.

The light emission efficiency was relatively expressed utilizing the light emission efficiency of Comparative Example 1 as 100% at a current density of 10 milliampere per square centimeter (mA/cm²). The relative element lifespan was relatively expressed by utilizing the numerical value of time of Comparative Example 1 as 100%, the time taken for the initial luminance to be deteriorated by 50% during substantially continuous driving.

TABLE 1

| Element | | | |
|---|---|---|---|
| manufacturing | Hole transport | Light emission | Element lifespan |
| example | layer material | efficiency (%) | (LT50) (%) |
| Example 1 | Compound 5 | 110% | 130% |
| Example 2 | Compound 8 | 108% | 140% |
| Example 3 | Compound 11 | 110% | 125% |
| Example 4 | Compound 49 | 111% | 120% |
| Example 5 | Compound 68 | 107% | 150% |
| Example 6 | Compound 82 | 106% | 120% |
| Example 7 | Compound 88 | 108% | 130% |
| Example 8 | Compound 90 | 106% | 140% |
| Example 9 | Compound 100 | 107% | 135% |
| Example 10 | Compound 112 | 108% | 130% |
| Example 11 | Compound 130 | 106% | 110% |
| Example 12 | Compound 154 | 107% | 110% |
| Example 13 | Compound 166 | 106% | 120% |
| Example 14 | Compound 169 | 107% | 120% |
| Example 15 | Compound 177 | 107% | 120% |
| Example 16 | Compound 178 | 107% | 110% |
| Comparative | Comparative | 100% | 100% |
| Example 1 | Example | | |
| | compound X-1 | | |
| Comparative | Comparative | 102% | 85% |
| Example 2 | Example | | |
| | compound X-2 | | |
| Comparative | Comparative | 96% | 95% |
| Example 3 | Example | | |
| | compound X-3 | | |
| Comparative | Comparative | 96% | 80% |
| Example 4 | Example | | |
| | compound X-4 | | |
| Comparative | Comparative | 95% | 80% |
| Example 5 | Example | | |
| | compound X-5 | | |
| Comparative | Comparative | 98% | 105% |
| Example 6 | Example | | |
| | compound X-6 | | |
| Comparative | Comparative | 105% | 70% |
| Example 7 | Example | | |
| | compound X-7 | | |
| Comparative | Comparative | 105% | 60% |
| Example 8 | Example | | |
| | compound X-8 | | |
| Comparative | Comparative | 105% | 40% |
| Example 9 | Example | | |
| | compound X-9 | | |
| Comparative | Comparative | 98% | 60% |
| Example 10 | Example | | |
| | compound X-10 | | |
| Comparative | Comparative | 96% | 70% |
| Example 11 | Example | | |
| | compound X-11 | | |
| Comparative | Comparative | 97% | 40% |
| Example 12 | Example | | |
| | compound X-12 | | |
| Comparative | Comparative | 102% | 100% |
| Example 13 | Example | | |
| | compound X-13 | | |
| Comparative | Comparative | 99% | 100% |
| Example 14 | Example | | |
| | compound X-14 | | |
| Comparative | Comparative | 99% | 100% |
| Example 15 | Example | | |
| | compound X-15 | | |
| Comparative | Comparative | 98% | 100% |
| Example 16 | Example | | |
| | compound X-16 | | |
| Comparative | Comparative | 98% | 100% |
| Example 17 | Example | | |
| | compound X-17 | | |
| Comparative | Comparative | 100% | 40% |
| Example 18 | Example | | |
| | compound X-18 | | |
| Comparative | Comparative | 100% | 50% |
| Example 19 | Example | | |
| | compound X-19 | | |
| Comparative | Comparative | 97% | 50% |
| Example 20 | Example | | |
| | compound X-20 | | |

Referring to the results of Table 1, Example 1 to Example 16 showed high-efficiency and long-lifespan element properties compared to Comparative Example 1 to Comparative Example 20. Example Compounds include a phenanthrene moiety which is directly bonded to a nitrogen atom at a specific bonding position, and a 3,4-substituted phenyl group which is directly bonded to a nitrogen atom at a specific bonding position or is bonded thereto through a linker, and may exhibit excellent or suitable charge transportability and material stability compared to other Comparative Example Compounds depending on such a specified substituent group and a specified substitution position.

For example, Example Compounds have excellent or suitable charge transportability and charge balance due to the molecular structural characteristics of Example Compounds, which are distinct from those Comparative Example Compounds. For example, it may be confirmed that the light emitting elements of Examples including the amine compound of one or more embodiments in the hole transport layer exhibit high-efficiency and long-lifespan properties.

An amine compound of one or more embodiments includes both (e.g., simultaneously) a phenanthrene group and a 3,4-substituted phenyl group, and has a structure in which the phenanthrene group and the 3,4-substituted phenyl group are bonded to nitrogen atoms of amine at specific positions. Accordingly, the amine compound of one or more embodiments may exhibit excellent or suitable material stability and high charge transportability. A light emitting element of one or more embodiments including the amine compound of one or more embodiments in a hole transport region may exhibit excellent or suitable light emission efficiency and long-lifespan properties. In one or more embodiments, a light emitting element of one or more embodiments which emits blue light may exhibit relatively high-efficiency and relatively long-lifespan properties by including the amine compound of one or more embodiments in a hole transport region.

A light emitting element of one or more embodiments includes an amine compound of one or more embodiments in a hole transport region, and thus, may exhibit relatively high-efficiency and relatively long-lifespan properties.

The amine compound of one or more embodiments may be utilized as a material for implementing improved properties of a light emitting element, which are high efficiency and long lifespan (e.g., by providing the light emitting element with relatively high efficiency and relatively long service life).

A display device of one or more embodiments may exhibit excellent or suitable display quality.

Although the present disclosure has been described with reference to embodiments of the present disclosure, it will be understood by those skilled in the art that one or more suitable modifications and changes in form and details may be made therein without departing from the spirit and scope of the present disclosure as set forth in the following claims and equivalents thereof.

Accordingly, the technical scope of the present disclosure is not intended to be limited to the contents set forth in the detailed description of the specification, but is intended to be defined by the appended claims, and equivalents thereof.

What is claimed is:

1. A light emitting element comprising:
a first electrode;
a second electrode on the first electrode;
a light emitting layer between the first electrode and the second electrode; and
a hole transport region between the first electrode and the light emitting layer, and comprising an amine compound represented by Formula 1: 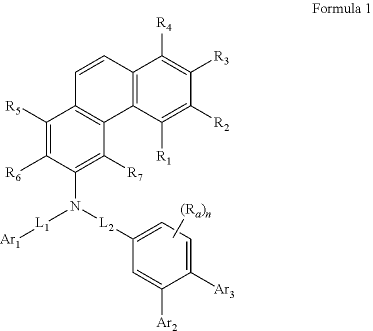
in Formula 1,
R₁ to R₇ are each independently a hydrogen atom, a deuterium atom, a halogen atom, a substituted or unsubstituted alkyl group having 1 to 15 carbons, a substituted or unsubstituted aryl group having 6 to 30 ring-forming carbon atoms, or a substituted or unsubstituted heteroaryl group having 2 to 30 ring-forming carbon atoms,
n is an integer of 1 to 3,
Rₐ is a hydrogen atom, a deuterium atom, a halogen atom, a substituted or unsubstituted alkyl group having 1 to 15 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or forms a ring by being bonded to an adjacent group,
L₁ and L₂ are each independently a direct linkage, a substituted or unsubstituted arylene group having 6 to 30 ring-forming carbon atoms, or a substituted or unsubstituted heteroarylene group having 2 to 30 ring-forming carbon atoms,
Ar₁ is a substituted or unsubstituted aryl group having 6 to 30 ring-forming carbon atoms, or a substituted or unsubstituted heteroaryl group having 2 to 30 ring-forming carbon atoms, and Ar₁ is not a fluorenyl group when L₁ is a direct linkage,
Ar₂ and Ar₃ are each independently a substituted or unsubstituted phenyl group, a substituted or unsubstituted biphenyl group, a substituted or unsubstituted terphenyl group, a substituted or unsubstituted naphthyl group, or a substituted or unsubstituted phenanthrenyl group, except when each of Ar₂ and Ar₃ includes a carbazole group as a substituent, and
each of Ar₁, Ar₂, and Ar₃ do not include benzophenanthrene.

2. The light emitting element of claim 1, wherein the hole transport region comprises at least one of a hole injection layer, a hole transport layer, an electron blocking layer, or a light emitting auxiliary layer, and wherein at least one of the hole injection layer, the hole transport layer, the electron blocking layer, or the light emitting auxiliary layer comprises the amine compound.

3. The light emitting element of claim 1, wherein the hole transport region comprises a hole injection layer on the first electrode, and a hole transport layer on the hole injection layer, and wherein the hole transport layer comprises the amine compound.

4. The light emitting element of claim 1, wherein the amine compound is represented by Formula 1A: 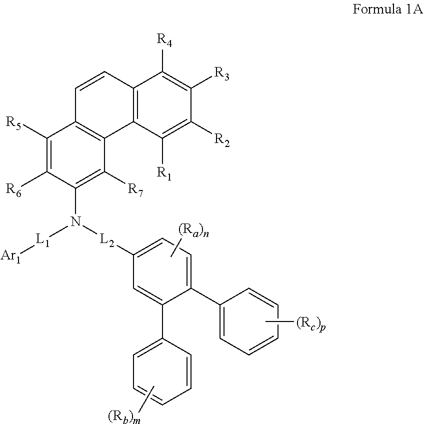
in Formula 1A,
m and p are each independently an integer of 1 to 5,
R_{b} and R_{c} are each independently a hydrogen atom, a deuterium atom, a halogen atom, a substituted or unsubstituted alkyl group having 1 to 15 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or form a ring by being bonded to an adjacent group, and
Ar₁, L₁, L₂, R₁ to R₇, Rₐ, and n are as defined in Formula 1.

5. The light emitting element of claim 1, wherein the Ar₁ is a substituted or unsubstituted phenyl group, a substituted or unsubstituted naphthyl group, a substituted or unsubstituted biphenyl group, a substituted or unsubstituted terphenyl group, a substituted or unsubstituted fluorenyl group, a substituted or unsubstituted dibenzoheterole group comprising O, S, N, or Si as a ring-forming hetero atom, or a substituted or unsubstituted benzonaphthoheterol group comprising O or S as a ring-forming hetero atom.

6. The light emitting element of claim 1, wherein at least one hydrogen atom of the amine compound is substituted with a deuterium atom.

7. The light emitting element of claim 1, wherein in Formula 1, each of R₁ to R₇, Rₐ, Ar₁, Ar₂, Ar₃, L₁, and L₂ do not include a substituted or unsubstituted amine group.

8. The light emitting element of claim 1, wherein the light emitting layer comprises a compound represented by Formula E-1: 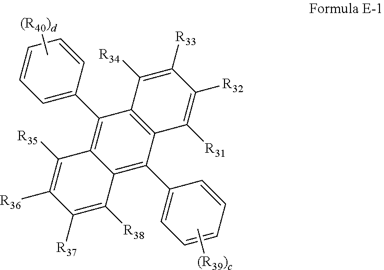
in Formula E-1,
c and d are each independently an integer of 0 to 5, and
R₃₁ to R₄₀ may each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted an alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or form a ring by being bonded to an adjacent group.

9. The light emitting element of claim 1, wherein the light emitting layer is configured to emit blue light.

10. The light emitting element of claim 1, wherein the amine compound is represented by any one selected from among compounds of Compound Group 1: 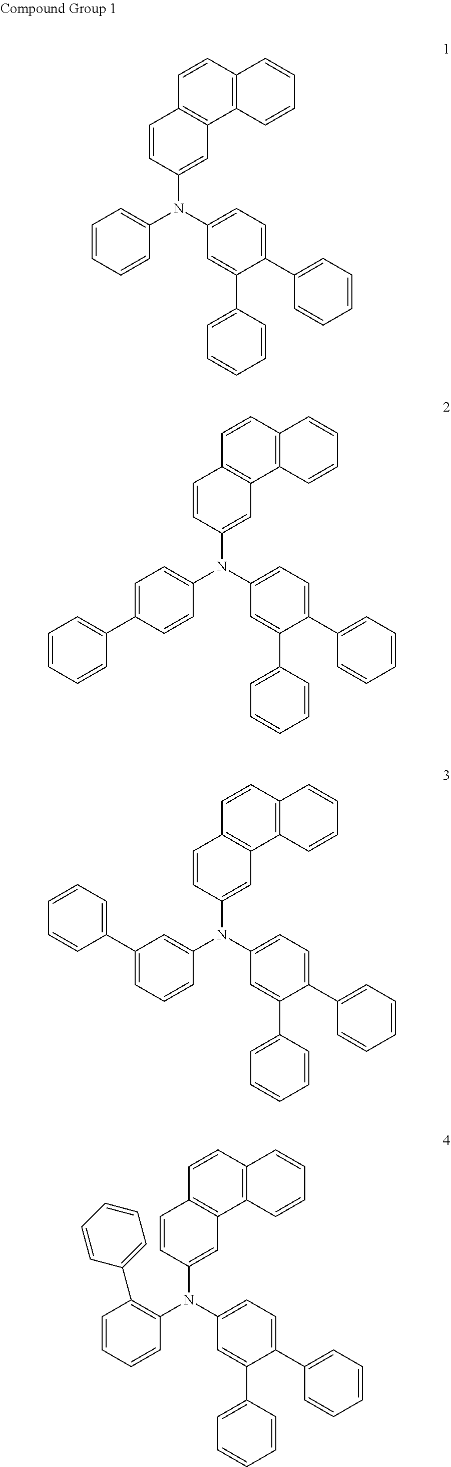 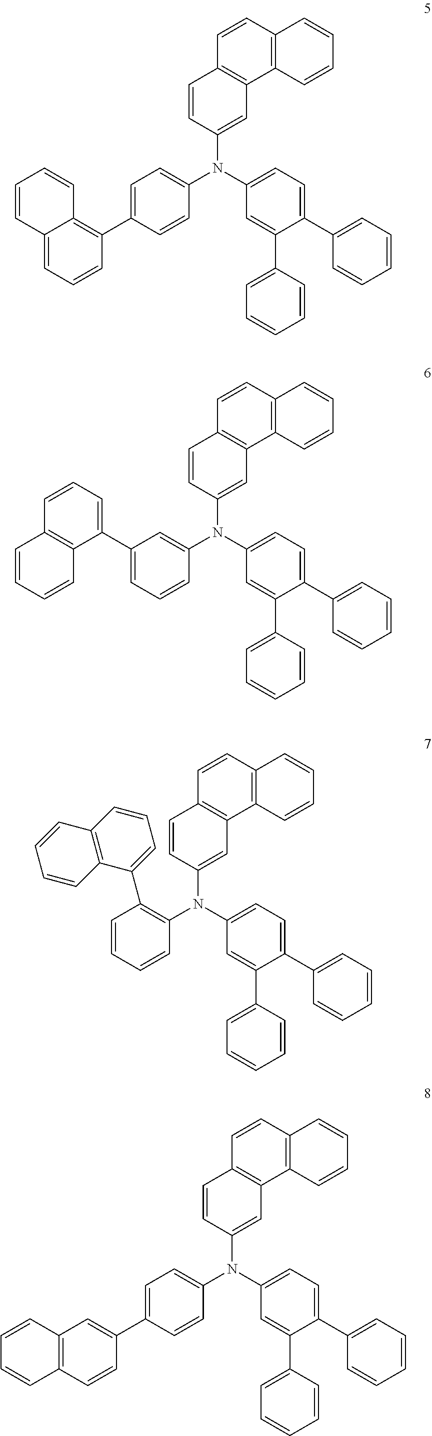 [SMILES: c1ccc(-c2ccc(N(c3ccc(-c4cccc5ccccc45)cc3)c3ccc4ccc5ccccc5c4c3)cc2-c2ccccc2)cc1 ; c1ccc(-c2ccc(N(c3cccc(-c4cccc5ccccc45)c3)c3ccc4ccc5ccccc5c4c3)cc2-c2ccccc2)cc1 ; c1ccc(-c2ccc(N(c3ccc4ccc5ccccc5c4c3)c3ccccc3-c3cccc4ccccc34)cc2-c2ccccc2)cc1 ; c1ccc(-c2ccc(N(c3ccc(-c4ccc5ccccc5c4)cc3)c3ccc4ccc5ccccc5c4c3)cc2-c2ccccc2)cc1] 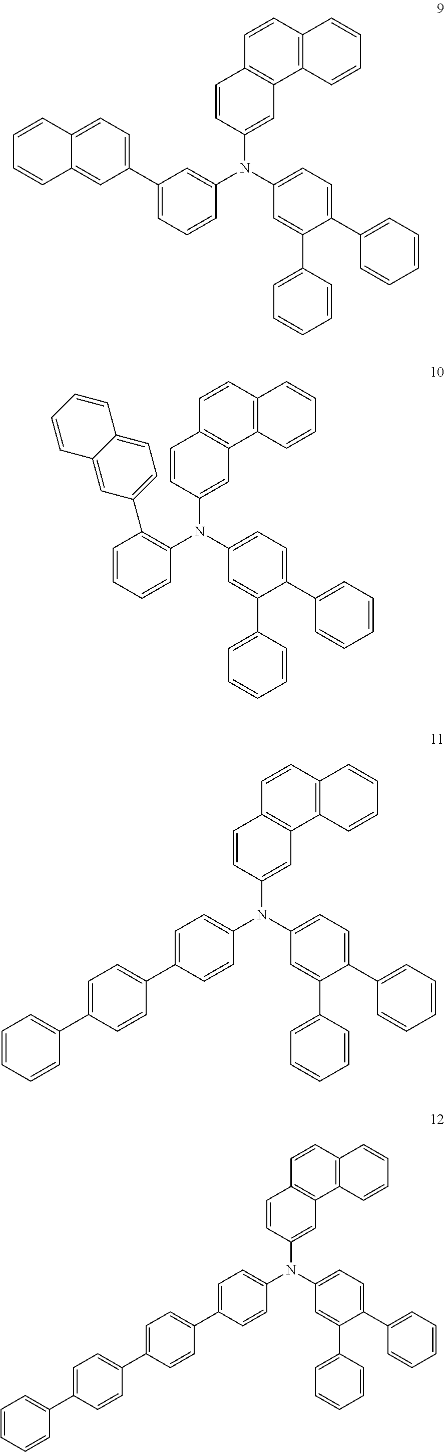 [SMILES: c1ccc(-c2ccc(N(c3cccc(-c4ccc5ccccc5c4)c3)c3ccc4ccc5ccccc5c4c3)cc2-c2ccccc2)cc1 ; c1ccc(-c2ccc(N(c3ccc4ccc5ccccc5c4c3)c3ccccc3-c3ccc4ccccc4c3)cc2-c2ccccc2)cc1 ; c1ccc(-c2ccc(-c3ccc(N(c4ccc(-c5ccccc5)c(-c5ccccc5)c4)c4ccc5ccc6ccccc6c5c4)cc3)cc2)cc1 ; c1ccc(-c2ccc(-c3ccc(-c4ccc(N(c5ccc(-c6ccccc6)c(-c6ccccc6)c5)c5ccc6ccc7ccccc7c6c5)cc4)cc3)cc2)cc1] 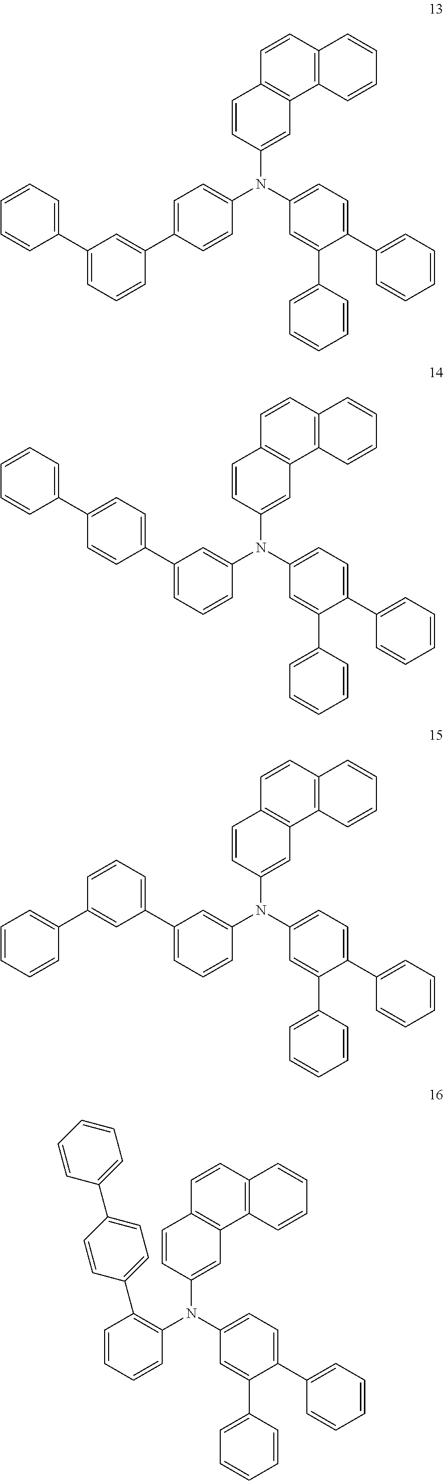 [SMILES: c1ccc(-c2cccc(-c3ccc(N(c4ccc(-c5ccccc5)c(-c5ccccc5)c4)c4ccc5ccc6ccccc6c5c4)cc3)c2)cc1 ; c1ccc(-c2ccc(-c3cccc(N(c4ccc(-c5ccccc5)c(-c5ccccc5)c4)c4ccc5ccc6ccccc6c5c4)c3)cc2)cc1 ; c1ccc(-c2cccc(-c3cccc(N(c4ccc(-c5ccccc5)c(-c5ccccc5)c4)c4ccc5ccc6ccccc6c5c4)c3)c2)cc1 ; c1ccc(-c2ccc(-c3ccccc3N(c3ccc(-c4ccccc4)c(-c4ccccc4)c3)c3ccc4ccc5ccccc5c4c3)cc2)cc1] 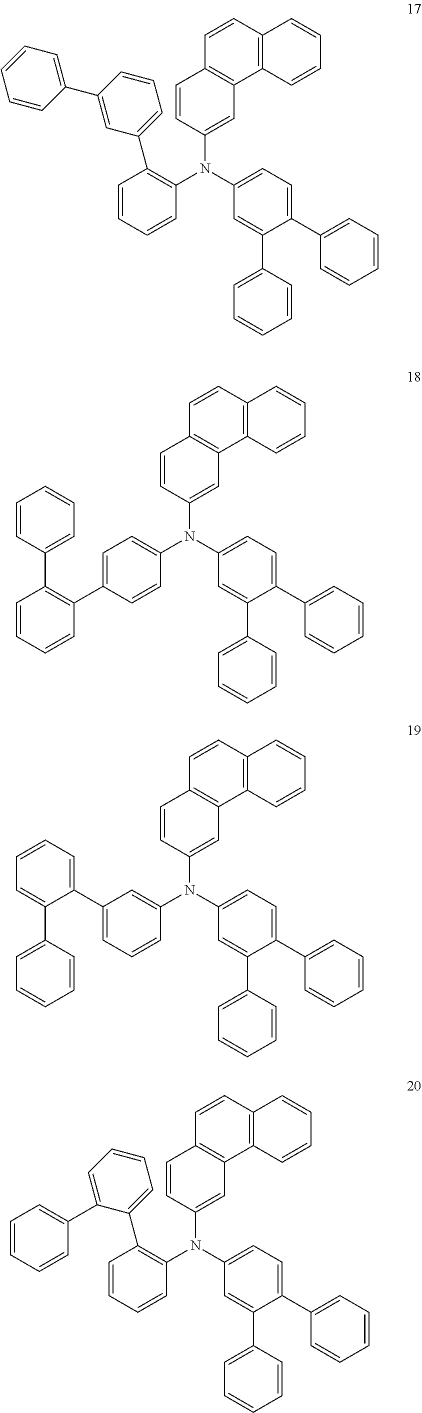 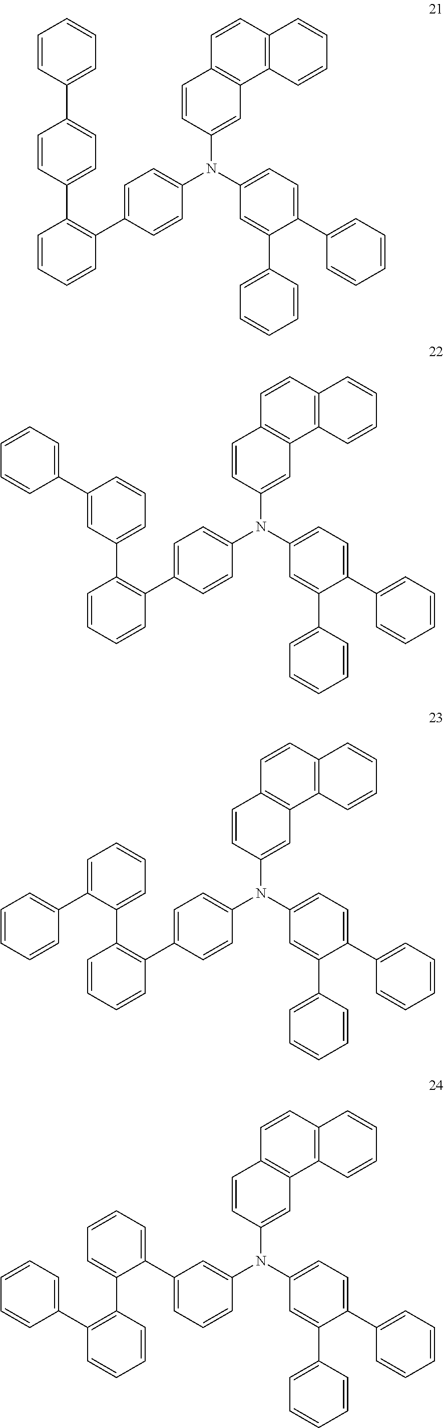 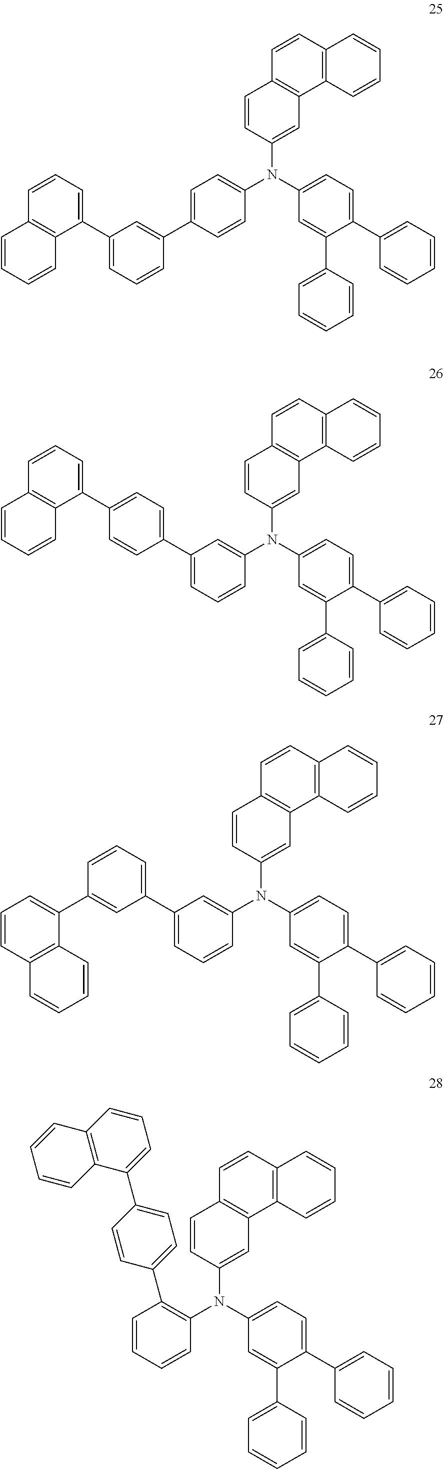 [SMILES: c1ccc(-c2ccc(N(c3ccc(-c4cccc(-c5cccc6ccccc56)c4)cc3)c3ccc4ccc5ccccc5c4c3)cc2-c2ccccc2)cc1 ; c1ccc(-c2ccc(N(c3cccc(-c4ccc(-c5cccc6ccccc56)cc4)c3)c3ccc4ccc5ccccc5c4c3)cc2-c2ccccc2)cc1 ; c1ccc(-c2ccc(N(c3cccc(-c4cccc(-c5cccc6ccccc56)c4)c3)c3ccc4ccc5ccccc5c4c3)cc2-c2ccccc2)cc1 ; c1ccc(-c2ccc(N(c3ccc4ccc5ccccc5c4c3)c3ccccc3-c3ccc(-c4cccc5ccccc45)cc3)cc2-c2ccccc2)cc1] 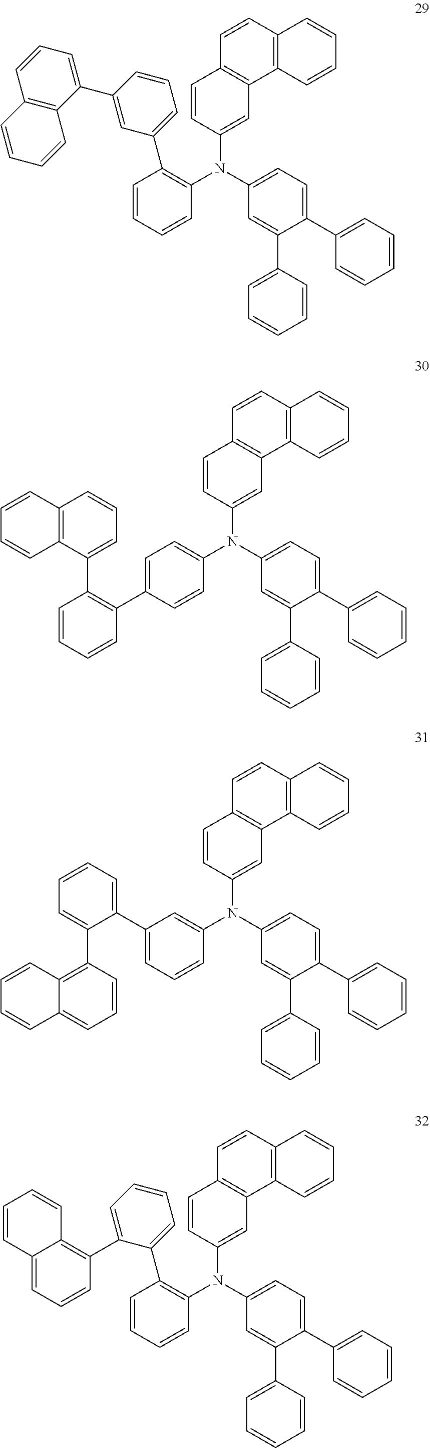 [SMILES: c1ccc(-c2ccc(N(c3ccc4ccc5ccccc5c4c3)c3ccccc3-c3cccc(-c4cccc5ccccc45)c3)cc2-c2ccccc2)cc1 ; c1ccc(-c2ccc(N(c3ccc(-c4ccccc4-c4cccc5ccccc45)cc3)c3ccc4ccc5ccccc5c4c3)cc2-c2ccccc2)cc1 ; c1ccc(-c2ccc(N(c3cccc(-c4ccccc4-c4cccc5ccccc45)c3)c3ccc4ccc5ccccc5c4c3)cc2-c2ccccc2)cc1 ; c1ccc(-c2ccc(N(c3ccc4ccc5ccccc5c4c3)c3ccccc3-c3ccccc3-c3cccc4ccccc34)cc2-c2ccccc2)cc1] 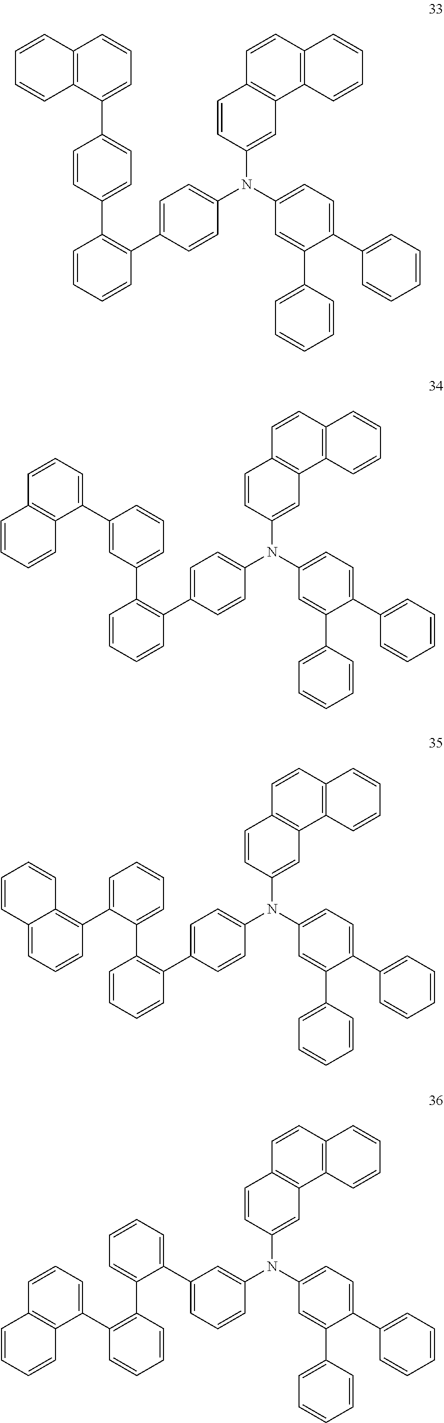 [SMILES: c1ccc(-c2ccc(N(c3ccc(-c4ccccc4-c4ccc(-c5cccc6ccccc56)cc4)cc3)c3ccc4ccc5ccccc5c4c3)cc2-c2ccccc2)cc1 ; c1ccc(-c2ccc(N(c3ccc(-c4ccccc4-c4cccc(-c5cccc6ccccc56)c4)cc3)c3ccc4ccc5ccccc5c4c3)cc2-c2ccccc2)cc1 ; c1ccc(-c2ccc(N(c3ccc(-c4ccccc4-c4ccccc4-c4cccc5ccccc45)cc3)c3ccc4ccc5ccccc5c4c3)cc2-c2ccccc2)cc1 ; c1ccc(-c2ccc(N(c3cccc(-c4ccccc4-c4ccccc4-c4cccc5ccccc45)c3)c3ccc4ccc5ccccc5c4c3)cc2-c2ccccc2)cc1] 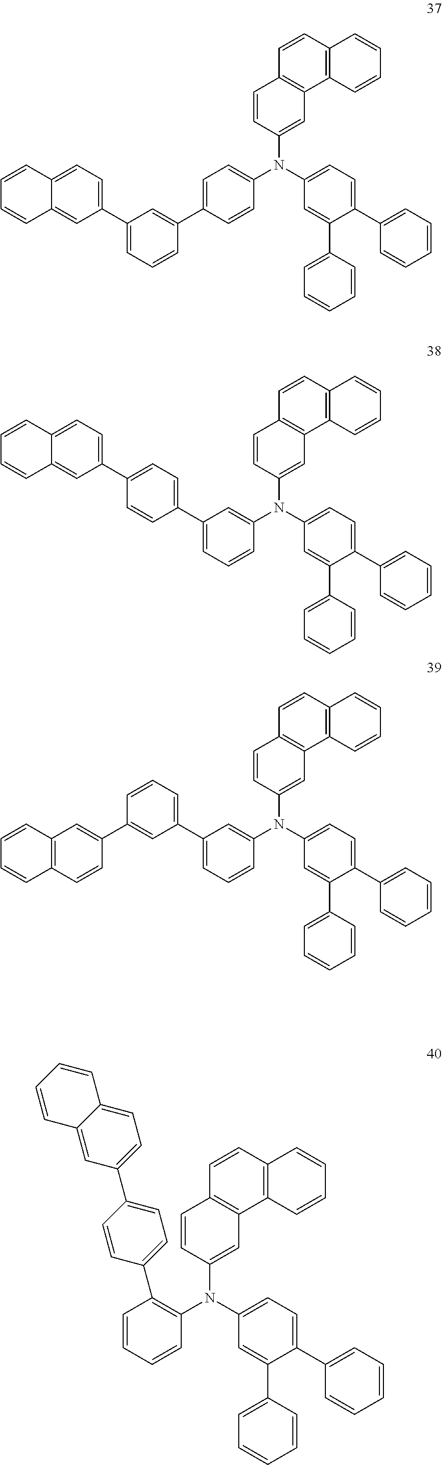 [SMILES: c1ccc(-c2ccc(N(c3ccc(-c4cccc(-c5ccc6ccccc6c5)c4)cc3)c3ccc4ccc5ccccc5c4c3)cc2-c2ccccc2)cc1 ; c1ccc(-c2ccc(N(c3cccc(-c4ccc(-c5ccc6ccccc6c5)cc4)c3)c3ccc4ccc5ccccc5c4c3)cc2-c2ccccc2)cc1 ; c1ccc(-c2ccc(N(c3cccc(-c4cccc(-c5ccc6ccccc6c5)c4)c3)c3ccc4ccc5ccccc5c4c3)cc2-c2ccccc2)cc1 ; c1ccc(-c2ccc(N(c3ccc4ccc5ccccc5c4c3)c3ccccc3-c3ccc(-c4ccc5ccccc5c4)cc3)cc2-c2ccccc2)cc1] 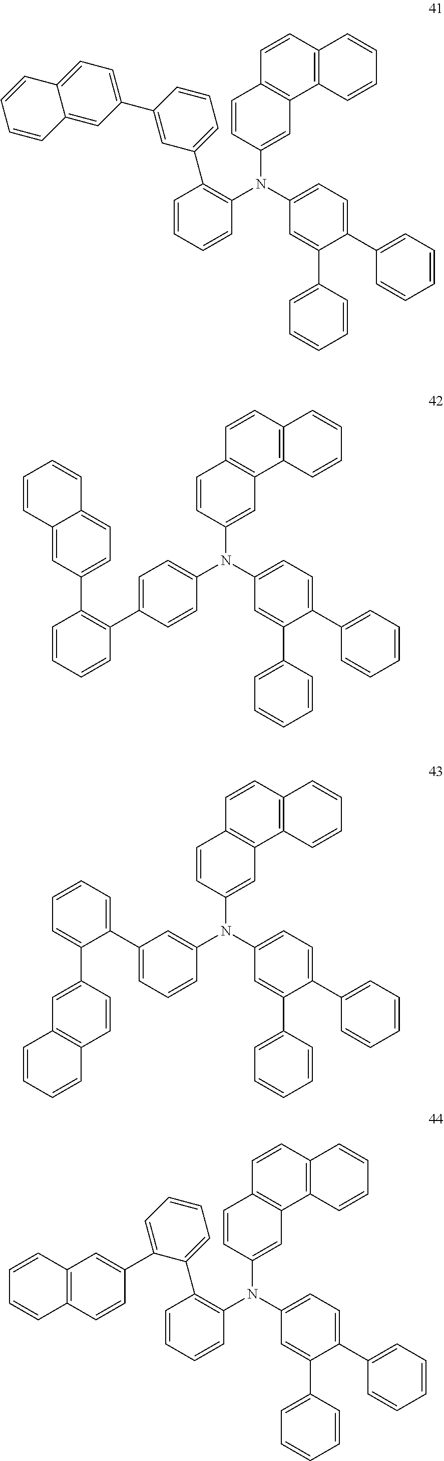 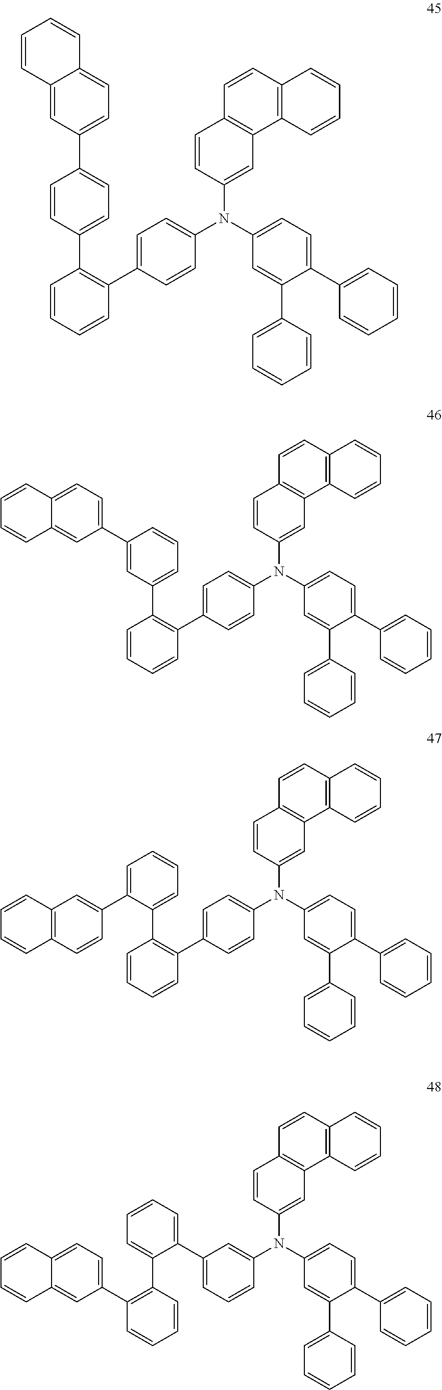 [SMILES: c1ccc(-c2ccc(N(c3ccc(-c4ccccc4-c4ccc(-c5ccc6ccccc6c5)cc4)cc3)c3ccc4ccc5ccccc5c4c3)cc2-c2ccccc2)cc1 ; c1ccc(-c2ccc(N(c3ccc(-c4ccccc4-c4cccc(-c5ccc6ccccc6c5)c4)cc3)c3ccc4ccc5ccccc5c4c3)cc2-c2ccccc2)cc1 ; c1ccc(-c2ccc(N(c3ccc(-c4ccccc4-c4ccccc4-c4ccc5ccccc5c4)cc3)c3ccc4ccc5ccccc5c4c3)cc2-c2ccccc2)cc1 ; c1ccc(-c2ccc(N(c3cccc(-c4ccccc4-c4ccccc4-c4ccc5ccccc5c4)c3)c3ccc4ccc5ccccc5c4c3)cc2-c2ccccc2)cc1] 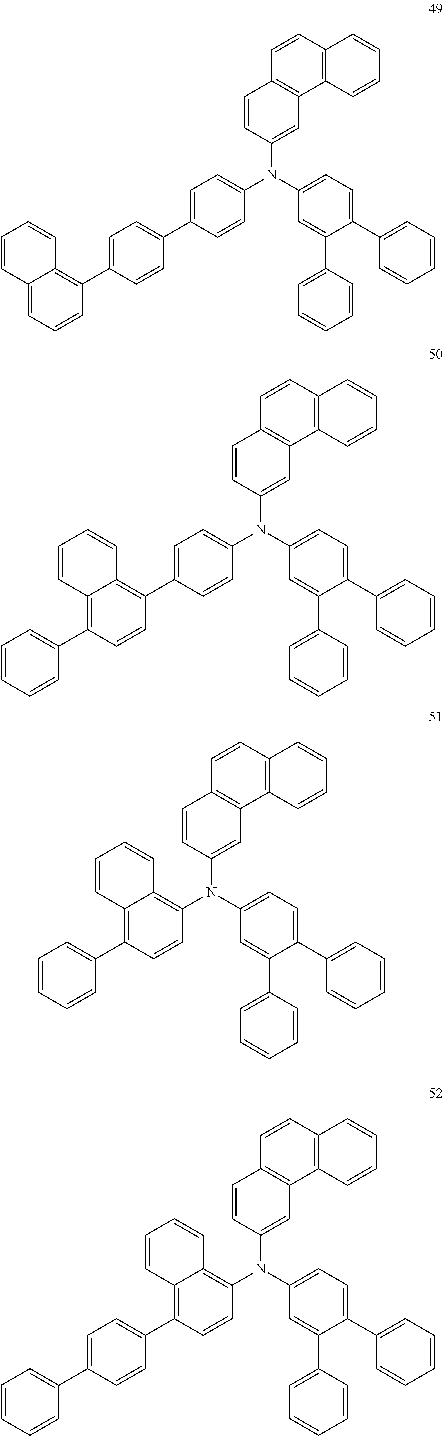 [SMILES: c1ccc(-c2ccc(N(c3ccc(-c4ccc(-c5cccc6ccccc56)cc4)cc3)c3ccc4ccc5ccccc5c4c3)cc2-c2ccccc2)cc1 ; c1ccc(-c2ccc(N(c3ccc(-c4ccc(-c5ccccc5)c5ccccc45)cc3)c3ccc4ccc5ccccc5c4c3)cc2-c2ccccc2)cc1 ; c1ccc(-c2ccc(N(c3ccc4ccc5ccccc5c4c3)c3ccc(-c4ccccc4)c4ccccc34)cc2-c2ccccc2)cc1 ; c1ccc(-c2ccc(-c3ccc(N(c4ccc(-c5ccccc5)c(-c5ccccc5)c4)c4ccc5ccc6ccccc6c5c4)c4ccccc34)cc2)cc1] 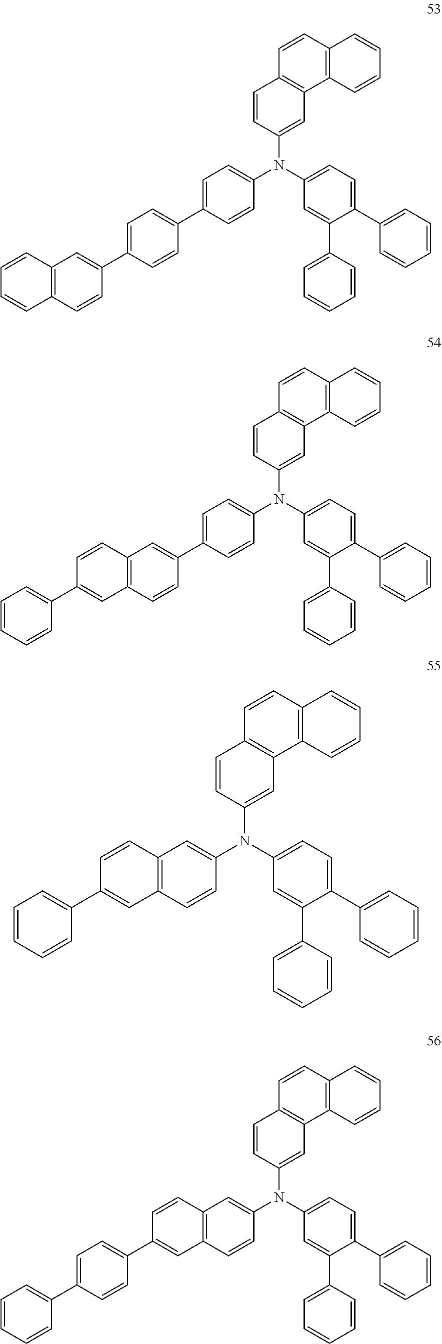 [SMILES: c1ccc(-c2ccc(N(c3ccc(-c4ccc(-c5ccc6ccccc6c5)cc4)cc3)c3ccc4ccc5ccccc5c4c3)cc2-c2ccccc2)cc1 ; c1ccc(-c2ccc3cc(-c4ccc(N(c5ccc(-c6ccccc6)c(-c6ccccc6)c5)c5ccc6ccc7ccccc7c6c5)cc4)ccc3c2)cc1 ; c1ccc(-c2ccc3cc(N(c4ccc(-c5ccccc5)c(-c5ccccc5)c4)c4ccc5ccc6ccccc6c5c4)ccc3c2)cc1 ; c1ccc(-c2ccc(-c3ccc4cc(N(c5ccc(-c6ccccc6)c(-c6ccccc6)c5)c5ccc6ccc7ccccc7c6c5)ccc4c3)cc2)cc1] 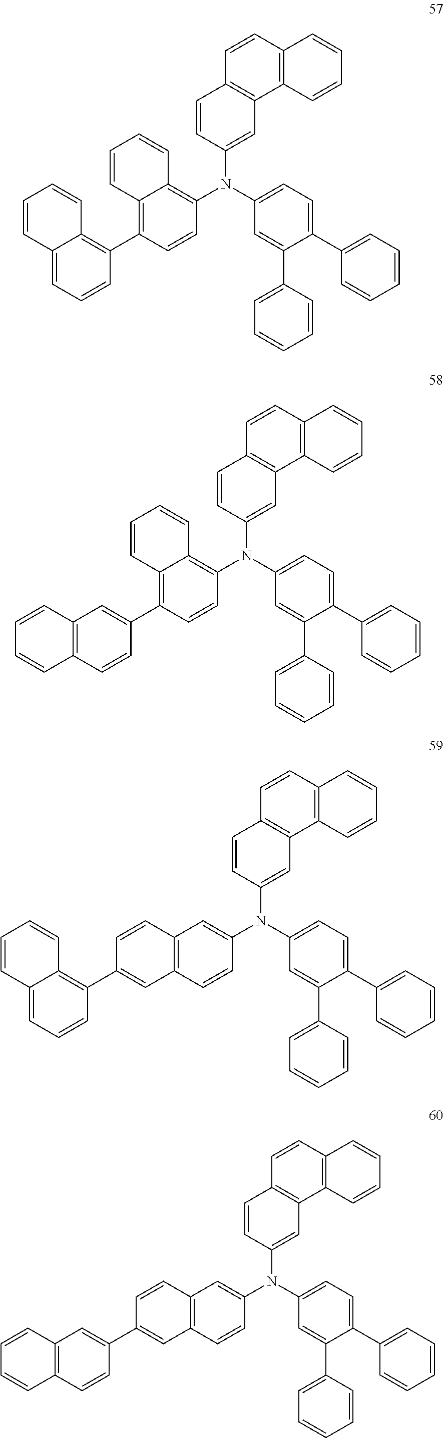 [SMILES: c1ccc(-c2ccc(N(c3ccc4ccc5ccccc5c4c3)c3ccc(-c4cccc5ccccc45)c4ccccc34)cc2-c2ccccc2)cc1 ; c1ccc(-c2ccc(N(c3ccc4ccc5ccccc5c4c3)c3ccc(-c4ccc5ccccc5c4)c4ccccc34)cc2-c2ccccc2)cc1 ; c1ccc(-c2ccc(N(c3ccc4cc(-c5cccc6ccccc56)ccc4c3)c3ccc4ccc5ccccc5c4c3)cc2-c2ccccc2)cc1 ; c1ccc(-c2ccc(N(c3ccc4cc(-c5ccc6ccccc6c5)ccc4c3)c3ccc4ccc5ccccc5c4c3)cc2-c2ccccc2)cc1] 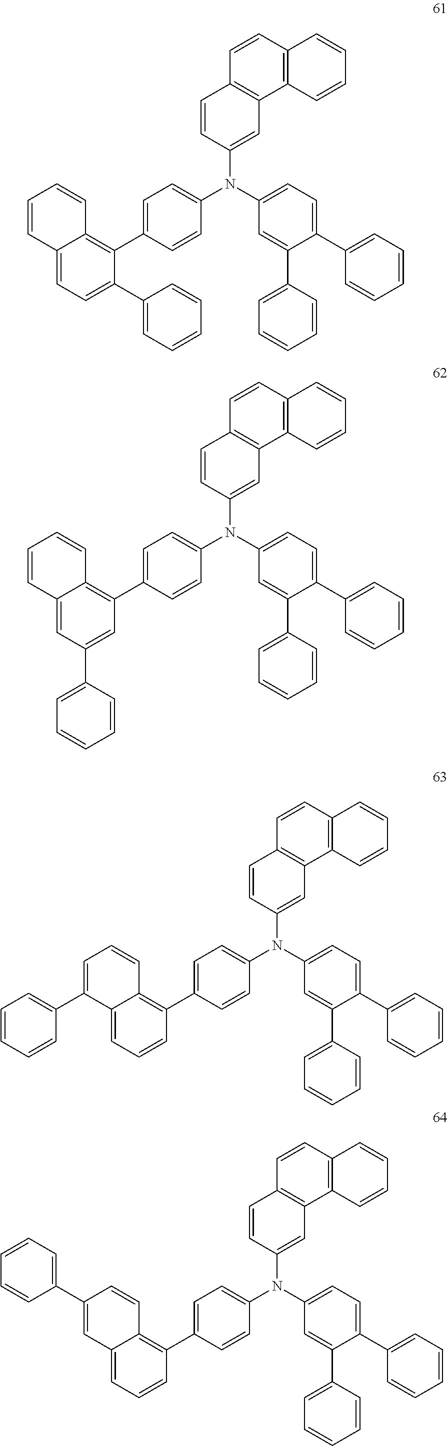 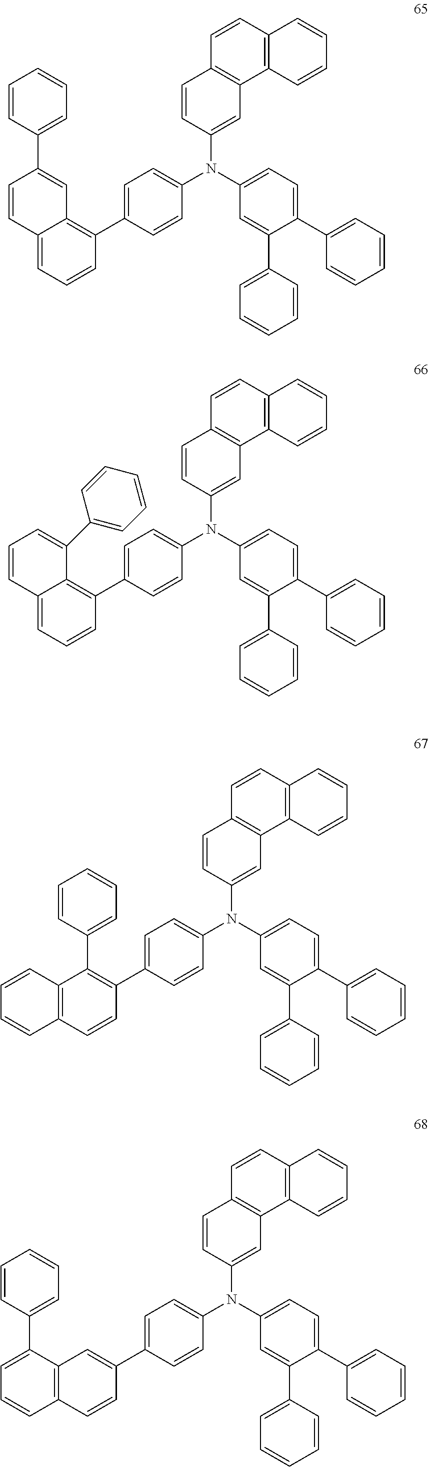 [SMILES: c1ccc(-c2ccc3cccc(-c4ccc(N(c5ccc(-c6ccccc6)c(-c6ccccc6)c5)c5ccc6ccc7ccccc7c6c5)cc4)c3c2)cc1 ; c1ccc(-c2ccc(N(c3ccc(-c4cccc5cccc(-c6ccccc6)c45)cc3)c3ccc4ccc5ccccc5c4c3)cc2-c2ccccc2)cc1 ; c1ccc(-c2ccc(N(c3ccc(-c4ccc5ccccc5c4-c4ccccc4)cc3)c3ccc4ccc5ccccc5c4c3)cc2-c2ccccc2)cc1 ; c1ccc(-c2ccc(N(c3ccc(-c4ccc5cccc(-c6ccccc6)c5c4)cc3)c3ccc4ccc5ccccc5c4c3)cc2-c2ccccc2)cc1] 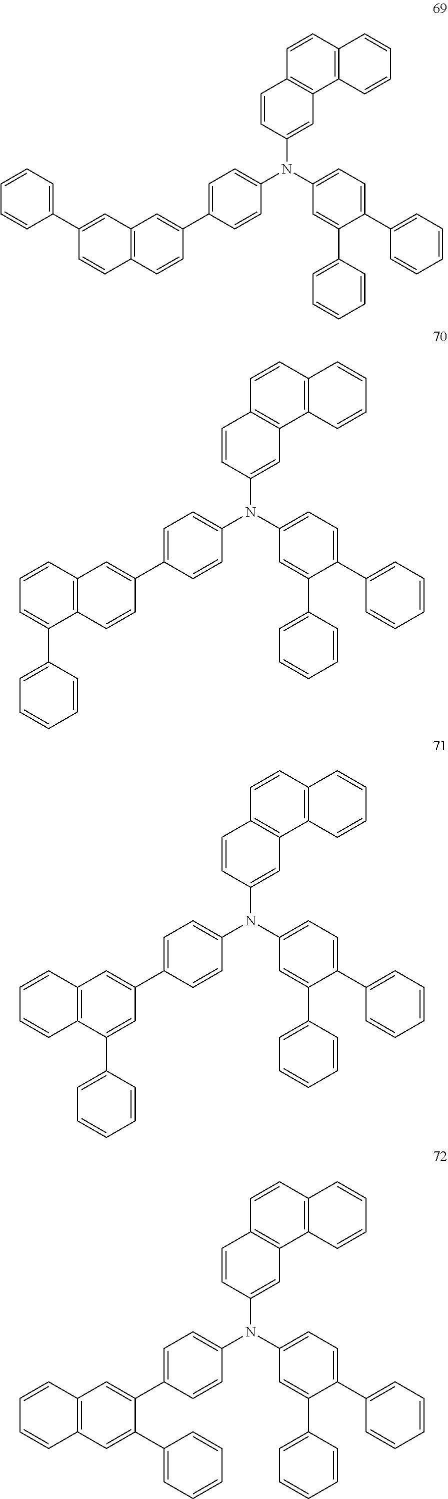 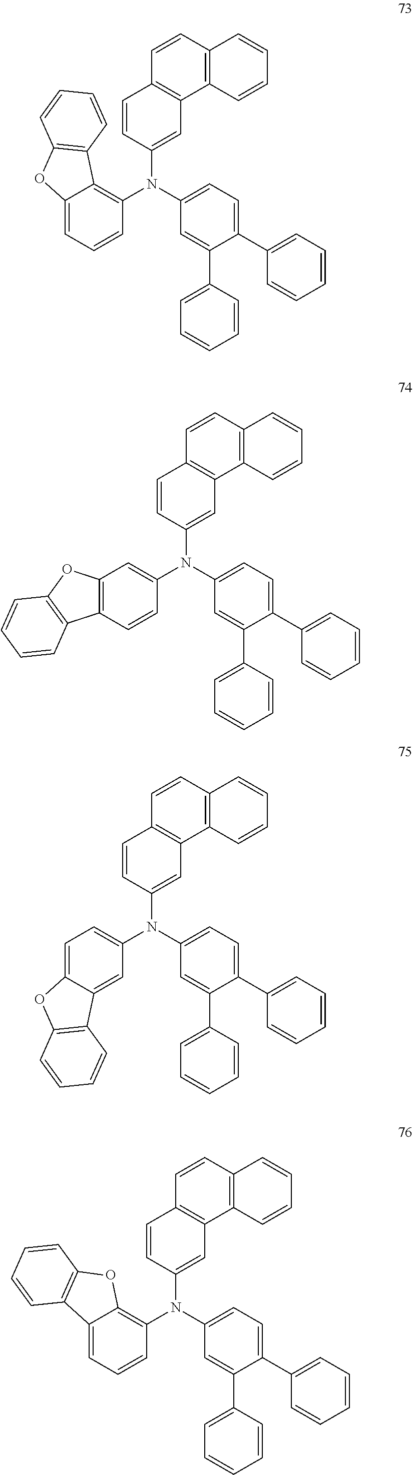 [SMILES: c1ccc(-c2ccc(N(c3ccc4ccc5ccccc5c4c3)c3cccc4oc5ccccc5c34)cc2-c2ccccc2)cc1 ; c1ccc(-c2ccc(N(c3ccc4c(c3)oc3ccccc34)c3ccc4ccc5ccccc5c4c3)cc2-c2ccccc2)cc1 ; c1ccc(-c2ccc(N(c3ccc4ccc5ccccc5c4c3)c3ccc4oc5ccccc5c4c3)cc2-c2ccccc2)cc1 ; c1ccc(-c2ccc(N(c3ccc4ccc5ccccc5c4c3)c3cccc4c3oc3ccccc34)cc2-c2ccccc2)cc1] 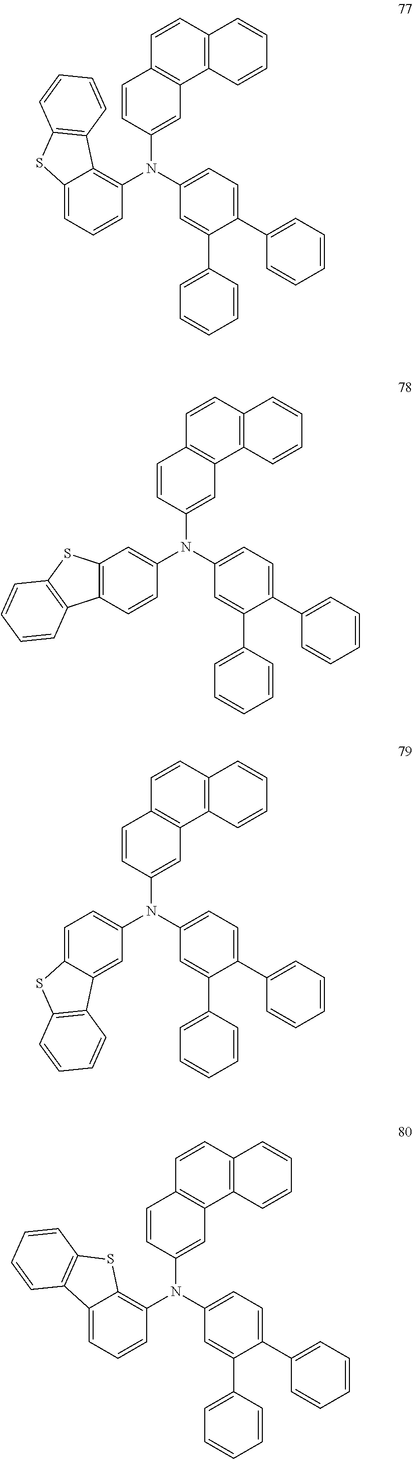 [SMILES: c1ccc(-c2ccc(N(c3ccc4ccc5ccccc5c4c3)c3cccc4sc5ccccc5c34)cc2-c2ccccc2)cc1 ; c1ccc(-c2ccc(N(c3ccc4c(c3)sc3ccccc34)c3ccc4ccc5ccccc5c4c3)cc2-c2ccccc2)cc1 ; c1ccc(-c2ccc(N(c3ccc4ccc5ccccc5c4c3)c3ccc4sc5ccccc5c4c3)cc2-c2ccccc2)cc1 ; c1ccc(-c2ccc(N(c3ccc4ccc5ccccc5c4c3)c3cccc4c3sc3ccccc34)cc2-c2ccccc2)cc1] 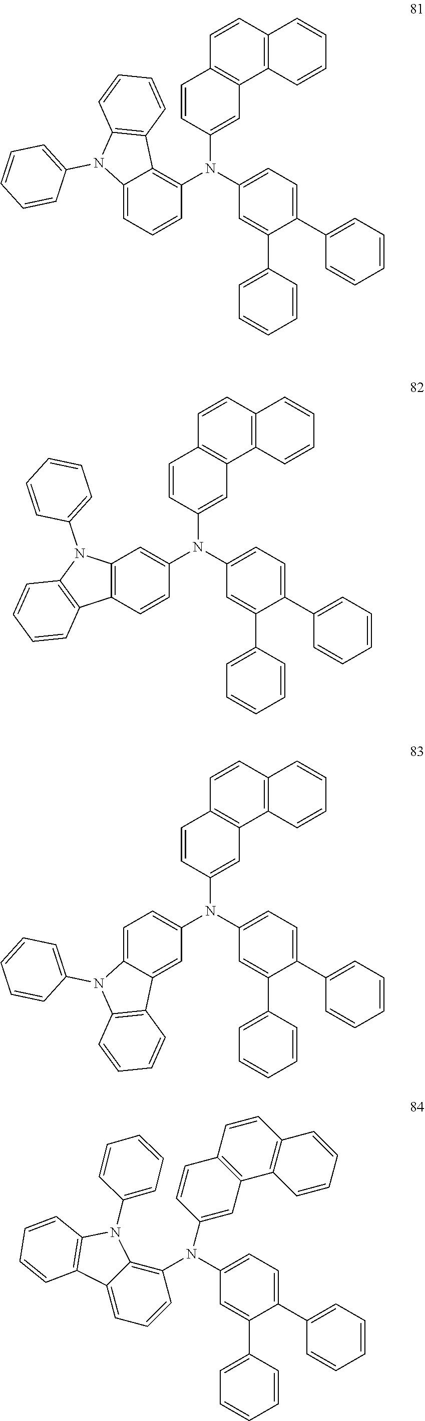 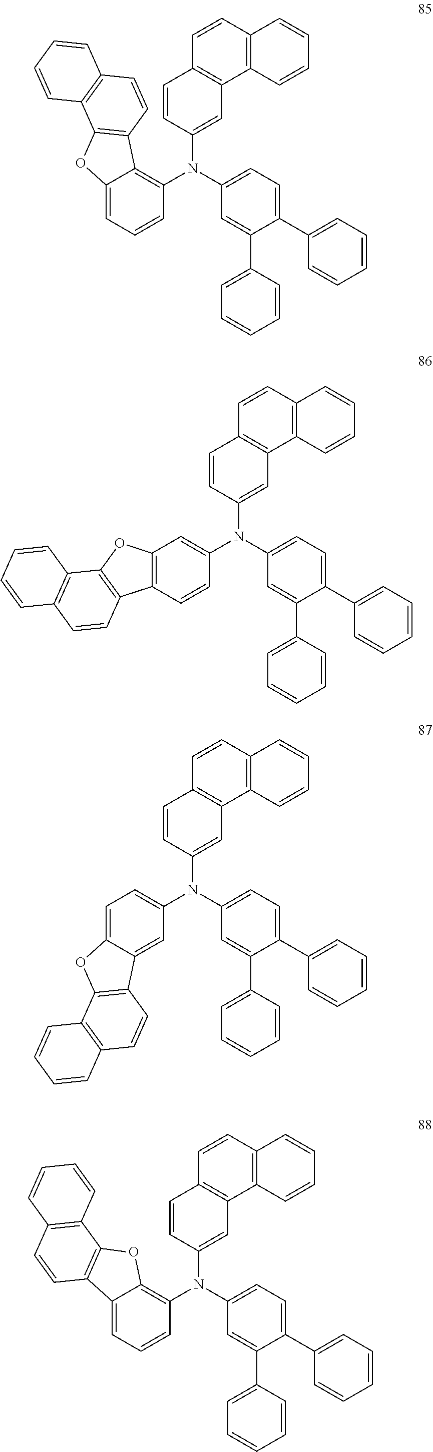 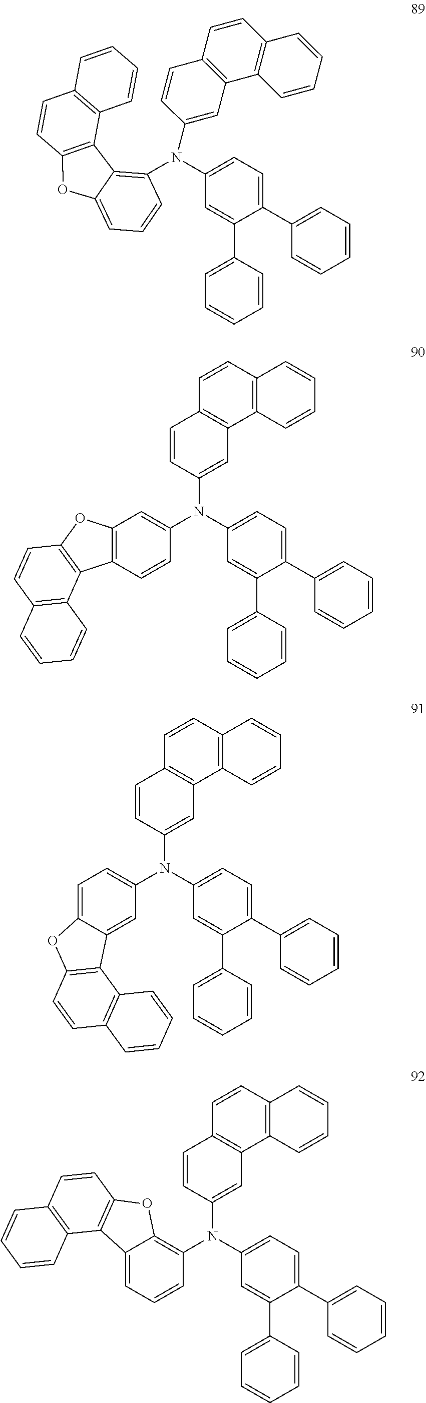 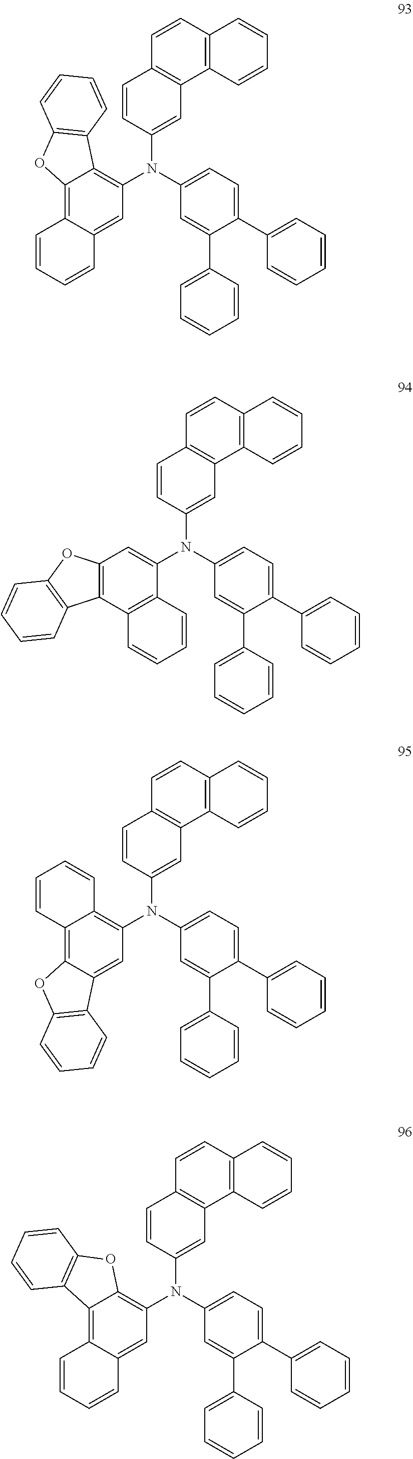 [SMILES: c1ccc(-c2ccc(N(c3ccc4ccc5ccccc5c4c3)c3cc4ccccc4c4oc5ccccc5c34)cc2-c2ccccc2)cc1 ; c1ccc(-c2ccc(N(c3ccc4ccc5ccccc5c4c3)c3cc4oc5ccccc5c4c4ccccc34)cc2-c2ccccc2)cc1 ; c1ccc(-c2ccc(N(c3ccc4ccc5ccccc5c4c3)c3cc4c5ccccc5oc4c4ccccc34)cc2-c2ccccc2)cc1 ; c1ccc(-c2ccc(N(c3ccc4ccc5ccccc5c4c3)c3cc4ccccc4c4c3oc3ccccc34)cc2-c2ccccc2)cc1] 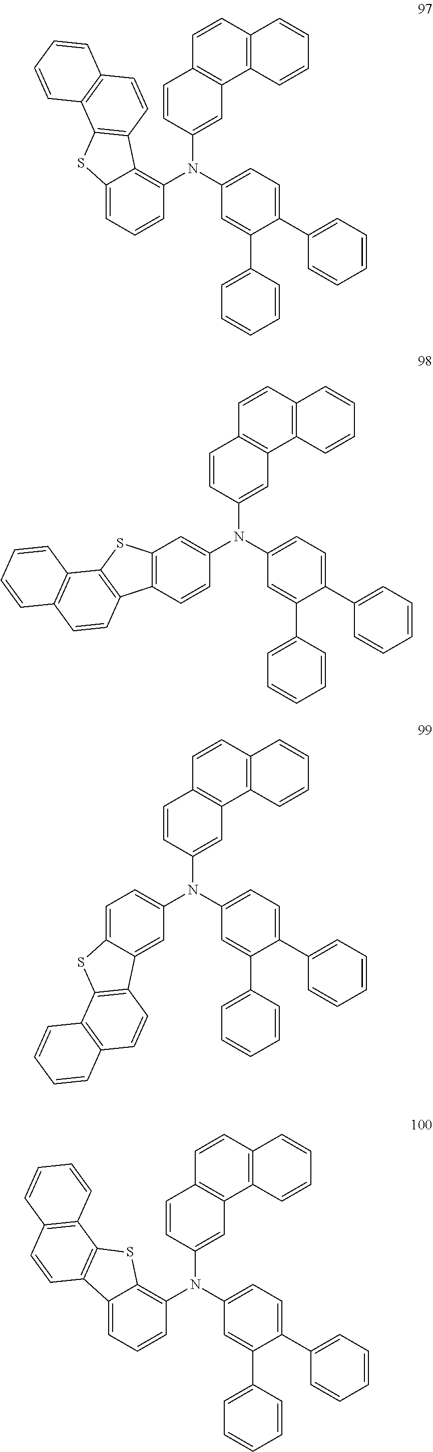 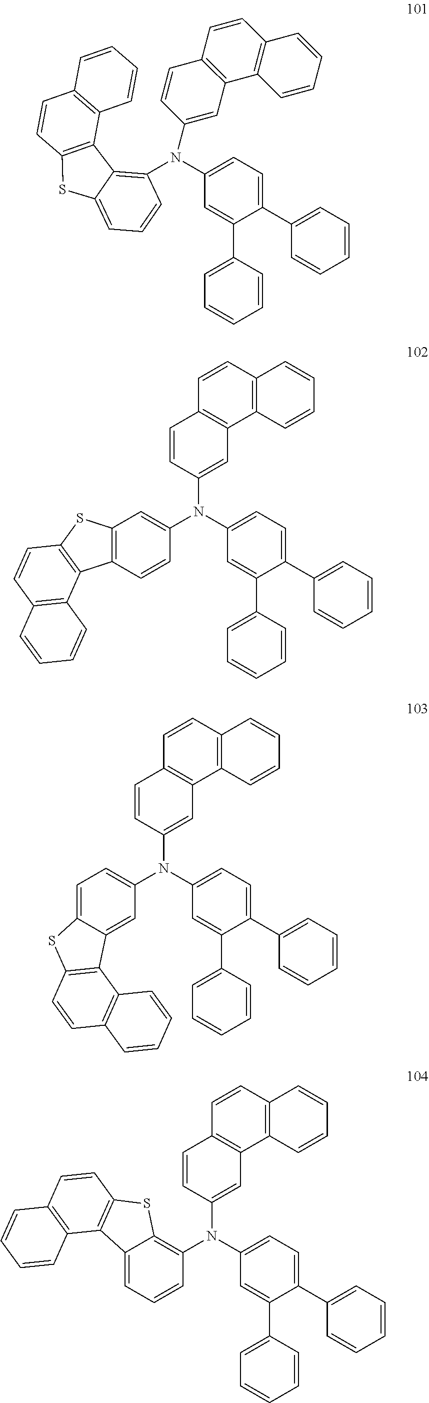 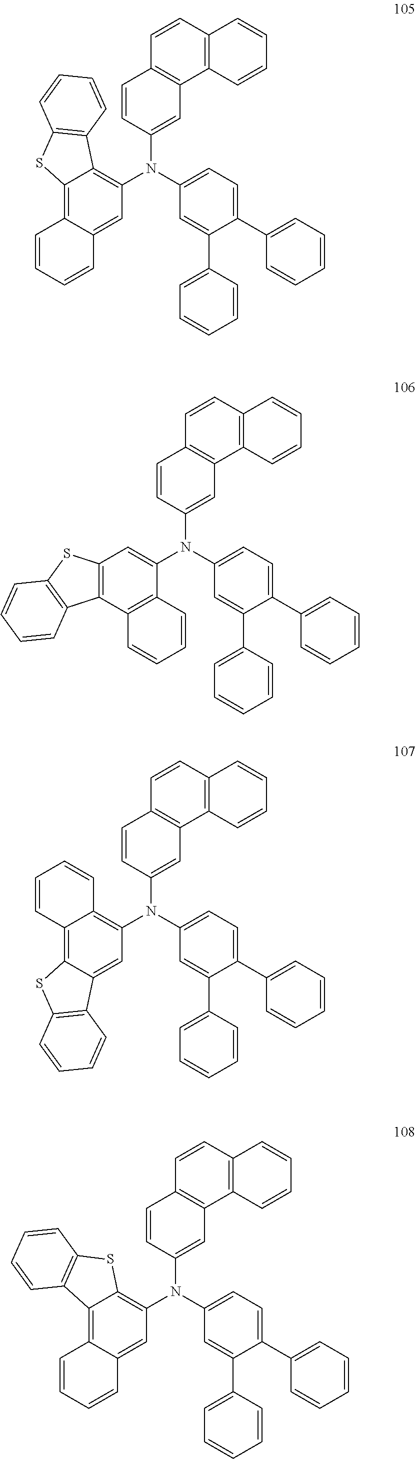 [SMILES: c1ccc(-c2ccc(N(c3ccc4ccc5ccccc5c4c3)c3cc4ccccc4c4sc5ccccc5c34)cc2-c2ccccc2)cc1 ; c1ccc(-c2ccc(N(c3ccc4ccc5ccccc5c4c3)c3cc4sc5ccccc5c4c4ccccc34)cc2-c2ccccc2)cc1 ; c1ccc(-c2ccc(N(c3ccc4ccc5ccccc5c4c3)c3cc4c5ccccc5sc4c4ccccc34)cc2-c2ccccc2)cc1 ; c1ccc(-c2ccc(N(c3ccc4ccc5ccccc5c4c3)c3cc4ccccc4c4c3sc3ccccc34)cc2-c2ccccc2)cc1] 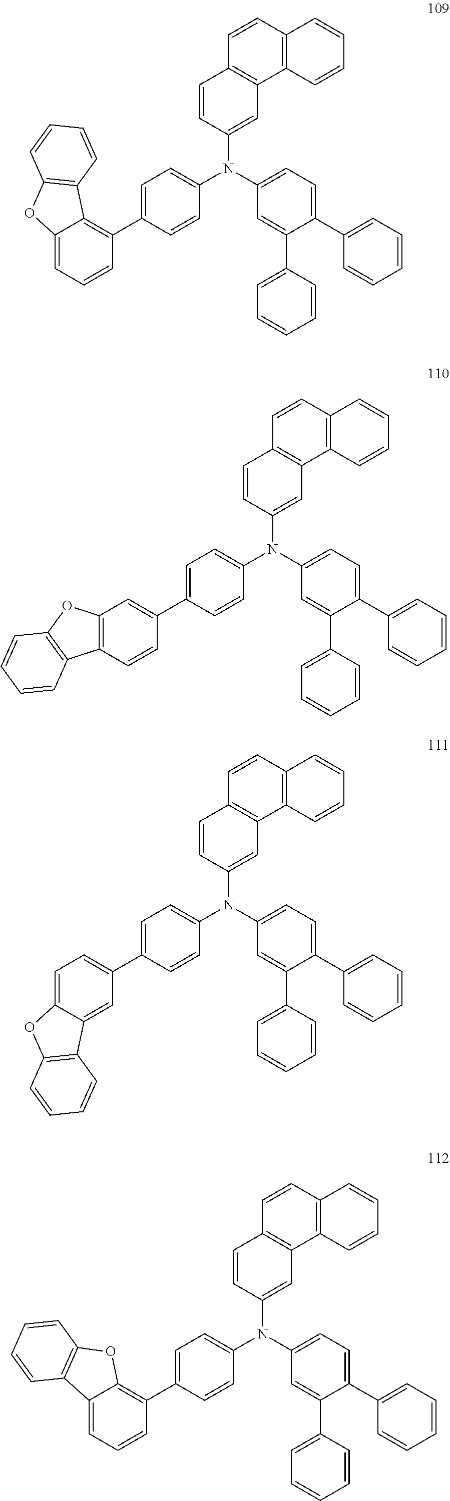 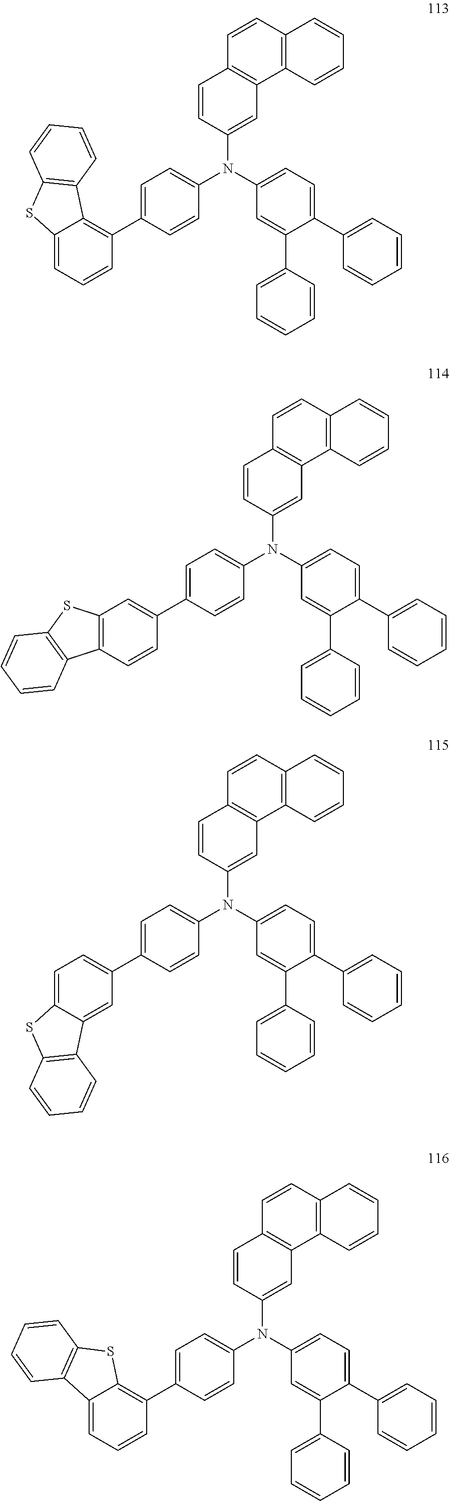 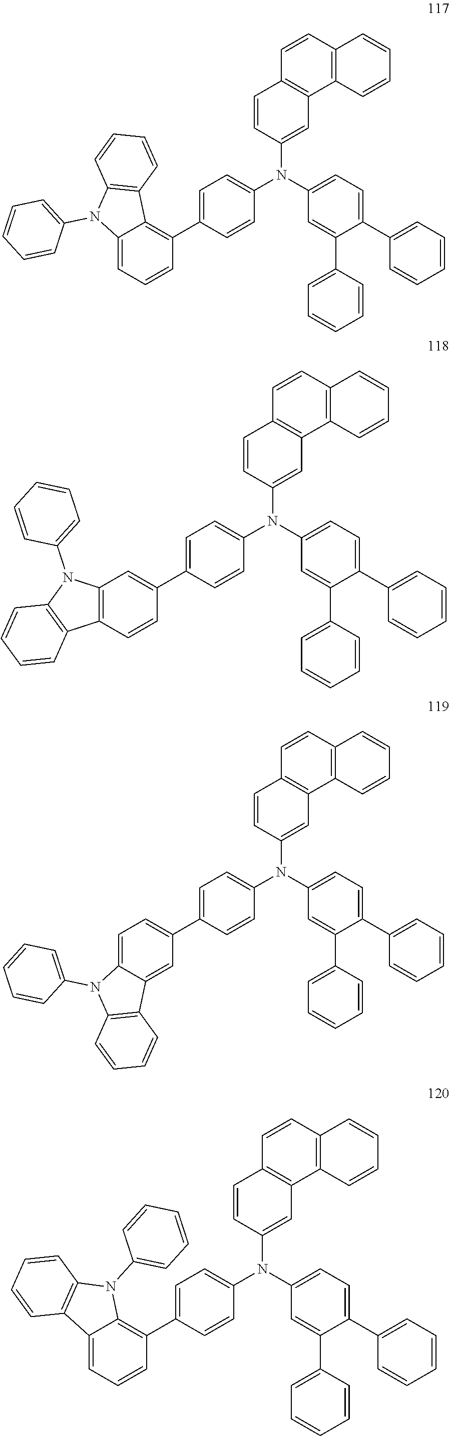 [SMILES: c1ccc(-c2ccc(N(c3ccc(-c4cccc5c4c4ccccc4n5-c4ccccc4)cc3)c3ccc4ccc5ccccc5c4c3)cc2-c2ccccc2)cc1 ; c1ccc(-c2ccc(N(c3ccc(-c4ccc5c6ccccc6n(-c6ccccc6)c5c4)cc3)c3ccc4ccc5ccccc5c4c3)cc2-c2ccccc2)cc1 ; c1ccc(-c2ccc(N(c3ccc(-c4ccc5c(c4)c4ccccc4n5-c4ccccc4)cc3)c3ccc4ccc5ccccc5c4c3)cc2-c2ccccc2)cc1 ; c1ccc(-c2ccc(N(c3ccc(-c4cccc5c6ccccc6n(-c6ccccc6)c45)cc3)c3ccc4ccc5ccccc5c4c3)cc2-c2ccccc2)cc1] 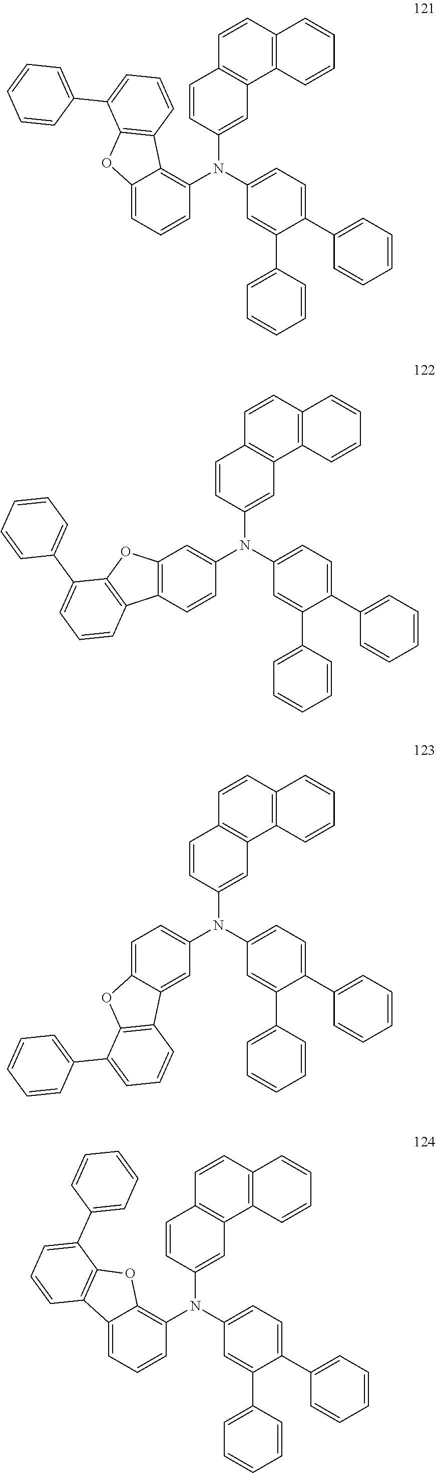 [SMILES: c1ccc(-c2ccc(N(c3ccc4ccc5ccccc5c4c3)c3cccc4oc5c(-c6ccccc6)cccc5c34)cc2-c2ccccc2)cc1 ; c1ccc(-c2ccc(N(c3ccc4c(c3)oc3c(-c5ccccc5)cccc34)c3ccc4ccc5ccccc5c4c3)cc2-c2ccccc2)cc1 ; c1ccc(-c2ccc(N(c3ccc4ccc5ccccc5c4c3)c3ccc4oc5c(-c6ccccc6)cccc5c4c3)cc2-c2ccccc2)cc1 ; c1ccc(-c2ccc(N(c3ccc4ccc5ccccc5c4c3)c3cccc4c3oc3c(-c5ccccc5)cccc34)cc2-c2ccccc2)cc1] 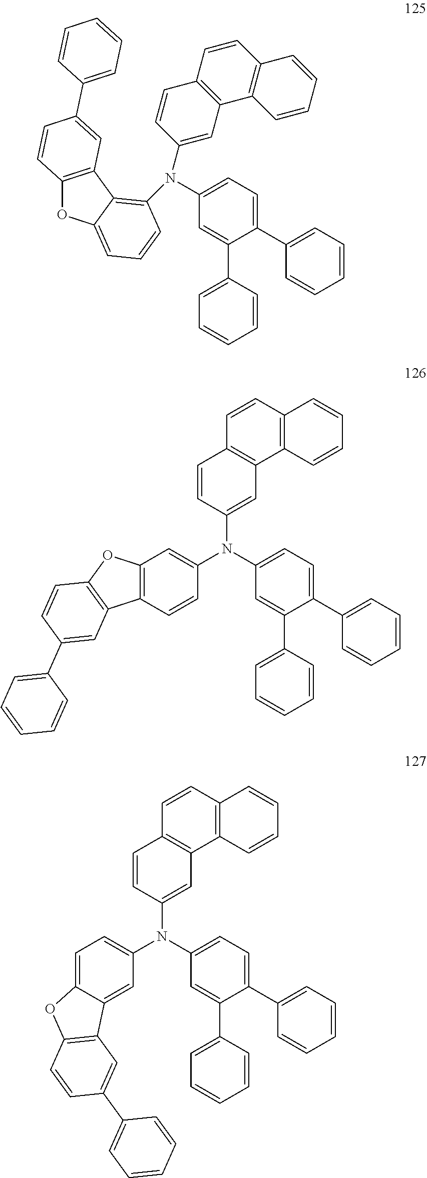 [SMILES: c1ccc(-c2ccc3oc4cccc(N(c5ccc(-c6ccccc6)c(-c6ccccc6)c5)c5ccc6ccc7ccccc7c6c5)c4c3c2)cc1 ; c1ccc(-c2ccc3oc4cc(N(c5ccc(-c6ccccc6)c(-c6ccccc6)c5)c5ccc6ccc7ccccc7c6c5)ccc4c3c2)cc1 ; c1ccc(-c2ccc3oc4ccc(N(c5ccc(-c6ccccc6)c(-c6ccccc6)c5)c5ccc6ccc7ccccc7c6c5)cc4c3c2)cc1] 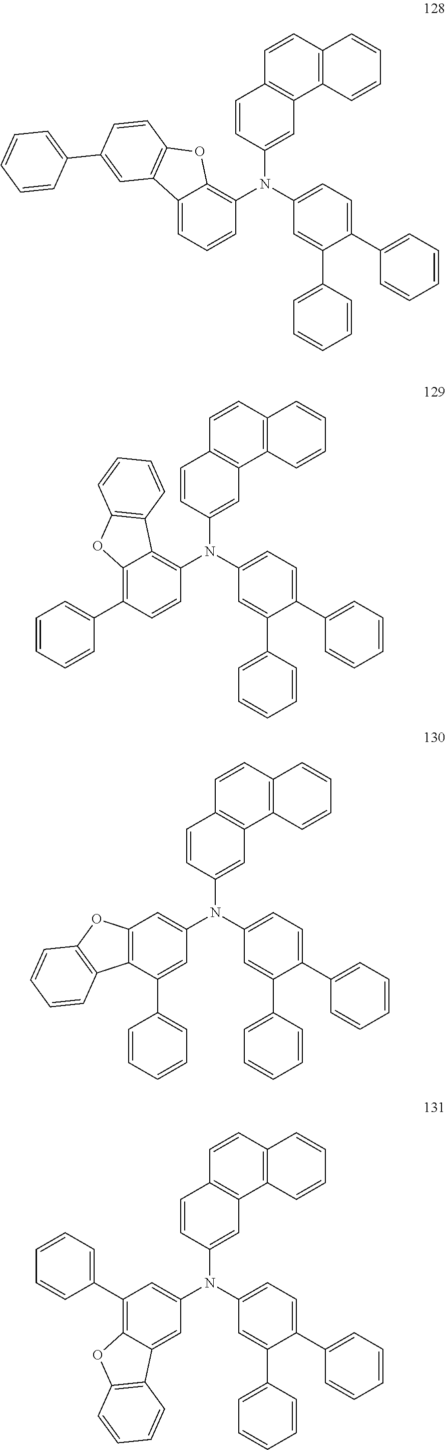 [SMILES: c1ccc(-c2ccc3oc4c(N(c5ccc(-c6ccccc6)c(-c6ccccc6)c5)c5ccc6ccc7ccccc7c6c5)cccc4c3c2)cc1 ; c1ccc(-c2ccc(N(c3ccc4ccc5ccccc5c4c3)c3ccc(-c4ccccc4)c4oc5ccccc5c34)cc2-c2ccccc2)cc1 ; c1ccc(-c2ccc(N(c3cc(-c4ccccc4)c4c(c3)oc3ccccc34)c3ccc4ccc5ccccc5c4c3)cc2-c2ccccc2)cc1 ; c1ccc(-c2ccc(N(c3ccc4ccc5ccccc5c4c3)c3cc(-c4ccccc4)c4oc5ccccc5c4c3)cc2-c2ccccc2)cc1] 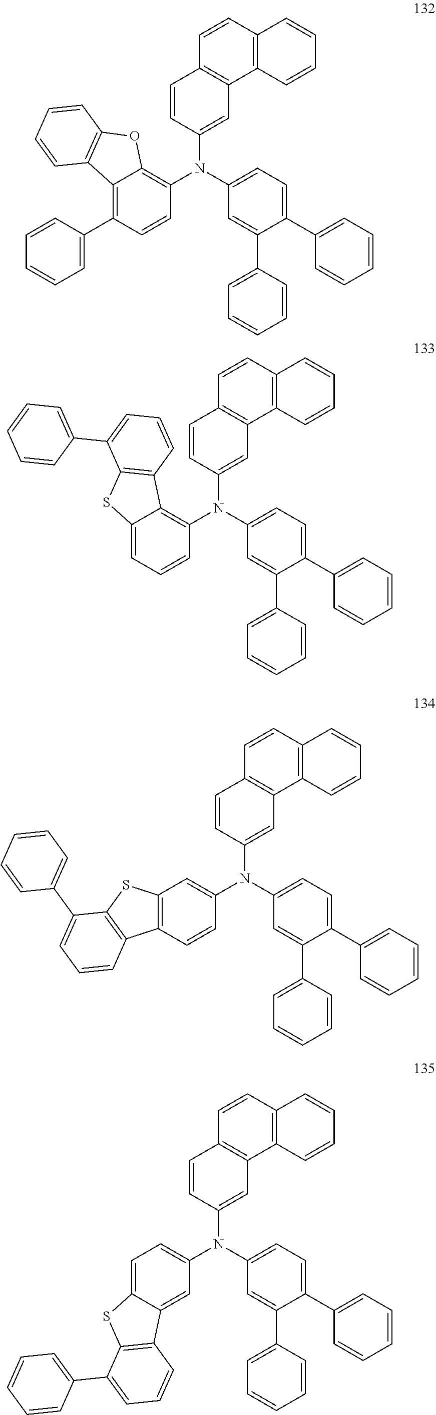 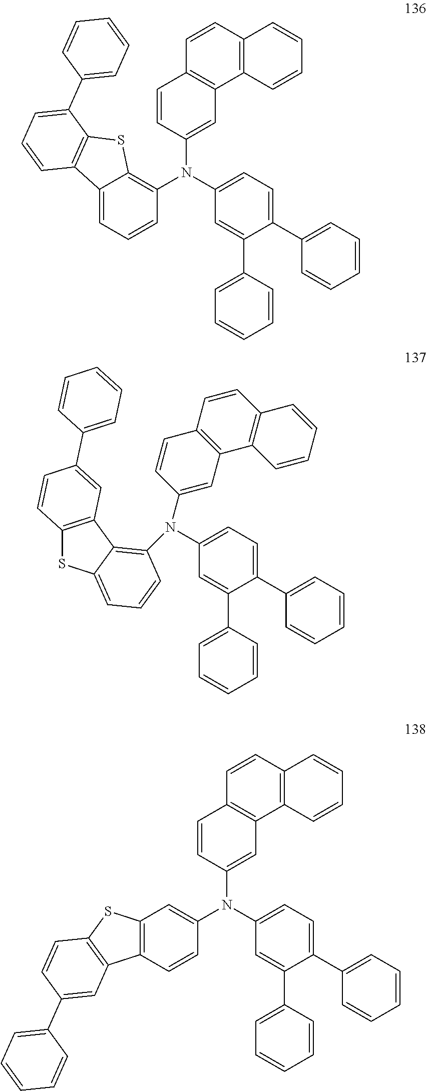 [SMILES: c1ccc(-c2ccc(N(c3ccc4ccc5ccccc5c4c3)c3cccc4c3sc3c(-c5ccccc5)cccc34)cc2-c2ccccc2)cc1 ; c1ccc(-c2ccc3sc4cccc(N(c5ccc(-c6ccccc6)c(-c6ccccc6)c5)c5ccc6ccc7ccccc7c6c5)c4c3c2)cc1 ; c1ccc(-c2ccc3sc4cc(N(c5ccc(-c6ccccc6)c(-c6ccccc6)c5)c5ccc6ccc7ccccc7c6c5)ccc4c3c2)cc1] 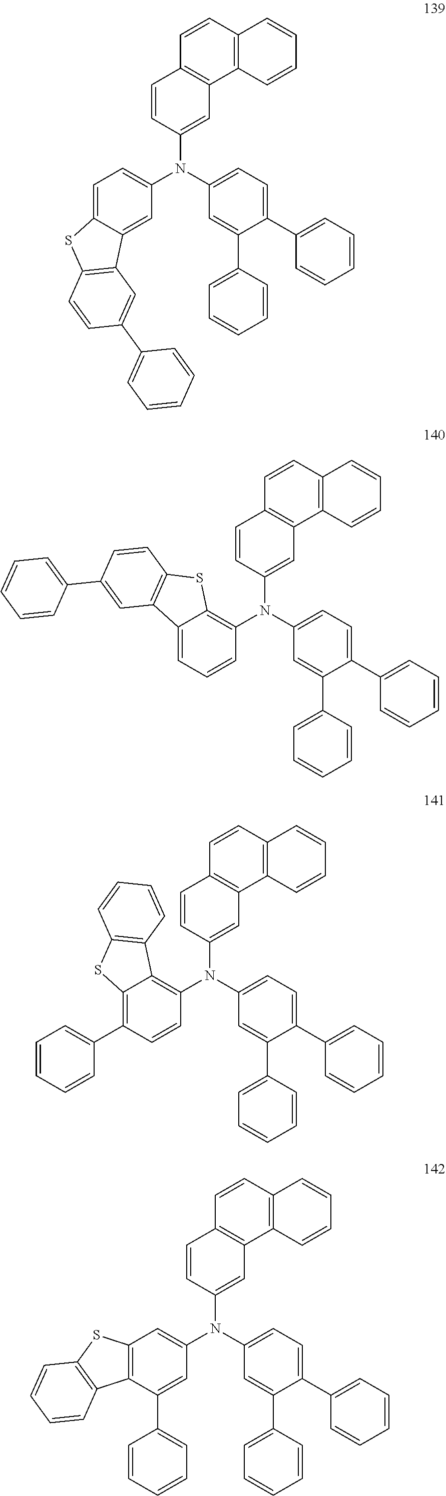 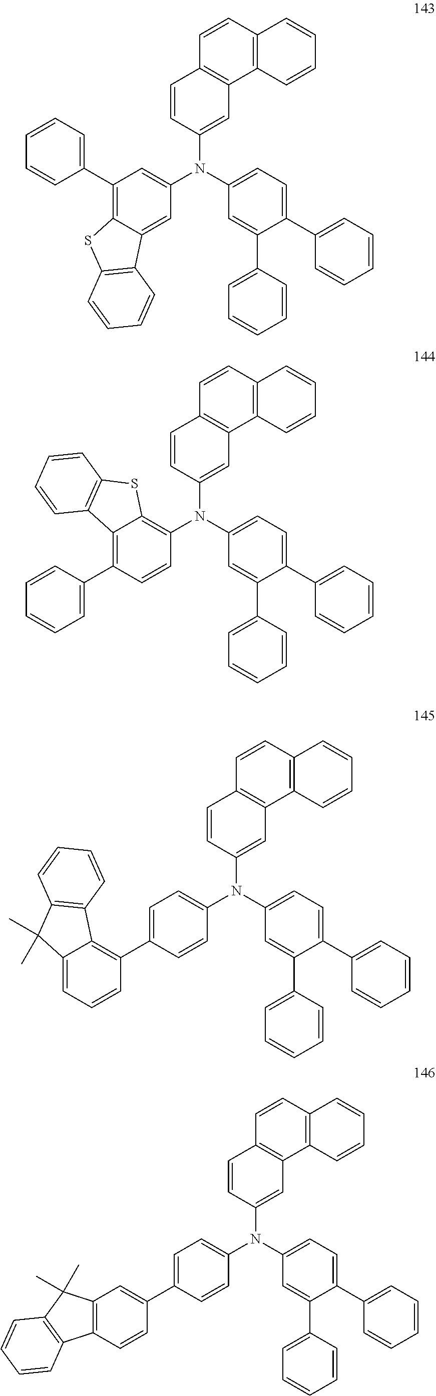 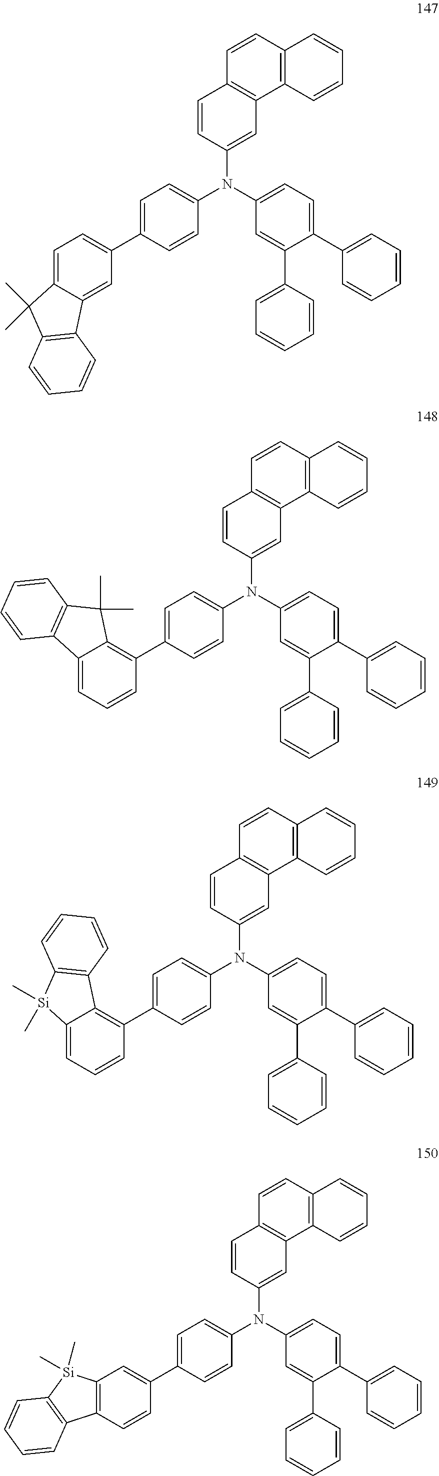 [SMILES: CC1(C)c2ccccc2-c2cc(-c3ccc(N(c4ccc(-c5ccccc5)c(-c5ccccc5)c4)c4ccc5ccc6ccccc6c5c4)cc3)ccc21 ; CC1(C)c2ccccc2-c2cccc(-c3ccc(N(c4ccc(-c5ccccc5)c(-c5ccccc5)c4)c4ccc5ccc6ccccc6c5c4)cc3)c21 ; C[Si]1(C)c2ccccc2-c2c(-c3ccc(N(c4ccc(-c5ccccc5)c(-c5ccccc5)c4)c4ccc5ccc6ccccc6c5c4)cc3)cccc21 ; C[Si]1(C)c2ccccc2-c2ccc(-c3ccc(N(c4ccc(-c5ccccc5)c(-c5ccccc5)c4)c4ccc5ccc6ccccc6c5c4)cc3)cc21] 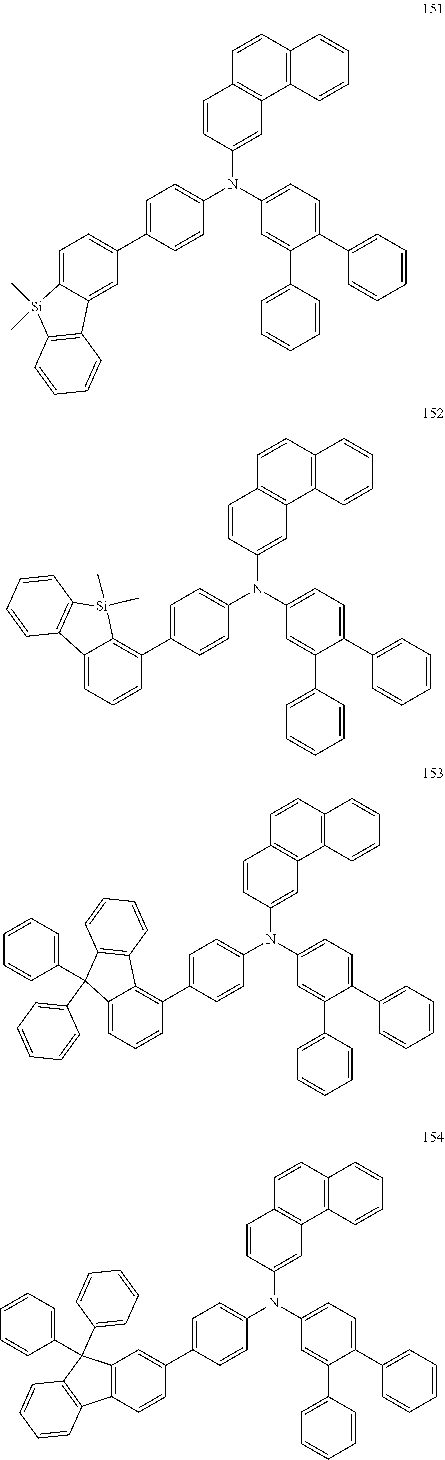 [SMILES: C[Si]1(C)c2ccccc2-c2cc(-c3ccc(N(c4ccc(-c5ccccc5)c(-c5ccccc5)c4)c4ccc5ccc6ccccc6c5c4)cc3)ccc21 ; C[Si]1(C)c2ccccc2-c2cccc(-c3ccc(N(c4ccc(-c5ccccc5)c(-c5ccccc5)c4)c4ccc5ccc6ccccc6c5c4)cc3)c21 ; c1ccc(-c2ccc(N(c3ccc(-c4cccc5c4-c4ccccc4C5(c4ccccc4)c4ccccc4)cc3)c3ccc4ccc5ccccc5c4c3)cc2-c2ccccc2)cc1 ; c1ccc(-c2ccc(N(c3ccc(-c4ccc5c(c4)C(c4ccccc4)(c4ccccc4)c4ccccc4-5)cc3)c3ccc4ccc5ccccc5c4c3)cc2-c2ccccc2)cc1] 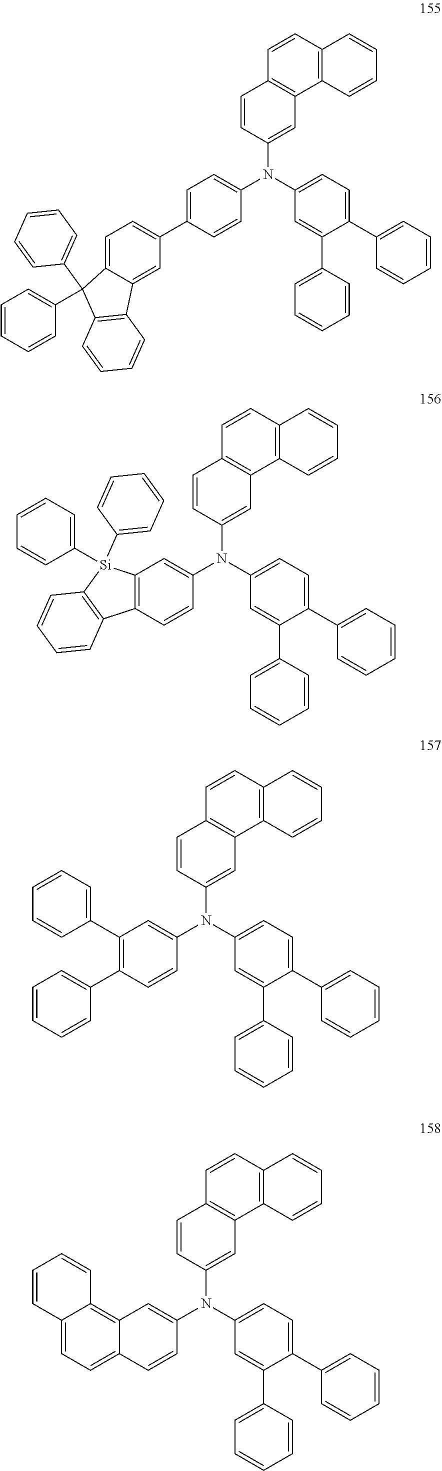 [SMILES: c1ccc(-c2ccc(N(c3ccc(-c4ccc5c(c4)-c4ccccc4C5(c4ccccc4)c4ccccc4)cc3)c3ccc4ccc5ccccc5c4c3)cc2-c2ccccc2)cc1 ; c1ccc(-c2ccc(N(c3ccc4c(c3)[Si](c3ccccc3)(c3ccccc3)c3ccccc3-4)c3ccc4ccc5ccccc5c4c3)cc2-c2ccccc2)cc1 ; c1ccc(-c2ccc(N(c3ccc(-c4ccccc4)c(-c4ccccc4)c3)c3ccc4ccc5ccccc5c4c3)cc2-c2ccccc2)cc1 ; c1ccc(-c2ccc(N(c3ccc4ccc5ccccc5c4c3)c3ccc4ccc5ccccc5c4c3)cc2-c2ccccc2)cc1] 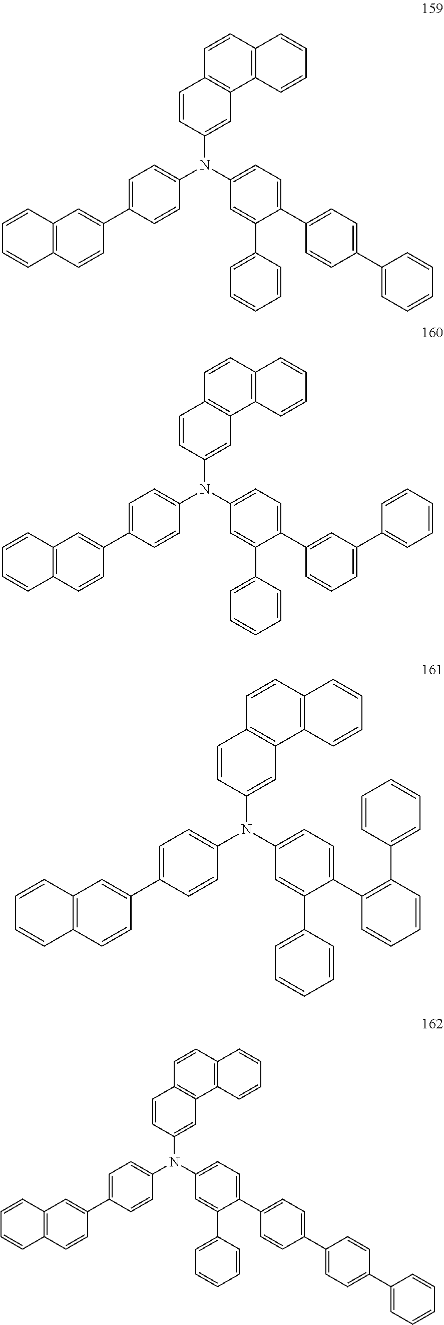 [SMILES: c1ccc(-c2ccc(-c3ccc(N(c4ccc(-c5ccc6ccccc6c5)cc4)c4ccc5ccc6ccccc6c5c4)cc3-c3ccccc3)cc2)cc1 ; c1ccc(-c2cccc(-c3ccc(N(c4ccc(-c5ccc6ccccc6c5)cc4)c4ccc5ccc6ccccc6c5c4)cc3-c3ccccc3)c2)cc1 ; c1ccc(-c2ccccc2-c2ccc(N(c3ccc(-c4ccc5ccccc5c4)cc3)c3ccc4ccc5ccccc5c4c3)cc2-c2ccccc2)cc1 ; c1ccc(-c2ccc(-c3ccc(-c4ccc(N(c5ccc(-c6ccc7ccccc7c6)cc5)c5ccc6ccc7ccccc7c6c5)cc4-c4ccccc4)cc3)cc2)cc1] 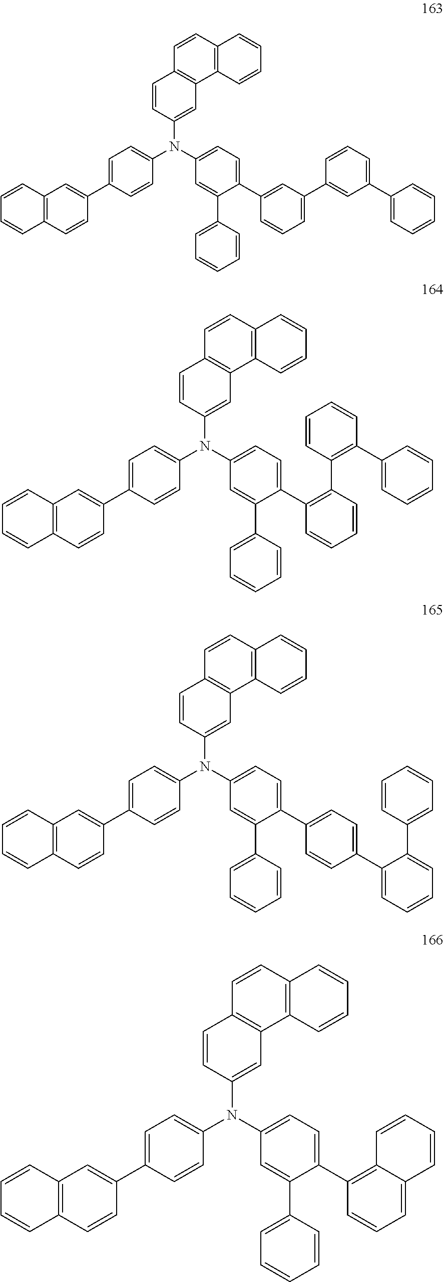 [SMILES: c1ccc(-c2cccc(-c3cccc(-c4ccc(N(c5ccc(-c6ccc7ccccc7c6)cc5)c5ccc6ccc7ccccc7c6c5)cc4-c4ccccc4)c3)c2)cc1 ; c1ccc(-c2ccccc2-c2ccccc2-c2ccc(N(c3ccc(-c4ccc5ccccc5c4)cc3)c3ccc4ccc5ccccc5c4c3)cc2-c2ccccc2)cc1 ; c1ccc(-c2ccccc2-c2ccc(-c3ccc(N(c4ccc(-c5ccc6ccccc6c5)cc4)c4ccc5ccc6ccccc6c5c4)cc3-c3ccccc3)cc2)cc1 ; c1ccc(-c2cc(N(c3ccc(-c4ccc5ccccc5c4)cc3)c3ccc4ccc5ccccc5c4c3)ccc2-c2cccc3ccccc23)cc1] 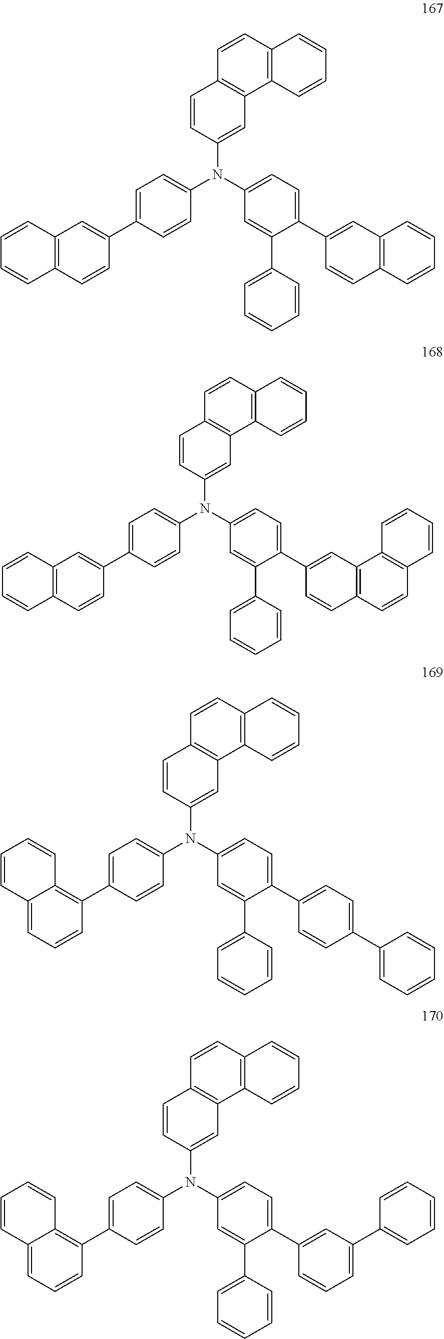 [SMILES: c1ccc(-c2cc(N(c3ccc(-c4ccc5ccccc5c4)cc3)c3ccc4ccc5ccccc5c4c3)ccc2-c2ccc3ccccc3c2)cc1 ; c1ccc(-c2cc(N(c3ccc(-c4ccc5ccccc5c4)cc3)c3ccc4ccc5ccccc5c4c3)ccc2-c2ccc3ccc4ccccc4c3c2)cc1 ; c1ccc(-c2ccc(-c3ccc(N(c4ccc(-c5cccc6ccccc56)cc4)c4ccc5ccc6ccccc6c5c4)cc3-c3ccccc3)cc2)cc1 ; c1ccc(-c2cccc(-c3ccc(N(c4ccc(-c5cccc6ccccc56)cc4)c4ccc5ccc6ccccc6c5c4)cc3-c3ccccc3)c2)cc1] 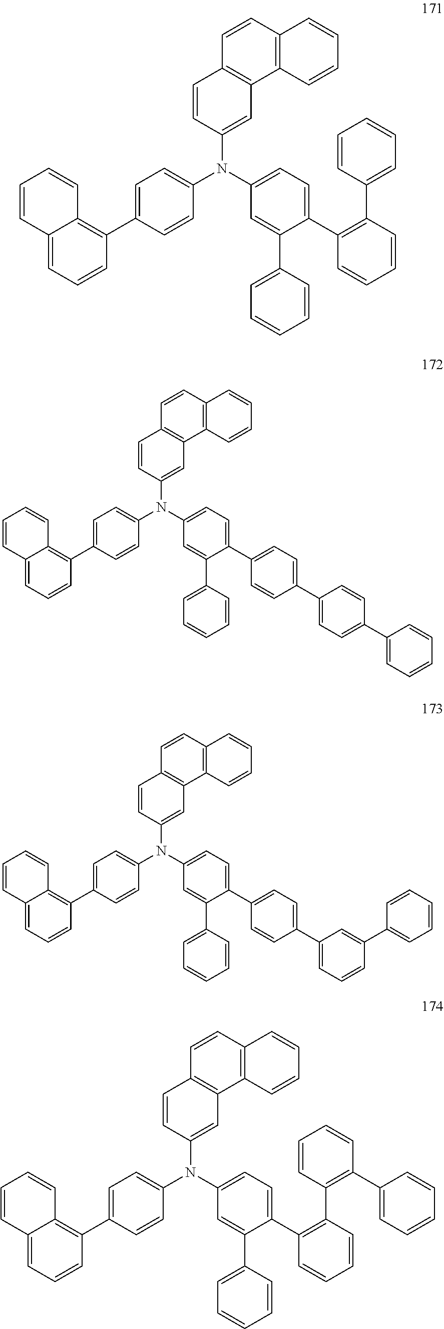 [SMILES: c1ccc(-c2ccccc2-c2ccc(N(c3ccc(-c4cccc5ccccc45)cc3)c3ccc4ccc5ccccc5c4c3)cc2-c2ccccc2)cc1 ; c1ccc(-c2ccc(-c3ccc(-c4ccc(N(c5ccc(-c6cccc7ccccc67)cc5)c5ccc6ccc7ccccc7c6c5)cc4-c4ccccc4)cc3)cc2)cc1 ; c1ccc(-c2cccc(-c3ccc(-c4ccc(N(c5ccc(-c6cccc7ccccc67)cc5)c5ccc6ccc7ccccc7c6c5)cc4-c4ccccc4)cc3)c2)cc1 ; c1ccc(-c2ccccc2-c2ccccc2-c2ccc(N(c3ccc(-c4cccc5ccccc45)cc3)c3ccc4ccc5ccccc5c4c3)cc2-c2ccccc2)cc1] 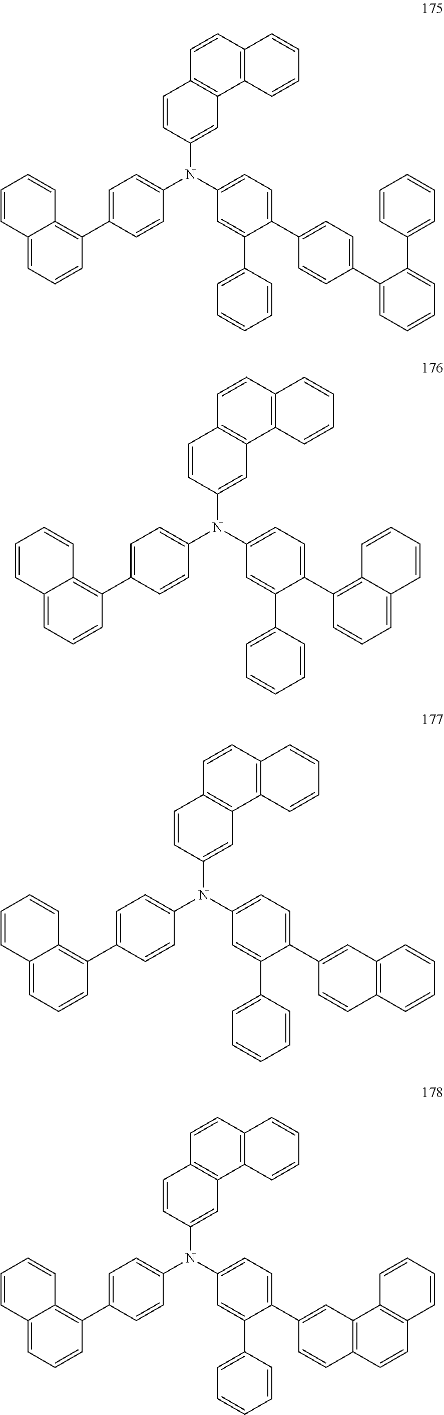 [SMILES: c1ccc(-c2ccccc2-c2ccc(-c3ccc(N(c4ccc(-c5cccc6ccccc56)cc4)c4ccc5ccc6ccccc6c5c4)cc3-c3ccccc3)cc2)cc1 ; c1ccc(-c2cc(N(c3ccc(-c4cccc5ccccc45)cc3)c3ccc4ccc5ccccc5c4c3)ccc2-c2cccc3ccccc23)cc1 ; c1ccc(-c2cc(N(c3ccc(-c4cccc5ccccc45)cc3)c3ccc4ccc5ccccc5c4c3)ccc2-c2ccc3ccccc3c2)cc1 ; c1ccc(-c2cc(N(c3ccc(-c4cccc5ccccc45)cc3)c3ccc4ccc5ccccc5c4c3)ccc2-c2ccc3ccc4ccccc4c3c2)cc1] 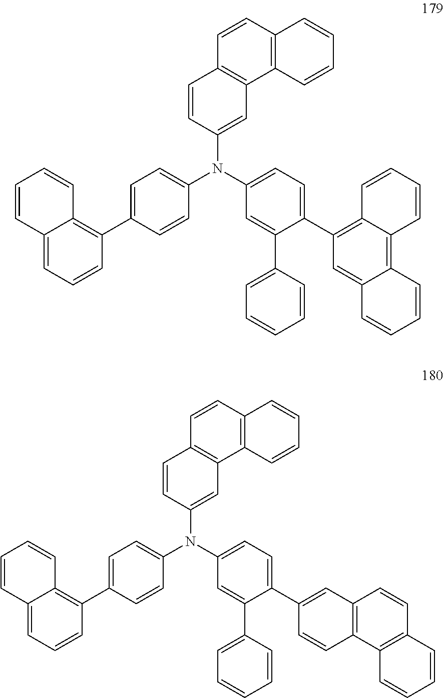 [SMILES: c1ccc(-c2cc(N(c3ccc(-c4cccc5ccccc45)cc3)c3ccc4ccc5ccccc5c4c3)ccc2-c2cc3ccccc3c3ccccc23)cc1 ; c1ccc(-c2cc(N(c3ccc(-c4cccc5ccccc45)cc3)c3ccc4ccc5ccccc5c4c3)ccc2-c2ccc3c(ccc4ccccc43)c2)cc1] 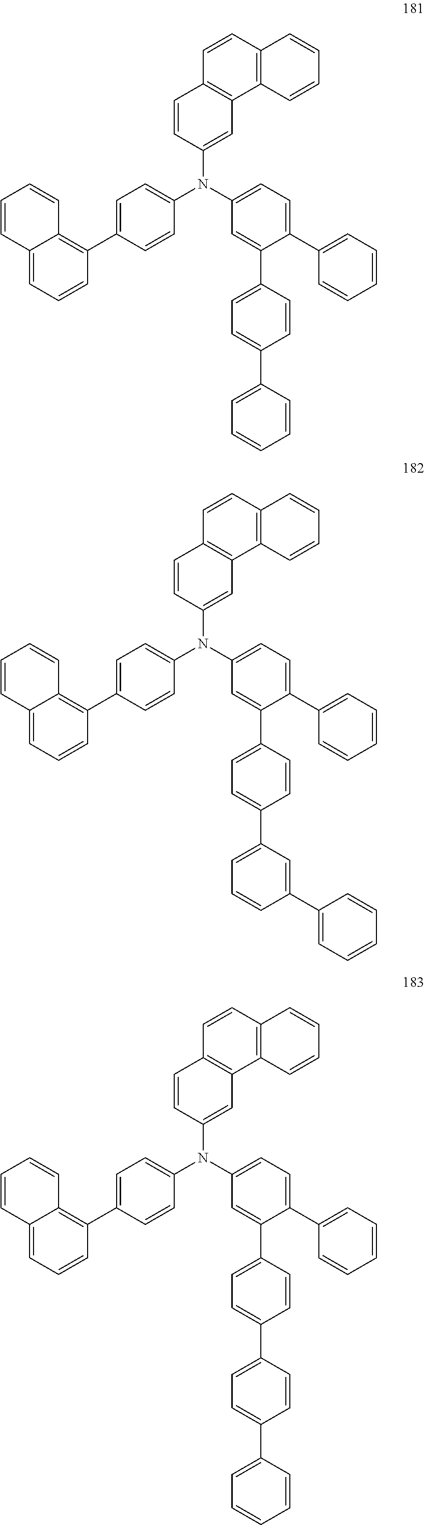 [SMILES: c1ccc(-c2ccc(-c3cc(N(c4ccc(-c5cccc6ccccc56)cc4)c4ccc5ccc6ccccc6c5c4)ccc3-c3ccccc3)cc2)cc1 ; c1ccc(-c2cccc(-c3ccc(-c4cc(N(c5ccc(-c6cccc7ccccc67)cc5)c5ccc6ccc7ccccc7c6c5)ccc4-c4ccccc4)cc3)c2)cc1 ; c1ccc(-c2ccc(-c3ccc(-c4cc(N(c5ccc(-c6cccc7ccccc67)cc5)c5ccc6ccc7ccccc7c6c5)ccc4-c4ccccc4)cc3)cc2)cc1] 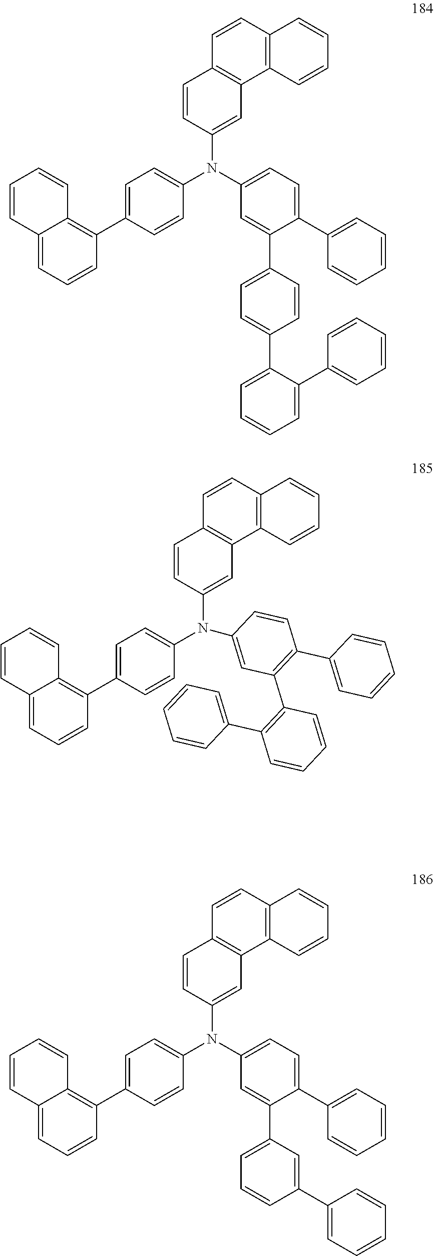 [SMILES: c1ccc(-c2ccccc2-c2ccc(-c3cc(N(c4ccc(-c5cccc6ccccc56)cc4)c4ccc5ccc6ccccc6c5c4)ccc3-c3ccccc3)cc2)cc1 ; c1ccc(-c2ccccc2-c2cc(N(c3ccc(-c4cccc5ccccc45)cc3)c3ccc4ccc5ccccc5c4c3)ccc2-c2ccccc2)cc1 ; c1ccc(-c2cccc(-c3cc(N(c4ccc(-c5cccc6ccccc56)cc4)c4ccc5ccc6ccccc6c5c4)ccc3-c3ccccc3)c2)cc1] 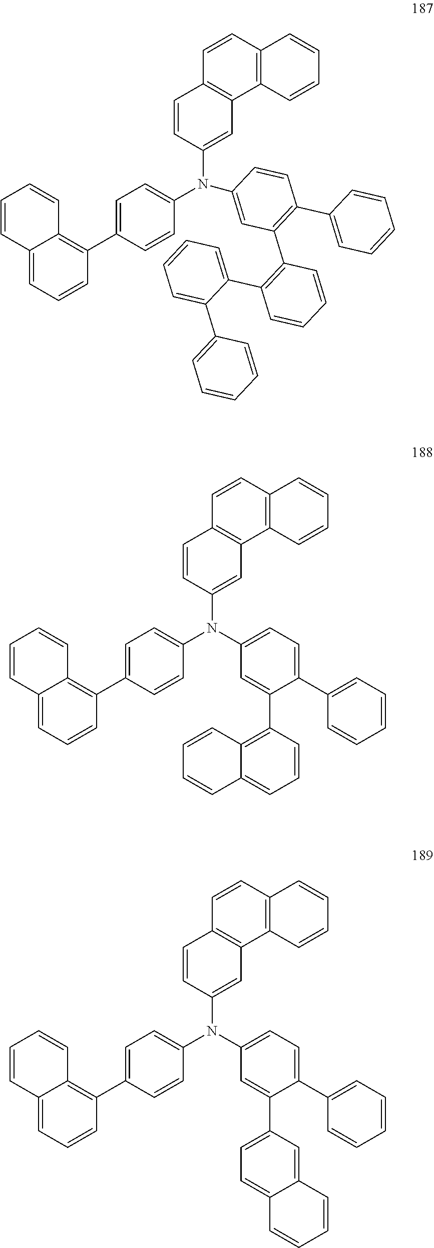 [SMILES: c1ccc(-c2ccccc2-c2ccccc2-c2cc(N(c3ccc(-c4cccc5ccccc45)cc3)c3ccc4ccc5ccccc5c4c3)ccc2-c2ccccc2)cc1 ; c1ccc(-c2ccc(N(c3ccc(-c4cccc5ccccc45)cc3)c3ccc4ccc5ccccc5c4c3)cc2-c2cccc3ccccc23)cc1 ; c1ccc(-c2ccc(N(c3ccc(-c4cccc5ccccc45)cc3)c3ccc4ccc5ccccc5c4c3)cc2-c2ccc3ccccc3c2)cc1] 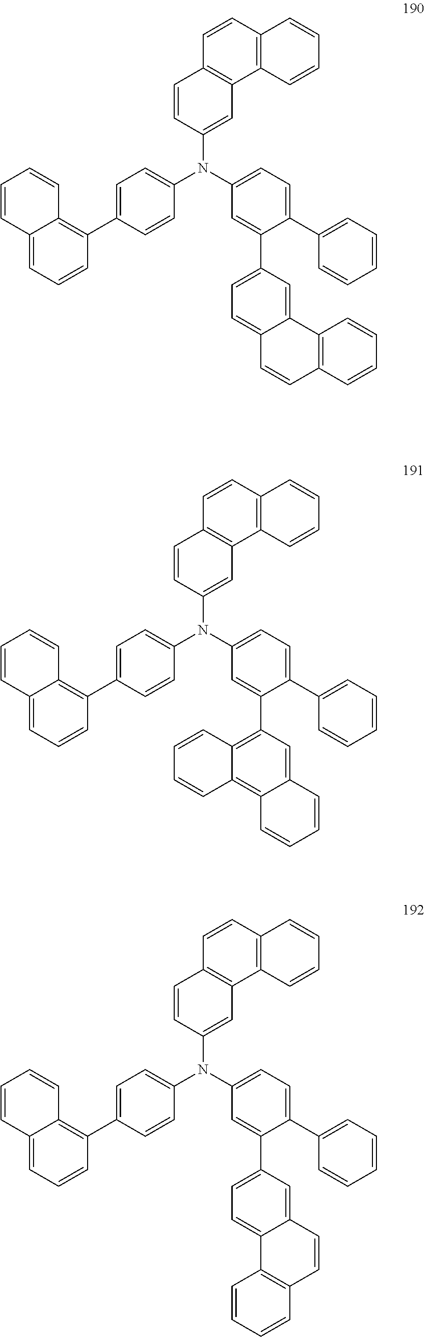 [SMILES: c1ccc(-c2ccc(N(c3ccc(-c4cccc5ccccc45)cc3)c3ccc4ccc5ccccc5c4c3)cc2-c2ccc3ccc4ccccc4c3c2)cc1 ; c1ccc(-c2ccc(N(c3ccc(-c4cccc5ccccc45)cc3)c3ccc4ccc5ccccc5c4c3)cc2-c2cc3ccccc3c3ccccc23)cc1 ; c1ccc(-c2ccc(N(c3ccc(-c4cccc5ccccc45)cc3)c3ccc4ccc5ccccc5c4c3)cc2-c2ccc3c(ccc4ccccc43)c2)cc1] 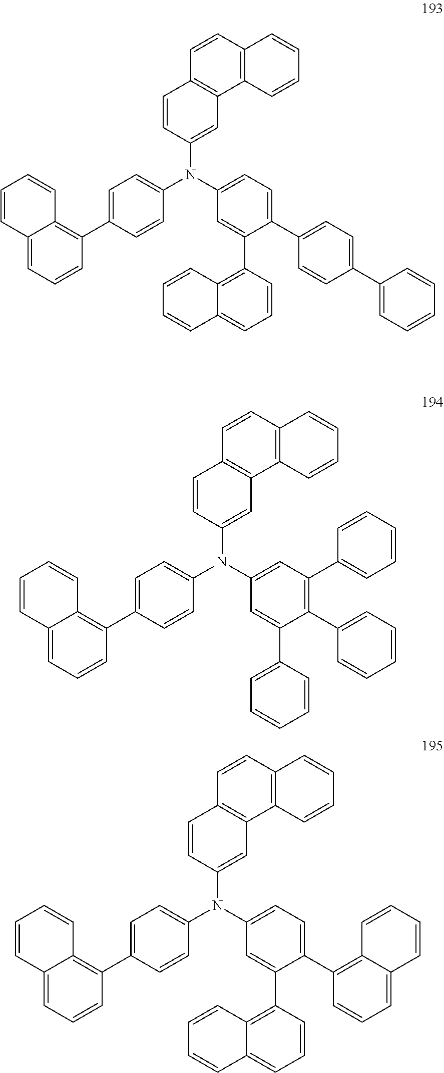 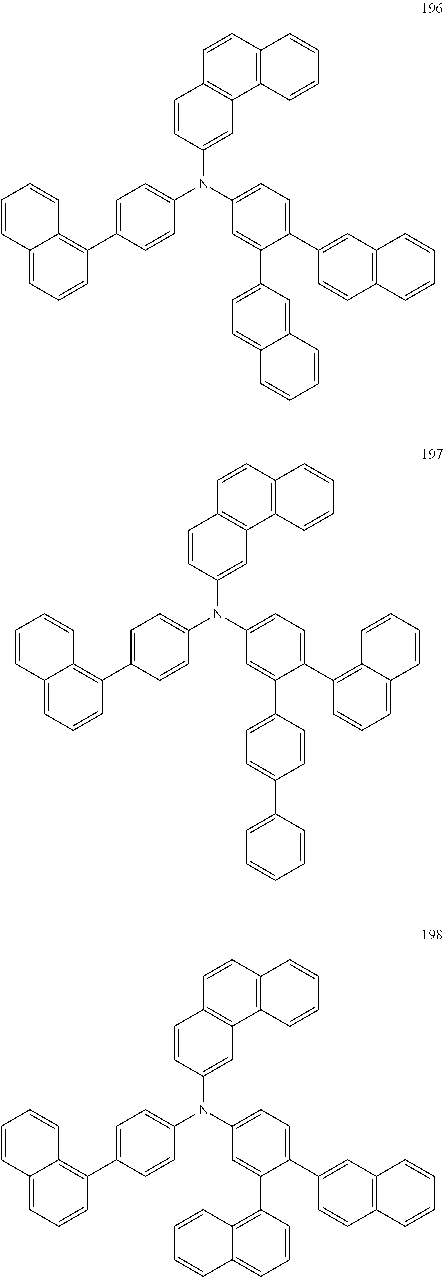 [SMILES: c1ccc2cc(-c3ccc(N(c4ccc(-c5cccc6ccccc56)cc4)c4ccc5ccc6ccccc6c5c4)cc3-c3ccc4ccccc4c3)ccc2c1 ; c1ccc(-c2ccc(-c3cc(N(c4ccc(-c5cccc6ccccc56)cc4)c4ccc5ccc6ccccc6c5c4)ccc3-c3cccc4ccccc34)cc2)cc1 ; c1ccc2cc(-c3ccc(N(c4ccc(-c5cccc6ccccc56)cc4)c4ccc5ccc6ccccc6c5c4)cc3-c3cccc4ccccc34)ccc2c1] 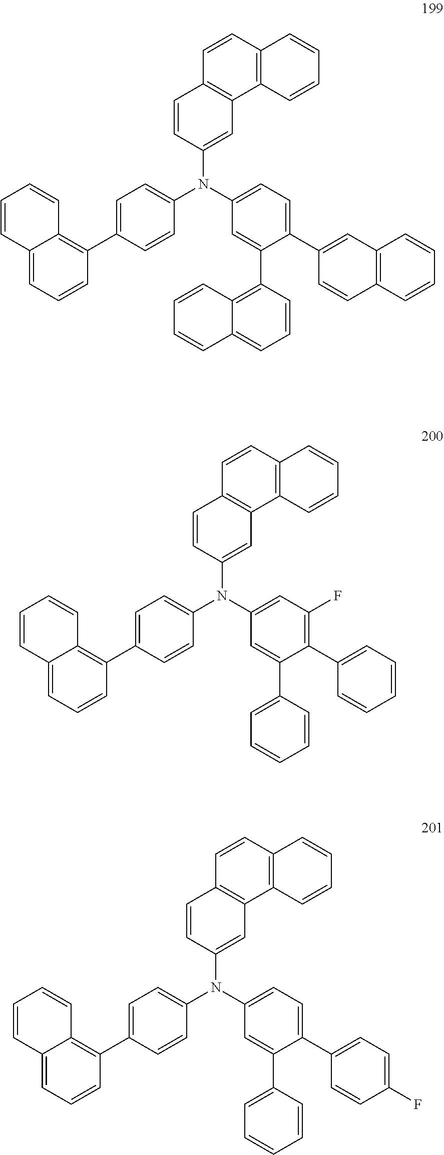 [SMILES: c1ccc2cc(-c3ccc(N(c4ccc(-c5cccc6ccccc56)cc4)c4ccc5ccc6ccccc6c5c4)cc3-c3cccc4ccccc34)ccc2c1 ; Fc1cc(N(c2ccc(-c3cccc4ccccc34)cc2)c2ccc3ccc4ccccc4c3c2)cc(-c2ccccc2)c1-c1ccccc1 ; Fc1ccc(-c2ccc(N(c3ccc(-c4cccc5ccccc45)cc3)c3ccc4ccc5ccccc5c4c3)cc2-c2ccccc2)cc1] 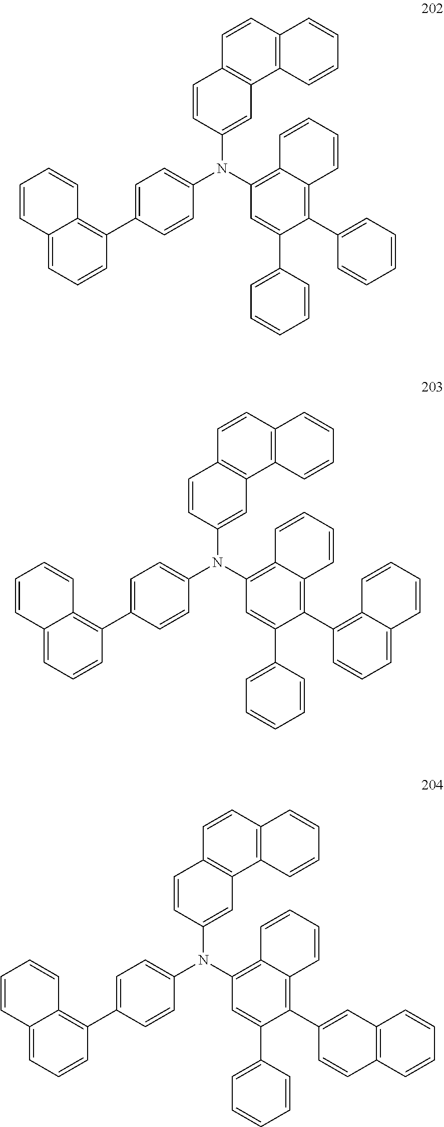 [SMILES: c1ccc(-c2cc(N(c3ccc(-c4cccc5ccccc45)cc3)c3ccc4ccc5ccccc5c4c3)c3ccccc3c2-c2ccccc2)cc1 ; c1ccc(-c2cc(N(c3ccc(-c4cccc5ccccc45)cc3)c3ccc4ccc5ccccc5c4c3)c3ccccc3c2-c2cccc3ccccc23)cc1 ; c1ccc(-c2cc(N(c3ccc(-c4cccc5ccccc45)cc3)c3ccc4ccc5ccccc5c4c3)c3ccccc3c2-c2ccc3ccccc3c2)cc1] 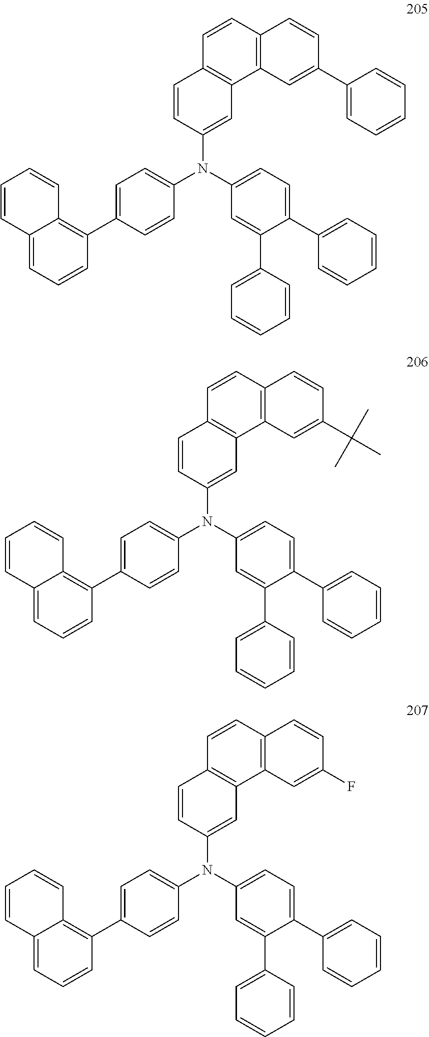 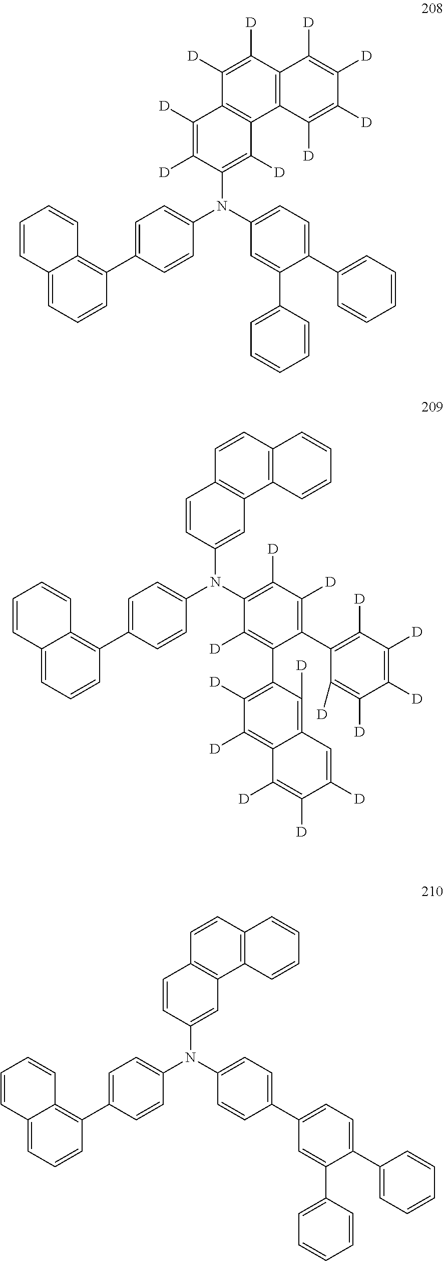 [SMILES: [2H]c1c([2H])c([2H])c2c(c1[2H])c([2H])c([2H])c1c([2H])c([2H])c(N(c3ccc(-c4cccc5ccccc45)cc3)c3ccc(-c4ccccc4)c(-c4ccccc4)c3)c([2H])c12 ; [2H]c1cc2c([2H])c(-c3c([2H])c(N(c4ccc(-c5cccc6ccccc56)cc4)c4ccc5ccc6ccccc6c5c4)c([2H])c([2H])c3-c3c([2H])c([2H])c([2H])c([2H])c3[2H])c([2H])c([2H])c2c([2H])c1[2H] ; c1ccc(-c2ccc(-c3ccc(N(c4ccc(-c5cccc6ccccc56)cc4)c4ccc5ccc6ccccc6c5c4)cc3)cc2-c2ccccc2)cc1] 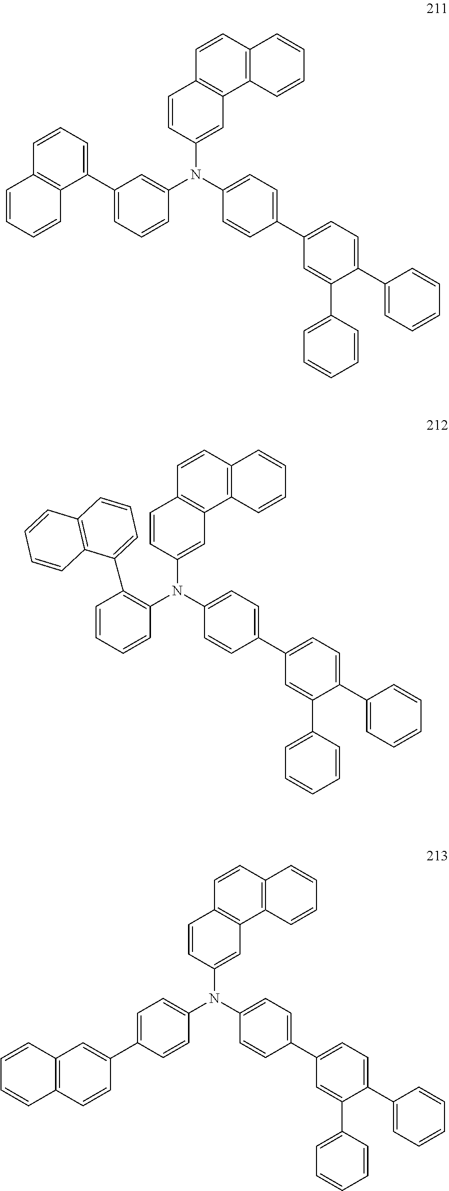 [SMILES: c1ccc(-c2ccc(-c3ccc(N(c4cccc(-c5cccc6ccccc56)c4)c4ccc5ccc6ccccc6c5c4)cc3)cc2-c2ccccc2)cc1 ; c1ccc(-c2ccc(-c3ccc(N(c4ccc5ccc6ccccc6c5c4)c4ccccc4-c4cccc5ccccc45)cc3)cc2-c2ccccc2)cc1 ; c1ccc(-c2ccc(-c3ccc(N(c4ccc(-c5ccc6ccccc6c5)cc4)c4ccc5ccc6ccccc6c5c4)cc3)cc2-c2ccccc2)cc1] 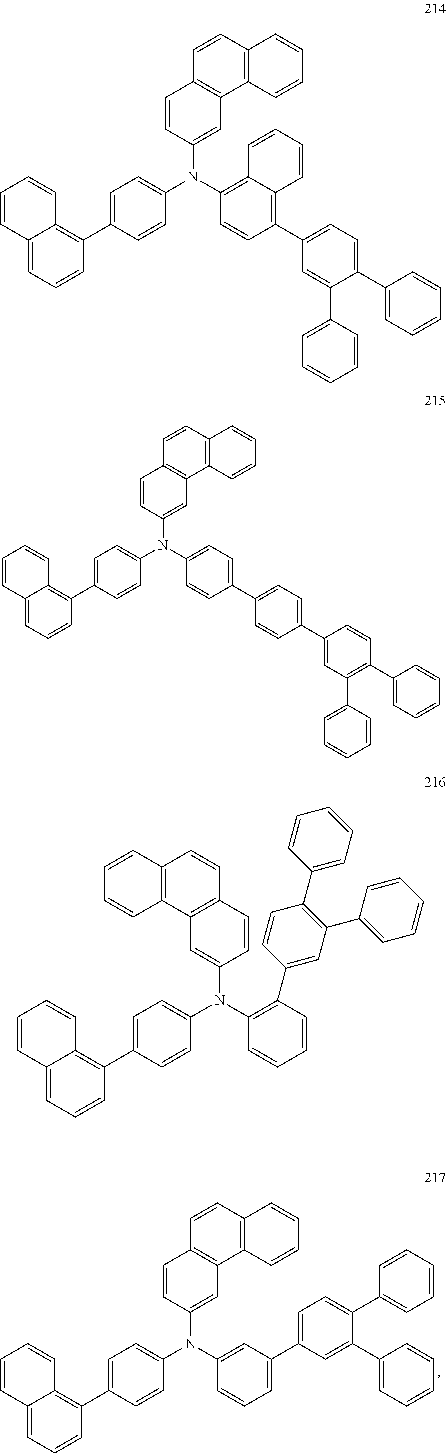 [SMILES: c1ccc(-c2ccc(-c3ccc(N(c4ccc(-c5cccc6ccccc56)cc4)c4ccc5ccc6ccccc6c5c4)c4ccccc34)cc2-c2ccccc2)cc1 ; c1ccc(-c2ccc(-c3ccc(-c4ccc(N(c5ccc(-c6cccc7ccccc67)cc5)c5ccc6ccc7ccccc7c6c5)cc4)cc3)cc2-c2ccccc2)cc1 ; c1ccc(-c2ccc(-c3ccccc3N(c3ccc(-c4cccc5ccccc45)cc3)c3ccc4ccc5ccccc5c4c3)cc2-c2ccccc2)cc1 ; c1ccc(-c2ccc(-c3cccc(N(c4ccc(-c5cccc6ccccc56)cc4)c4ccc5ccc6ccccc6c5c4)c3)cc2-c2ccccc2)cc1]
in Compound Group 1, D is a deuterium atom.

11. An amine compound represented by Formula 1: 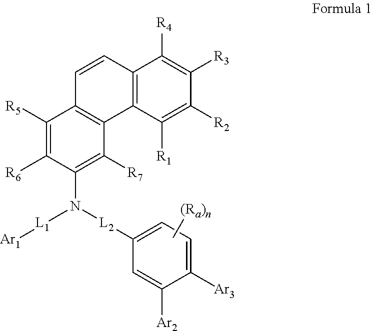
in Formula 1,
R₁ to R₇ are each independently a hydrogen atom, a deuterium atom, a halogen atom, a substituted or unsubstituted alkyl group having 1 to 15 carbons, a substituted or unsubstituted aryl group having 6 to 30 ring-forming carbon atoms, or a substituted or unsubstituted heteroaryl group having 2 to 30 ring-forming carbon atoms,
n is an integer of 1 to 3,
Rₐ is a hydrogen atom, a deuterium atom, a halogen atom, a substituted or unsubstituted alkyl group having 1 to 15 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or forms a ring by being bonded to an adjacent group,
L₁ and L₂ are each independently a direct linkage, a substituted or unsubstituted arylene group having 6 to 30 ring-forming carbon atoms, or a substituted or unsubstituted heteroarylene group having 2 to 30 ring-forming carbon atoms,
Ar₁ is a substituted or unsubstituted aryl group having 6 to 30 ring-forming carbon atoms, or a substituted or unsubstituted heteroaryl group having 2 to 30 ring-forming carbon atoms, and Ar₁ is not a fluorenyl group when L₁ is a direct linkage,
Ar₂ and Ar₃ are each independently a substituted or unsubstituted phenyl group, a substituted or unsubstituted biphenyl group, a substituted or unsubstituted terphenyl group, a substituted or unsubstituted naphthyl group, or a substituted or unsubstituted phenanthrenyl group, except when each of Ar₂ and Ar₃ includes a carbazole group as a substituent, and
each of Ar₁, Ar₂, and Ar₃ do not include benzophenanthrene.

12. The amine compound of claim 11, wherein the amine compound is represented by Formula 1A: 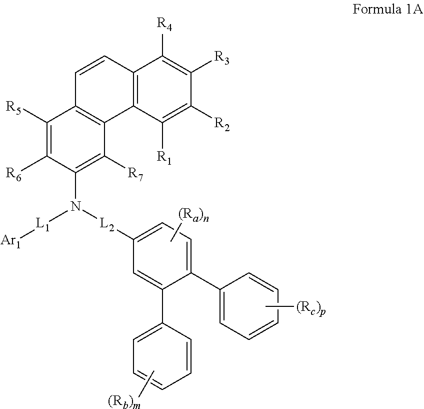
in Formula 1 A,
m and p are each independently an integer of 1 to 5,
R_{b} and R_{c} are each independently a hydrogen atom, a deuterium atom, a halogen atom, a substituted or unsubstituted alkyl group having 1 to 15 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or form a ring by being bonded to an adjacent group, and
Ar₁, L₁, L₂, R₁ to R₇, Rₐ, and n are as defined in Formula 1.

13. The amine compound of claim 11, wherein the amine compound is represented by Formula 1-1 or Formula 1-2: 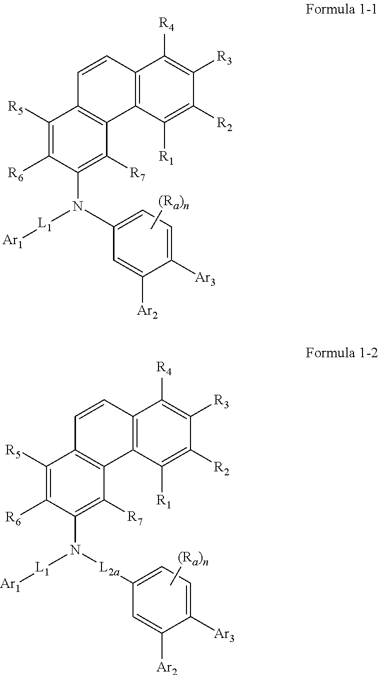
in Formula 1-2, L₂a is a substituted or unsubstituted phenylene group, or a substituted or unsubstituted divalent biphenyl group, and
in Formula 1-1 and Formula 1-2, Ar₁, Ar₂, Ar₃, L₁, R₁ to R₇, Rₐ, and n are as defined in Formula 1.

14. The amine compound of claim 11, wherein the Ani is a substituted or unsubstituted phenyl group, a substituted or unsubstituted naphthyl group, a substituted or unsubstituted biphenyl group, a substituted or unsubstituted terphenyl group, a substituted or unsubstituted fluorenyl group, a substituted or unsubstituted dibenzoheterole group comprising O, S, N, or Si as a ring-forming hetero atom, or a substituted or unsubstituted benzonaphthoheterol group comprising O or S as a ring-forming hetero atom.

15. The amine compound of claim 11, wherein in Formula 1, at least one hydrogen atom is substituted with a deuterium atom.

16. The amine compound of claim 11, wherein in Formula 1, each of R₁ to R₇, Rₐ, Ar₁, Ar₂, Ar₃, L₁, and L₂ do not include a substituted or unsubstituted amine group.

17. The amine compound of claim 11, wherein the amine compound is represented by any one selected from among compounds of Compound Group 1: 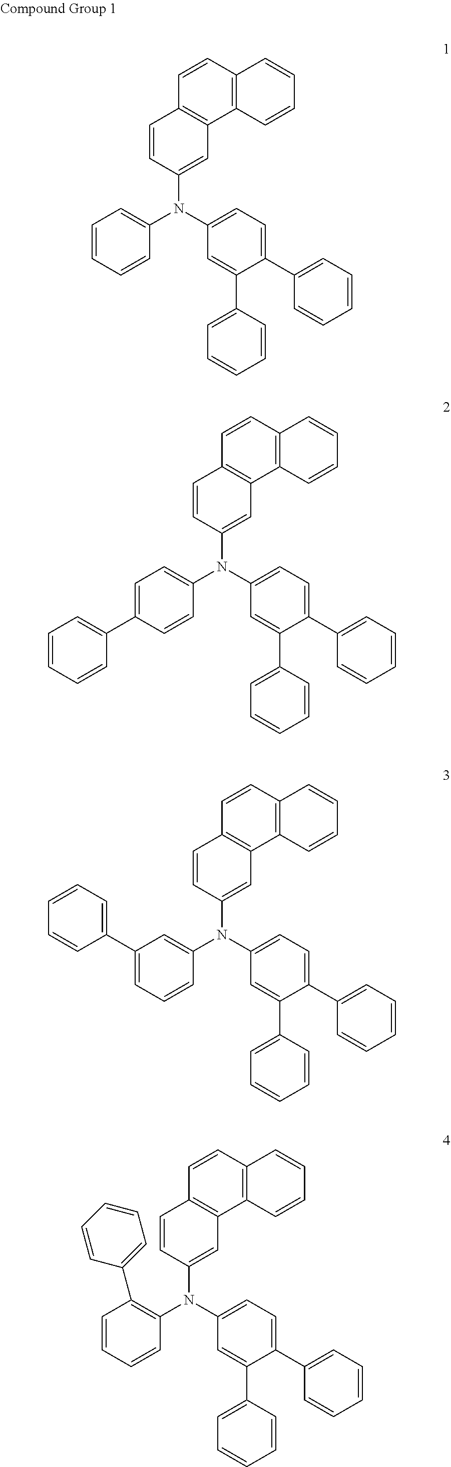 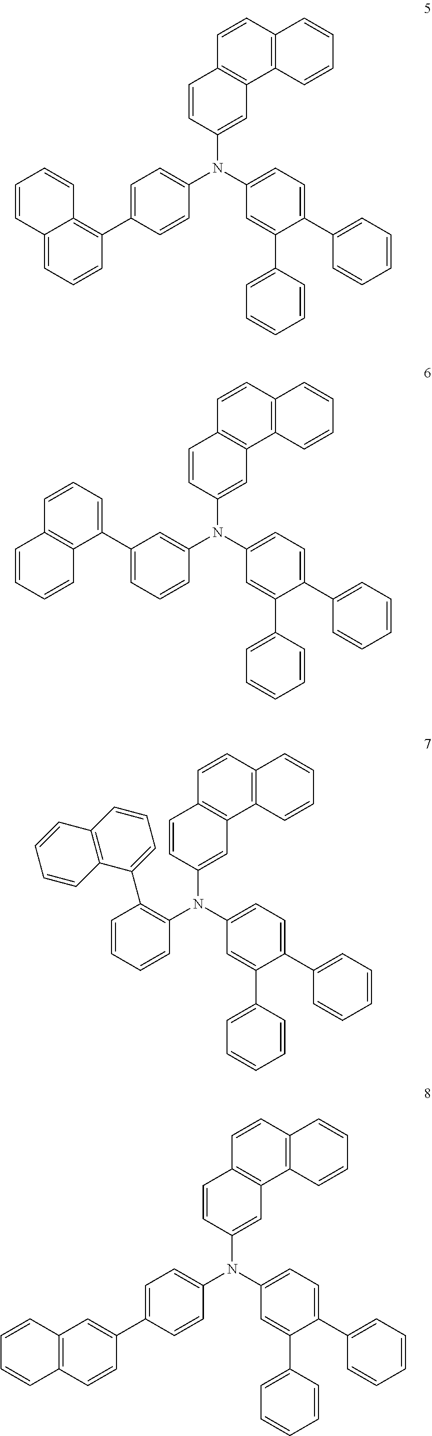 [SMILES: c1ccc(-c2ccc(N(c3ccc(-c4cccc5ccccc45)cc3)c3ccc4ccc5ccccc5c4c3)cc2-c2ccccc2)cc1 ; c1ccc(-c2ccc(N(c3cccc(-c4cccc5ccccc45)c3)c3ccc4ccc5ccccc5c4c3)cc2-c2ccccc2)cc1 ; c1ccc(-c2ccc(N(c3ccc4ccc5ccccc5c4c3)c3ccccc3-c3cccc4ccccc34)cc2-c2ccccc2)cc1 ; c1ccc(-c2ccc(N(c3ccc(-c4ccc5ccccc5c4)cc3)c3ccc4ccc5ccccc5c4c3)cc2-c2ccccc2)cc1] 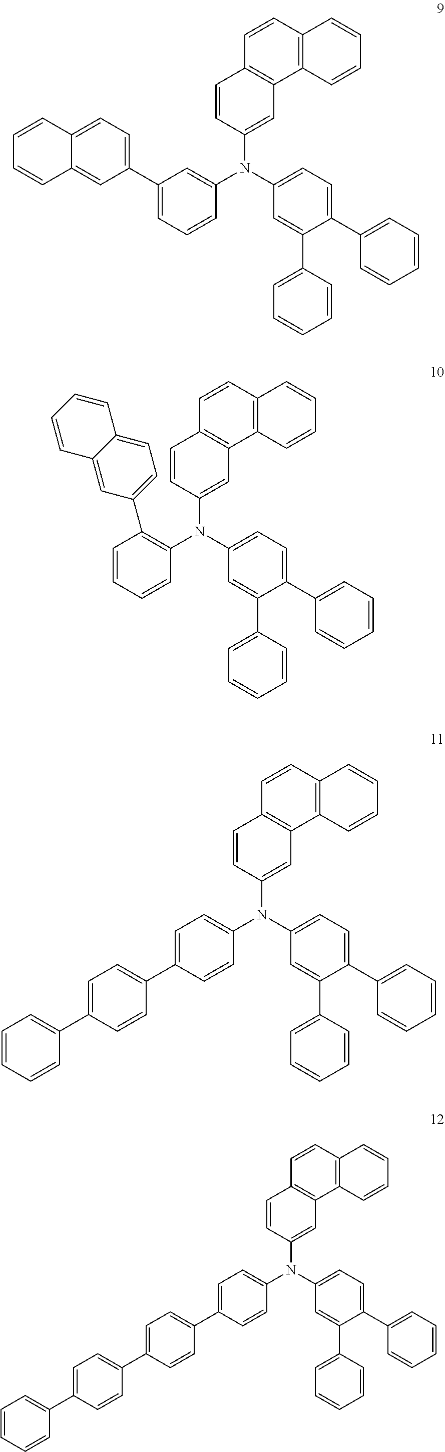 [SMILES: c1ccc(-c2ccc(N(c3cccc(-c4ccc5ccccc5c4)c3)c3ccc4ccc5ccccc5c4c3)cc2-c2ccccc2)cc1 ; c1ccc(-c2ccc(N(c3ccc4ccc5ccccc5c4c3)c3ccccc3-c3ccc4ccccc4c3)cc2-c2ccccc2)cc1 ; c1ccc(-c2ccc(-c3ccc(N(c4ccc(-c5ccccc5)c(-c5ccccc5)c4)c4ccc5ccc6ccccc6c5c4)cc3)cc2)cc1 ; c1ccc(-c2ccc(-c3ccc(-c4ccc(N(c5ccc(-c6ccccc6)c(-c6ccccc6)c5)c5ccc6ccc7ccccc7c6c5)cc4)cc3)cc2)cc1] 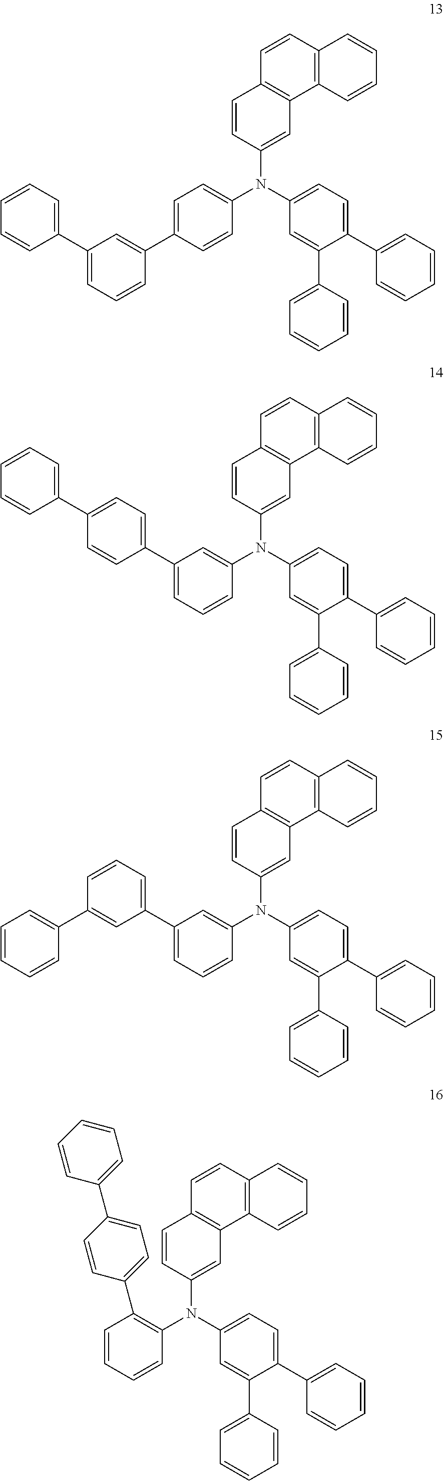 [SMILES: c1ccc(-c2cccc(-c3ccc(N(c4ccc(-c5ccccc5)c(-c5ccccc5)c4)c4ccc5ccc6ccccc6c5c4)cc3)c2)cc1 ; c1ccc(-c2ccc(-c3cccc(N(c4ccc(-c5ccccc5)c(-c5ccccc5)c4)c4ccc5ccc6ccccc6c5c4)c3)cc2)cc1 ; c1ccc(-c2cccc(-c3cccc(N(c4ccc(-c5ccccc5)c(-c5ccccc5)c4)c4ccc5ccc6ccccc6c5c4)c3)c2)cc1 ; c1ccc(-c2ccc(-c3ccccc3N(c3ccc(-c4ccccc4)c(-c4ccccc4)c3)c3ccc4ccc5ccccc5c4c3)cc2)cc1] 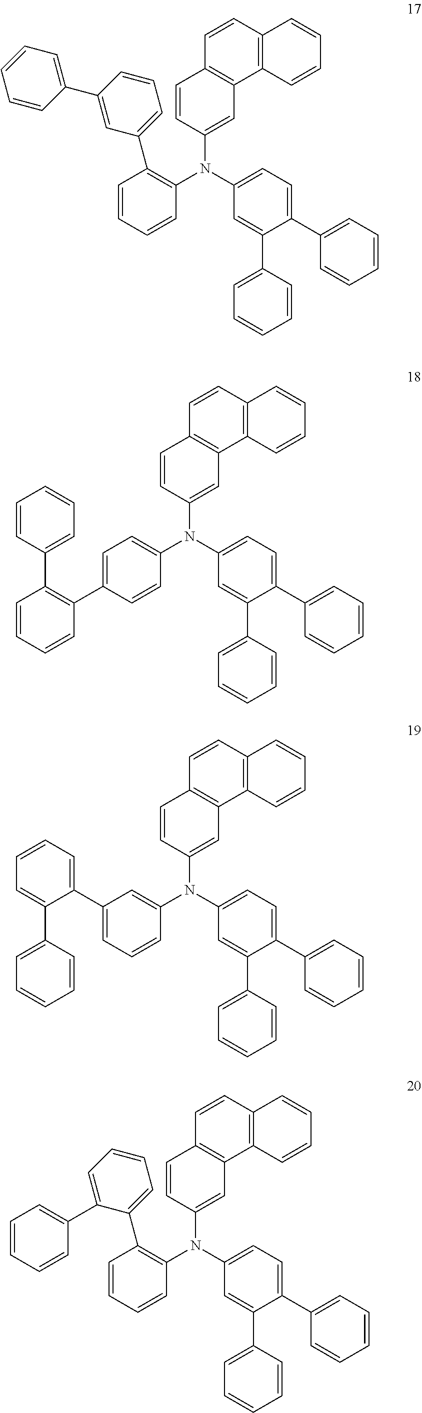 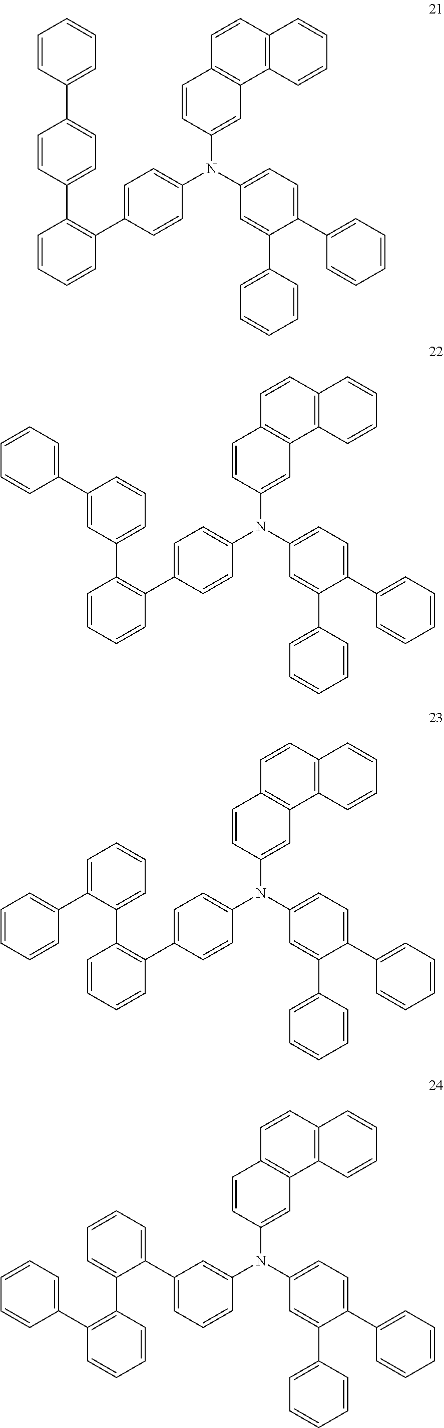 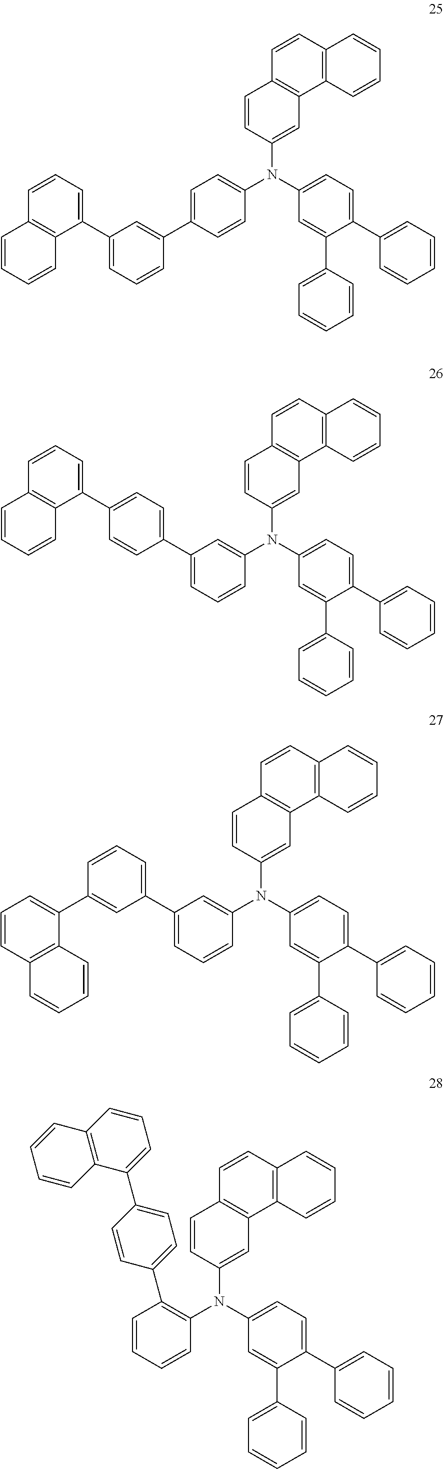 [SMILES: c1ccc(-c2ccc(N(c3ccc(-c4cccc(-c5cccc6ccccc56)c4)cc3)c3ccc4ccc5ccccc5c4c3)cc2-c2ccccc2)cc1 ; c1ccc(-c2ccc(N(c3cccc(-c4ccc(-c5cccc6ccccc56)cc4)c3)c3ccc4ccc5ccccc5c4c3)cc2-c2ccccc2)cc1 ; c1ccc(-c2ccc(N(c3cccc(-c4cccc(-c5cccc6ccccc56)c4)c3)c3ccc4ccc5ccccc5c4c3)cc2-c2ccccc2)cc1 ; c1ccc(-c2ccc(N(c3ccc4ccc5ccccc5c4c3)c3ccccc3-c3ccc(-c4cccc5ccccc45)cc3)cc2-c2ccccc2)cc1] 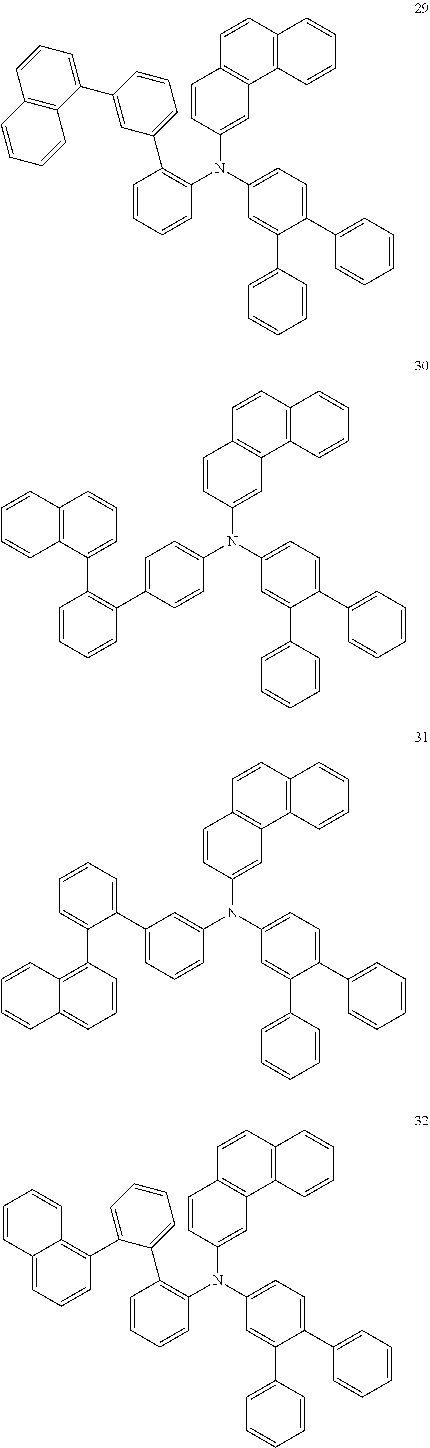 [SMILES: c1ccc(-c2ccc(N(c3ccc4ccc5ccccc5c4c3)c3ccccc3-c3cccc(-c4cccc5ccccc45)c3)cc2-c2ccccc2)cc1 ; c1ccc(-c2ccc(N(c3ccc(-c4ccccc4-c4cccc5ccccc45)cc3)c3ccc4ccc5ccccc5c4c3)cc2-c2ccccc2)cc1 ; c1ccc(-c2ccc(N(c3cccc(-c4ccccc4-c4cccc5ccccc45)c3)c3ccc4ccc5ccccc5c4c3)cc2-c2ccccc2)cc1 ; c1ccc(-c2ccc(N(c3ccc4ccc5ccccc5c4c3)c3ccccc3-c3ccccc3-c3cccc4ccccc34)cc2-c2ccccc2)cc1] 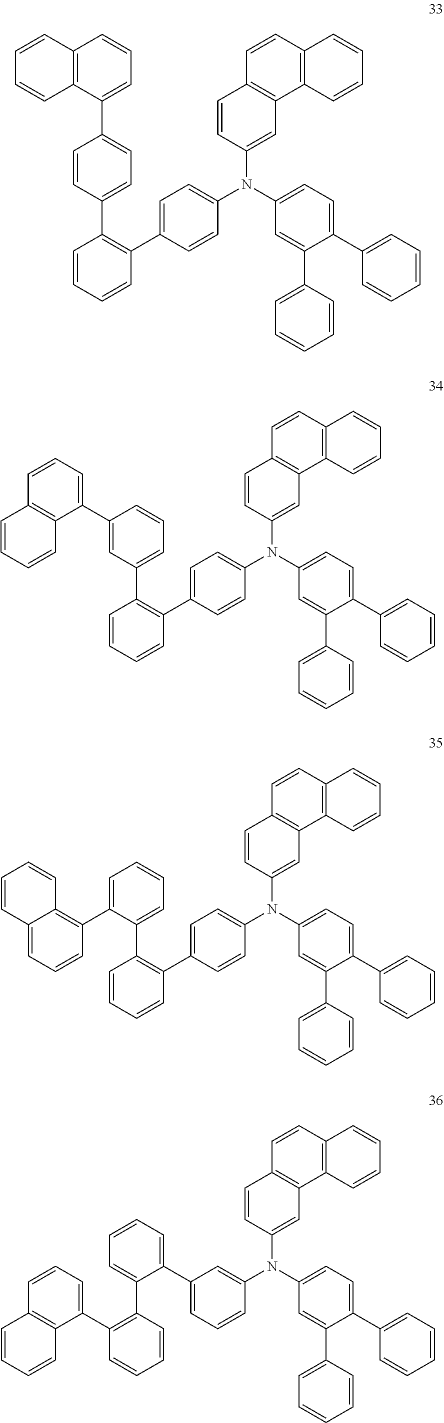 [SMILES: c1ccc(-c2ccc(N(c3ccc(-c4ccccc4-c4ccc(-c5cccc6ccccc56)cc4)cc3)c3ccc4ccc5ccccc5c4c3)cc2-c2ccccc2)cc1 ; c1ccc(-c2ccc(N(c3ccc(-c4ccccc4-c4cccc(-c5cccc6ccccc56)c4)cc3)c3ccc4ccc5ccccc5c4c3)cc2-c2ccccc2)cc1 ; c1ccc(-c2ccc(N(c3ccc(-c4ccccc4-c4ccccc4-c4cccc5ccccc45)cc3)c3ccc4ccc5ccccc5c4c3)cc2-c2ccccc2)cc1 ; c1ccc(-c2ccc(N(c3cccc(-c4ccccc4-c4ccccc4-c4cccc5ccccc45)c3)c3ccc4ccc5ccccc5c4c3)cc2-c2ccccc2)cc1] 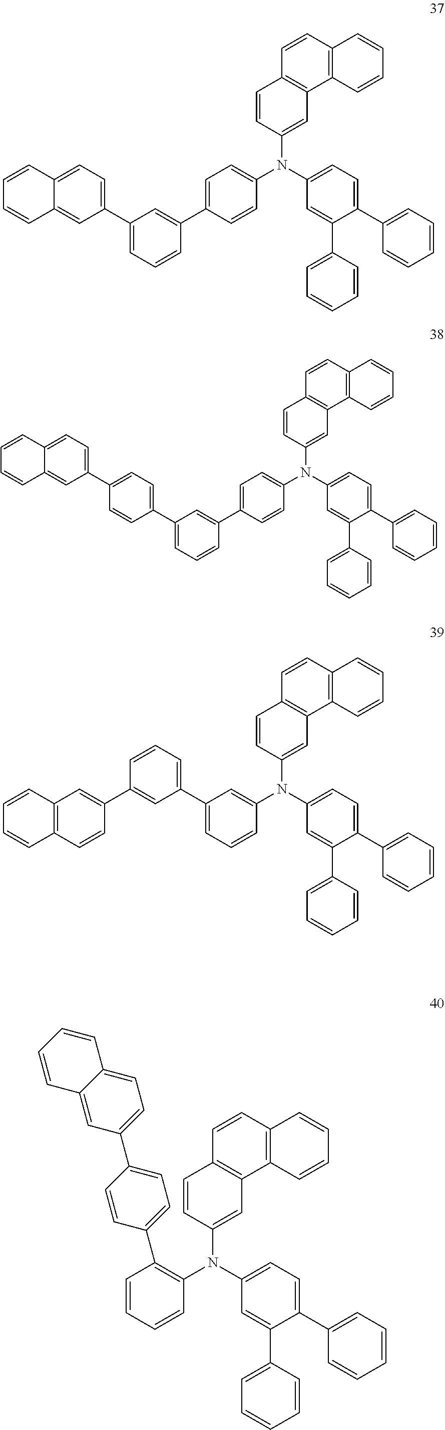 [SMILES: c1ccc(-c2ccc(N(c3ccc(-c4cccc(-c5ccc6ccccc6c5)c4)cc3)c3ccc4ccc5ccccc5c4c3)cc2-c2ccccc2)cc1 ; c1ccc(-c2ccc(N(c3ccc(-c4cccc(-c5ccc(-c6ccc7ccccc7c6)cc5)c4)cc3)c3ccc4ccc5ccccc5c4c3)cc2-c2ccccc2)cc1 ; c1ccc(-c2ccc(N(c3cccc(-c4cccc(-c5ccc6ccccc6c5)c4)c3)c3ccc4ccc5ccccc5c4c3)cc2-c2ccccc2)cc1 ; c1ccc(-c2ccc(N(c3ccc4ccc5ccccc5c4c3)c3ccccc3-c3ccc(-c4ccc5ccccc5c4)cc3)cc2-c2ccccc2)cc1] 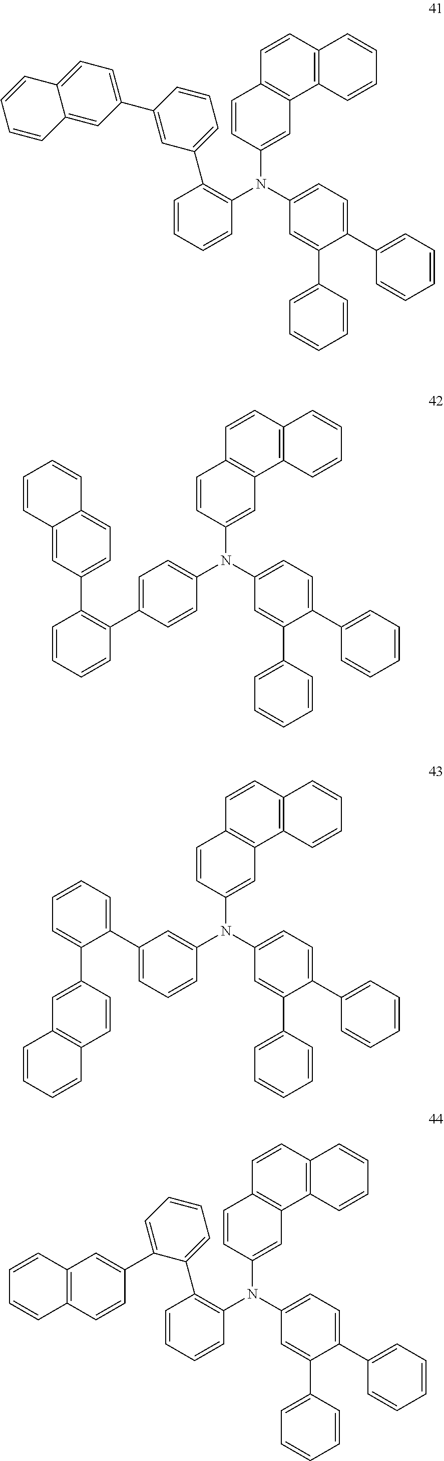 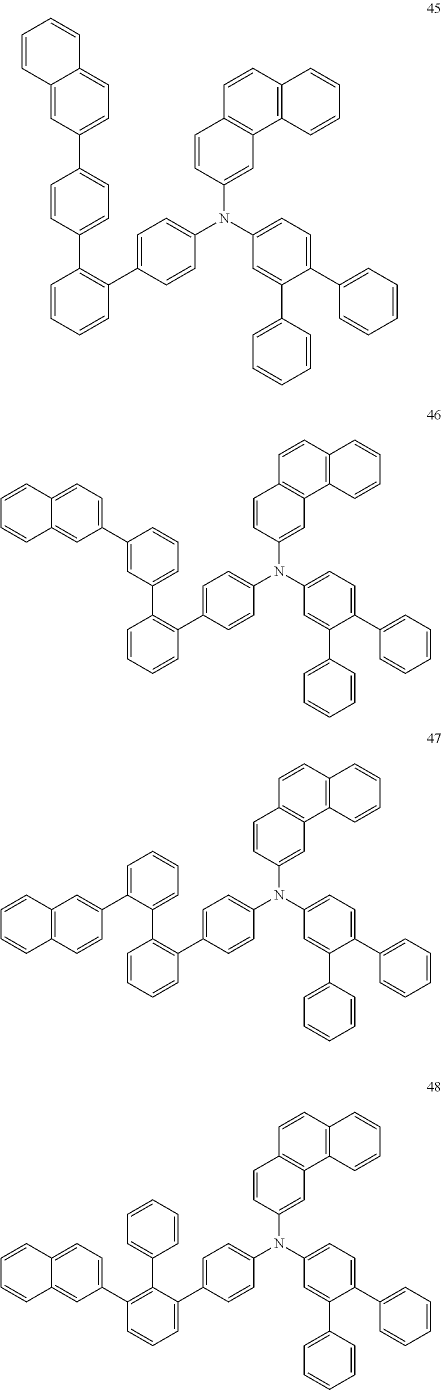 [SMILES: c1ccc(-c2ccc(N(c3ccc(-c4ccccc4-c4ccc(-c5ccc6ccccc6c5)cc4)cc3)c3ccc4ccc5ccccc5c4c3)cc2-c2ccccc2)cc1 ; c1ccc(-c2ccc(N(c3ccc(-c4ccccc4-c4cccc(-c5ccc6ccccc6c5)c4)cc3)c3ccc4ccc5ccccc5c4c3)cc2-c2ccccc2)cc1 ; c1ccc(-c2ccc(N(c3ccc(-c4ccccc4-c4ccccc4-c4ccc5ccccc5c4)cc3)c3ccc4ccc5ccccc5c4c3)cc2-c2ccccc2)cc1 ; c1ccc(-c2ccc(N(c3ccc(-c4cccc(-c5ccc6ccccc6c5)c4-c4ccccc4)cc3)c3ccc4ccc5ccccc5c4c3)cc2-c2ccccc2)cc1] 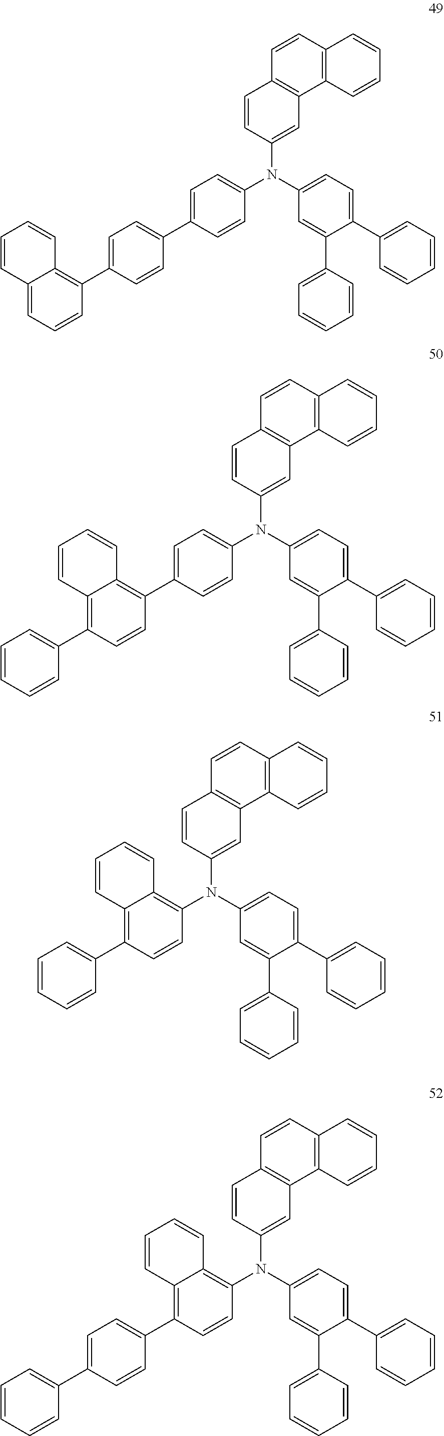 [SMILES: c1ccc(-c2ccc(N(c3ccc(-c4ccc(-c5cccc6ccccc56)cc4)cc3)c3ccc4ccc5ccccc5c4c3)cc2-c2ccccc2)cc1 ; c1ccc(-c2ccc(N(c3ccc(-c4ccc(-c5ccccc5)c5ccccc45)cc3)c3ccc4ccc5ccccc5c4c3)cc2-c2ccccc2)cc1 ; c1ccc(-c2ccc(N(c3ccc4ccc5ccccc5c4c3)c3ccc(-c4ccccc4)c4ccccc34)cc2-c2ccccc2)cc1 ; c1ccc(-c2ccc(-c3ccc(N(c4ccc(-c5ccccc5)c(-c5ccccc5)c4)c4ccc5ccc6ccccc6c5c4)c4ccccc34)cc2)cc1] 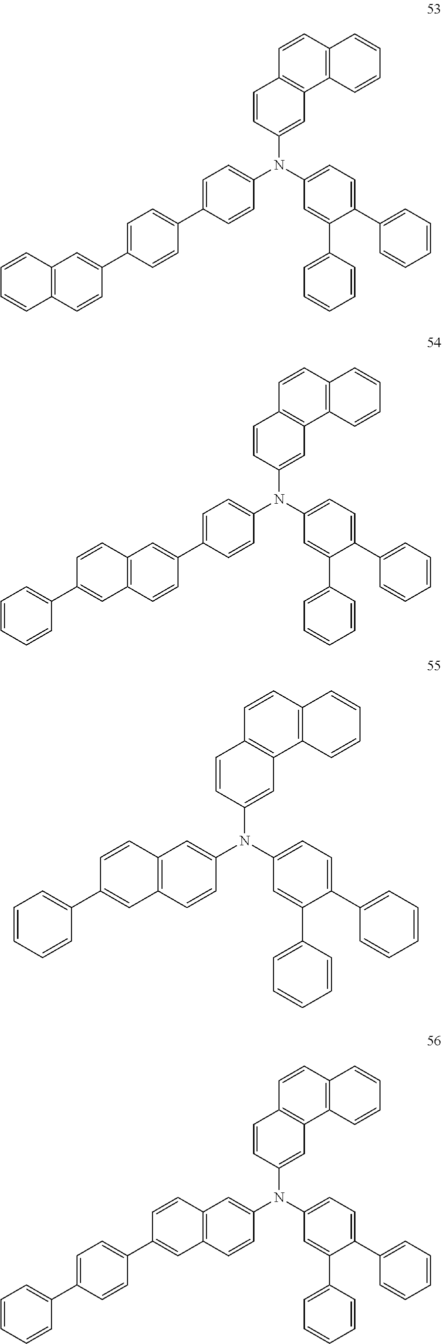 [SMILES: c1ccc(-c2ccc(N(c3ccc(-c4ccc(-c5ccc6ccccc6c5)cc4)cc3)c3ccc4ccc5ccccc5c4c3)cc2-c2ccccc2)cc1 ; c1ccc(-c2ccc3cc(-c4ccc(N(c5ccc(-c6ccccc6)c(-c6ccccc6)c5)c5ccc6ccc7ccccc7c6c5)cc4)ccc3c2)cc1 ; c1ccc(-c2ccc3cc(N(c4ccc(-c5ccccc5)c(-c5ccccc5)c4)c4ccc5ccc6ccccc6c5c4)ccc3c2)cc1 ; c1ccc(-c2ccc(-c3ccc4cc(N(c5ccc(-c6ccccc6)c(-c6ccccc6)c5)c5ccc6ccc7ccccc7c6c5)ccc4c3)cc2)cc1] 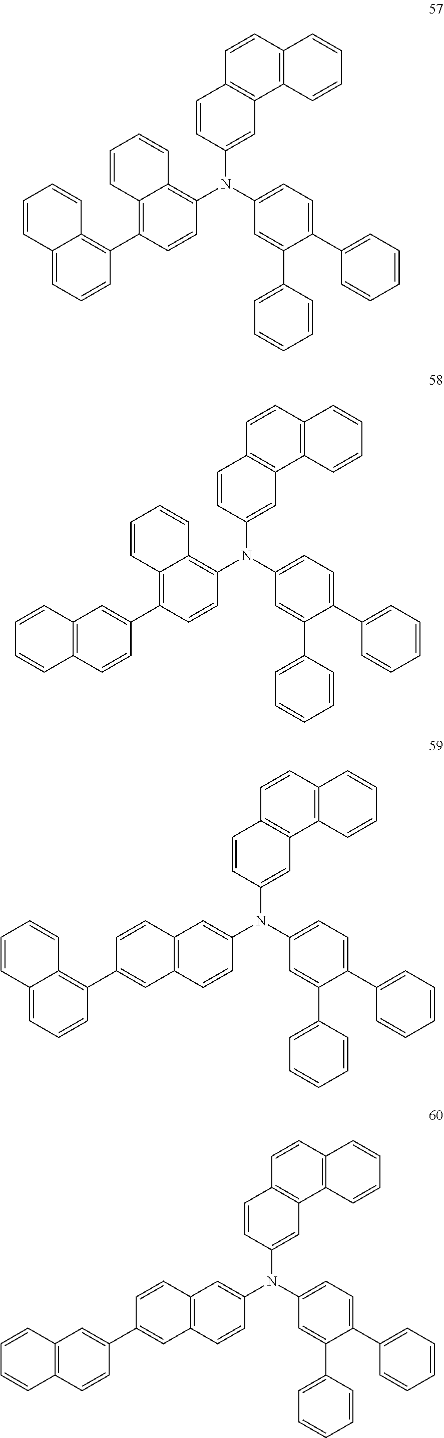 [SMILES: c1ccc(-c2ccc(N(c3ccc4ccc5ccccc5c4c3)c3ccc(-c4cccc5ccccc45)c4ccccc34)cc2-c2ccccc2)cc1 ; c1ccc(-c2ccc(N(c3ccc4ccc5ccccc5c4c3)c3ccc(-c4ccc5ccccc5c4)c4ccccc34)cc2-c2ccccc2)cc1 ; c1ccc(-c2ccc(N(c3ccc4cc(-c5cccc6ccccc56)ccc4c3)c3ccc4ccc5ccccc5c4c3)cc2-c2ccccc2)cc1 ; c1ccc(-c2ccc(N(c3ccc4cc(-c5ccc6ccccc6c5)ccc4c3)c3ccc4ccc5ccccc5c4c3)cc2-c2ccccc2)cc1] 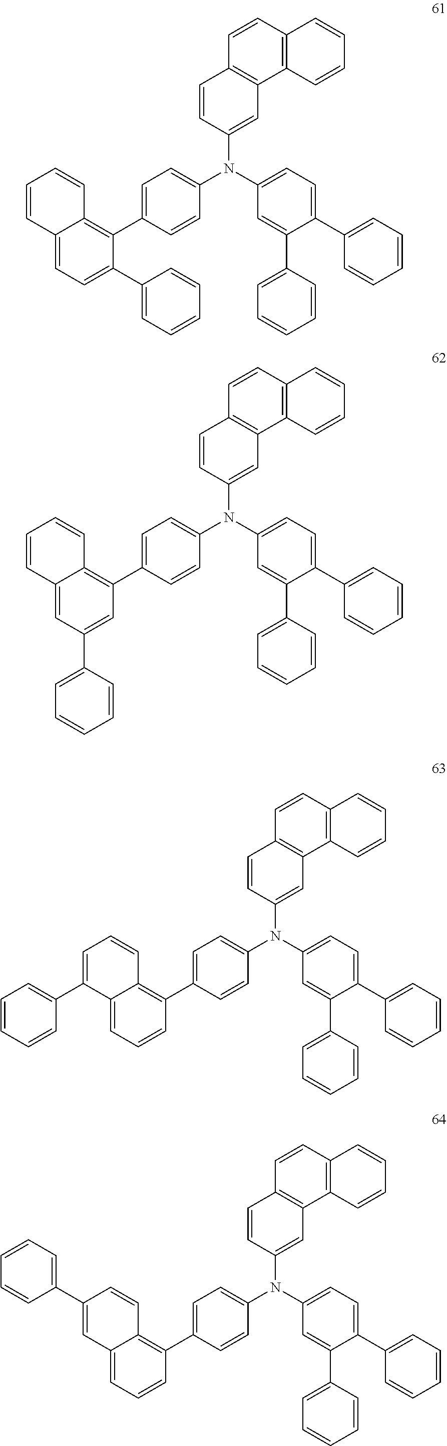 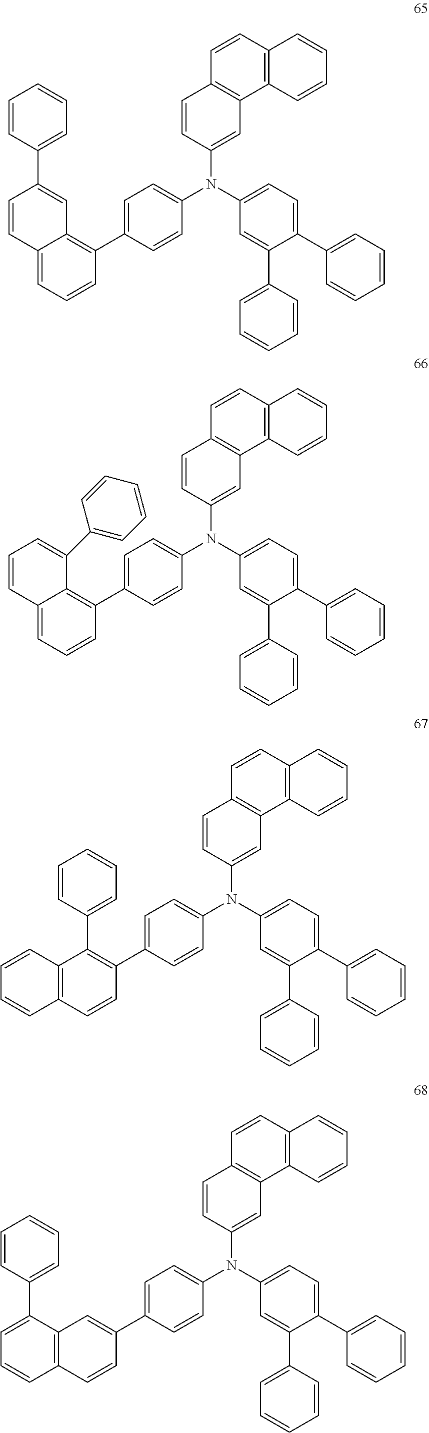 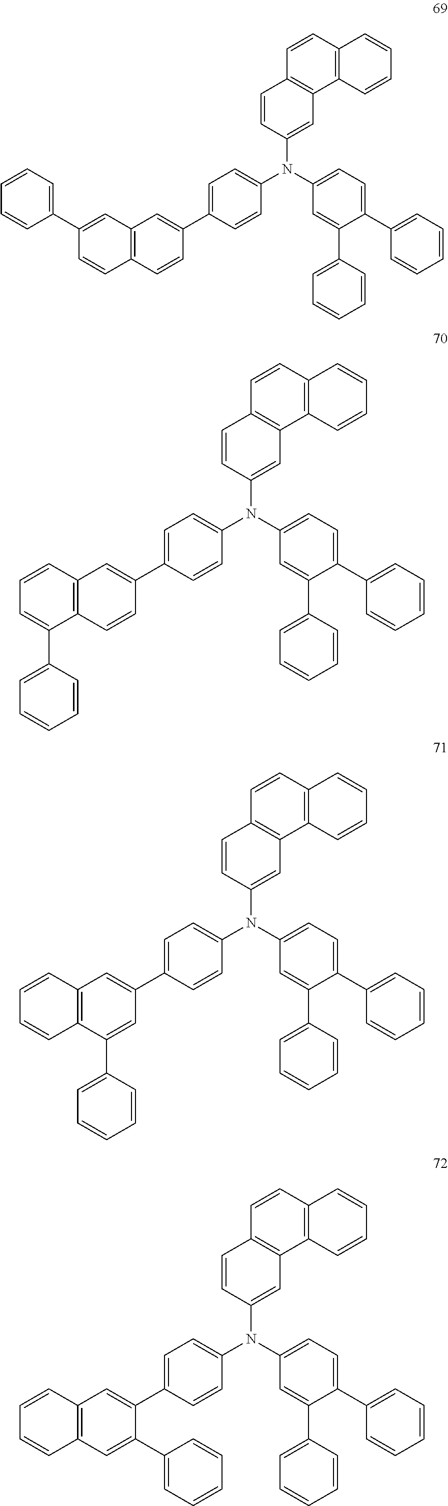 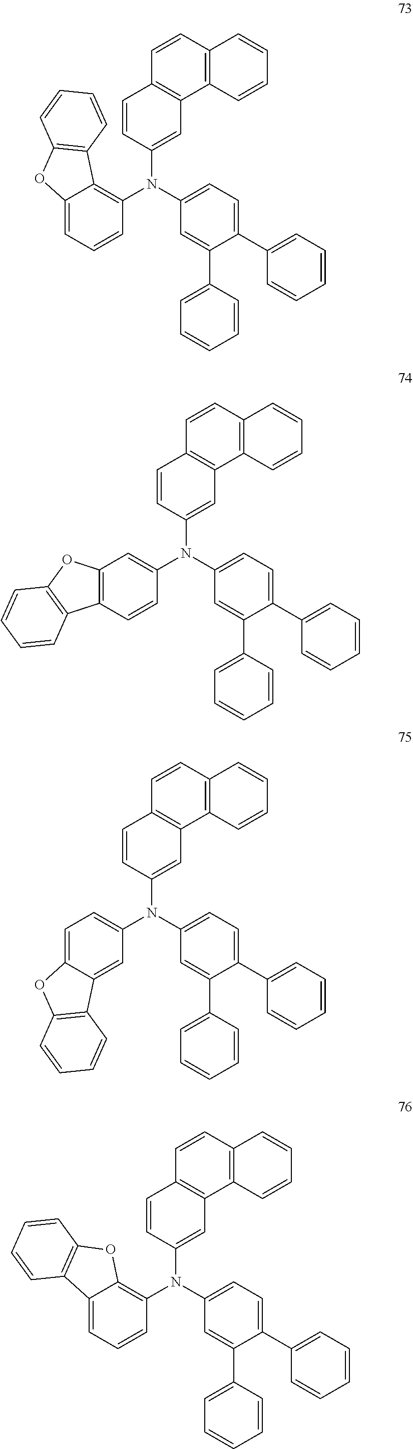 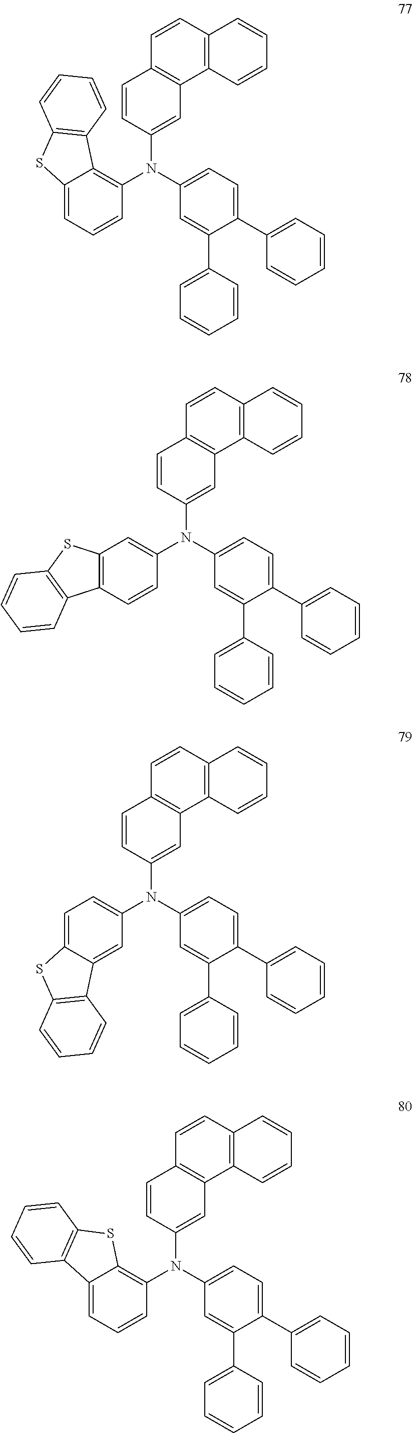 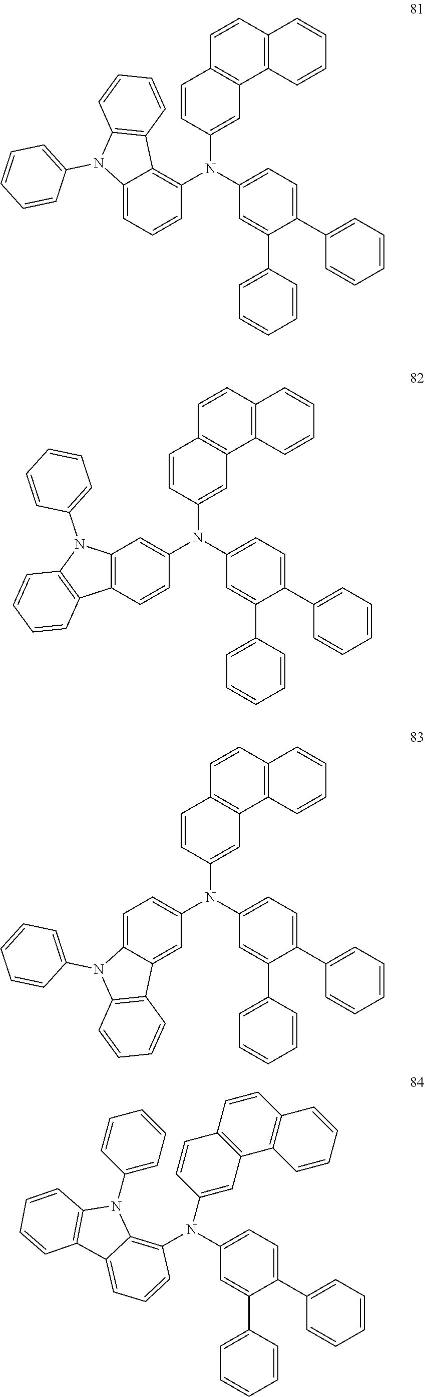 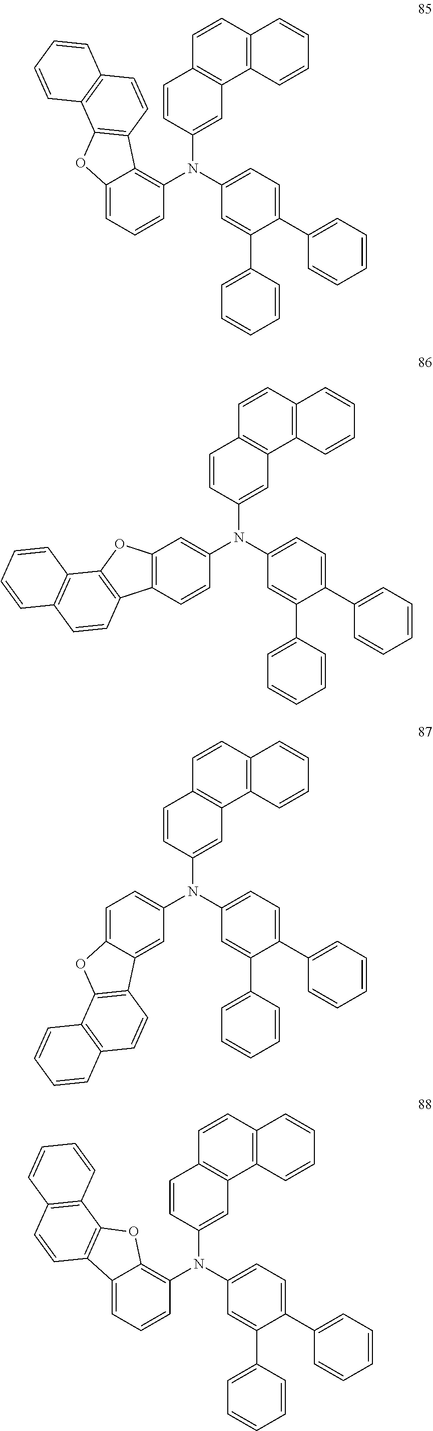 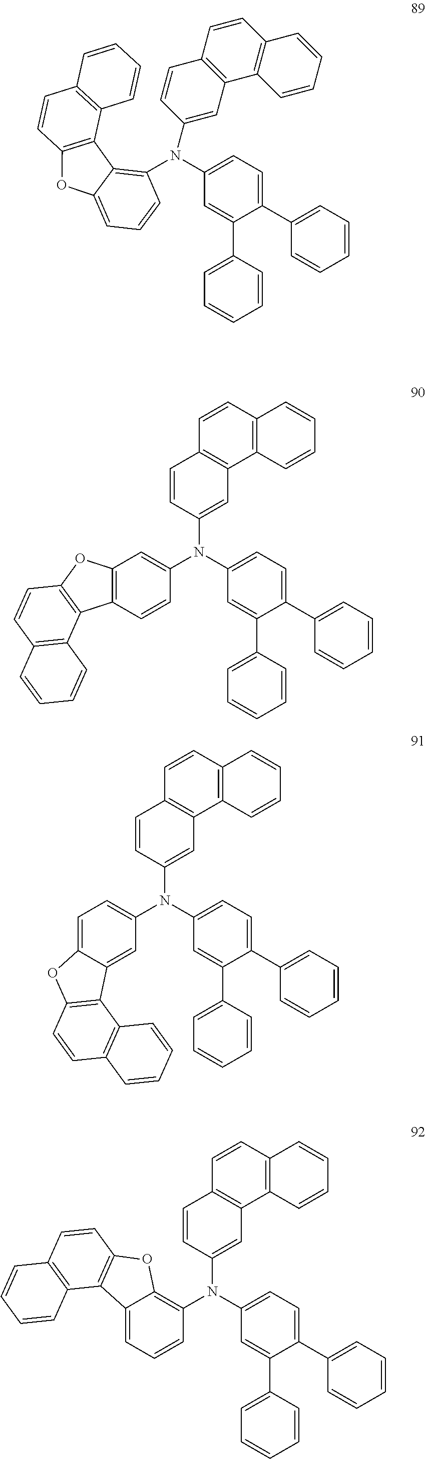 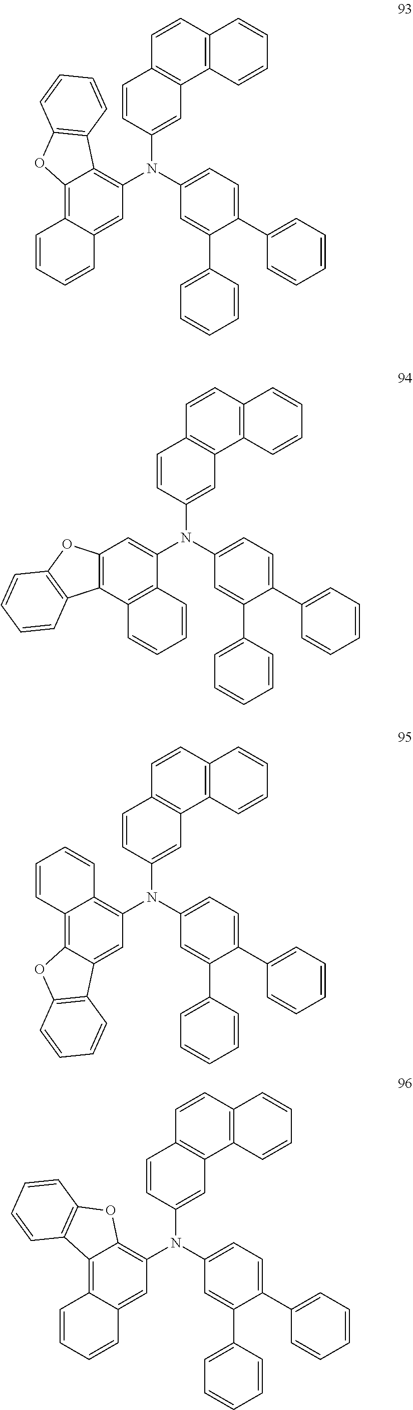 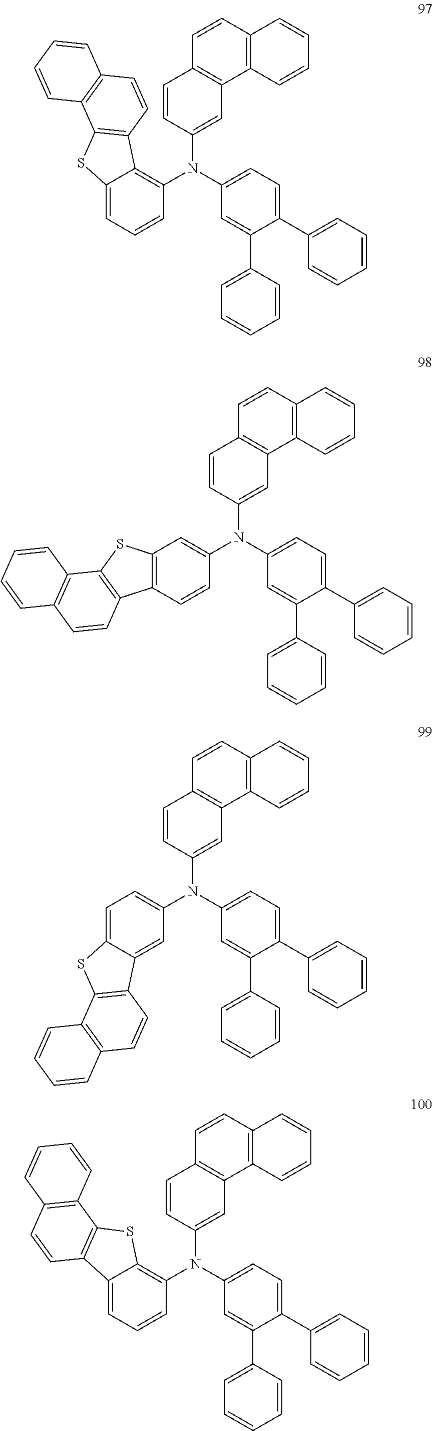 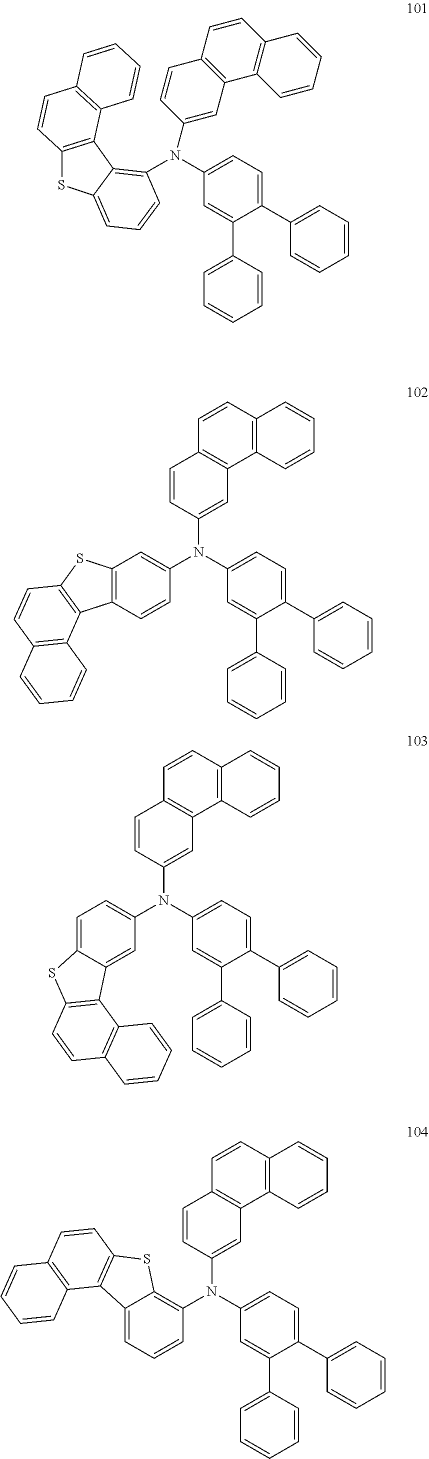 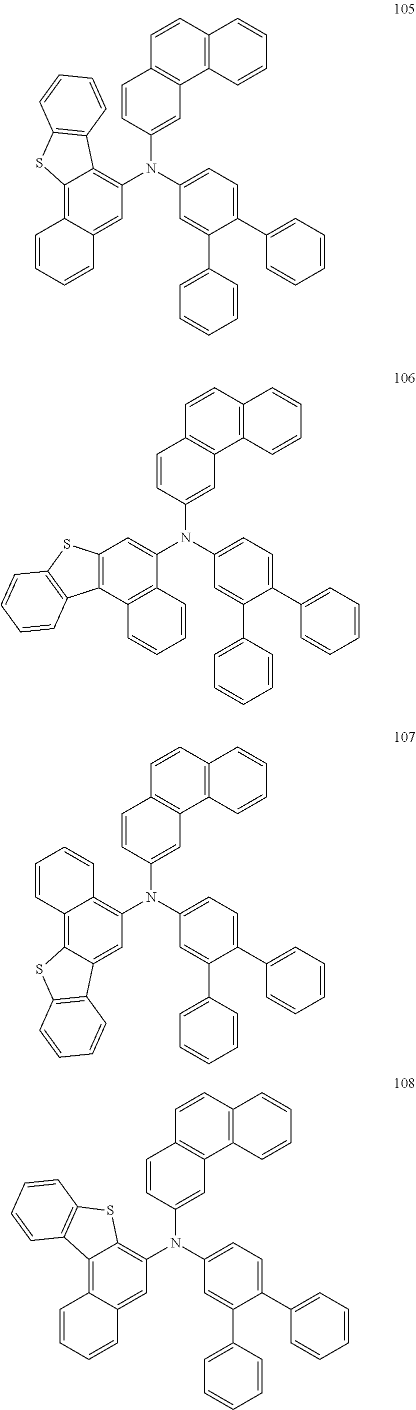 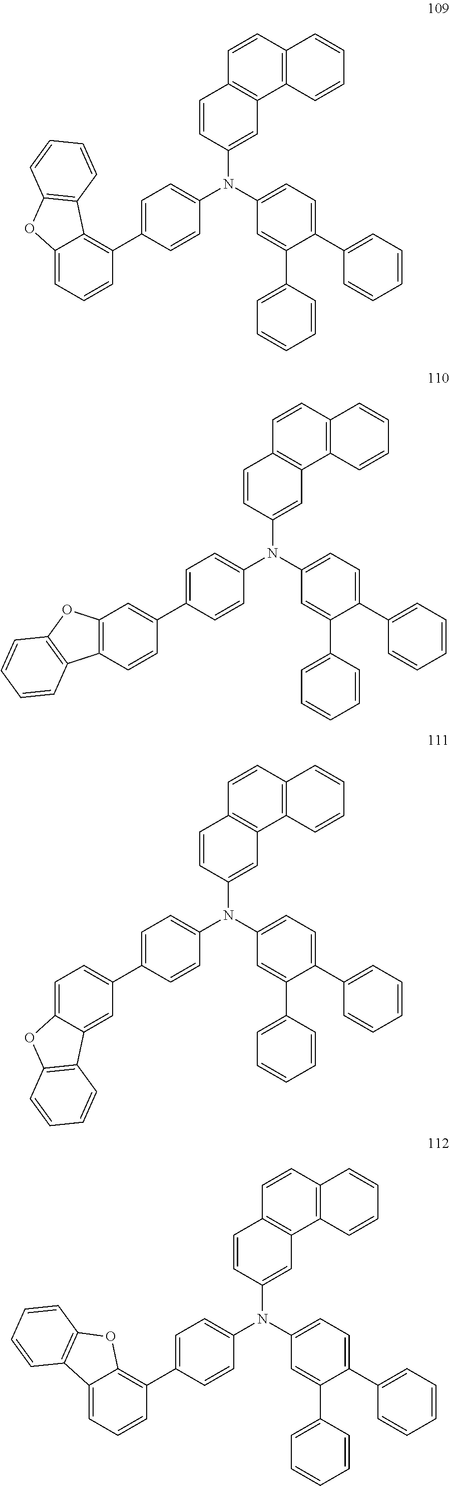 [SMILES: c1ccc(-c2ccc(N(c3ccc(-c4cccc5oc6ccccc6c45)cc3)c3ccc4ccc5ccccc5c4c3)cc2-c2ccccc2)cc1 ; c1ccc(-c2ccc(N(c3ccc(-c4ccc5c(c4)oc4ccccc45)cc3)c3ccc4ccc5ccccc5c4c3)cc2-c2ccccc2)cc1 ; c1ccc(-c2ccc(N(c3ccc(-c4ccc5oc6ccccc6c5c4)cc3)c3ccc4ccc5ccccc5c4c3)cc2-c2ccccc2)cc1 ; c1ccc(-c2ccc(N(c3ccc(-c4cccc5c4oc4ccccc45)cc3)c3ccc4ccc5ccccc5c4c3)cc2-c2ccccc2)cc1] 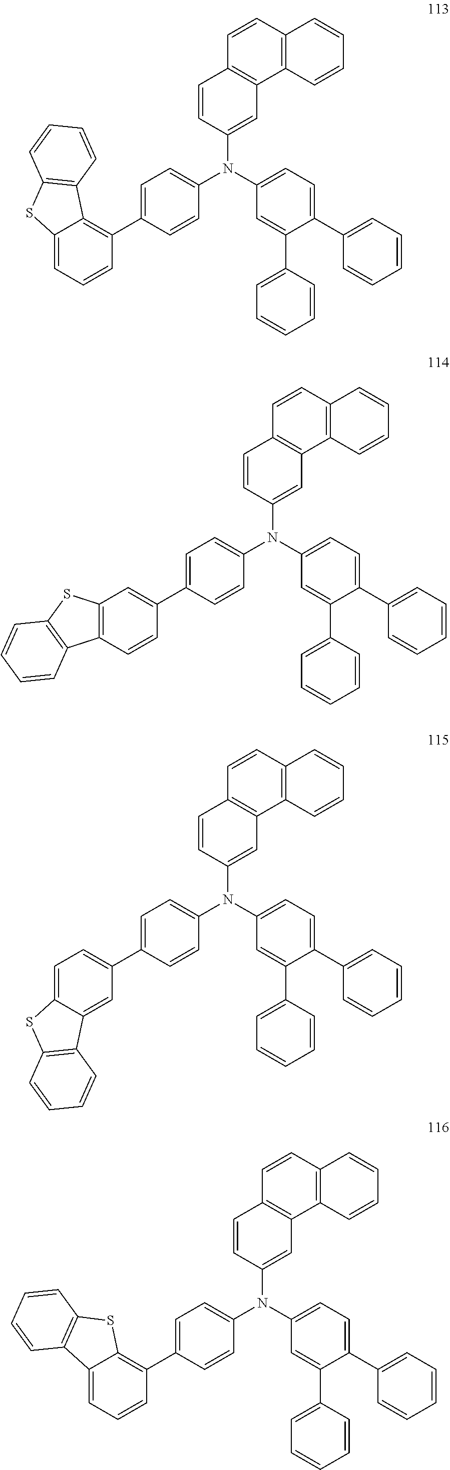 [SMILES: c1ccc(-c2ccc(N(c3ccc(-c4cccc5sc6ccccc6c45)cc3)c3ccc4ccc5ccccc5c4c3)cc2-c2ccccc2)cc1 ; c1ccc(-c2ccc(N(c3ccc(-c4ccc5c(c4)sc4ccccc45)cc3)c3ccc4ccc5ccccc5c4c3)cc2-c2ccccc2)cc1 ; c1ccc(-c2ccc(N(c3ccc(-c4ccc5sc6ccccc6c5c4)cc3)c3ccc4ccc5ccccc5c4c3)cc2-c2ccccc2)cc1 ; c1ccc(-c2ccc(N(c3ccc(-c4cccc5c4sc4ccccc45)cc3)c3ccc4ccc5ccccc5c4c3)cc2-c2ccccc2)cc1] 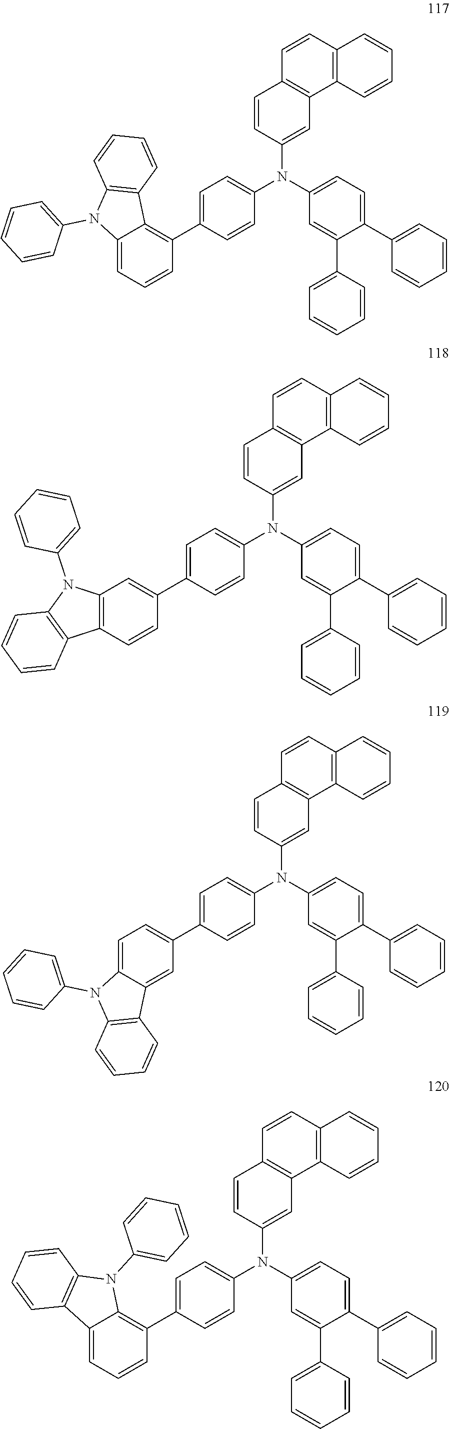 [SMILES: c1ccc(-c2ccc(N(c3ccc(-c4cccc5c4c4ccccc4n5-c4ccccc4)cc3)c3ccc4ccc5ccccc5c4c3)cc2-c2ccccc2)cc1 ; c1ccc(-c2ccc(N(c3ccc(-c4ccc5c6ccccc6n(-c6ccccc6)c5c4)cc3)c3ccc4ccc5ccccc5c4c3)cc2-c2ccccc2)cc1 ; c1ccc(-c2ccc(N(c3ccc(-c4ccc5c(c4)c4ccccc4n5-c4ccccc4)cc3)c3ccc4ccc5ccccc5c4c3)cc2-c2ccccc2)cc1 ; c1ccc(-c2ccc(N(c3ccc(-c4cccc5c6ccccc6n(-c6ccccc6)c45)cc3)c3ccc4ccc5ccccc5c4c3)cc2-c2ccccc2)cc1] 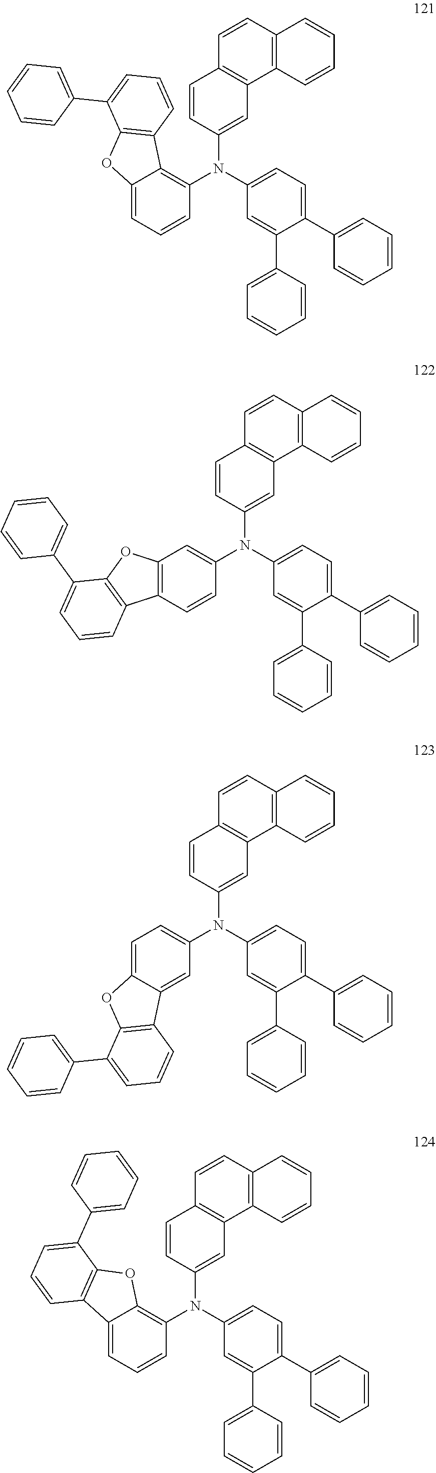 [SMILES: c1ccc(-c2ccc(N(c3ccc4ccc5ccccc5c4c3)c3cccc4oc5c(-c6ccccc6)cccc5c34)cc2-c2ccccc2)cc1 ; c1ccc(-c2ccc(N(c3ccc4c(c3)oc3c(-c5ccccc5)cccc34)c3ccc4ccc5ccccc5c4c3)cc2-c2ccccc2)cc1 ; c1ccc(-c2ccc(N(c3ccc4ccc5ccccc5c4c3)c3ccc4oc5c(-c6ccccc6)cccc5c4c3)cc2-c2ccccc2)cc1 ; c1ccc(-c2ccc(N(c3ccc4ccc5ccccc5c4c3)c3cccc4c3oc3c(-c5ccccc5)cccc34)cc2-c2ccccc2)cc1] 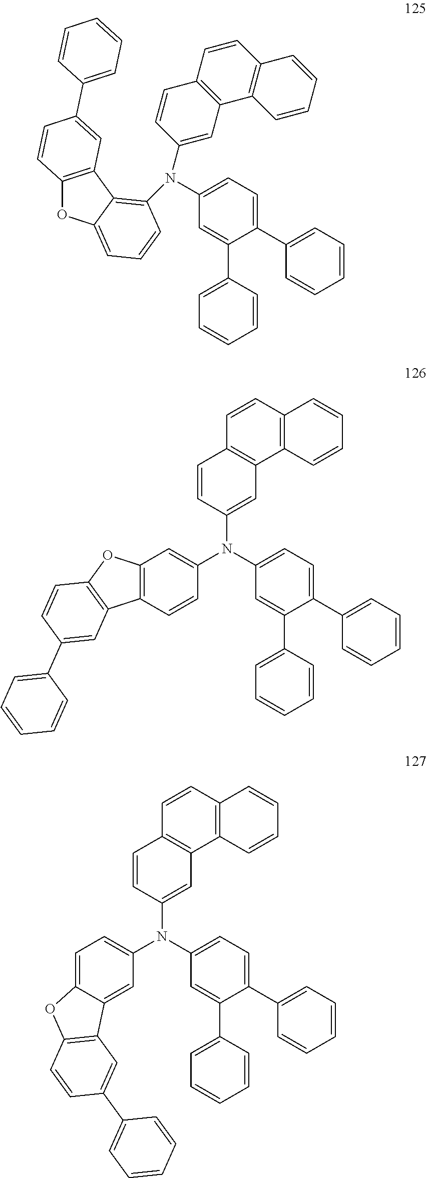 [SMILES: c1ccc(-c2ccc3oc4cccc(N(c5ccc(-c6ccccc6)c(-c6ccccc6)c5)c5ccc6ccc7ccccc7c6c5)c4c3c2)cc1 ; c1ccc(-c2ccc3oc4cc(N(c5ccc(-c6ccccc6)c(-c6ccccc6)c5)c5ccc6ccc7ccccc7c6c5)ccc4c3c2)cc1 ; c1ccc(-c2ccc3oc4ccc(N(c5ccc(-c6ccccc6)c(-c6ccccc6)c5)c5ccc6ccc7ccccc7c6c5)cc4c3c2)cc1] 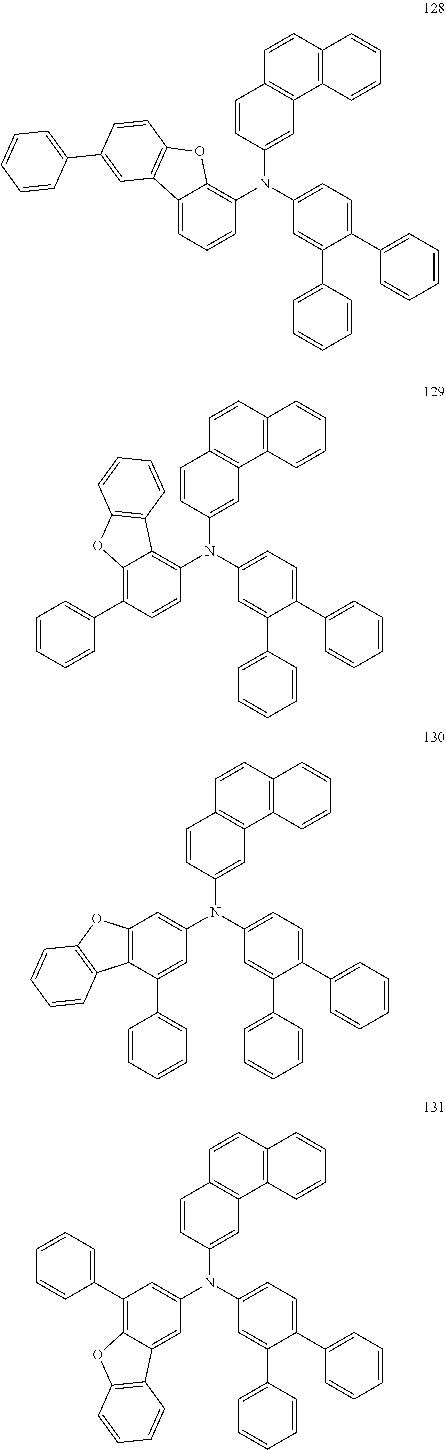 [SMILES: c1ccc(-c2ccc3oc4c(N(c5ccc(-c6ccccc6)c(-c6ccccc6)c5)c5ccc6ccc7ccccc7c6c5)cccc4c3c2)cc1 ; c1ccc(-c2ccc(N(c3ccc4ccc5ccccc5c4c3)c3ccc(-c4ccccc4)c4oc5ccccc5c34)cc2-c2ccccc2)cc1 ; c1ccc(-c2ccc(N(c3cc(-c4ccccc4)c4c(c3)oc3ccccc34)c3ccc4ccc5ccccc5c4c3)cc2-c2ccccc2)cc1 ; c1ccc(-c2ccc(N(c3ccc4ccc5ccccc5c4c3)c3cc(-c4ccccc4)c4oc5ccccc5c4c3)cc2-c2ccccc2)cc1] 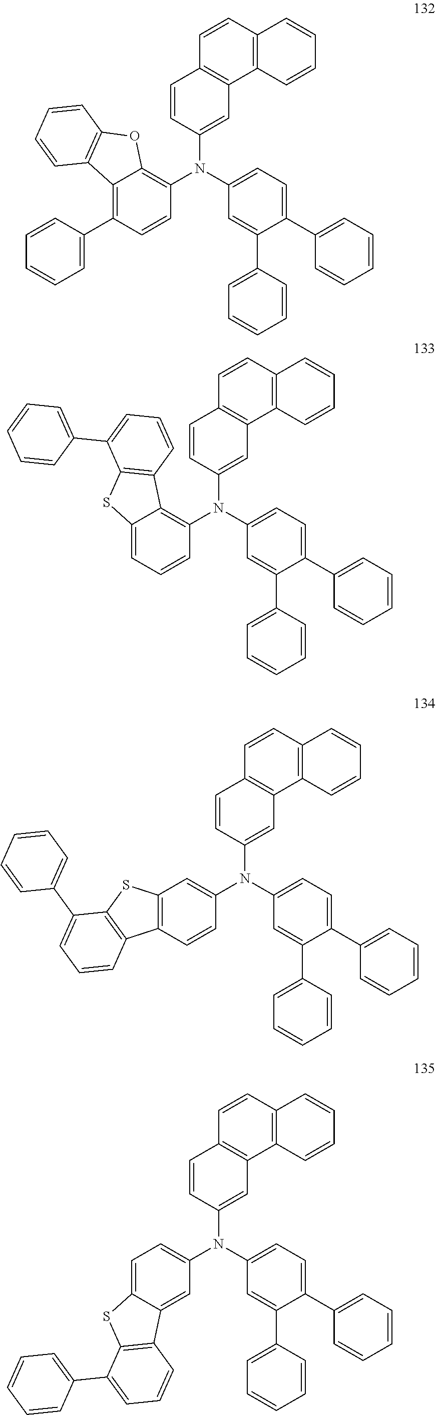 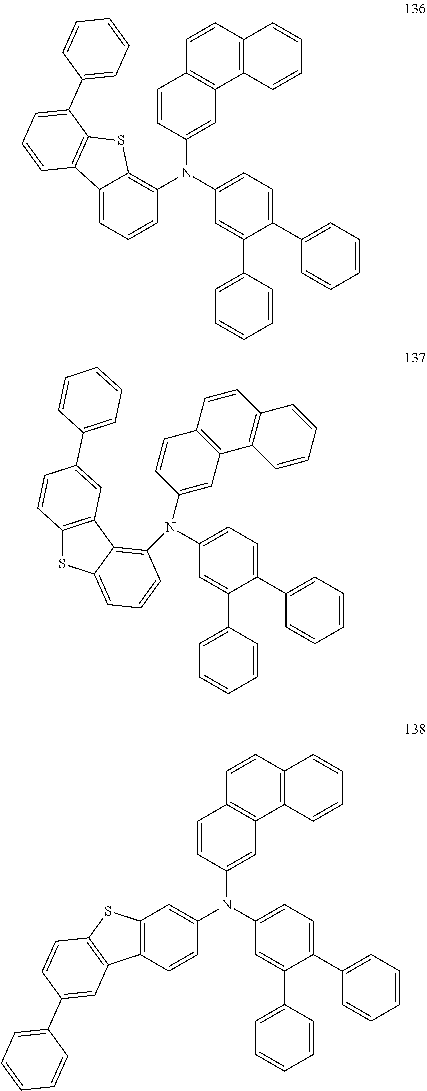 [SMILES: c1ccc(-c2ccc(N(c3ccc4ccc5ccccc5c4c3)c3cccc4c3sc3c(-c5ccccc5)cccc34)cc2-c2ccccc2)cc1 ; c1ccc(-c2ccc3sc4cccc(N(c5ccc(-c6ccccc6)c(-c6ccccc6)c5)c5ccc6ccc7ccccc7c6c5)c4c3c2)cc1 ; c1ccc(-c2ccc3sc4cc(N(c5ccc(-c6ccccc6)c(-c6ccccc6)c5)c5ccc6ccc7ccccc7c6c5)ccc4c3c2)cc1] 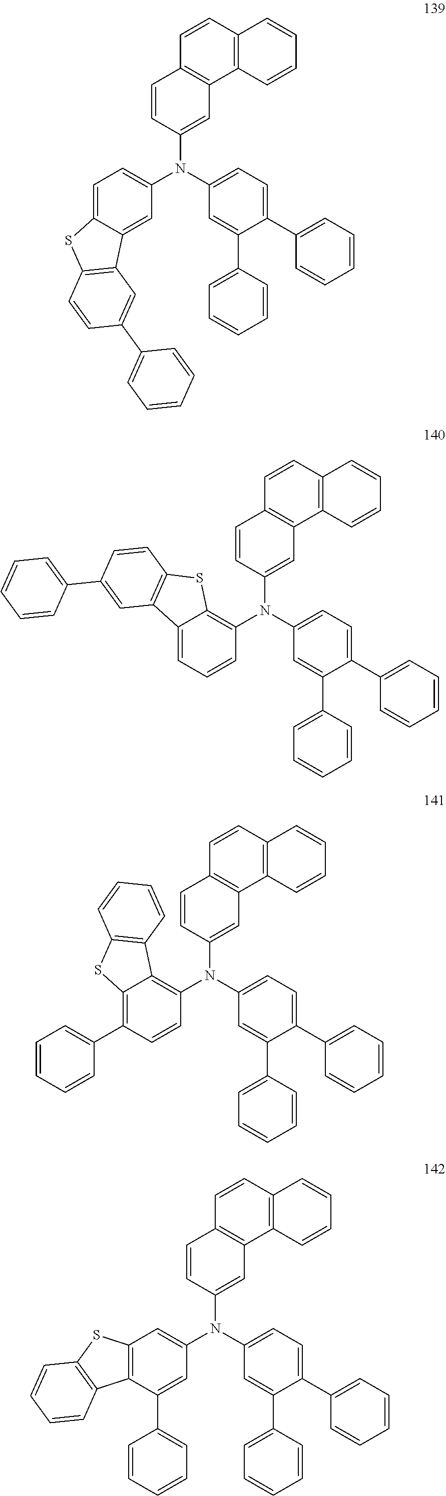 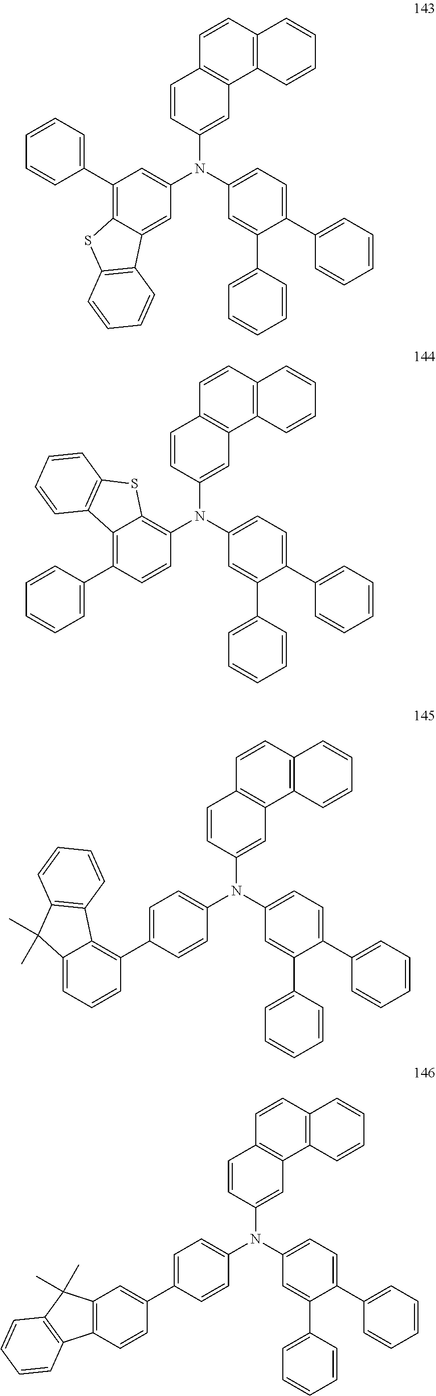 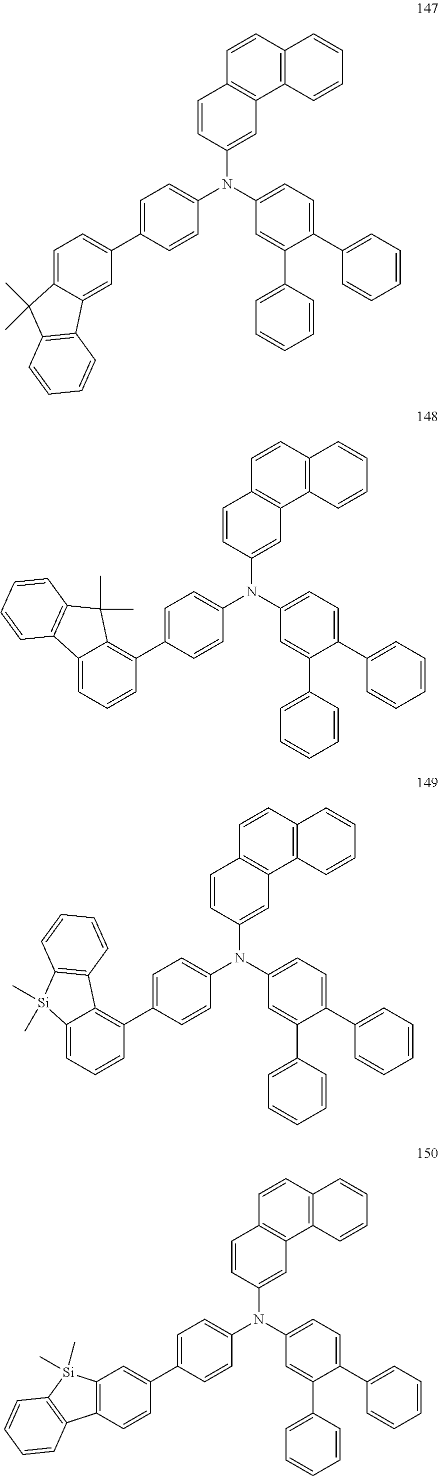 [SMILES: CC1(C)c2ccccc2-c2cc(-c3ccc(N(c4ccc(-c5ccccc5)c(-c5ccccc5)c4)c4ccc5ccc6ccccc6c5c4)cc3)ccc21 ; CC1(C)c2ccccc2-c2cccc(-c3ccc(N(c4ccc(-c5ccccc5)c(-c5ccccc5)c4)c4ccc5ccc6ccccc6c5c4)cc3)c21 ; C[Si]1(C)c2ccccc2-c2c(-c3ccc(N(c4ccc(-c5ccccc5)c(-c5ccccc5)c4)c4ccc5ccc6ccccc6c5c4)cc3)cccc21 ; C[Si]1(C)c2ccccc2-c2ccc(-c3ccc(N(c4ccc(-c5ccccc5)c(-c5ccccc5)c4)c4ccc5ccc6ccccc6c5c4)cc3)cc21] 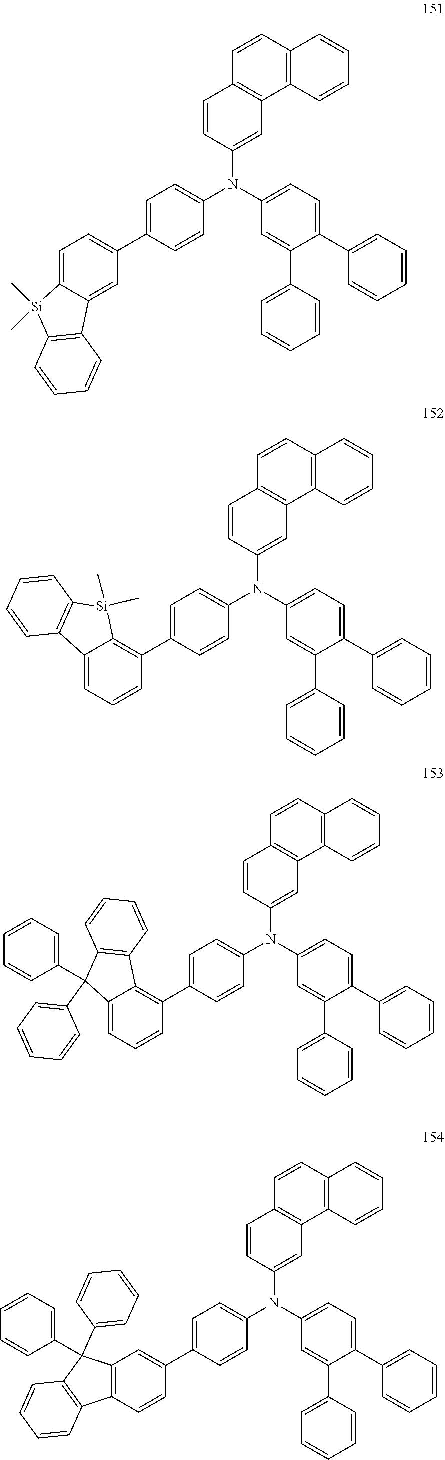 [SMILES: C[Si]1(C)c2ccccc2-c2cc(-c3ccc(N(c4ccc(-c5ccccc5)c(-c5ccccc5)c4)c4ccc5ccc6ccccc6c5c4)cc3)ccc21 ; C[Si]1(C)c2ccccc2-c2cccc(-c3ccc(N(c4ccc(-c5ccccc5)c(-c5ccccc5)c4)c4ccc5ccc6ccccc6c5c4)cc3)c21 ; c1ccc(-c2ccc(N(c3ccc(-c4cccc5c4-c4ccccc4C5(c4ccccc4)c4ccccc4)cc3)c3ccc4ccc5ccccc5c4c3)cc2-c2ccccc2)cc1 ; c1ccc(-c2ccc(N(c3ccc(-c4ccc5c(c4)C(c4ccccc4)(c4ccccc4)c4ccccc4-5)cc3)c3ccc4ccc5ccccc5c4c3)cc2-c2ccccc2)cc1] 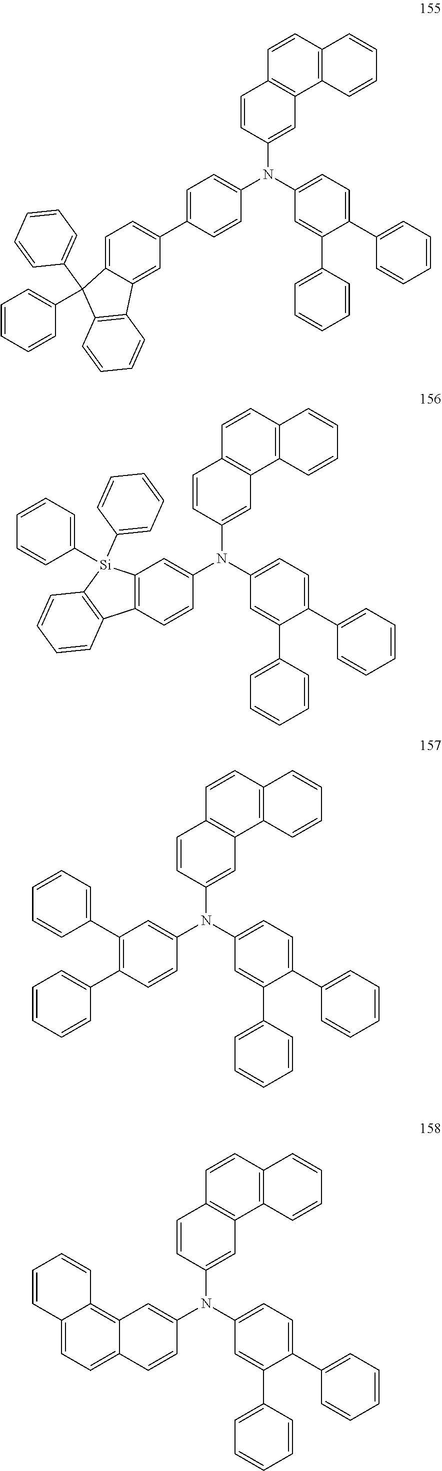 [SMILES: c1ccc(-c2ccc(N(c3ccc(-c4ccc5c(c4)-c4ccccc4C5(c4ccccc4)c4ccccc4)cc3)c3ccc4ccc5ccccc5c4c3)cc2-c2ccccc2)cc1 ; c1ccc(-c2ccc(N(c3ccc4c(c3)[Si](c3ccccc3)(c3ccccc3)c3ccccc3-4)c3ccc4ccc5ccccc5c4c3)cc2-c2ccccc2)cc1 ; c1ccc(-c2ccc(N(c3ccc(-c4ccccc4)c(-c4ccccc4)c3)c3ccc4ccc5ccccc5c4c3)cc2-c2ccccc2)cc1 ; c1ccc(-c2ccc(N(c3ccc4ccc5ccccc5c4c3)c3ccc4ccc5ccccc5c4c3)cc2-c2ccccc2)cc1] 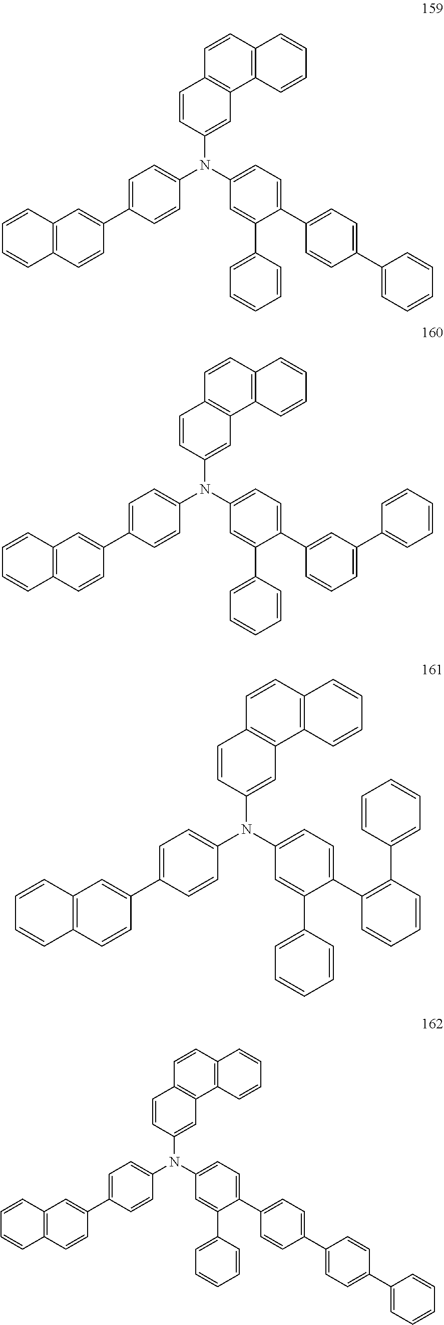 [SMILES: c1ccc(-c2ccc(-c3ccc(N(c4ccc(-c5ccc6ccccc6c5)cc4)c4ccc5ccc6ccccc6c5c4)cc3-c3ccccc3)cc2)cc1 ; c1ccc(-c2cccc(-c3ccc(N(c4ccc(-c5ccc6ccccc6c5)cc4)c4ccc5ccc6ccccc6c5c4)cc3-c3ccccc3)c2)cc1 ; c1ccc(-c2ccccc2-c2ccc(N(c3ccc(-c4ccc5ccccc5c4)cc3)c3ccc4ccc5ccccc5c4c3)cc2-c2ccccc2)cc1 ; c1ccc(-c2ccc(-c3ccc(-c4ccc(N(c5ccc(-c6ccc7ccccc7c6)cc5)c5ccc6ccc7ccccc7c6c5)cc4-c4ccccc4)cc3)cc2)cc1] 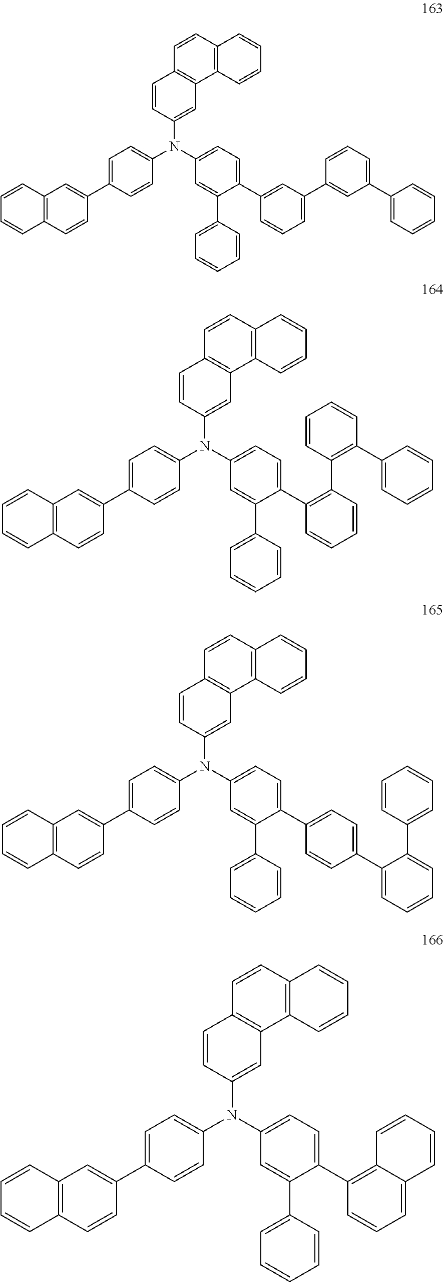 [SMILES: c1ccc(-c2cccc(-c3cccc(-c4ccc(N(c5ccc(-c6ccc7ccccc7c6)cc5)c5ccc6ccc7ccccc7c6c5)cc4-c4ccccc4)c3)c2)cc1 ; c1ccc(-c2ccccc2-c2ccccc2-c2ccc(N(c3ccc(-c4ccc5ccccc5c4)cc3)c3ccc4ccc5ccccc5c4c3)cc2-c2ccccc2)cc1 ; c1ccc(-c2ccccc2-c2ccc(-c3ccc(N(c4ccc(-c5ccc6ccccc6c5)cc4)c4ccc5ccc6ccccc6c5c4)cc3-c3ccccc3)cc2)cc1 ; c1ccc(-c2cc(N(c3ccc(-c4ccc5ccccc5c4)cc3)c3ccc4ccc5ccccc5c4c3)ccc2-c2cccc3ccccc23)cc1] 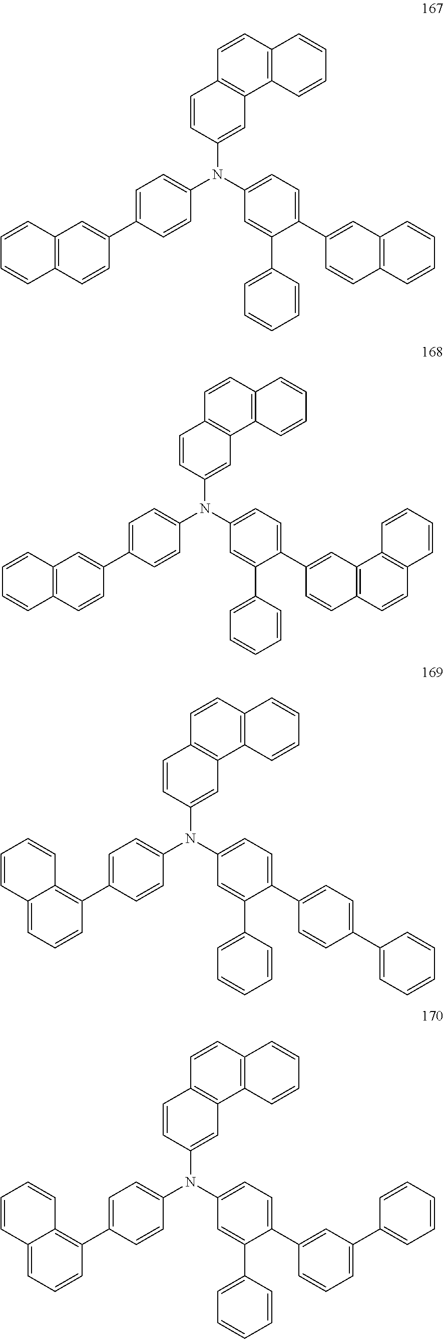 [SMILES: c1ccc(-c2cc(N(c3ccc(-c4ccc5ccccc5c4)cc3)c3ccc4ccc5ccccc5c4c3)ccc2-c2ccc3ccccc3c2)cc1 ; c1ccc(-c2cc(N(c3ccc(-c4ccc5ccccc5c4)cc3)c3ccc4ccc5ccccc5c4c3)ccc2-c2ccc3ccc4ccccc4c3c2)cc1 ; c1ccc(-c2ccc(-c3ccc(N(c4ccc(-c5cccc6ccccc56)cc4)c4ccc5ccc6ccccc6c5c4)cc3-c3ccccc3)cc2)cc1 ; c1ccc(-c2cccc(-c3ccc(N(c4ccc(-c5cccc6ccccc56)cc4)c4ccc5ccc6ccccc6c5c4)cc3-c3ccccc3)c2)cc1] 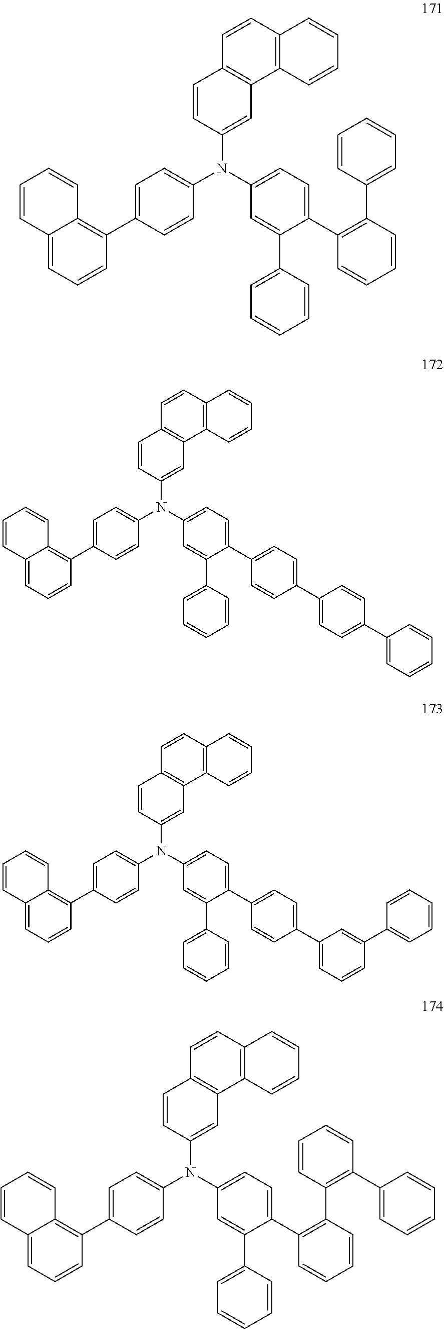 [SMILES: c1ccc(-c2ccccc2-c2ccc(N(c3ccc(-c4cccc5ccccc45)cc3)c3ccc4ccc5ccccc5c4c3)cc2-c2ccccc2)cc1 ; c1ccc(-c2ccc(-c3ccc(-c4ccc(N(c5ccc(-c6cccc7ccccc67)cc5)c5ccc6ccc7ccccc7c6c5)cc4-c4ccccc4)cc3)cc2)cc1 ; c1ccc(-c2cccc(-c3ccc(-c4ccc(N(c5ccc(-c6cccc7ccccc67)cc5)c5ccc6ccc7ccccc7c6c5)cc4-c4ccccc4)cc3)c2)cc1 ; c1ccc(-c2ccccc2-c2ccccc2-c2ccc(N(c3ccc(-c4cccc5ccccc45)cc3)c3ccc4ccc5ccccc5c4c3)cc2-c2ccccc2)cc1] 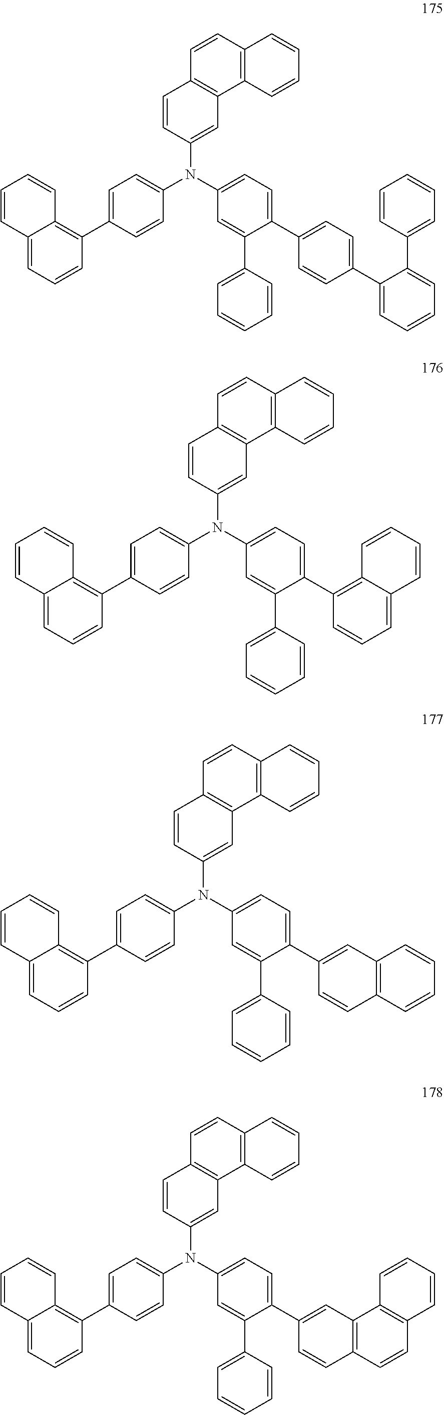 [SMILES: c1ccc(-c2ccccc2-c2ccc(-c3ccc(N(c4ccc(-c5cccc6ccccc56)cc4)c4ccc5ccc6ccccc6c5c4)cc3-c3ccccc3)cc2)cc1 ; c1ccc(-c2cc(N(c3ccc(-c4cccc5ccccc45)cc3)c3ccc4ccc5ccccc5c4c3)ccc2-c2cccc3ccccc23)cc1 ; c1ccc(-c2cc(N(c3ccc(-c4cccc5ccccc45)cc3)c3ccc4ccc5ccccc5c4c3)ccc2-c2ccc3ccccc3c2)cc1 ; c1ccc(-c2cc(N(c3ccc(-c4cccc5ccccc45)cc3)c3ccc4ccc5ccccc5c4c3)ccc2-c2ccc3ccc4ccccc4c3c2)cc1] 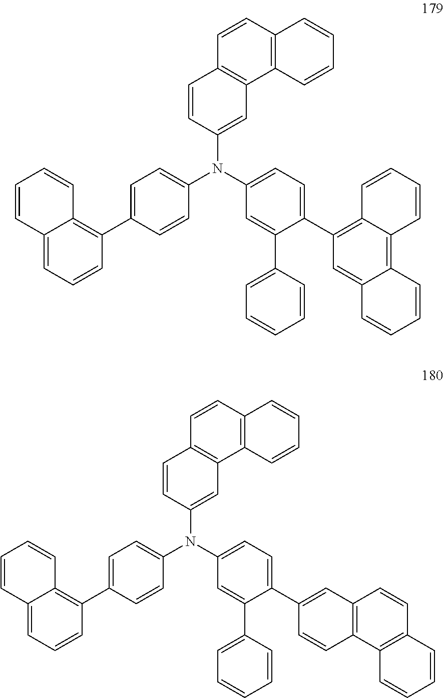 [SMILES: c1ccc(-c2cc(N(c3ccc(-c4cccc5ccccc45)cc3)c3ccc4ccc5ccccc5c4c3)ccc2-c2cc3ccccc3c3ccccc23)cc1 ; c1ccc(-c2cc(N(c3ccc(-c4cccc5ccccc45)cc3)c3ccc4ccc5ccccc5c4c3)ccc2-c2ccc3c(ccc4ccccc43)c2)cc1] 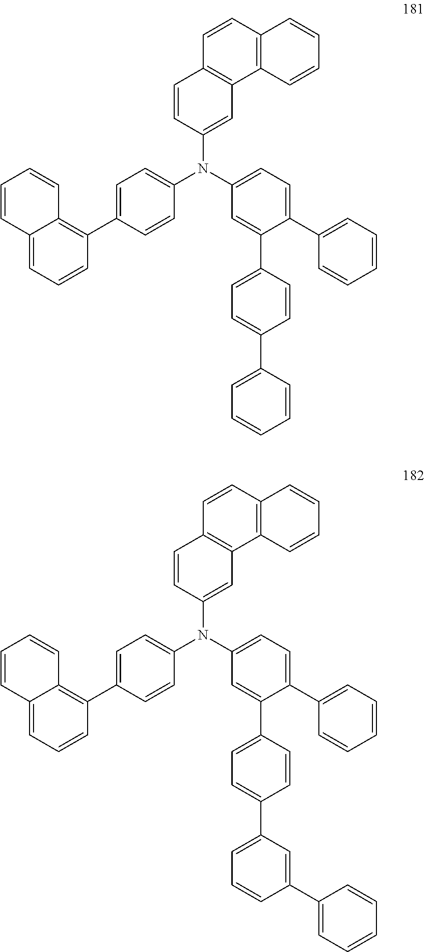 [SMILES: c1ccc(-c2ccc(-c3cc(N(c4ccc(-c5cccc6ccccc56)cc4)c4ccc5ccc6ccccc6c5c4)ccc3-c3ccccc3)cc2)cc1 ; c1ccc(-c2cccc(-c3ccc(-c4cc(N(c5ccc(-c6cccc7ccccc67)cc5)c5ccc6ccc7ccccc7c6c5)ccc4-c4ccccc4)cc3)c2)cc1] 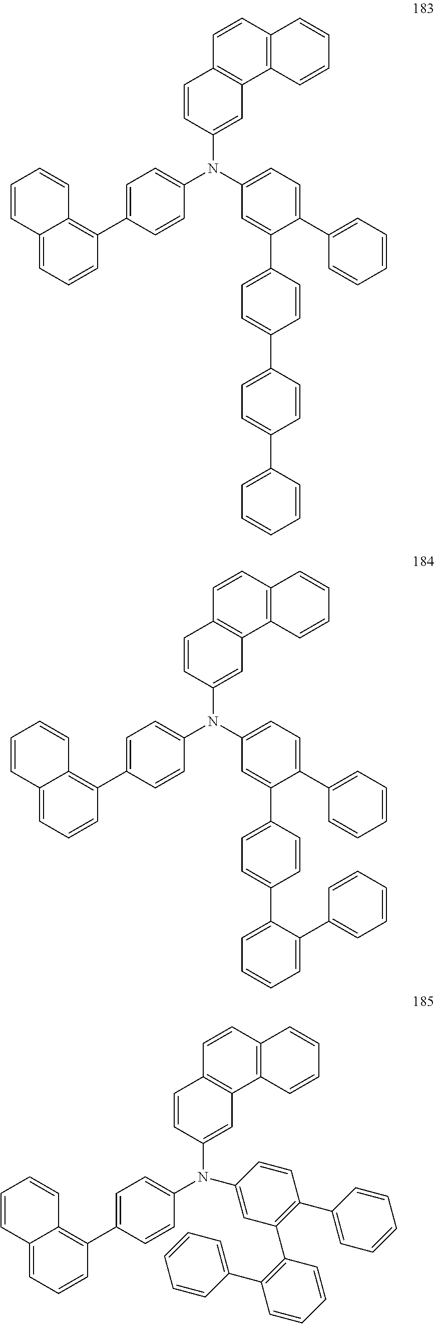 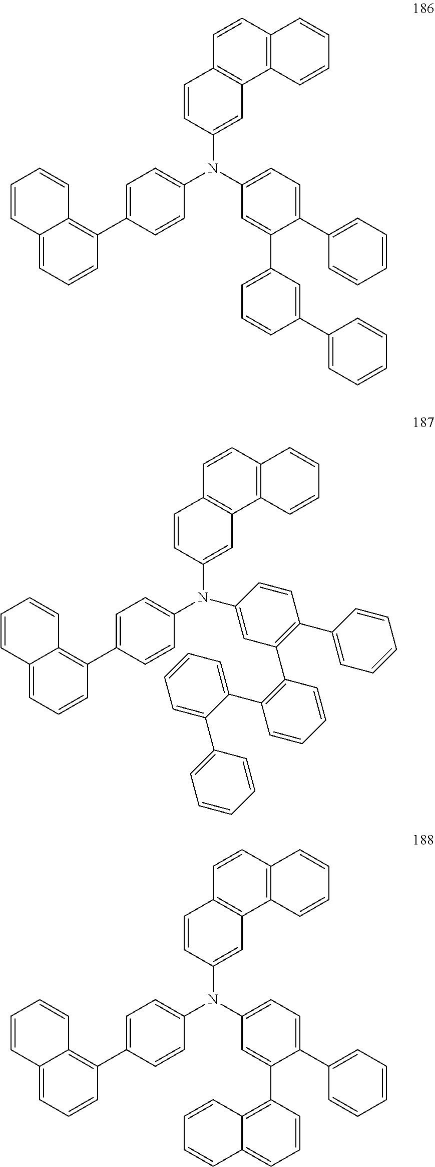 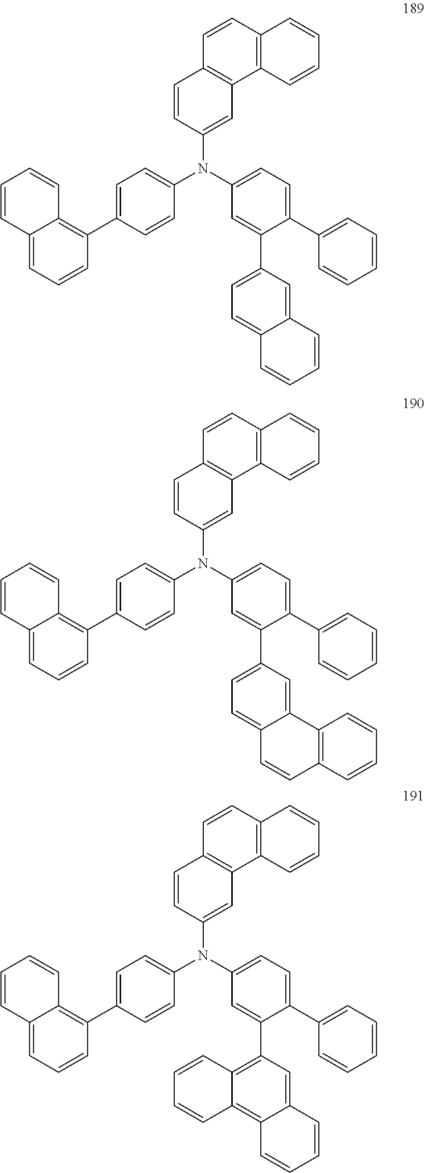 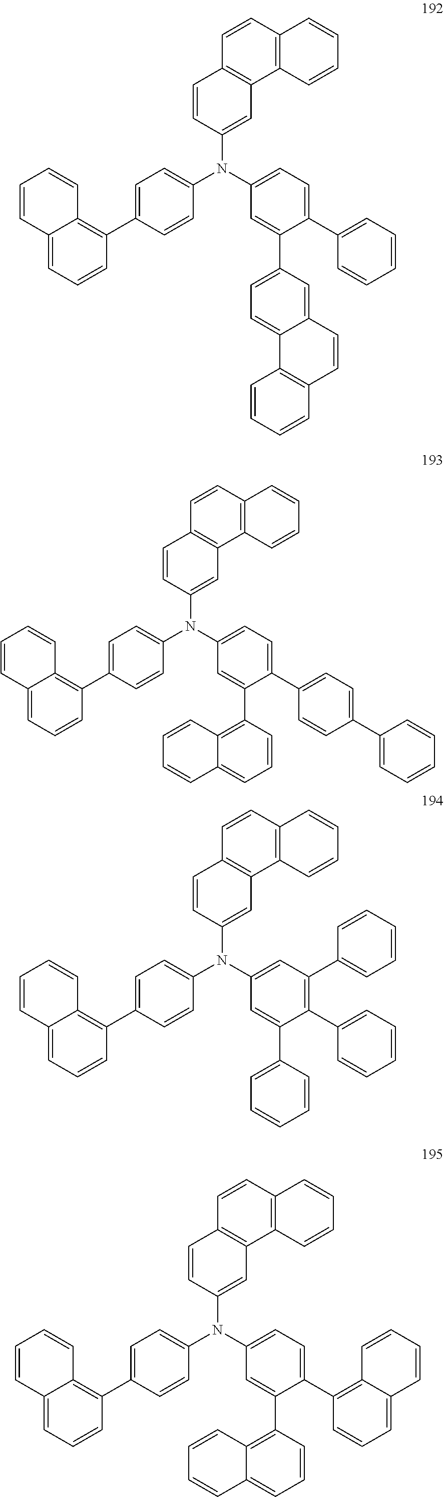 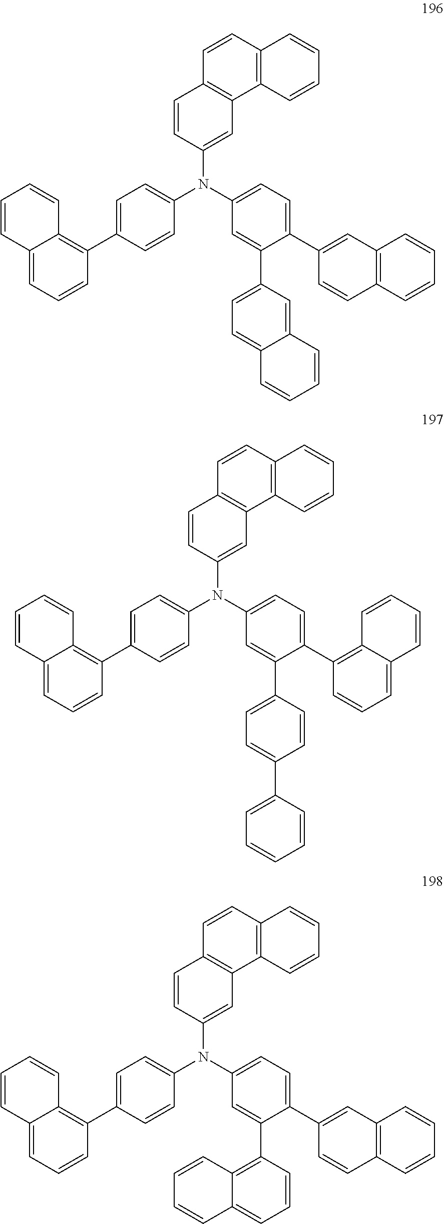 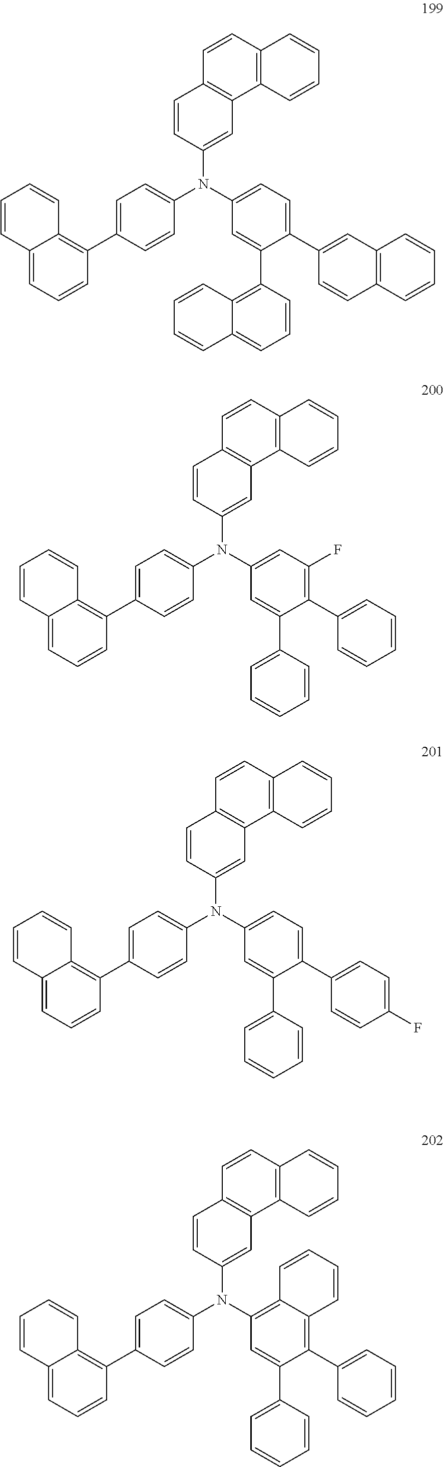 [SMILES: c1ccc2cc(-c3ccc(N(c4ccc(-c5cccc6ccccc56)cc4)c4ccc5ccc6ccccc6c5c4)cc3-c3cccc4ccccc34)ccc2c1 ; Fc1cc(N(c2ccc(-c3cccc4ccccc34)cc2)c2ccc3ccc4ccccc4c3c2)cc(-c2ccccc2)c1-c1ccccc1 ; Fc1ccc(-c2ccc(N(c3ccc(-c4cccc5ccccc45)cc3)c3ccc4ccc5ccccc5c4c3)cc2-c2ccccc2)cc1 ; c1ccc(-c2cc(N(c3ccc(-c4cccc5ccccc45)cc3)c3ccc4ccc5ccccc5c4c3)c3ccccc3c2-c2ccccc2)cc1] 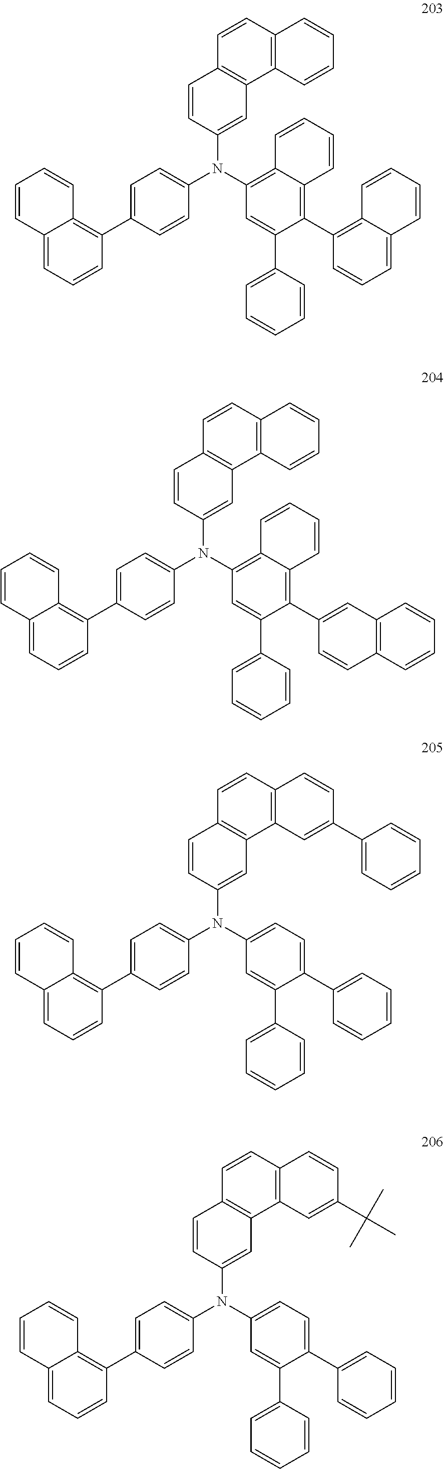 [SMILES: c1ccc(-c2cc(N(c3ccc(-c4cccc5ccccc45)cc3)c3ccc4ccc5ccccc5c4c3)c3ccccc3c2-c2cccc3ccccc23)cc1 ; c1ccc(-c2cc(N(c3ccc(-c4cccc5ccccc45)cc3)c3ccc4ccc5ccccc5c4c3)c3ccccc3c2-c2ccc3ccccc3c2)cc1 ; c1ccc(-c2ccc3ccc4ccc(N(c5ccc(-c6cccc7ccccc67)cc5)c5ccc(-c6ccccc6)c(-c6ccccc6)c5)cc4c3c2)cc1 ; CC(C)(C)c1ccc2ccc3ccc(N(c4ccc(-c5cccc6ccccc56)cc4)c4ccc(-c5ccccc5)c(-c5ccccc5)c4)cc3c2c1] 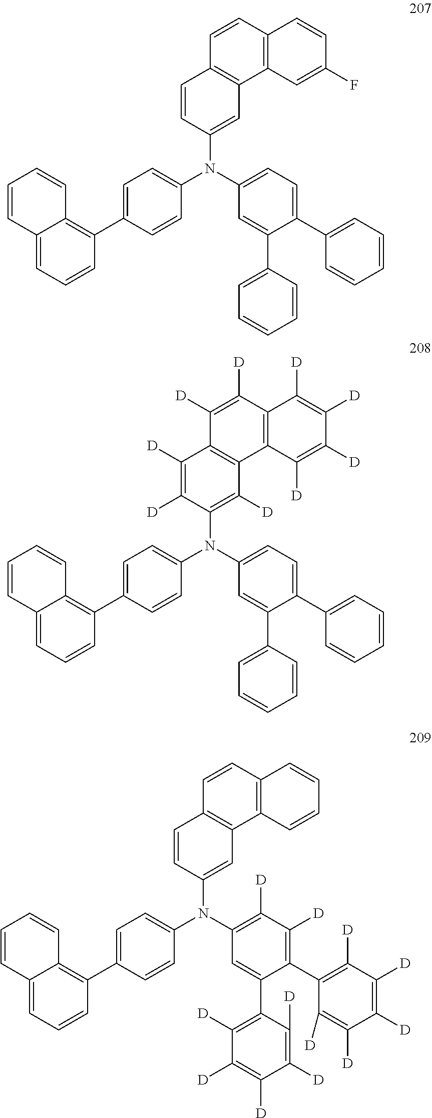 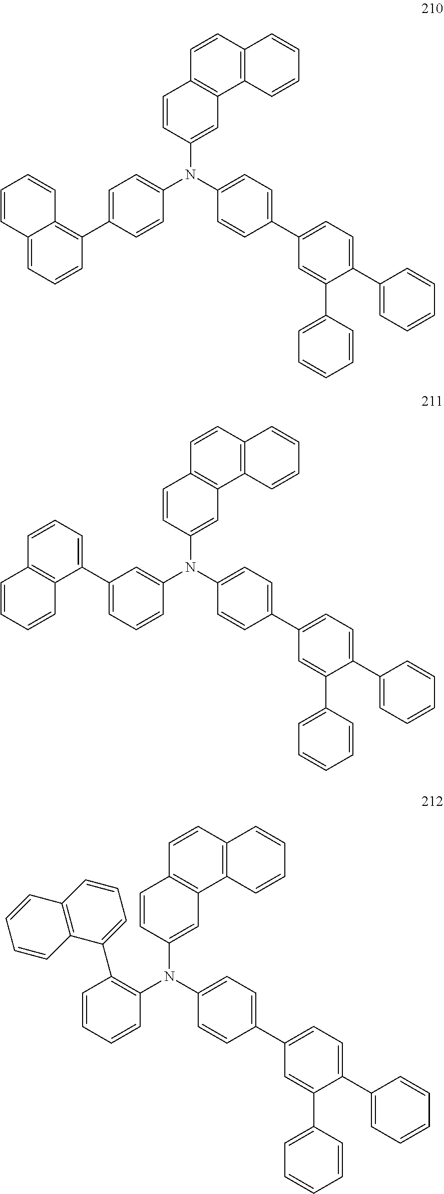 [SMILES: c1ccc(-c2ccc(-c3ccc(N(c4ccc(-c5cccc6ccccc56)cc4)c4ccc5ccc6ccccc6c5c4)cc3)cc2-c2ccccc2)cc1 ; c1ccc(-c2ccc(-c3ccc(N(c4cccc(-c5cccc6ccccc56)c4)c4ccc5ccc6ccccc6c5c4)cc3)cc2-c2ccccc2)cc1 ; c1ccc(-c2ccc(-c3ccc(N(c4ccc5ccc6ccccc6c5c4)c4ccccc4-c4cccc5ccccc45)cc3)cc2-c2ccccc2)cc1] 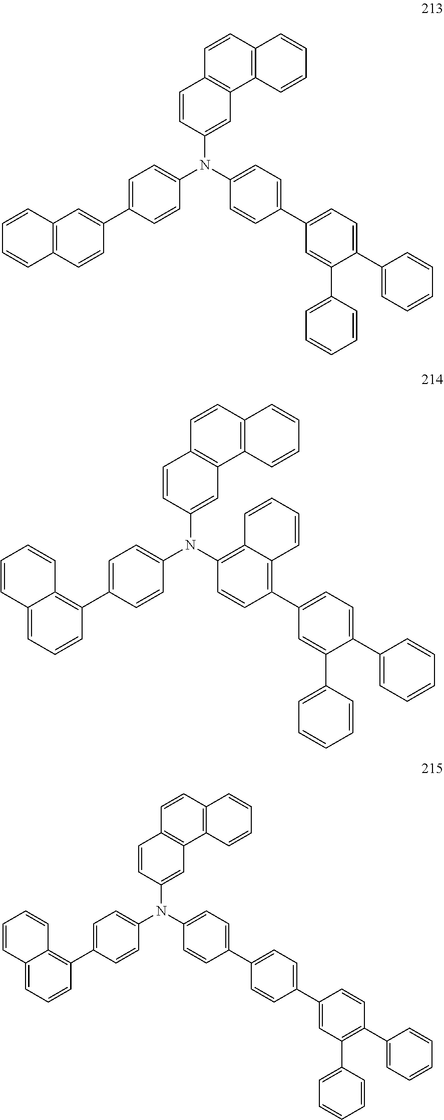 [SMILES: c1ccc(-c2ccc(-c3ccc(N(c4ccc(-c5ccc6ccccc6c5)cc4)c4ccc5ccc6ccccc6c5c4)cc3)cc2-c2ccccc2)cc1 ; c1ccc(-c2ccc(-c3ccc(N(c4ccc(-c5cccc6ccccc56)cc4)c4ccc5ccc6ccccc6c5c4)c4ccccc34)cc2-c2ccccc2)cc1 ; c1ccc(-c2ccc(-c3ccc(-c4ccc(N(c5ccc(-c6cccc7ccccc67)cc5)c5ccc6ccc7ccccc7c6c5)cc4)cc3)cc2-c2ccccc2)cc1] 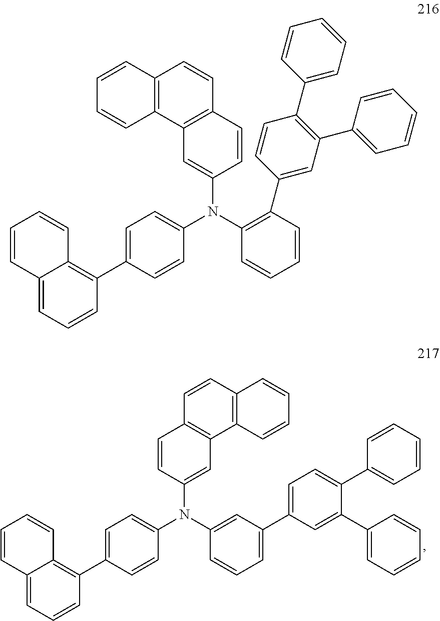 [SMILES: c1ccc(-c2ccc(-c3ccccc3N(c3ccc(-c4cccc5ccccc45)cc3)c3ccc4ccc5ccccc5c4c3)cc2-c2ccccc2)cc1 ; c1ccc(-c2ccc(-c3cccc(N(c4ccc(-c5cccc6ccccc56)cc4)c4ccc5ccc6ccccc6c5c4)c3)cc2-c2ccccc2)cc1]
in Compound Group 1, D is a deuterium atom.

18. A display device comprising:
a base layer;
a circuit layer on the base layer; and
a display element layer on the circuit layer, and comprising a light emitting element, wherein the light emitting element comprises a first electrode, a second electrode on the first electrode, a light emitting layer between the first electrode and the second electrode, and a hole transport region between the first electrode and the light emitting layer, and comprising an amine compound represented by Formula 1: 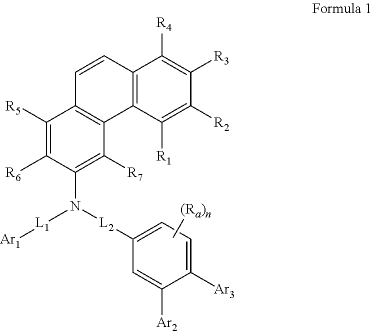
in Formula 1,
R₁ to R₇ are each independently a hydrogen atom, a deuterium atom, a halogen atom, a substituted or unsubstituted alkyl group having 1 to 15 carbons, a substituted or unsubstituted aryl group having 6 to 30 ring-forming carbon atoms, or a substituted or unsubstituted heteroaryl group having 2 to 30 ring-forming carbon atoms,
n is an integer of 1 to 3,
Rₐ is a hydrogen atom, a deuterium atom, a halogen atom, a substituted or unsubstituted alkyl group having 1 to 15 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or forms a ring by being bonded to an adjacent group,
L₁ and L₂ are each independently a direct linkage, a substituted or unsubstituted arylene group having 6 to 30 ring-forming carbon atoms, or a substituted or unsubstituted heteroarylene group having 2 to 30 ring-forming carbon atoms,
Ar₁ is a substituted or unsubstituted aryl group having 6 to 30 ring-forming carbon atoms, or a substituted or unsubstituted heteroaryl group having 2 to 30 ring-forming carbon atoms, and Ar₁ is not a fluorenyl group when L₁ is a direct linkage,
Ar₂ and Ar₃ are each independently a substituted or unsubstituted phenyl group, a substituted or unsubstituted biphenyl group, a substituted or unsubstituted terphenyl group, a substituted or unsubstituted naphthyl group, or a substituted or unsubstituted phenanthrenyl group, except when each of Ar₂ and Ar₃ includes a carbazole group as a substituent, and
each of Ar₁, Ar₂, and Ar₃ do not include benzophenanthrene.

19. The display device of claim 18, wherein the light emitting element is configured to emit blue light.

20. The display device of claim 18, the display device further comprising a light control layer comprising a quantum dot.
